(19) United States
(12) Patent Application Publication
(10) Pub. No.: US 2024/0182547 A1
(43) Pub. Date: Jun. 6, 2024

(54) ANTI-RSV ANTIBODY AND PHARMACEUTICAL COMPOSITION COMPRISING SAME

(71) Applicant: MOGAM INSTITUTE FOR BIOMEDICAL RESEARCH, Yongin-si Gyeonggi-do (KR)

(72) Inventors: Dong Sik KIM, Yongin-si Gyeonggi-do (KR); Shin A JANG, Yongin-si Gyeonggi-do (KR); Young Woo HAN, Yongin-si Gyeonggi-do (KR); Su A LEE, Yongin-si Gyeonggi-do (KR); Woo Hyun KIM, Yongin-si Gyeonggi-do (KR)

(73) Assignee: MOGAM INSTITUTE FOR BIOMEDICAL RESEARCH, Yongin-si Gyeonggi-do (KR)

(21) Appl. No.: 18/002,881

(22) PCT Filed: May 7, 2021
(86) PCT No.: PCT/KR2021/005742

§ 371 Date: Dec. 22, 2022

(30) Foreign Application Priority Data
Jun. 25, 2020 (KR) 10-2020-0077914

(51) Int. Cl. C07K 16/10; A61K 39/00

(52) U.S. Cl. CPC C07K 16/10; A61K 39/00; C07K 2317/24; C07K 2317/76

(57) ABSTRACT

The present invention relates to an anti-respiratory syncytial virus (RSV) antibody and a pharmaceutical composition comprising the same, and particularly, to an anti-RSV antibody specifically binding to an F-protein of RSV, and a pharmaceutical composition for use in preventing or treating an RSV infection. The anti-RSV antibody according to the present invention can effectively prevent RSV infection and has excellent efficacy for alleviating and treating symptoms of RSV infection.

### CROSS-REFERENCE TO RELATED APPLICATIONS

This application is a 35 U.S.C. 371 National Phase Entry Application from PCT/KR2021/005742 filed May 7, 2021, designating the United States, which claims priority to and the benefit of Korean Patent Application No. 10-2020-0077914, filed on Jun. 25, 2020, the disclosures of which are incorporated herein by reference in their entirety.

### INCORPORATION-BY-REFERENCE OF SEQUENCE LISTING

The instant application contains a Sequence Listing which has been submitted via EFS-Web and is hereby incorporated by reference in its entirety. Said Sequence Listing, created on Nov. 16, 2022, is named SOP115992US_Sequence Listing.TXT and is 315,392 bytes in size.

### TECHNICAL FIELD

The present invention relates to an anti-respiratory syncytial virus (RSV) antibody and a pharmaceutical composition comprising the same, and particularly, to an anti-RSV antibody specifically binding to an F-protein of RSV, and a pharmaceutical composition for use in preventing or treating an RSV infection.

### BACKGROUND

Respiratory Syncytial Virus (RSV) is a virus that belongs to the paramyxovirus family and causes acute respiratory infections. RSV is highly toxic and easily spreadable, and causes a runny nose, sore throat, cough and sputum as main symptoms, accompanied by nasal congestion, hoarseness, wheezing, and vomiting. RSV infections show mild symptoms (mainly upper respiratory tract infection), like a cold, in adults, but they can cause lower respiratory tract diseases such as bronchiolitis and pneumonia in infants of one year old or younger and often cause pneumonia in newborns.

Currently, no RSV prevention vaccines or specific antiviral agents have been developed, and except special cases, a method of alleviating infectious symptoms has been used as a treatment method. However, for a high-risk group including premature infants with a lung or heart disease, a product, named Synagis, for injecting mass-produced anti-RSV antibody into the body is commonly used, but has disadvantages of monthly administration during the RSV epidemic period since the half-life of an antibody is about one month, and its high cost.

RSV includes two major surface glycoproteins, the G-protein and F-protein. While the F-protein facilitated the fusion of the virus and the cell membrane, the G-protein mediates the binding of the virus to a cell receptor and allows the penetration of a ribonucleoprotein of the virus into the cell cytoplasm. Particularly, the F-protein has several sites involved in infection.

It was demonstrated that antibodies produced against the RSV F or G-protein neutralize RSV with high efficiency in vitro and have a preventive effect in vivo (refer to Walsh et al., (1986) J. Gen. Microbiol. 67:505; Beeler et al., (1989) J. Virol. 63:2941-2950, Garcia-Borenno et al., (1989) J. Virol. 63:925-932, Taylor et al., (1984) Immunology 52:137-142). Antibodies against the RSV F-protein are also effective in inhibiting the fusion of RSV-infected cells and neighboring uninfected cells.

However, to prevent RSV infections, it is essential to simultaneously block all sites and proteins related to the infections. When all sites are not completely blocked, cell infections may occur through unblocked sites.

Accordingly, the present inventors have endeavored to develop anti-RSV antibodies for effectively preventing and treating an RSV infection and thus developed a novel anti-RSV antibody having excellent efficacy.

### SUMMARY OF THE INVENTION

The present invention is directed to providing an antibody or an antigen-binding fragment thereof specifically binding to respiratory syncytial virus (RSV) selected from the group consisting of the following (i) to (vii):
(i) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 2, and heavy chain CDR3 comprising the amino acid sequence of SEQ ID NO: 3, and a light chain variable domain which comprises light chain CDR1 comprising the amino acid sequence of SEQ ID NO: 4, light chain CDR2 comprising the amino acid sequence of SEQ ID NO: 5, and light chain CDR3 comprising the amino acid sequence of SEQ ID NO: 6;
(ii) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 2, and heavy chain CDR3 comprising the amino acid sequence of SEQ ID NO: 3, and a light chain variable domain which comprises light chain CDR1 comprising the amino acid sequence of SEQ ID NO: 7, light chain CDR2 comprising the amino acid sequence of SEQ ID NO: 5, and light chain CDR3 comprising the amino acid sequence of SEQ ID NO: 6;
(iii) an antibody specifically binding to RSV, comprising a heavy chain variable domain which comprises heavy chain CDR1 comprising the amino acid sequence of SEQ ID NO: 8, heavy chain CDR2 comprising the amino acid sequence of SEQ ID NO: 9, and heavy chain CDR3 comprising the amino acid sequence of SEQ ID NO: 10, and a light chain variable domain which comprises light chain CDR1 comprising the amino acid sequence of SEQ ID NO: 7, light chain CDR2 comprising the amino acid sequence of SEQ ID NO: 5, and light chain CDR3 comprising the amino acid sequence of SEQ ID NO: 6;
(iv) an antibody specifically binding to RSV, comprising a heavy chain variable domain which comprises heavy chain CDR1 comprising the amino acid sequence of SEQ ID NO: 11, heavy chain CDR2 comprising the amino acid sequence of SEQ ID NO: 12, and heavy chain CDR3 comprising the amino acid sequence of SEQ ID NO: 3, and a light chain variable domain which comprises light chain CDR1 comprising the amino acid sequence of SEQ ID NO: 13, light chain CDR2 comprising the amino acid sequence of SEQ ID NO: 5, and light chain CDR3 comprising the amino acid sequence of SEQ ID NO: 6;
(v) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 2, and heavy chain CDR3 comprising the amino acid sequence of SEQ ID NO: 3, and a light chain variable domain which comprises light chain CDR1 comprising the amino acid sequence of SEQ ID NO: 14, light chain CDR2 comprising the amino acid sequence of SEQ ID NO: 15, and light chain CDR3 comprising the amino acid sequence of SEQ ID NO: 6;
(vi) an antibody specifically binding to RSV, comprising a heavy chain variable domain which comprises heavy chain CDR1 comprising the amino acid sequence of SEQ ID NO: 8, heavy chain CDR2 comprising the amino acid sequence of SEQ ID NO: 9, and heavy chain CDR3 comprising the amino acid sequence of SEQ ID NO: 3, and a light chain variable domain which comprises light chain CDR1 comprising the amino acid sequence of SEQ ID NO: 14, light chain CDR2 comprising the amino acid sequence of SEQ ID NO: 5, and light chain CDR3 comprising the amino acid sequence of SEQ ID NO: 6; and
(vii) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 16, and heavy chain CDR3 comprising the amino acid sequence of SEQ ID NO: 17, and a light chain variable domain which comprises light chain CDR1 comprising the amino acid sequence of SEQ ID NO: 14, light chain CDR2 comprising the amino acid sequence of SEQ ID NO: 18, and light chain CDR3 comprising the amino acid sequence of SEQ ID NO: 6.

The present invention is also directed to providing an antibody or an antigen-binding fragment thereof specifically binding to RSV, selected from the group consisting of the following (viii) to (xv):
(viii) an antibody specifically binding to RSV, comprising a heavy chain variable domain comprising the amino acid sequence of SEQ ID NO: 19 and a light chain variable domain comprising the amino acid sequence of SEQ ID NO: 20;
(ix) an antibody specifically binding to RSV, comprising a heavy chain variable domain comprising the amino acid sequence of SEQ ID NO: 21 and a light chain variable domain comprising the amino acid sequence of SEQ ID NO: 22;
(x) an antibody specifically binding to RSV, comprising a heavy chain variable domain comprising the amino acid sequence of SEQ ID NO: 23 and a light chain variable domain comprising the amino acid sequence of SEQ ID NO: 24;
(xi) an antibody specifically binding to RSV, comprising a heavy chain variable domain comprising the amino acid sequence of SEQ ID NO: 25 and a light chain variable domain comprising the amino acid sequence of SEQ ID NO: 26;
(xii) an antibody specifically binding to RSV, comprising a heavy chain variable domain comprising the amino acid sequence of SEQ ID NO: 27 and a light chain variable domain comprising the amino acid sequence of SEQ ID NO: 28;
(xiii) an antibody specifically binding to RSV, comprising a heavy chain variable domain comprising the amino acid sequence of SEQ ID NO: 29 and a light chain variable domain comprising the amino acid sequence of SEQ ID NO: 30;
(xiv) an antibody specifically binding to RSV, comprising a heavy chain variable domain comprising the amino acid sequence of SEQ ID NO: 31 and a light chain variable domain comprising the amino acid sequence of SEQ ID NO: 24; and
(xv) an antibody specifically binding to RSV, comprising a heavy chain variable domain comprising the amino acid sequence of SEQ ID NO: 32 and a light chain variable domain comprising the amino acid sequence of SEQ ID NO: 33.

The present invention is also directed to providing an antibody or an antigen-binding fragment thereof specifically binding to RSV, selected from the group consisting of the following (xvi) to (xxiii):
(xvi) an antibody specifically binding to RSV, comprising a heavy chain variable domain encoded by the nucleotide sequence of SEQ ID NO: 34 and a light chain variable domain encoded by the nucleotide sequence of SEQ ID NO: 35;
(xvii) an antibody specifically binding to RSV, comprising a heavy chain variable domain encoded by the nucleotide sequence of SEQ ID NO: 36 and a light chain variable domain encoded by the nucleotide sequence of SEQ ID NO: 37;
(xviii) an antibody specifically binding to RSV, comprising a heavy chain variable domain encoded by the nucleotide sequence of SEQ ID NO: 38 and a light chain variable domain encoded by the nucleotide sequence of SEQ ID NO: 39;
(xix) an antibody specifically binding to RSV, comprising a heavy chain variable domain encoded by the nucleotide sequence of SEQ ID NO: 40 and a light chain variable domain encoded by the nucleotide sequence of SEQ ID NO: 41;
(xx) an antibody specifically binding to RSV, comprising a heavy chain variable domain encoded by the nucleotide sequence of SEQ ID NO: 42 and a light chain variable domain encoded by the nucleotide sequence of SEQ ID NO: 43;
(xxi) an antibody specifically binding to RSV, comprising a heavy chain variable domain encoded by the nucleotide sequence of SEQ ID NO: 44 and a light chain variable domain encoded by the nucleotide sequence of SEQ ID NO: 45;
(xxii) an antibody specifically binding to RSV, comprising a heavy chain variable domain encoded by the nucleotide sequence of SEQ ID NO: 46 and a light chain variable domain encoded by the nucleotide sequence of SEQ ID NO: 39; and
(xxiii) an antibody specifically binding to RSV, comprising a heavy chain variable domain encoded by the nucleotide sequence of SEQ ID NO: 47 and a light chain variable domain encoded by the nucleotide sequence of SEQ ID NO: 48.

The present invention is also directed to providing a polynucleotide encoding a light chain variable domain and a light chain variable domain of the aforementioned antibody.

The present invention is also directed to providing an expression vector comprising the polynucleotide.

The present invention is also directed to providing a host cell transformed with the expression vector.

The present invention is also directed to providing a method of preparing an antibody or an antigen-binding fragment thereof specifically binding to respiratory syncytial virus (RSV), comprising the step of culturing the host cell.

The present invention is also directed to providing a pharmaceutical composition for use in preventing or treating respiratory syncytial virus (RSV) infection, comprising the aforementioned antibody or antigen-binding fragment thereof.

To achieve the above-described purposes, the present invention provides an antibody or an antigen-binding fragment thereof specifically binding to respiratory syncytial virus (RSV) selected from the group consisting of the following (i) to (vii):
(i) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 2, and heavy chain CDR3 comprising the amino acid sequence of SEQ ID NO: 3, and a light chain variable domain which comprises light chain CDR1 comprising the amino acid sequence of SEQ ID NO: 4, light chain CDR2 comprising the amino acid sequence of SEQ ID NO: 5, and light chain CDR3 comprising the amino acid sequence of SEQ ID NO: 6;
(ii) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 2, and heavy chain CDR3 comprising the amino acid sequence of SEQ ID NO: 3, and a light chain variable domain which comprises light chain CDR1 comprising the amino acid sequence of SEQ ID NO: 7, light chain CDR2 comprising the amino acid sequence of SEQ ID NO: 5, and light chain CDR3 comprising the amino acid sequence of SEQ ID NO: 6;
(iii) an antibody specifically binding to RSV, comprising a heavy chain variable domain which comprises heavy chain CDR1 comprising the amino acid sequence of SEQ ID NO: 8, heavy chain CDR2 comprising the amino acid sequence of SEQ ID NO: 9, and heavy chain CDR3 comprising the amino acid sequence of SEQ ID NO: 10, and a light chain variable domain which comprises light chain CDR1 comprising the amino acid sequence of SEQ ID NO: 7, light chain CDR2 comprising the amino acid sequence of SEQ ID NO: 5, and light chain CDR3 comprising the amino acid sequence of SEQ ID NO: 6;
(iv) an antibody specifically binding to RSV, comprising a heavy chain variable domain which comprises heavy chain CDR1 comprising the amino acid sequence of SEQ ID NO: 11, heavy chain CDR2 comprising the amino acid sequence of SEQ ID NO: 12, and heavy chain CDR3 comprising the amino acid sequence of SEQ ID NO: 3, and a light chain variable domain which comprises light chain CDR1 comprising the amino acid sequence of SEQ ID NO: 13, light chain CDR2 comprising the amino acid sequence of SEQ ID NO: 5, and light chain CDR3 comprising the amino acid sequence of SEQ ID NO: 6;
(v) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 2, and heavy chain CDR3 comprising the amino acid sequence of SEQ ID NO: 3, and a light chain variable domain which comprises light chain CDR1 comprising the amino acid sequence of SEQ ID NO: 14, light chain CDR2 comprising the amino acid sequence of SEQ ID NO: 15, and light chain CDR3 comprising the amino acid sequence of SEQ ID NO: 6;
(vi) an antibody specifically binding to RSV, comprising a heavy chain variable domain which comprises heavy chain CDR1 comprising the amino acid sequence of SEQ ID NO: 8, heavy chain CDR2 comprising the amino acid sequence of SEQ ID NO: 9, and heavy chain CDR3 comprising the amino acid sequence of SEQ ID NO: 3, and a light chain variable domain which comprises light chain CDR1 comprising the amino acid sequence of SEQ ID NO: 14, light chain CDR2 comprising the amino acid sequence of SEQ ID NO: 5, and light chain CDR3 comprising the amino acid sequence of SEQ ID NO: 6; and
(vii) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 16, and heavy chain CDR3 comprising the amino acid sequence of SEQ ID NO: 17, and a light chain variable domain which comprises light chain CDR1 comprising the amino acid sequence of SEQ ID NO: 14, light chain CDR2 comprising the amino acid sequence of SEQ ID NO: 18, and light chain CDR3 comprising the amino acid sequence of SEQ ID NO: 6.

The present invention also provides an antibody or an antigen-binding fragment thereof specifically binding to RSV, selected from the group consisting of the following (viii) to (xv):
(viii) an antibody specifically binding to RSV, comprising a heavy chain variable domain comprising the amino acid sequence of SEQ ID NO: 19 and a light chain variable domain comprising the amino acid sequence of SEQ ID NO: 20;
(ix) an antibody specifically binding to RSV, comprising a heavy chain variable domain comprising the amino acid sequence of SEQ ID NO: 21 and a light chain variable domain comprising the amino acid sequence of SEQ ID NO: 22;
(x) an antibody specifically binding to RSV, comprising a heavy chain variable domain comprising the amino acid sequence of SEQ ID NO: 23 and a light chain variable domain comprising the amino acid sequence of SEQ ID NO: 24;
(xi) an antibody specifically binding to RSV, comprising a heavy chain variable domain comprising the amino acid sequence of SEQ ID NO: 25 and a light chain variable domain comprising the amino acid sequence of SEQ ID NO: 26;
(xii) an antibody specifically binding to RSV, comprising a heavy chain variable domain comprising the amino acid sequence of SEQ ID NO: 27 and a light chain variable domain comprising the amino acid sequence of SEQ ID NO: 28;
(xiii) an antibody specifically binding to RSV, comprising a heavy chain variable domain comprising the amino acid sequence of SEQ ID NO: 29 and a light chain variable domain comprising the amino acid sequence of SEQ ID NO: 30;
(xiv) an antibody specifically binding to RSV, comprising a heavy chain variable domain comprising the amino acid sequence of SEQ ID NO: 31 and a light chain variable domain comprising the amino acid sequence of SEQ ID NO: 24; and
(xv) an antibody specifically binding to RSV, comprising a heavy chain variable domain comprising the amino acid sequence of SEQ ID NO: 32 and a light chain variable domain comprising the amino acid sequence of SEQ ID NO: 33.

The present invention also provides an antibody or an antigen-binding fragment thereof specifically binding to RSV, selected from the group consisting of the following (xvi) to (xxiii):
(xvi) an antibody specifically binding to RSV, comprising a heavy chain variable domain encoded by the nucleotide sequence of SEQ ID NO: 34 and a light chain variable domain encoded by the nucleotide sequence of SEQ ID NO: 35;
(xvii) an antibody specifically binding to RSV, comprising a heavy chain variable domain encoded by the nucleotide sequence of SEQ ID NO: 36 and a light chain variable domain encoded by the nucleotide sequence of SEQ ID NO: 37;
(xviii) an antibody specifically binding to RSV, comprising a heavy chain variable domain encoded by the nucleotide sequence of SEQ ID NO: 38 and a light chain variable domain encoded by the nucleotide sequence of SEQ ID NO: 39;
(xix) an antibody specifically binding to RSV, comprising a heavy chain variable domain encoded by the nucleotide sequence of SEQ ID NO: 40 and a light chain variable domain encoded by the nucleotide sequence of SEQ ID NO: 41;
(xx) an antibody specifically binding to RSV, comprising a heavy chain variable domain encoded by the nucleotide sequence of SEQ ID NO: 42 and a light chain variable domain encoded by the nucleotide sequence of SEQ ID NO: 43;
(xxi) an antibody specifically binding to RSV, comprising a heavy chain variable domain encoded by the nucleotide sequence of SEQ ID NO: 44 and a light chain variable domain encoded by the nucleotide sequence of SEQ ID NO: 45;
(xxii) an antibody specifically binding to RSV, comprising a heavy chain variable domain encoded by the nucleotide sequence of SEQ ID NO: 46 and a light chain variable domain encoded by the nucleotide sequence of SEQ ID NO: 39; and
(xxiii) an antibody specifically binding to RSV, comprising a heavy chain variable domain encoded by the nucleotide sequence of SEQ ID NO: 47 and a light chain variable domain encoded by the nucleotide sequence of SEQ ID NO: 48.

The present invention also provides a polynucleotide encoding a light chain variable domain and a light chain variable domain of the aforementioned antibody.

The present invention also provides an expression vector comprising the polynucleotide.

The present invention also provides a host cell transformed with the expression vector.

The present invention also provides a method of preparing an antibody or an antigen-binding fragment thereof specifically binding to respiratory syncytial virus (RSV), comprising the step of culturing the host cell.

The present invention also provides a pharmaceutical composition for use in preventing or treating respiratory syncytial virus (RSV) infection, comprising the aforementioned antibody or antigen-binding fragment thereof.

The anti-RSV antibody according to the present invention can effectively prevent RSV infection and has excellent efficacy for alleviating and treating symptoms of RSV infection.

### BRIEF DESCRIPTION OF THE DRAWINGS

FIG. 1 shows the SDS-PAGE results for DS-Cav1 (lane 1), sc9-10 (A-type; lane 2), DS-Cav1 (B-type), sc9-10 variants (sc9-10 A149C Y458C and sc9-10 N138GC N428C), and post-F-protein;

FIG. 2 is a result of analyzing the expression level of an RSV F-protein;

FIG. 3A shows a B cell isolation process, FIG. 3B shows a ratio of isolated cells with respect to the total cell number, and FIG. 3C shows a process of isolating cells, which are double positive for RSV F;

FIG. 4a, FIG. 4b and FIG. 4c are a result of constructing a chromium single sample V(D)J library;

FIG. 5 shows the results of VH/V_{κ}/V_{λ} PCR and scFv overlapping PCR;

FIGS. 6 to 10 show test results for the neutralizing ability of selected RSV antibodies;

FIG. 11 shows the RSV neutralizing antibody IC₅₀ result for an MG9112A-screening antibody; and

FIGS. 12 to 17 show the results of measuring the affinity for a recombinant F-protein with respect to a selected anti-F antibody.

### DETAILED DESCRIPTION

The term “antibody” used herein refers to an immunoglobulin and an immunoglobulin fragment, which includes any fragment including more than a part of a variable domain of an immunoglobulin molecule retaining the specific binding ability of a full-length immunoglobulin which is naturally occurring, or partially or entirely synthesized, e.g., recombinantly prepared. Therefore, an antibody includes any protein having a binding domain, which is homologous to or substantially homologous to an immunoglobulin antigen-binding domain (antibody binding site). An antibody includes antibody fragments, e.g., anti-RSV antibody fragments. Therefore, the antibodies provided in the present invention include synthetic antibodies, recombinantly prepared antibodies, multispecific antibodies (e.g., bispecific antibodies), human antibodies, non-human antibodies, humanized antibodies, chimeric antibodies, intrabodies, and antibody fragments, but the present invention is not limited thereto. For example, the antibodies provided in the present invention include a Fab fragment, a Fab′ fragment, a F(ab′)2 fragment, a Fv fragment, a disulfide-stabilized Fv (dsFv), a Fd fragment, a Fd′ fragment, a single-chain Fv (scFv), a single-chain Fab (scFab), a diabody, an anti-idiotype (anti-Id) antibody, or any antigen-binding fragment thereof. The antibodies provided in the present invention include components of any immunoglobulin types (e.g., IgG, IgM, IgD, IgE, IgA, and IgY), any categories (e.g., IgG1, IgG2, IgG3, IgG4, IgA1, and IgA2), or subclasses (e.g., IgG2a, and IgG2b).

The term “antibody fragment” or “antigen-binding fragment” of an antibody, used herein, includes at least a part (e.g., one or more CDRs and/or one or more antibody-binding sites) of a variable domain of an antibody binding to an antigen, smaller than a full-length antibody, and accordingly, it refers to any part of a full-length antibody, retaining at least some of the binding specificity and the specific binding ability of the full-length antibody. Therefore, the antigen-binding fragment refers to an antibody fragment containing an antigen-binding region that binds to the same antigen as an antibody form which the antibody fragment is derived. The antibody fragment includes not only an antibody derivative prepared by enzymatic treatment of a full-length antibody, but also a derivative prepared by synthesis, for example, recombination. The antibody fragment is included in an antibody. Examples of the antibody fragments include, but not limited to, Fab, Fab′, F(ab′)2, single-chain Fv (scFv), Fv, dsFv, a diabody, and Fd and Fd′ fragments, and other fragments, such as modified fragments. The fragment may include, for example, multiple chains linked together by a disulfide linkage and/or peptide linker. Antibody fragments generally include approximately 50 or more amino acids, and typically approximately 200 or more amino acids.

The antigen-binding fragment includes any antibody fragment that generates an antibody (that is, with a Ka of approximately 10⁷-10⁸ M⁻¹ or more) immunospecifically binding to an antigen during insertion into an antibody framework (like that prepared by substitution of a corresponding region).

The term “neutralizing antibody” used herein is any antibody that binds to a pathogen to hinder the ability of the pathogen to infect cells/cause a disease in a subject, or an antigen-binding fragment thereof. Examples of the neutralizing antibodies are neutralizing antibodies binding to a viral, bacterial, and fungal pathogens. Typically, the neutralizing antibody provided in the present invention binds to the surface of a pathogen. When a pathogen is a virus, a neutralizing antibody binding to the virus binds to a surface protein of the virus. According to the classification of viruses, the surface protein may be a capsid protein (e.g., a capsid protein of a non-enveloped virus) or a viral envelope protein (e.g., viral envelope protein of an enveloped virus). In some examples, the protein may be a glycoprotein. The ability to inhibit viral infection may be measured by in vitro neutralization analysis, for example, plaque reduction analysis using Vero host cells.

The term “surface protein” of a pathogen, used herein, is any protein located on the outer surface of the pathogen. The surface protein may be partially or entirely exposed to an external environment (that is, an outer surface). An example of the surface protein is a membrane protein, such as a protein located on a viral envelope surface or a bacterial outer membrane (e.g., a membrane glycoprotein). The membrane protein may be a transmembrane protein (i.e., proteins that passes through a lipid bilayer) or a non-transmembrane cell surface-associated protein (e.g., anchored or covalently attached to a membrane surface, like attachment of other proteins to the surface of a pathogen). An example of other surface proteins includes a viral capsid protein of a non-enveloped virus at least partially exposed to an external environment.

The term “epitope” used herein refers to any antigenic determinant on an antigen to which a paratope of the antibody binds. The epitope determinant generally includes a chemically active surface grouping of a molecule, for example, an amino acid or sugar side chain, and generally has a specific charge property, as well as a specific 3D structural property.

The term “phage display” used herein refers to the expression of a polypeptide on the surface of a filamentous bacteriophage.

The term “panning” used herein refers to an affinity-based selection for separating a molecule having specificity for a binding partner, for example, a captured molecule (e.g., an antigen), or a phage indicating the region, part or location of an amino acid or nucleotide.

The “specifically binding” or “immunospecifically binding” related to an antibody used herein or an antigen-binding fragment thereof is interchangeably used, and refers to the ability of an antibody or antigen-binding fragment to form one or more non-covalent bonds with a cognate antigen by non-covalent interaction between the antibody-binding site(s) of an antibody and an antigen.

The term “polypeptide” used herein refers to two or more amino acids which are covalently bonded. The term “polypeptide” and “protein” are interchangeably used in the present invention. The term “peptide” used herein refers to a polypeptide having a length of 2 to approximately 40 amino acids. In addition, the term “amino acid” used herein is an organic compound with an amino group and a carboxyl group. The polypeptide includes two or more amino acids.

The term “polynucleotide” and “nucleic acid molecule” used herein refers to an oligomer or polymer including two or more linked nucleotides or nucleotide derivatives, which are generally bound with each other by a phosphodiester bond, such as deoxyribonucleic acid (DNA) and ribonucleic acid (RNA).

The term “expression” used herein refers to a process of generating a polypeptide by the transcription and translation of a polynucleotide. The expression level of a polypeptide may be assessed by any method known in the art, for example, a method of determining a polypeptide amount from host cells. The method may include, but is not limited to, the quantification of a polypeptide in a cell lysate by ELISA, staining with Coomassie blue after electrophoresis, Lowry protein assay, and Bradford protein assay.

The term “host cell” used herein is a cell used to accommodate, maintain, reproduce, and amplify a vector. Host cells may also be used to express a polypeptide encoded by a vector. A nucleic acid included in the vector is replicated when the host cell divides and the nucleic acid is amplified. In one example, a host cell is a gene package, which expresses various polypeptides on its surface. In another example, a host cell is infected with a gene package. For example, a host cell may be a phage-displayed compatible host cell, which is transformed with a phage or phagemid vector to accommodate a phage package expressing fusion proteins including variant polypeptides.

The term “vector” used herein is a replicable nucleic acid capable of expressing one or more exogenous proteins upon transformation into a suitable host cell. The description on a vector includes vectors into which a nucleic acid encoding a polypeptide or a fragment thereof can be generally introduced by restriction enzyme digestion and ligation. The description on a vector also includes vectors having a nucleic acid encoding a polypeptide. A vector is used to introduce a nucleic acid encoding a polypeptide into a host cell for the amplification of a nucleic acid, or the expression/display of a polypeptide encoded by a nucleic acid. A vector generally remains in an episomal state, and may be designed to accomplish the integration of a gene or a part thereof into the chromosome of a genome. In addition, the present invention considers vectors, which are artificial chromosomes, for example, a yeast artificial chromosome and a mammalian artificial chromosome. The selection and use of such a vehicle is widely known in the art.

The vector includes a “virus vector” or “viral vector.” The viral vector is an engineered virus operably linked to a foreign gene to transport (as a vehicle or shuttle) the foreign gene into cells.

The “expression vector” used herein includes a vector that can express DNA operably linked to a regulatory sequence capable of performing the expression of a DNA fragment, for example, a promoter region. The additional segments may include promoter and terminator sequences and arbitrarily include one or more replication origins, one or more selection markers, an enhancer, and a polyadenylation sequence. An expression vector may be generally derived from plasmid or viral DNA, or include both. Therefore, an expression vector means a recombinant DNA or RNA construct, for example, such as a plasmid, phage, recombinant virus, or other vectors causing the expression of cloned DNA upon introduction into a suitable host cell. An appropriate expression vector is well known to those of ordinary skill in the art, and includes those replicable in eukaryotic cells and/or prokaryotic cells and those remaining episomal, or those integrated into the host cell genome.

The term “modification” used herein refers to modification of an amino acid sequence of a polypeptide or a nucleotide sequence in a nucleic acid molecule, and includes deletion, insertion and substitution of each of amino acids or nucleotides. A method of modifying a polypeptide is that common used by those of ordinary skill in the art, may be, for example, recombinant DNA technology.

The term “infection” and “RSV infection” used herein refer to a pathological state derived from not only all stages of the RSV life cycle in a host (invasion and replication by RSV in cells or living tissues, but the present invention is not limited thereto), but also infiltration and replication by RSV. The infiltration and proliferation by RSV include, but not limited to, the following steps: docking of RSV particles to cells, fusion of the cell membrane and the virus, introduction of viral genetic information into cells, expression of RSV proteins, generation of new RSV particles, and release of RSV particles from cells. RSV infection may be upper respiratory tract RSV infection (URI), lower respiratory tract RSV infection (LRI), or a combination thereof. In some examples, a pathological condition resulting from the infiltration and replication by RSV is an acute RSV disease.

The “acute RSV disease” used herein refers to a clinically serious disease occurring in the lungs or lower respiratory tract as a result of RSV infection, and it may be manifested as pneumonia and/or bronchitis. Here, the signs of this disease may include, for example, hypoxia, suffocation, dyspnea, difficulty in breathing, gasping respiration, stridor, and cyanosis. An acute RSV disease requires an affected subject to obtain medical intervention, for example, hospitalization, oxygenation, endotracheal insertion and/or ventilation.

The “treatment” of a subject with a disease or condition, used herein, means that a subject's symptoms are partially or entirely alleviated, or remain in a static state after treatment. Accordingly, the treatment includes prevention, therapy and/or curing. The prevention means the prevention of a potential disease, and/or the prevention of the aggravation of symptoms or disease progression. The treatment also includes any pharmaceutical use of any antibody provided herein or an antigen-binding fragment thereof, or a composition provided herein.

The term “prevention” used herein refers to a method of reducing the risk of developing a disease or state.

The term “pharmaceutically effective drug (preparation)” used herein includes a conventional therapeutic drug including any medicine or bioactive agent, for example, an anesthetic, a vasoconstrictor, a dispersant, a small molecule drug and a therapeutic protein, but the present invention is not limited thereto.

The term “subject” used herein refers to mammals, for example, mammals including a human. The animals of the present invention include any animals, for example, primates including a human, a gorilla and a monkey; rodents including a mouse and a rat; poultry such as chicken; ruminants such as goats, cattle, deer and sheep; sheep and other animals such as a pig. Non-human animals exclude humans as a considered animal.

One aspect of the present invention provides an antibody or an antigen-binding fragment thereof specifically binding to respiratory syncytial virus (RSV) selected from the group consisting of the following (i) to (vii):
(i) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 2, and heavy chain CDR3 comprising the amino acid sequence of SEQ ID NO: 3, and a light chain variable domain which comprises light chain CDR1 comprising the amino acid sequence of SEQ ID NO: 4, light chain CDR2 comprising the amino acid sequence of SEQ ID NO: 5, and light chain CDR3 comprising the amino acid sequence of SEQ ID NO: 6;
(ii) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 2, and heavy chain CDR3 comprising the amino acid sequence of SEQ ID NO: 3, and a light chain variable domain which comprises light chain CDR1 comprising the amino acid sequence of SEQ ID NO: 7, light chain CDR2 comprising the amino acid sequence of SEQ ID NO: 5, and light chain CDR3 comprising the amino acid sequence of SEQ ID NO: 6;
(iii) an antibody specifically binding to RSV, comprising a heavy chain variable domain which comprises heavy chain CDR1 comprising the amino acid sequence of SEQ ID NO: 8, heavy chain CDR2 comprising the amino acid sequence of SEQ ID NO: 9, and heavy chain CDR3 comprising the amino acid sequence of SEQ ID NO: 10, and a light chain variable domain which comprises light chain CDR1 comprising the amino acid sequence of SEQ ID NO: 7, light chain CDR2 comprising the amino acid sequence of SEQ ID NO: 5, and light chain CDR3 comprising the amino acid sequence of SEQ ID NO: 6;
(iv) an antibody specifically binding to RSV, comprising a heavy chain variable domain which comprises heavy chain CDR1 comprising the amino acid sequence of SEQ ID NO: 11, heavy chain CDR2 comprising the amino acid sequence of SEQ ID NO: 12, and heavy chain CDR3 comprising the amino acid sequence of SEQ ID NO: 3, and a light chain variable domain which comprises light chain CDR1 comprising the amino acid sequence of SEQ ID NO: 13, light chain CDR2 comprising the amino acid sequence of SEQ ID NO: 5, and light chain CDR3 comprising the amino acid sequence of SEQ ID NO: 6;
(v) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 2, and heavy chain CDR3 comprising the amino acid sequence of SEQ ID NO: 3, and a light chain variable domain which comprises light chain CDR1 comprising the amino acid sequence of SEQ ID NO: 14, light chain CDR2 comprising the amino acid sequence of SEQ ID NO: 15, and light chain CDR3 comprising the amino acid sequence of SEQ ID NO: 6;
(vi) an antibody specifically binding to RSV, comprising a heavy chain variable domain which comprises heavy chain CDR1 comprising the amino acid sequence of SEQ ID NO: 8, heavy chain CDR2 comprising the amino acid sequence of SEQ ID NO: 9, and heavy chain CDR3 comprising the amino acid sequence of SEQ ID NO: 3, and a light chain variable domain which comprises light chain CDR1 comprising the amino acid sequence of SEQ ID NO: 14, light chain CDR2 comprising the amino acid sequence of SEQ ID NO: 5, and light chain CDR3 comprising the amino acid sequence of SEQ ID NO: 6; and
(vii) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 16, and heavy chain CDR3 comprising the amino acid sequence of SEQ ID NO: 17, and a light chain variable domain which comprises light chain CDR1 comprising the amino acid sequence of SEQ ID NO: 14, light chain CDR2 comprising the amino acid sequence of SEQ ID NO: 18, and light chain CDR3 comprising the amino acid sequence of SEQ ID NO: 6.

Another aspect of the present invention also provides an antibody or an antigen-binding fragment thereof specifically binding to RSV, selected from the group consisting of the following (viii) to (xv):
(viii) an antibody specifically binding to RSV, comprising a heavy chain variable domain comprising the amino acid sequence of SEQ ID NO: 19 and a light chain variable domain comprising the amino acid sequence of SEQ ID NO: 20;
(ix) an antibody specifically binding to RSV, comprising a heavy chain variable domain comprising the amino acid sequence of SEQ ID NO: 21 and a light chain variable domain comprising the amino acid sequence of SEQ ID NO: 22;
(x) an antibody specifically binding to RSV, comprising a heavy chain variable domain comprising the amino acid sequence of SEQ ID NO: 23 and a light chain variable domain comprising the amino acid sequence of SEQ ID NO: 24;
(xi) an antibody specifically binding to RSV, comprising a heavy chain variable domain comprising the amino acid sequence of SEQ ID NO: 25 and a light chain variable domain comprising the amino acid sequence of SEQ ID NO: 26;
(xii) an antibody specifically binding to RSV, comprising a heavy chain variable domain comprising the amino acid sequence of SEQ ID NO: 27 and a light chain variable domain comprising the amino acid sequence of SEQ ID NO: 28;
(xiii) an antibody specifically binding to RSV, comprising a heavy chain variable domain comprising the amino acid sequence of SEQ ID NO: 29 and a light chain variable domain comprising the amino acid sequence of SEQ ID NO: 30;
(xiv) an antibody specifically binding to RSV, comprising a heavy chain variable domain comprising the amino acid sequence of SEQ ID NO: 31 and a light chain variable domain comprising the amino acid sequence of SEQ ID NO: 24; and
(xv) an antibody specifically binding to RSV, comprising a heavy chain variable domain comprising the amino acid sequence of SEQ ID NO: 32 and a light chain variable domain comprising the amino acid sequence of SEQ ID NO: 33.

Still another aspect of the present invention provides an antibody or an antigen-binding fragment thereof specifically binding to RSV, selected from the group consisting of the following (xvi) to (xxiii):
(xvi) an antibody specifically binding to RSV, comprising a heavy chain variable domain encoded by the nucleotide sequence of SEQ ID NO: 34 and a light chain variable domain encoded by the nucleotide sequence of SEQ ID NO: 35;
(xvii) an antibody specifically binding to RSV, comprising a heavy chain variable domain encoded by the nucleotide sequence of SEQ ID NO: 36 and a light chain variable domain encoded by the nucleotide sequence of SEQ ID NO: 37;
(xviii) an antibody specifically binding to RSV, comprising a heavy chain variable domain encoded by the nucleotide sequence of SEQ ID NO: 38 and a light chain variable domain encoded by the nucleotide sequence of SEQ ID NO: 39;
(xix) an antibody specifically binding to RSV, comprising a heavy chain variable domain encoded by the nucleotide sequence of SEQ ID NO: 40 and a light chain variable domain encoded by the nucleotide sequence of SEQ ID NO: 41;
(xx) an antibody specifically binding to RSV, comprising a heavy chain variable domain encoded by the nucleotide sequence of SEQ ID NO: 42 and a light chain variable domain encoded by the nucleotide sequence of SEQ ID NO: 43;
(xxi) an antibody specifically binding to RSV, comprising a heavy chain variable domain encoded by the nucleotide sequence of SEQ ID NO: 44 and a light chain variable domain encoded by the nucleotide sequence of SEQ ID NO: 45;
(xxii) an antibody specifically binding to RSV, comprising a heavy chain variable domain encoded by the nucleotide sequence of SEQ ID NO: 46 and a light chain variable domain encoded by the nucleotide sequence of SEQ ID NO: 39; and
(xxiii) an antibody specifically binding to RSV, comprising a heavy chain variable domain encoded by the nucleotide sequence of SEQ ID NO: 47 and a light chain variable domain encoded by the nucleotide sequence of SEQ ID NO: 48.

The antibody according to the present invention may include an amino acid sequence having a 80% or more, preferably, 90% or more, more preferably 95% or more, and most preferably 99% or more homology with the amino acid sequence(s) of a heavy chain, a light chain, a heavy chain variable domain and/or a light chain variable domain of any one antibody selected from the group consisting of i) to xv).

The antibody or antigen-binding fragment of the present invention may specifically bind to RSV. Here, RSV to which the antibody or antigen-binding fragment binds may be a surface protein of RSV, such as the F-protein.

In the light chain and heavy chain variable domains, as long as the characteristics consistent with the purpose of the present invention, for example, the affinity to and specificity for RSV, are maintained, some amino acids can be substituted, inserted and/or deleted. For example, in the light chain and/or heavy chain variable domain(s), the conservative substitution of an amino acid may occur. The conservative substitution refers to the substitution with another amino acid residue having similar characteristics to the original amino acid sequence.

For example, lysine, arginine, and histidine have similar characteristics due to having basic side chains, and aspartic acid and glutamic acid have similar characteristics due to having acidic side chains. In addition, glycine, asparagine, glutamine, serine, threonine, tyrosine, cysteine, and tryptophan have similar properties due to having uncharged polar side chains, and alanine, valine, leucine, threonine, isoleucine, proline, phenylalanine, and methionine have similar characteristics due to having non-polar side chains, whereas tyrosine, phenylalanine, tryptophan, and histidine have similar characteristics due to having aromatic side chains. Therefore, it is obvious to those of ordinary skill in the art that, even if amino acid substitution occurs in groups having similar characteristics to those described above, there will be no significant change in characteristics. For this reason, as long as the characteristics of the antibody are maintained, the antibody in which a modification by conservative substitution occurs in a variable domain is also included in the scope of the present invention.

The antibody or antigen-binding fragment of the present invention may be a humanized antibody. The term “humanized antibody” used herein refers to a chimera antibody containing the minimal sequence derived from an immunoglobulin of a non-human antibody such as a mouse, and may mean an antibody in which all parts except the sequence corresponding to the hypervariable domain are substituted with the sequence of a human antibody. Here, the term “hypervariable domain (HVR)” refers to a variable domain showing hypervariability in an antibody sequence, or forming a structurally defined loop. Among the explanatory methods describing it, the Kabat's complementarity-determining region (CDR) is most commonly used as a method of classifying domains based on sequence variability.

An antibody fragment may be used as the antibody as long as the antibody's function is maintained. The antibody or antibody fragment may be a single-chain antibody, a diabody, a triabody, a tetrabody, a Fab fragment, a Fab′ fragment, a F(ab′)2 fragment, Fd, scFv, a domain antibody, a minibody, a scab, an IgD antibody, an IgE antibody, an IgM antibody, an IgG1 antibody, an IgG2 antibody, an IgG3 antibody, an IgG4 antibody, a derivative of an antibody constant domain, or an artificial antibody based on a protein scaffold, maintaining the binding function to RSV, but the present invention is not limited thereto.

The antibody or antigen-binding site of the present invention may be a neutralizing antibody. The neutralizing antibody refers to any antibody that binds to a pathogen, thereby hindering the ability of a pathogen to infect cells or cause a disease in a subject, or an antigen-binding fragment thereof.

Another aspect of the present invention provides a polynucleotide encoding a light chain variable domain and a light chain variable domain of the antibody or antigen-binding fragment, and an expression vector comprising the same.

Specifically, a polynucleotide encoding the amino acid sequence of the anti-RSV antibody may be used separately or in the form of being inserted into one vector and a polynucleotide encoding a heavy chain or its variable domain may be used separately or in the form of being inserted into one vector.

An expression vector suitable for the production of the anti-RSV antibody may include a signal sequence for membrane targeting or secretion, in addition to expression regulatory elements such as a promoter, an initiation codon, a termination codon, a polyadenylation signal, and an enhancer. The initiation codon and termination codon are generally considered as parts of a polynucleotide sequence encoding an immunogenic target protein, and necessarily act in an individual when a gene construct is administered and must be in-frame with the coding sequence. A general promoter may be constitutive or inducible. As a promoter, a lac, tac, T3, or T7 promoter for prokaryotic cells, simian virus 40 (SV40), or a mouse mammary tumor virus (MMTV) promoter may be used, but the present invention is not limited thereto.

The expression vector may include a selective marker for selecting host cells containing the same. The selective marker is for selecting cells transformed with a vector, and may be a marker that imparts a selectable phenotype such as drug resistance, auxotrophy, resistance to a cytotoxic agent, or the expression of a surface protein. In an environment treated with a selective agent, since only cells expressing a selection marker survive, transformed cells may be selected. In addition, when the vector is a replicable expression vector, it may include a replication origin, which is a specific nucleic acid sequence from which replication is initiated.

As a recombinant expression vector for inserting a foreign gene, various types of vectors such as a plasmid, a virus, and a cosmid may be used. While the type of recombinant vector is not particularly limited as long as it serves to express a desired gene and produce a desired protein in various types of host cells of prokaryotic and/or eukaryotic cells, it is preferable to use a vector capable of possessing a promoter exhibiting potent activity and strong expression ability and massively producing a foreign protein in a form similar to the natural state.

Still another aspect of the present invention provides a host cell transformed with the expression vector. The expression vector may be inserted into a host cell to form a transformant.

The term “transformation into a host cell” used herein may include any method for introducing a nucleic acid into an organism, a cell, tissue or an organ, and may be performed by selecting suitable standard technology according to host cells as known in the art. Specifically, electroporation, protoplast fusion, calcium phosphate (CaPO₄) precipitation, calcium chloride (CaCl₂)) precipitation, agitation with a silicon carbide fiber, Agrobacterium-mediated transformation, PEG, dextran sulfate, Lipofectamine, and drying/suppression-mediated transformation may be used, but the present invention is not limited thereto.

Yet another aspect of the present invention provides a method of preparing an antibody or an antigen-binding fragment thereof specifically binding to respiratory syncytial virus (RSV), comprising the step of culturing the host cell. Specifically, the method of preparing an antibody may include preparing a recombinant vector by inserting a nucleotide sequence encoding the anti-RSV antibody; transforming host cells with the recombinant vector and culturing the resulting product; and isolating and purifying a humanized antibody from the cultured transformant.

Yet another aspect of the present invention provides a pharmaceutical composition for use in preventing or treating RSV infection, comprising the aforementioned antibody or antigen-binding fragment thereof.

The pharmaceutical composition may further include a pharmaceutically acceptable carrier. For oral administration, a binder, a lubricant, a disintegrant, an excipient, a solubilizer, a dispersant, a stabilizer, a suspending agent, a coloring agent, or a fragrance may be used, for injectables, a buffer, a preservative, a pain reliver, a solubilizer, an isotonic agent, and a stabilizer may be mixed and used, and for topical administration, a base material, an excipient, a lubricant, or a preservative may be used.

The pharmaceutical composition of the present invention may be prepared in various forms by being mixed with the above-described pharmaceutically acceptable carrier. For example, for oral administration, the pharmaceutical composition of the present invention may be prepared in various dosage forms such as a tablet, a troche, a capsule, an elixir, a suspension, a syrup and a wafer, and for injectables, the pharmaceutical composition or vaccine composition of the present invention may be prepared in a unit dose ampoule or multiple dose forms.

In addition, the pharmaceutical composition may include a surfactant that can improve membrane permeability. Such a surfactant may be derived from a steroid, a cationic lipid such as N-[1-(2,3-dioleyloxy)propyl-N,N,N-trimethylammonium chloride (DOTMA), or various types of compounds such as cholesterol hemisuccinate or phosphatidyl glycerol, but the present invention is not limited thereto.

The pharmaceutical composition may be administered together or sequentially with the above-described pharmaceutical or physiological component. Alternatively, the pharmaceutical composition may be administered in combination with an additional conventional therapeutic agent, or sequentially or simultaneously administered with a conventional therapeutic agent. Such administration may be performed once or multiple times. Taking all the factors into consideration, it is important to administer the pharmaceutical composition in an amount capable of obtaining the maximum effect in the minimum amount without side effects, and the amount may be easily determined by those of ordinary skill in the art.

The term “individual” used herein refers to a mammal suffering from a condition or disease that can be alleviated, suppressed or treated by administering the pharmaceutical composition, or having a risk thereof, and preferably, a human.

The term “administration” used herein refers to the introduction of a predetermined material into an individual by an appropriate method, and the pharmaceutical composition may be administered through any route that can reach target tissue. Such an administration method may be oral administration, intraperitoneal administration, intravenous administration, intramuscular administration, subcutaneous administration, intradermal administration, oral administration, topical administration, intranasal administration, intrapulmonary administration, or intrarectal administration, but the present invention is not limited thereto. However, when orally administered, proteins are digested, so it may be preferable that an oral composition must be formulated to coat an active ingredient or protect it from being degraded in the stomach. In addition, the pharmaceutical composition may be administered by any device to allow the active ingredient to move to target cells.

Hereinafter, the present invention will be described in further detail with reference to examples. The examples are merely provided to more specifically explain the present invention, and it will be obvious to those of ordinary skill in the art that the scope of the present invention is not limited to the examples according to the gist of the present invention.

### Experimental Materials: Reagents and Instruments

### 1. Preparation of F-Protein

TABLE 1

| Product | Supplier | Cat. # |
|---|---|---|
| PBS 1×, w/o Ca⁺⁺, Mg⁺⁺ 1 L | Lonza | 17-516Q |
| Molecular Grade Water | Corning | 46-000-CM |
| XL1-Blue | Stratagene | 200228 |
| LB Broth | Amresco | J833-1L |
| Carbenicillin | Sigma | C3416 |
| Kanamycin Sulfate | Amresco | A20219 |
| NotI | New England | R0189S |
| | Biolabs | |
| HindIII | New England | R0104S |
| | Biolabs | |
| In-fusion HD Cloning Kit | Clontech | 639650 |
| T4 Ligase | New England | M0202L |
| | Biolabs | |
| LB Agar LOP ™ Plate + Carbenicilin75 | Narae Biotech | LN004CA |
| LB Agar LOP ™ Plate + Kanamycin 50 | Narae Biotech | LN004K |
| Expi293F ™ Cells | Gibco ® | A14524 |
| ExpiFectamine ™ 293 Transfection Kits | Gibco ® | 14524 |
| Opti-MEMIReduced Serum Medium | Life | 31985-070 |
| | Technologies | |
| Bacto Tryptone | DIFCO | 211705 |
| Bacto Yeast Extract | DIFCO | 212750 |
| MOPS | Sigma | M3183 |
| NuPAGE MOPS SDS Running Buffer | NOVEX | NP0001 |
| (20X) | | |
| Tris Base | Merck | CAS 77-86-1 |
| | Millipore | |
| Imidazole | Sigma | 12399 |
| Sodium Chloride | Sigma | S7653 |
| Ni-NTA Agarose | QIAGEN | 30210 |
| MabSelect ™Xtra | GE Healthcare | 17-5269-02 |
| Protein A IgG binding buffer | Pierce | 21007 |
| IgG Elution buffer | Pierce | 21009 |
| PageBlue Protein Staining Solution | Pierce | 24620 |
| Tween-20 | Sigma Aldrich | P1379 |
| TMB Microwell Peroxidase Substrate | KPL | 52-00-03 |
| TMB Stop Solution | KPL | 50-85-06 |
| Reagent/Kinetics buffer(10X) | FORTEBIO | 18-1092 |
| EDC | GE Healthcare | BR-1000-50 |
| NHS | GE Healthcare | BR-1000-50 |
| 10 mM Acetate buffer | GE Healthcare | BR-1003-51 |
| 1.0M Ethanolamine-HCl pH 8.5 | GE Healthcare | BR-1000-50 |
| HBS-EP buffer | GE Healthcare | BR-1001-88 |

TABLE 2

| Product | Supplier | Cat. No. |
|---|---|---|
| Qiaprep Spin Miniprep Kit | Qiagen | 27106 |
| Qiaprep Plasmid Midi Kit | Corning | 12945 |
| Gel Extraction Kit | Qiagen | 28706 |
| PCR Purification Kit | Qiagen | 28106 |
| 1L Erlenmeyer Culture Flask | Corning | 431147 |
| 225 mL Graduated Conical Tube | Corning | 352075 |
| Disposable 10 mL Polypropylene Columns | Pierce | 29924 |
| NuPAGE ® Novex 4-12% Bis-Tris Gel | Invitrogen | NP0321BOX |
| 1000 mL Vacuum Filter/Bottle, 0.22 | Corning | 431098 |
| μm (PES) | | |
| Zeba ™ Spin Desalting Columns | Thermo | 89891 |
| | Scientific | |
| Vivaspin20 membrane 50.000 MWCO | Sartorus | VS2032 |
| 37° C. Incubator | N-Biotek | SJP-250MI |
| 37° C. Shaking Incubator | Vision | VS-8489SR |
| | Scientific | |
| | Co., Ltd. | |
| Clean Bench | Nok Woo | N/A |
| | Industry | |
| CO₂ Incubator | N-Biotek | NB-206CXXL |
| Electrophoration Cuvette 0.2 cm | BTX | 620 |
| 50 mL Cornical tube | SPL | 50050 |
| Micro centrifugation Tube | SPL | 60115 |
| 15 mL Cornical tube | SPL | 50015 |
| pH meter (SevenEasy) | Mettler | N/A |
| | Toledo | |
| Biosensors/Anti-Human Fc Capture | FORTEBIO | 18-5064 |
| (AHC) | | |
| Greiner 96well plates | GREINER | 655209 |
| | BIO-ONE | |

### 2. Construction and Screening of Human B Cell-Derived Library

TABLE 3

| Product | Supplier | Cat. # |
|---|---|---|
| Anti-Human CD3 FITC (OKT3) | eBioscience | 11-0037 |
| ANTI-HUMAN CD8 | TONBO | 35-0087-T100 |
| ANTIBODY FITC | BIOS | |
| Anti-human CD14 FITC | TONBO | 35-0149-T100 |
| | BIOS | |
| Streptavidin, R-phycoerythrin | Invitrogen | S866 |
| conjugate (SAPE) | | |
| Anti-Human CD19, PerCP-cyanine5.5 | eBioscience | 45-0199 |
| Anti-human CD20 PerCP-Cy5.5 | BD | 332781 |
| Mouse Anti-human CD27, PE-Cy7 | BD | 560609 |
| Streptavidin-APC | eBioscience | 17-4317-82 |
| RPMI-1640 Medium (ATCC | GIBCO | A10491-01 |
| Modification) | | |
| Antibiotic-Antimyotic (100X) | GIBCO | 15240-062 |
| 55 mM 2-Mercaptoethanol (1,000X) | Thermo | 21985023 |
| | Fisher | |
| Fetal Bovine Serum, certified, | GIBCO | 16000044 |
| US origin | | |
| GlutaMAX Supplement | Invitrogen | 35050-061 |
| Ficoll-Paque PLUS | GE | 17-1440-03 |
| SPRIselect Reagent | Beckman | B23318 |
| | Coulter | |
| T4 DNA Ligase | Invitrogen | 15224017 |
| SepMate-50 | STEMCELL | ST86450 |
| | Technologies | |
| | Inc | |
| Dimethyl sulfoxide | Sigma | D2650 |
| | Aldrich | |
| SuperScript ®IV First-Strand | Invitrogen | 18091050 |
| Synthesis S | | |
| Expand High FidelityPLUS PCR | Roche | 03300226001 |
| System | | |
| Deoxynucleotide (dNTP) Solution | NEB | N0447L |
| Mix | | |
| Sheath Fluid | BD | 342003 |
| 21343EZ-Link ®NHS-Biotin | Thermo | 21336 |
| Reagents | Fisher | |
| SPRIselect Reagent | Beckman | B23318 |
| | Coulter | |
| Chromium Single Cell 5 prime | 10X | 1000014 |
| Library Gel | Genomics | |
| Chromium Single Cell A Chip Kit | 10X | 1000008 |
| 16 rxns | Genomics | |
| PBS 1×, w/o Ca⁺⁺, Mg⁺⁺ 1 L | Lonza | 17-516Q |
| Molecular Grade Water | Corning | 46-000-CM |
| XL1-Blue | Stratagene | 200228 |
| T7 Express Strains | New England | C3010 |
| | Biolabs | |
| LB Broth | Amresco | J833-1L |
| Carbenicillin | Sigma | C3416 |
| Kanamycin Sulfate | Amresco | A20219 |
| NotI | New England | R0189S |
| | Biolabs | |
| Nco I | New England | R0193S |
| | Biolabs | |
| T4 Ligase | New England | M0202L |
| | Biolabs | |
| LB Agar LOP ™ Plate + | Narae | LN004CA |
| Carbenicilin75 | Biotech | |
| LB Agar LOP ™ Plate + | Narae | LN004K |
| Kanamycin 50 | Biotech | |
| Bacto Tryptone | DIFCO | 211705 |
| Bacto Yeast Extract | DIFCO | 212750 |
| MOPS | Sigma | M3183 |
| NuPAGE MOPS SDS Running | NOVEX | NP0001 |
| Buffer (20X) | | |
| Tris Base | Merck | CAS 77-86-1 |
| | Millipore | |
| Boric acid | Sigma | B6768 |
| Sodium Chloride | Sigma | S7653 |
| Sodium Azide | Sigma | S8032 |
| Bovine Serum Albumins | Sigma | A2058 |
| Ethanol | VWR | E193-500ML |
| | Lifescience | |
| 0.5M EDTA (pH 8.0) | Ambion | AM9260G |
| Glycerol | Sigma | G7893 |
| | Aldrich | |
| MabSelect ™Xtra | GE | 17-5269-02 |
| | Healthcare | |
| Protein A IgG binding buffer | Pierce | 21007 |
| IgG Elution buffer | Pierce | 21009 |
| PageBlue Protein Staining Solution | Pierce | 24620 |
| Tween-20 | Sigma | P1379 |
| | Aldrich | |
| TMB Microwell Peroxidase Substrate | KPL | 52-00-03 |
| TMB Stop Solution | KPL | 50-85-06 |
| Dithiothreitol (DTT), 0.1M Solution | USB | 707265ML |

TABLE 4

| Product | Supplier | Cat. No. |
|---|---|---|
| PCR cleanup kit | MN | 740609.10 |
| QuickExtract ™RNA Extraction Kit | Lucigen | QER090150 |
| SMARTer ®RACE 5′/3′ Kit | Takara Bio | 634859 |
| | USA Inc | |
| Chromium Single Cell V(D)J | 10X Genomics | 1000016 |
| Enrichment Kit, Human B Cell, 96 rxns | | |
| Chromium i7 Multiplex Kit 96 rxns | 10X Genomics | 120262 |
| Chromium Single Cell A Chip Kit 16 | 10X Genomics | 1000009 |
| rxns | | |
| Qubit ™dsDNA HS Assay Kit | Thermo Fisher | Q32854 |
| Eppendorf PCR Tubes 0.2 ml | Eppendorf | 0030124359 |
| High Sensitivity DNA Kit | Agilent | 5067-4626 |
| In-fusion HD Cloning Kit | Clontech | 639650 |
| Qiaprep Spin Miniprep Kit | Qiagen | 27106 |
| Gel Extraction Kit | Qiagen | 28706 |
| PCR Purification Kit | Qiagen | 28106 |
| 1L Erlenmeyer Culture Flask | Corning | 431147 |
| 225 mL Graduated Conical Tube | Corning | 352075 |
| Disposable 10 mL Polypropylene | Pierce | 29924 |
| Columns | | |
| NuPAGE ® Novex 4-12% Bis-Tris Gel | Invitrogen | NP0321BOX |
| 1000 mL Vacuum Filter/Bottle, 0.22 | Corning | 431098 |
| μm (PES) | | |
| Vivaspin20 membrane 50.000 MWCO | Sartorus | VS2032 |
| 37° C. Incubator | N-Biotek | SJP-250MI |
| 37° C. Shaking Incubator | Vision | VS-8489SR |
| | Scientific | |
| | Co., Ltd. | |
| Clean Bench | Nok Woo | N/A |
| | Industry | |
| CO₂ Incubator | N-Biotek | NB-206CXXL |
| Electrophoration Cuvette 0.2 cm | BTX | 620 |
| 50 mL Cornical tube | SPL | 50050 |
| Micro centrifugation Tube | SPL | 60115 |
| 15 mL Cornical tube | SPL | 50015 |
| PCR tube | Bioneer | T1C-028-R |
| Internal cryogenic vial | Corning | 431417 |
| pH meter (SevenEasy) | Mettler | N/A |
| | Toledo | |
| BD LSRFIRTESSA | BD | 649225 |
| NanoDrop ™2000 Spectrophotometer | Thermo | ND-2000 |
| | Fisher | |

### 3. Preparation and Selection of Anti-RSV Antibody

TABLE 5

| Product | Supplier | Cat. # |
|---|---|---|
| PBS 1×, w/o Ca⁺⁺, Mg⁺⁺ 1 L | Lonza | 17-516Q |
| Molecular Grade Water | Corning | 46-000-CM |
| XL1-Blue | Stratagene | 200228 |
| LB Broth | Amresco | J833-1L |
| Carbenicillin | Sigma | C3416 |
| Kanamycin Sulfate | Amresco | A20219 |
| LB Agar LOP ™ Plate + Carbenicilin75 | Narae | LN004CA |
| | Biotech | |
| Bacto Tryptone | DIFCO | 211705 |
| Bacto Yeast Extract | DIFCO | 212750 |
| MOPS | Sigma | M3183 |
| Tris Base | Merck | CAS 77-86-1 |
| | Millipore | |
| Sodium Chloride | Sigma | S7653 |
| NuPAGE MOPS SDS Running Buffer (20X) | NOVEX | NP0001 |
| Protein A IgG binding buffer | Pierce | 21007 |
| IgG Elution buffer | Pierce | 21009 |
| PageBlue Protein Staining Solution | Pierce | 24620 |
| Protein A IgG binding buffer | Pierce | 21007 |
| SuperScript ®IV First-Strand | Invitrogen | 18091050 |
| Synthesis S | | |
| Expand High FidelityPLUS PCR System | Roche | 03300226001 |
| Deoxynucleotide (dNTP) Solution Mix | NEB | N0447L |
| Tween-20 | Sigma | P1379 |
| | Aldrich | |
| TMB Microwell Peroxidase Substrate | KPL | 52-00-03 |
| TMB Stop Solution | KPL | 50-85-06 |

TABLE 6

| Product | Supplier | Cat. No. |
|---|---|---|
| Qiaprep Spin Miniprep Kit | Qiagen | 27106 |
| Qiaprep Plasmid Midi Kit | Corning | 12945 |
| Gel Extraction Kit | Qiagen | 28706 |
| PCR Purification Kit | Qiagen | 28106 |
| 1L Erlenmeyer Culture Flask | Corning | 431147 |
| 225 mL Graduated Conical Tube | Corning | 352075 |
| Disposable 10 mL Polypropylene | Pierce | 29924 |
| Columns | | |
| NuPAGE ® Novex 4-12% Bis-Tris Gel | Invitrogen | NP0321BOX |
| 1000 mL Vacuum Filter/Bottle, 0.22 | Corning | 431098 |
| μm (PES) | | |
| Zeba ™ Spin Desalting Columns | Thermo | 89891 |
| | Scientific | |
| Vivaspin20 membrane 50.000 MWCO | Sartorus | VS2032 |
| 37° C. Incubator | N-Biotek | SJP-250MI |
| 37° C. Shaking Incubator | Vision | VS-8489SR |
| | Scientific | |
| | Co., Ltd. | |
| Clean Bench | Nok Woo | N/A |
| | Industry | |
| CO₂ Incubator | N-Biotek | NB-206CXXL |
| Electrophoration Cuvette 0.2 cm | BTX | 620 |
| 50 mL Cornical tube | SPL | 50050 |
| Micro Centrifuge Tube | SPL | 60115 |
| 15 mL Cornical tube | SPL | 50015 |
| pH meter (SevenEasy) | Mettler | N/A |
| | Toledo | |

### Experimental Method

### 1. Preparation of RSV F-Protein Antigen

### 1.1. Transformation and Protein Expression

100 ng of a F-protein subtype gene expression vector (pcIW) was added to competent cells (competent sample) and mixed. The resulting mixture was transferred to a cuvette for electroporation, and then an electric shock was applied with a gap of 2 mm and a voltage of 2.5 kV. The cells were recovered in a shaking incubator with 1 mL of LB media for 1 hour at 37° C. The resulting cells were spread on an LB plate containing carbenicillin (75 μg/mL) for transformation. The obtained plasmid was sequenced to confirm whether a desired gene was cloned in a vector.

The day before expression, Expi293 cells (host cells) were sub-cultured at 2×10⁶ samples/mL. After confirming that the number of cells reached 3×10⁶ samples/mL or more on the day of expression, a mixture containing 30 μg of prepared DNA in 1.5 mL of Opti-MEM medium and a mixture containing 80 μL of ExpiFectamine in 1.5 mL of a medium were prepared and incubated for 5 minutes at room temperature. Afterward, the two mixtures were mixed and incubated for 20 to 30 minutes, and then the mixture was added to the prepared cells (25.5 mL). Enhancer 1 and Enhancer 2 were added at 150 μL and 1.5 mL 16 to 20 hours after expression, respectively, and expressed for 5 to 7 days at 37° C. in a shaking incubator.

### 1.2. Protein Purification and Concentration

1 mL of a Ni-NTA agarose resin washed with PBS was added to a protein-expressing supernatant, and inverted at 4° C. for 2 hours and at room temperature for 30 minutes. After transferring to a column for purification, the resulting mixture was sufficiently washed with an over 20-fold amount of a washing buffer (His-tag). Subsequently, 500 μL of an eluent was acquired six or more times using an elution buffer, and then the concentration of each sample was measured at a wavelength of 280 nm.

For buffer concentration and concentration, 10 mL of a PBS buffer was added to a Vivaspin20 membrane, centrifuged at 6500 rpm for 5 minutes to wash the membrane. After adding 10 mL of the PBS buffer, the sample was centrifuged at 6500 rpm for 10 minutes (repeated three times to contain a total of 30 mL of PBS). After centrifugation until approximately 500 μL of the sample remained, the resulting filtrate was collected to measure a concentration.

The prepared protein was confirmed using SDS-PAGE. The sample and a 4× non-reducing sample buffer were mixed in a volume ratio of 3:1, heated at 90° C. for 3 minutes and then cooled, followed by loading 3 μg of the prepared sample to each well of a 4 to 12% Bis-Tris gel and performing electrophoresis. The gel was isolated and stained with a PageBlue Protein Staining Solution for 30 minutes or more, followed by destaining with distilled water (DW) and confirming the size and purity of the protein.

### 2. Construction of Human B Cell-Derived Library

2.1. Isolation of human PBMCs

Peripheral blood mononuclear cells (PBMCs) were isolated by collecting blood samples from 40 healthy adults, 30 RSV convalescent infants, and 10 healthy infants.

After 35 mL each of the blood samples collected from the healthy adult donors was mixed with 35 mL of 2% FBS/PBS in a 1:1 ratio, the resulting mixture was dispensed into two 50-ml conical tubes (SPL, 50050). 15 mL of Ficoll-Paque Plus (GE, 17-1440-03) was added to each of four SepMate tubes (STEMCELL Technologies Inc, ST86450), a mixture of the blood and PBS (Lonza, 17-516Q) was overlaid by 17.5 mL, and centrifuged at 1200 g for 10 minutes (25° C., Acc5/Dec 5). After removing a plasma, the supernatant was collected in a new 50 ml conical tube (SPL, 50050) and centrifuged at 300× g for 8 minutes (25° C., Acc5/Dec 5). The resulting product was washed with 20 mL of 2% FBS/PBS, and centrifuged at 300×g for 8 minutes (25° C., Acc5/Dec 5). A supernatant was removed. Cells were resuspended with 10 mL of complete media (RPMI GIBCO A10491-01, 10% FBS GIBCO 16000044, 1% GlutaMAX Invitrogen 35050-061, 1% antibiotic-antimyotic GIBCO 15240-062, 1000× 2-mercaptoethanol Thermo Fisher 21985023), and transferred to a new 15-mL conical tube (SPL, 50015). 50 μL of the resuspended cells was counted, and the number of stored samples was determined (1×10⁷ cells/mL, 1 mL/vial). Remaining cells were centrifuged at 300×g for 8 minutes (25° C., Acc5/Dec 5). After resuspending the cells with FBS (GIBCO, 16000044) in a volume half the vial volume, the other half of the volume was filled with 20% DMSO/FBS and dispensed into an internal cryogenic vial (Corning, 431417). The vial was cryopreserved using CRF and then stored in liquid nitrogen.

4 mL of PBS (Lonza, 17-516Q) was added to 2 mL of the blood samples collected from the RSV convalescent and healthy infants and then gently inverted. 3 mL of Ficoll-Paque Plus (GE, 17-1440-03) was added to a 15 mL tube, and 6 mL of PBS-diluted blood was layered. After centrifugation (Brake: off) at room temperature and 1500 rpm for 25 minutes, a plasma and approximately 2 mL of a buffy coat of the Ficoll-Paque Plus interface were transferred to a new 15-mL tube (SPL, 50015). 10 mL of PBS (Lonza, 17-516Q) was added to the buffy coat and centrifuged (Brake on [9]) at 1200 rpm for 10 minutes, and a washing process for removing the supernatant was repeated twice. The supernatant-removed PBMCs were suspended in 1 mL of Complete RPMI (RPMI GIBCO A10491-01, 10% FBS GIBCO 16000044, 1% GlutaMAX Invitrogen 35050-061, 1% antibiotic-antimyotic GIBCO 15240-062, 1000× 2-mercaptoethanol Thermo Fisher 21985023), and the cells were counted using an Adam counter. After the supernatant was removed by centrifugation, the cells were suspended with FBS (GIBCO, 16000044) to be 2˜4×10⁷ cell/mL, 20% DMSO/FBS was carefully mixed with FBS in the same volume (1:1) ratio, and then the mixture was transferred to an internal cryogenic vial (Corning, 431417). The vial was cryopreserved using CRF and then stored in liquid nitrogen.

### 2.2. Isolation of B Cells Specific for RSV F-Protein

7 PBMC vials (donor #2, 4, 11, 23, 29, 39, 43, total cell number of 4.47×10⁷ cells) were resuspended in RPMI (GIBCO, A10491-01), and centrifuged at 1500 rpm for 10 minutes (25° C.) to discard the supernatant. The remaining cells were resuspended with 1 mL of 2% FBS/PBS, and centrifuged at 1,000 rpm for 3 minutes to discard the supernatant. PBMC cells were resuspended with 0.5 mL of 2% FBS/PBS such that a biotinylated RSV F (DS-Cav1) protein became 200 nM. The cells were reacted by tapping at 4° C. for 1 hour to prevent them from settling. After centrifugation at 1000 rpm for 3 minutes, the supernatant was discarded, and a process of washing the cells with 1 mL of 2% FBS/PBS and centrifuging the cells was repeated twice. 2.5 μL each of labeled antibodies (anti-human CD3 FITC eBioscience 11-0037, anti-human CD8 FITC TONBO BIOS 35-0087-T100, anti-human CD14 FITC TONBO BIOS 35-0149-T100, Streptavidin R-PE conjugate Invitrogen S866, anti-human CD19 Per-CP-cyanine5.5 eBioscience 45-0199, anti-human 20 PerCP-Cy5.5 BD 332781, mouse anti-human CD27 PE-Cy7 BD 560609, Streptavidin-APC eBioscience 17-4317-82) were added to 0.5 mL of 2% FBS/PBS and well mixed to manufacture an antibody mixture, followed by resuspension of the supernatant-removed cells and reaction at 4° C. for 20 minutes. After centrifugation at 1000 rpm for 3 minutes, the supernatant was discarded, and a process of washing the cells with 1 mL of 2% FBS/PBS and performing centrifugation was repeated twice, followed by resuspension with 0.5 mL of 2% FBS/PBS and sorting with FACSAriaII. Here, as a sorting gate, SSC/FSC→PBMC→CD3⁻CD8⁻CD14⁻/CD19⁺CD20⁺→CD27⁺→PE⁺/APC⁺ (for the control B cells, PE⁻/APC⁻) was selected, and approximately 1,000 cells were collected at a level of approximately 0.1 to 0.5% of CD27+B cells in 0.5 mL of 2% FBS/PBS.

### 2.3. Construction of B Cell-Derived V(D)J Library

Using a SMARTer RACE 5′ Kit (Takara Bio USA Inc, 634859), RT-PCR was performed. Isolated B cells were centrifuged at 1,500 rpm for 10 minutes, resuspended with 60 μL of Quick extraction buffer (Lucigen, QER090150), and reacted at −80° C. overnight. 4 μL of 5×RT buffer (Invitrogen, 18091050), 0.5 μL of DTT (USB, 707265 μL, 100 mM), and dNTPs (NEB, N0447L, 20 mM) were mixed to prepare a cDNA synthesis reaction mixture, and then the mixture was left at room temperature. 10 μL of cells lysed with Quick extraction buffer (Lucigen, QER090150) and 1 μL of a 5′-CDC primer were transferred to a PCR tube (Bioneer, T1C-028-R) to allow a reaction at 72° C. for 3 minutes and at 42° C. for 2 minutes, followed by spinning down. 0.5 μL of an RNase inhibitor and 2 μL of SMARTScribe Reverse Transcriptase were mixed with 5.5 μL of a prepared cDNA synthesis reaction mixture, and left at room temperature. 1 μL of SMARTer II A oligonucleotide was put into the tube in which the reaction had been completed, and 8 μL of the prepared mixture was added. After spin-down, RT-PCR was performed. RT-PCR was performed at 42° C. for 90 minutes and at 70° C. for 10 minutes. 10 μL of Tricine-EDTA buffer was added to the tube in which the reaction had been completed and diluted.

After 15.5 μL of PCR Grade H₂O, 25 μL of 2× SeqAmp Buffer, and 1 μL of SeqAmp DNA polymerase were mixed to prepare 41.5 μL of a mixture, 2.5 μL of 5′-RACE-finished cDNA, 5 μL of 10× UPM, and 1 μL of 5′-GSP, that is, RT-IgGKLA (10 μM) were added and gently mixed, followed by performing PCR. PCR was performed by repeating 25 cycles at 94° C. for 30 seconds, at 68° C. for 30 seconds, and 72° C. for 3 minutes. An RT-IgGKLA primer was prepared by mixing four types of sequences shown in Table 7 in a 1:1:1:1 ratio.

TABLE 7

| Name | Sequences (5′-3′) | SEQ ID NO: |
|---|---|---|
| RT- | GTGTGCACGCCGCTGGTC | 334 |
| IgGKLA | | |
| | GTGGGAAGTTTCTGGCGG | 335 |
| | TCAC | |
| | TGGAGGGCGTTATCCACC | 336 |
| | TTCC | |
| | GTGCTCCCTTCATGCGTG | 337 |
| | ACC | |

The PCR-finished reaction product was subjected to PCR cleanup. 100 μL of NTI buffer (MN, 740609.10) was added and loaded on a PCR clean-up column (MN, 740609.10), followed by centrifugation. After the removal of the flow-through, a washing process of adding 700 μL of buffer NT3 (MN, 740609.10) and performing centrifugation again was repeated twice, followed by centrifugation for 1 minute. Afterward, the column was transferred to a new tube, incubated for 1 minute at room temperature with 20 μL of Buffer NE and then centrifuged, thereby obtaining cDNA.

### 2.4. Construction Chromium Single Cell V(D)J Library

A Chromium Single Cell V(D)J Enrichment Human B cell Kit (10× Genomics, 1000016) was used. Sorted cells were centrifuged at 1500 rpm for 10 minutes, and resuspended with 60 μL of PBSF (1×PBS, 2% FBS). The resuspension volume was determined so that the concentration became 100 to 2000 cells/μL and a minimum of 40 μL (volume used in counting (30 ml)+volume used in experiment (10 μL)) was obtained. In this experiment, unsorted PBMCs and the control B cell sample were resuspended in 60 μL, but in the case of RSV positive B cells, only 1,000 cells were sorted and diluted to 10 μL, followed by carrying out an experiment immediately without counting.

Counting of B cells specific for the RSV F-protein was sampled enough to perform three times. Counting was performed three times for each sample, and the average value of the results of counting a total of 9 times was obtained. Since the number of sorted cells was approximately 1,000, 31.7 μL of cells not mixed with nuclease-free water (Corning, 46-000-CM) were prepared. In the case of the control B cells, a target cell recovery number was set to 2,000 (since targeted cell recovery efficiency is approximately 50%, the target cell recovery number was set to approximately 1,000, twice the level of sorted cells) at a concentration of 600 cells/μL, and 5.8 μL of cells and 25.9 μL of nuclease-free water (Corning, 46-000-CM) were mixed. For unsorted control PBMCs, as the concentration of a cell stock was 1,400 cells/μL and a targeted cell recovery number was 20000 (for PBMCs, since a B cell population is approximately 10%, 10-fold the target cell recovery number of the B cells was taken), the cells were mixed with 24.8 μL of a cell stock and 7.9 μL of nuclease-free water (Corning, 46-000-CM). The amount of cells to be mixed may be changed according to the number of sorted cells and the desired degree of target cell recovery, and the kit guide was referred for each volume. For each sample, 50 μL of RT reagent mix, 5.9 μL of Poly-dT RT primer, 2.4 μL of Additive A, and 10 μL of RT enzyme Mix B were mixed by pipetting 15 times, allowed to settle and then stored on ice.

After binding of Chromium Chip A (10× Genomics, 100009) to a 10× chip holder, 90 μL, 40 μL, and 270 μL of a 50% glycerol solution (Glycerol, Sigma Aldrich G5516) were added to wells that would not be used, marked 1, 2 and 3 on their bottom, respectively. Cells mixed with nuclease-free water (Corning, 46-000-CM) were mixed with the RT-reaction mixture prepared on ice, and then 90 μL of the cell mixture was added to the No. 1 well. 40 μL of gel beads vortexed for 30 seconds were added to the No. 2 well, and 135 μL of partitioning oil was added to the No. 3 well twice (a total of 270 μL). After installing a 10× gasket, GEMs were formed by performing the Single Cell program of the Chromium controller.

After storing the PCR tube on ice, 100 μL of GEMs formed by the Single Cell program were transferred to a PCR tube strip (Eppendorf, 0030124359), and GEM-RT incubation was performed. GEM-RT was performed in a thermal cycler in which a lid temperature was set to 53° C. at 53° C. for 45 minutes, at 85° C. for 5 minutes and at 4° C. hold.

After GEM-RT, 125 μL of a recovery agent was added to each sample and then incubated for 60 seconds. After the layers of the mixture were separated, a tip was inserted into the bottom of the tube to remove 125 μL of the recovery agent/partitioning oil (pink) layers. 200 μL of a Dynabeads cleanup mix prepared by mixing 9 μL of nuclease-free water (Corning, 46-000-CM), 182 μL of Buffer Sample Clean Up 1, 4 μL of Dynabeads MyOne SILANE, and 5 μL of Additive A was added to each sample. After mixing by pipetting, the resulting mixture was incubated for 10 minutes at room temperature.

After incubation, the tube was located in a 10× Magnetic Separator/High position, and the supernatant of the mixture was removed. 300 μL of 80% ethanol (VWR, E193-500 mL) was added to the pellet, and then the ethanol was removed. 200 μL of 80% ethanol was added, and after 30 seconds, the ethanol was removed. After centrifugation, the tube was placed in the low position of the magnet, and the remaining ethanol was removed. After performing air drying for 1 minute, the magnet was removed. 35.5 μL of Elution Solution I (98 μL of Buffer EB, 1 μL of 10% Tween 20 (Sigma Aldrich, P1379), and 1 μL of Additive A) was added and mixed by pipetting. After incubation at room temperature for 1 minute, the tube was located in the low position of the magnet, and the supernatant was transferred to a new tube.

65 μL of cDNA Amplification Mix was added to the GEM-RT Cleanup-finished sample. The cDNA Amplification Mix was prepared by mixing 8 μL of nuclease-free water, 50 μL of an amplification master mix, 5 μL of cDNA Additive, and 2 μL of cDNA Primer Mix. The sample was mixed using a pipette, allowed to settle, and subjected to cDNA amplification PCR. PCR was carried out repeatedly in a thermal cycler in which a lid temperature was set to 105° C. for 14 cycles of 98° C. for 45 seconds, 98° C. for 20 seconds, 67° C. for 30 seconds, and 72° C. for 1 minute, and then at 72° C. for 1 minute and at 4° C. hold.

60 μL of a vortexed SPRIselect reagent (Beckman Coulter, B23318) was added to each of the PCR-completed samples and mixed with a pipette. After incubation at room temperature for 5 minutes, the tube was placed in the high position of the magnet, and the supernatant was removed. 30 seconds after putting 200 μL of 80% ethanol into the pellet, a washing process of removing ethanol was performed a total of three times. After centrifugation, the tube was placed in the low position of the magnet, the remaining ethanol was removed, and air drying was carried out for 1 minute. After the removal of the magnet, 45.5 μL of Buffer EB was added and mixed using a pipette for 15 seconds, and incubated at room temperature for 2 minutes. After placing the tube in the high position of the magnet, 45 μL of the supernatant was transferred to a new tube. 1 μL of the sample transferred to the new tube was taken to perform QC and quantification using Agilent Tapestation.

2 μL of the sample was transferred to a new tube and mixed with 33 μL of nuclease-free water (Corning, 46-000-CM). 65 μL of Target Enrichment 1 Reaction Mix prepared by mixing 5 μL of nuclease-free water, 50 μL an amplification master mix, 5 μL of cDNA Additive, and 1 5 μL of B Cell Mix was added to each tube, thereby preparing 100 μL of a PCR reaction mixture, and then PCR was carried out. PCR was performed in a thermal cycler in which a lid temperature was set to 105° C. at 98° C. for 45 seconds, performed repeatedly for 8 cycles of 98° C. for 20 seconds, 67° C. for 30 seconds, and 72° C. for 1 minute (6 cycles for the control B cells), and then performed at 72° C. for 1 minute and at 4° C. hold.

80 μL of a vortexed SPRIselect reagent (Beckman Coulter, B23318) was added to each of the PCR-completed samples and mixed with a pipette. After incubation at room temperature for 5 minutes, the tube was placed in the high position of the magnet, and the supernatant was removed. 30 seconds after putting 200 μL of 80% ethanol (Ethanol, VWR, E193-500 mL) into the pellet, a washing process of removing ethanol was performed a total of three times. After centrifugation, the tube was placed in the low position of the magnet, the remaining ethanol was removed, and air drying was carried out for 1 minute. After the removal of the magnet, 35.5 μL of Buffer EB was added and mixed using a pipette for 15 seconds, and incubated at room temperature for 2 minutes. After placing the tube in the low position of the magnet, 35 μL of the supernatant was transferred to a new tube. 65 μL of Target Enrichment 2 Reaction Mix prepared by mixing 5 μL of nuclease-free water (Corning, 46-000-CM), 50 μL of an amplification master mix, 5 μL of cDNA Additive, and 25 μL of B cell Mix was added to 35 μL of the sample and mixed using a pipette, followed by performing 2nd PCR. PCR was performed in a thermal cycler in which a lid temperature was set to 105° C. at 98° C. for 45 seconds, repeated for 10 cycles of 98° C. for 20 seconds, 67° C. for 30 seconds, and 72° C. for 1 minute (8 cycles for the control B cells), and performed at 72° C. for 1 minute and at 4° C. hold.

50 μL of a vortexed SPRIselect reagent (Beckman Coulter, B23318) was added to each of the PCR-completed samples and mixed with a pipette. After incubation at room temperature for 5 minutes, the tube was placed in the low position of the magnet, and the supernatant was removed. 30 μL of a SPRIselect reagent (Beckman Coulter, B23318) was added to the sample transferred to the new tube, mixed and reacted at room temperature for 5 minutes. The tube was placed in the high position of the magnet, and 170 μL of the supernatant was removed. 30 seconds after putting 200 μL of 80% ethanol (VWR, E193-500 mL) into the pellet, a washing process of removing ethanol was performed a total of three times. After centrifugation, the tube was placed in the low position of the magnet, the remaining ethanol was removed. After removing the magnet, 45.5 μL of Buffer EB was added and mixed using a pipette for 15 seconds, and incubated at room temperature for 2 minutes. After placing the tube in the low position of the magnet, 35 μL of the supernatant was transferred to a new tube. 1 μL of the sample contained in the new tube was taken and diluted 1:5, followed by performing QC and quantification using Agilent Tapestation. The sample for which the above procedure had been completed was used to construct a phage-displayed scFv library.

After 50 ng of the sample was transferred to a new tube and diluted with nuclease-free water to 20 μL, a Fragmentation Mix prepared by mixing 5 μL of fragmentation buffer, 10 μL of a fragmentation enzyme blend, and 15 μL of nuclease-free water was added to each sample, and a fragmentation reaction was carried out using a pre-chilled thermal cycler. The reaction was performed in a thermal cycler in which a lid temperature was set to 65° C. under conditions of 4° C. hold (skip immediately after the addition of the sample), 32° C. for 2 minutes, 65° C. for 30 minutes, and 4° C. hold. 50 μL of Adaptor Ligation Mix prepared by mixing 17.5 μL of nuclease-free water, 20 μL of a ligation buffer, 10 μL of a DNA ligase, and 2.5 μL of Adaptor Mix was added to 50 μL of the reaction-completed sample, mixed using a pipette and incubated using a thermal cycler. The incubation was performed in a thermal cycler in which a lid temperature was set to 30° C. under conditions of 20° C. for 15 minutes and 4° C. hold.

After a ligation reaction, a cleanup was carried out. 80 μL of the vortexed SPRIselect reagent (Beckman Coulter, B23318) was added to each sample and mixed using a pipette. After incubation at room temperature for 5 minutes, the tube was placed in a high position of the magnet and the supernatant was removed. 30 seconds after putting 200 μL of 80% ethanol into the pellet, a washing process of removing ethanol was performed a total of three times. After centrifugation, the tube was placed in the low position of the magnet, the remaining ethanol was removed and air drying was carried out for 1 minute. After the removal of the magnet, 30.5 μL of Buffer EB was added and mixed using a pipette for 15 seconds, followed by incubation at room temperature for 2 minutes. After placing the tube in the low position of the magnet, 30 μL of the supernatant was transferred to a new tube, and then sample index PCR was carried out.

30 μL of a sample index PCR mix prepared by mixing 8 μL of nuclease-free water, 50 μL of an amplification master mix, and 2 μL of SI-PCR Primer was added to 60 μL of each sample, and 10 μL of individual Chromium i7 Sample Index was added to each tube. After mixing using a pipette, spin down was performed and sample index PCR was performed. PCR was performed in a thermal cycler in which a lid temperature was set to 105° C. at 98° C. for 45 seconds, repeated for 9 cycles under conditions of 98° C. for 20 seconds, 54° C. for 30 seconds and 72° C. for 20 seconds, and then performed at 72° C. for 1 minute and at 4° C. hold.

80 μL of a SPRIselect reagent (Beckman Coulter, B23318) was added to the sample index PCR-completed sample, and mixed using a pipette. After incubation at room temperature for 5 minutes, the tube was placed in the high position of the magnet and the supernatant was removed. 30 seconds after putting 200 μL of 80% ethanol (VWR, E193-500 mL) into the pellet, a washing process of removing ethanol was performed a total of three times. After centrifugation, the tube was placed in the low position of the magnet, the remaining ethanol was removed and air drying was carried out for 1 minute. After the removal of the magnet, 35.5 μL of Buffer EB was added and mixed using a pipette for 15 seconds, followed by incubation at room temperature for 2 minutes. After placing the tube in the low position of the magnet, 35 μL of the supernatant was transferred to a new tube. 1 μL of the sample contained in the new tube was prepared as a 1:10 dilution, and QC and quantification were carried out using Agilent Tapestation. Samples for which the production of a single cell V(D)J enriched library was completed were subjected to HT-sequencing.

2.5. Construction of Phage Displayed scFv Library

1st PCR and nested PCR were performed using Roche PLUS #03300226001. Primers used for PCR were prepared by mixing the sequences in Table 8 in the same ratio.

TABLE 8

| | | SEQ | | SEQ |
|---|---|---|---|---|
| | | ID | | ID |
| Name | Sequences (5′-3′) | NO: | Sequences (5′-3′) | NO: |
| 1^{st} For | CCATGGACTGGACCTGGAG | 338 | GCTTCCTCCTCCTTTGGATCTCTG | 339 |
| | ATGGACATACTTTGTTCCACG | 340 | CTSCTGCTCTGGGYTCC | 341 |
| | ATGGAGTTTGGGCTGAGCTGG | 342 | GTCCTGGGCCCAGTCTG | 343 |
| | ATGGAATTGGGGCTGAGCTG | 344 | CCTGGGCTCTGCTSCTCCTC | 345 |
| | ATGGAGTTGGGACTGAGCTG | 346 | GTTCTGTGGTTTCTTCTGAGCTG | 347 |
| | ATGGAACTGGGGCTCCG | 348 | GTGGCCTCCTATGWGCTGAC | 349 |
| | ATGAAACACCTGTGGTTCTTCC | 350 | ACAGGGTCTCTCTCCCAG | 351 |
| | ATGGGGTCAACCGCCATC | 352 | ACAGGTCTCTGTGCTCTGC | 353 |
| | ATGCAAGTGGGGGCCTC | 354 | ATTCYCAGRCTGTGGTGAC | 355 |
| | ATGTCTGTCTCCTTCCTCATC | 356 | GCTCACTGCACAGGTTCTTGG | 357 |
| | GCTCAGCTCCTGGGGCT | 358 | TCCCTCTCSCAGSCTGTG | 359 |
| | CTTCCTCCTGCTACTCTGGCTC | 360 | CAGTGGTCCAGGCAGGG | 361 |
| | TTTCTCTGTTGCTCTGGATCTCTG | 362 | | |
| 1^{st} | GCCTGAGTTCCACGACACC | 363 | CTGTACTTTGGCCTCTCTGGGATAG | 364 |
| Rev | | | | |
| | CTGTCCGCTTTCGCTCCAG | 365 | TTCCACTGCTCRGGCGTCAG | 366 |
| Nested | CAGGTSCAGCTGGTGCAGTCTGG | 367 | CAGGTGCAGCTGCAGGAGTCGG | 368 |
| For | | | | |
| (VH) | | | | |
| | CAGGTCACCTTGAAGGAGTCTGGTCC | 369 | CAGGTGCAGCTACAGCAGTGGG | 370 |
| | GAGGTGCAGCTGGTGGAGTCTGG | 371 | GARGTGCAGCTGGTGCAGTCTGG | 372 |
| | GAGGTGCAGCTGTTGGAGTCTGG | 373 | CAGGTACAGCTGCAGCAGTCAGG | 374 |
| Nested | CAGTCTGTGYTGACKCAGCC | 375 | CAGGCAGGGCTGACTCAGC | 376 |
| For (VL) | | | | |
| | CAGTCTGCCCTGACTCAGCC | 377 | GACATCCAGWTGACCCAGTCTCC | 378 |
| | TCCTATGAGCTGACWCAGCCA | 379 | GATATTGTGATGACCCAGWCTCCACTC | 380 |
| | TCTTCTGAGCTGACTCAGGACC | 381 | GAAATTGTGTTGACRCAGTCTCCAG | 382 |
| | CAGCYTGTGCTGACTCAATC | 383 | GACATCGTGATGACCCAGTCTCC | 384 |
| | CWGSCTGTGCTGACTCAGCC | 385 | GAAACGACACTCACGCAGTCTCC | 386 |
| | AATTTTATGCTGACTCAGCCCCAC | 387 | GAAATTGTGCTGACWCAGTCTCCAG | 388 |
| | CAGRCTGTGGTGACYCAGGAG | 389 | GACATTGTGCTGACCCAGTCTC | 390 |
| Nested | GGGAAGTAGTCCTTGACCAGGC | 391 | GCACACAACAGAGGCAGTTCCAG | 392 |
| Rev | | | | |
| | TCACACTGAGTGGCTCCTGG | 393 | TGCTGGCCGCRTACTTGTTGTTG | 394 |

1st PCR was performed by mixing 10 μL of Expand High Fidelity^{PLUS} Reaction Buffer (5×) with 7.5 mM MgCl₂, 1 μL of dNTP (NEB, 10 mM), 2 μL of 1st For primer (10 μM), 2 μL of 1st Rev primer (10 μM), 30.5 μL of water, 0.5 μL of Expand High Fidelity^{PLUS} Enzyme blend, and 4 μL of cDNA. PCR was carried out repeatedly for 40 cycles under conditions of 94° C. for 2 minutes, 94° C. for 30 seconds, 53° C. for 1 minute, and 72° C. for 1 minute, and performed at 72° C. for 7 minutes. After PCR was completed, PCR cleanup was performed. 100 μL of NTI buffer was added, and then the resulting sample was loaded on a PCR clean-up column (MN, 740609.10) to run centrifugation. After removing the flow-through, a washing process of adding 700 μL of Buffer NT3 (MN, 740609.10) and performing re-centrifugation was repeated twice, and centrifugation was then performed 1 minute to dry the membrane. Subsequently, the column was transferred to a new tube, 20 μL of Buffer NE (MN, 740609.10) was added to the column, and the column was incubated for 1 minute at room temperature and then centrifuged, thereby obtaining cDNA.

For amplification of heavy chain cDNA, 10 μL of Expand High Fidelity^{PLUS} Reaction Buffer(5×) with 7.5 mM MgCl₂, 1 μL of dNTP (NEB, 10 mM), 2 μL of nested For VH primer (10 μM), 2 μL of nested Rev primer (10 μM), 30.5 μL of water, 0.5 μL of Expand High Fidelity^{PLUS} Enzyme blend, and 4 μL of 1st PCR product were mixed to perform 1st PCR. PCR was performed at 94° C. for 2 minutes, repeated for 40 cycles of 94° C. for 30 seconds, 53° C. for 1 minute and 72° C. for 1 minute, and then at 72° C. for 7 minutes. For amplification of light chain cDNA, in the same manner as above, 10 μL of Expand High Fidelity^{PLUS} Reaction Buffer(5×) with 7.5 mM MgCl₂, 1 μL of dNTP (NEB, 10 mM), 2 μL of nested For VH primer (10 μM), 2 μL of nested Rev primer (10 μM), 30.5 μL of water, 0.5 μL of Expand High Fidelity^{PLUS} Enzyme blend, and 4 μL of 1st PCR product were mixed to perform 1st PCR. PCR was performed at 94° C. for 2 minutes, repeated for 40 cycles of 94° C. for 30 seconds, 53° C. for 1 minute and 72° C. for 1 minute, and then at 72° C. for 7 minutes. After PCR was completed, PCR cleanup was performed. 100 μL of NTI buffer (MN, 740609.10) was added, and the resulting sample was loaded on a PCR clean-up column (MN, 740609.10) to centrifuge. After removing the flow-through, a washing process of adding 700 μL of Buffer NT3 (MN, 740609.10) and performing re-centrifugation was repeated twice, and centrifugation was then performed 1 minute to dry the membrane. Subsequently, the column was transferred to a new tube, 20 μL of Buffer NE (MN, 740609.10) was added to the column, and the column was incubated for 1 minute at room temperature and then centrifuged, thereby obtaining cDNA.

VH/V_{κ}/V_{λ} PCR and scFv overlapping PCR were performed using Roche PLUS #03300226001. Primers used for PCR were prepared by mixing the sequences in Table 9 in the same ratio.

TABLE 9

| | | SEQ ID | | SEQ |
|---|---|---|---|---|
| Name | Sequences (5′-3′) | NO: | Sequences (5′-3′) | ID NO: |
| VH | ccgtggcccaggcggccCAGGTGCAGCT | 395 | ccgtggcccaggcggccCAGGTGCAGCT | 396 |
| For | GGTGCAGTCT | | GGTGGAGTCT | |
| | ccgtggcccaggcggccCAGGTYCAGCT | 397 | ccgtggcccaggcggccGAGGTACAGC | 398 |
| | KGTGCAGTCT | | TCGTGGAGTCC | |
| | ccgtggcccaggcggccCAGGTTCAGCT | 399 | ccgtggcccaggcggccAGGTGGAGCT | 400 |
| | GGTGCAGTCT | | GATAGAGTCC | |
| | ccgtggcccaggcggccCAGGTCCAGCT | 401 | ccgtggcccaggcggccCAGGTRCAGCT | 402 |
| | GGTACAGTCT | | GGTGGAGTCT | |
| | ccgtggcccaggcggccCAGGTCCAGCT | 403 | ccgtggcccaggcggccGAGGATCAGC | 404 |
| | GGTGCAGTCT | | TGGTGGAGTCT | |
| | ccgtggcccaggcggccCAGATGCAGCT | 405 | ccgtggcccaggcggccGAGGTGCAGC | 406 |
| | GGTGCAGTCT | | TGGTGGAGWCT | |
| | ccgtggcccaggcggccCAAATGCAGCT | 407 | ccgtggcccaggcggccSAGGTGCAGCT | 408 |
| | GGTGCAGTCT | | GGTGGAGTCT | |
| | ccgtggcccaggcggccGAGGTCCAGCT | 409 | ccgtggcccaggcggccGAGGTGCAGC | 410 |
| | GGTACAGTCT | | TGGTGGAGTCY | |
| | ccgtggcccaggcggccCAGRTCACCTT | 411 | ccgtggcccaggcggccCAGGTGCAGCT | 412 |
| | GAAGGAGTCT | | GCAGGAGTYG | |
| | ccgtggcccaggcggccCAGGTCACCTT | 413 | ccgtggcccaggcggccCAGGTGCAGCT | 414 |
| | GAAGGAGTCT | | GCAGGAGTCG | |
| | ccgtggcccaggcggccCAGGTCACCTT | 415 | ccgtggcccaggcggccCAGCTGCAGCT | 416 |
| | GARGGAGTCT | | GCAGGAGTCC | |
| | ccgtggcccaggcggccGAGGTGCAGC | 417 | ccgtggcccaggcggccCAGGTGCAGCT | 418 |
| | TGGTGGAGTCT | | GCAGGASTCG | |
| | ccgtggcccaggcggccGAAGTGCAGC | 419 | ccgtggcccaggcggccCAGGTGCRGCT | 420 |
| | TGGTGGAGTCT | | GCAGGAGTCG | |
| | ccgtggcccaggcggccCAGGTGCAGCT | 421 | ccgtggcccaggcggccCAGGTGCAGCT | 422 |
| | GKTGGAGTCT | | RCARSAGTSG | |
| | ccgtggcccaggcggccGAGGTGCAKC | 423 | ccgtggcccaggcggccCAGSTGCAGCT | 424 |
| | TGGTGGAGTCT | | GCAGGAGTCG | |
| | ccgtggcccaggcggccGAGGTRCARCT | 425 | ccgtggcccaggcggccGAAGTGCAGC | 426 |
| | GGTGGAGTCT | | TGGTGCAGTCY | |
| | ccgtggcccaggcggccACAGTGCAGCT | 427 | ccgtggcccaggcggccGAGGTGCAGC | 428 |
| | GGTGGAGTCT | | TGGTGCAGTCT | |
| | ccgtggcccaggcggccGAGGTGCARCT | 429 | ccgtggcccaggcggccCAGGTACAGCT | 430 |
| | GGTGGAGTCT | | GCAGCAGTCA | |
| | ccgtggcccaggcggccGAGGTGCAGC | 431 | ccgtggcccaggcggccCAGGTGCAGCT | 432 |
| | TGKTGGAGTCT | | GGTGCAATCT | |
| | ccgtggcccaggcggccGAGAYGCAGC | 433 | ccgtggcccaggcggccCTGCAGCTGGT | 434 |
| | TGGTGGAGTCT | | GCAGTCTGGG | |
| | ccgtggcccaggcggccGAGGTGGAGC | 435 | ccgtggcccaggcggccCAGGTGCAGCT | 436 |
| | TGATAGAGCCC | | GGTGCAGTCT | |
| JH | gcctgaaccacctccgccagatccgccacctccT | 437 | gcctgaaccacctccgccagatccgccacctccT | 438 |
| Rev | GAGGAGAC GGTGACCAGGGTG | | GAGGAGAC GGTGACCAGGGT | |
| | gcctgaaccacctccgccagatccgccacctccT | 439 | gcctgaaccacctccgccagatccgccacctccT | 440 |
| | GAGGAGAC | | GAGGAGACGGTGACCGTGGTC | |
| | AGTGACCAGGGTGC | | | |
| | gcctgaaccacctccgccagatccgccacctccT | 441 | | |
| | GAAGAGAC | | | |
| | GGTGACCATTGTCCCTT | | | |
| Vκ | TCTGGCGGAGGTGGTTCAGGCG | 442 | TCTGGCGGAGGTGGTTCAGGCG | 443 |
| For | GTGGAGGCTCGGACATCCAGAT | | GTGGAGGCTCGGATGTTGTGAT | |
| | GACCCAGTCTCCT | | GACTCAGTCTCCA | |
| | TCTGGCGGAGGTGGTTCAGGCG | 444 | TCTGGCGGAGGTGGTTCAGGCG | 445 |
| | GTGGAGGCTCGGCCATCCAGAT | | GTGGAGGCTCGGAGATTGTGAT | |
| | GACCCAGTCTCCA | | GACCCAGACTCCA | |
| | TCTGGCGGAGGTGGTTCAGGCG | 446 | TCTGGCGGAGGTGGTTCAGGCG | 447 |
| | GTGGAGGCTCGGYCATCYGGAT | | GTGGAGGCTCGGAAATTGTAAT | |
| | GACCCAGTCTCCA | | GACACAGTCTCAA | |
| | TCTGGCGGAGGTGGTTCAGGCG | 448 | TCTGGCGGAGGTGGTTCAGGCG | 449 |
| | GTGGAGGCTCGGACATCCAGTT | | GTGGAGGCTCGGAAATTGTGTT | |
| | GACCCAGTCTCCA | | GACACAGTCTCCA | |
| | TCTGGCGGAGGTGGTTCAGGCG | 450 | TCTGGCGGAGGTGGTTCAGGCG | 451 |
| | GTGGAGGCTCGGACATCCAGAT | | GTGGAGGCTCGGAAATAGTGAT | |
| | GACCCAGTCTCCA | | GASGCAGTCTCCA | |
| | TCTGGCGGAGGTGGTTCAGGCG | 452 | TCTGGCGGAGGTGGTTCAGGCG | 453 |
| | | | GTGGAGGCTCGGAAATTGTGTT | |
| | GTGGAGGCTCGGCCATCCAGTT | | GACRCAGTCTCCA | |
| | GACCCAGTCTCCA | | | |
| | TCTGGCGGAGGTGGTTCAGGCG | 454 | TCTGGCGGAGGTGGTTCAGGCG | 455 |
| | GTGGAGGCTCGRACATCCAGAT | | GTGGAGGCTCGGACATCGTGAT | |
| | GACCCAGTCTCCA | | GACCCAGTCTCCA | |
| | TCTGGCGGAGGTGGTTCAGGCG | 456 | TCTGGCGGAGGTGGTTCAGGCG | 457 |
| | GTGGAGGCTCGGACATCCAGAT | | GTGGAGGCTCGGAAACGACACT | |
| | GAYCCAGTCTCCA | | CACGCAGTCTCCA | |
| | TCTGGCGGAGGTGGTTCAGGCG | 458 | TCTGGCGGAGGTGGTTCAGGCG | 459 |
| | GTGGAGGCTCGGACATCCAGAT | | GTGGAGGCTCGGAAATTGTGCT | |
| | GAYCCAGTCTCCA | | GACTCAGTCTCCA | |
| | TCTGGCGGAGGTGGTTCAGGCG | 460 | TCTGGCGGAGGTGGTTCAGGCG | 461 |
| | GTGGAGGCTCGGATATTGTGAT | | GTGGAGGCTCGGATGTTGTGAT | |
| | GACCCAGCATCTG | | GACACAGTCTCCA | |
| | TCTGGCGGAGGTGGTTCAGGCG | 462 | TCTGGCGGAGGTGGTTCAGGCG | 463 |
| | GTGGAGGCTCGGATATTGTGAT | | GTGGAGGCTCGGACATTGTGCT | |
| | GACCCAGACTCCA | | GACCCAGTCTCCA | |
| | TCTGGCGGAGGTGGTTCAGGCG | 464 | | |
| | GTGGAGGCTCGGATATTGTGAT | | | |
| | GACTCAGTCTCCA | | | |
| Jκ | TGCTGGCCGGCCTGGCCTCGTT | 465 | TGCTGGCCGGCCTGGCCTCGTT | 466 |
| Rev | TGATCTCCAGCTTGGTCCCCTG | | TGATCTCCACCTTGGTCCCTCC | |
| | TGCTGGCCGGCCTGGCCTCGTT | 467 | TGCTGGCCGGCCTGGCCTCGTT | 468 |
| | TGATATCCACTTTGGTCCCAGG | | TAATCTCCAGTCGTGTCCCTTGG | |
| | GCC | | CC | |
| Vλ | TCTGGCGGAGGTGGTTCAGGCG | 469 | TCTGGCGGAGGTGGTTCAGGCG | 470 |
| For | GTGGAGGCTCGCAGTCTGTGCT | | GTGGAGGCTCGCAGTCTGCCCT | |
| | GACTCAGCCACC | | GACTCAGCCTCA | |
| | TCTGGCGGAGGTGGTTCAGGCG | 471 | TCTGGCGGAGGTGGTTCAGGCG | 472 |
| | | | GTGGAGGCTCGTCCTATGAGCT | |
| | GTGGAGGCTCGCAGTCTGTSST | | GAYRCAGCCACCC | |
| | GACGCAGCCG | | | |
| | TCTGGCGGAGGTGGTTCAGGCG | 473 | TCTGGCGGAGGTGGTTCAGGCG | 474 |
| | GTGGAGGCTCGCAGTCTGTGTT | | GTGGAGGCTCGTCCTCCATGCT | |
| | GACGCAGCCGC | | GACTCAGGAGCCA | |
| | TCTGGCGGAGGTGGTTCAGGCG | 475 | TCTGGCGGAGGTGGTTCAGGCG | 476 |
| | GTGGAGGCTCGCAGYCTGTGCT | | GTGGAGGCTCGTCCTATGAGCT | |
| | GACTCAGCCAC | | GACACAGCCAYCCTC | |
| | TCTGGCGGAGGTGGTTCAGGCG | 477 | TCTGGCGGAGGTGGTTCAGGCG | 478 |
| | GTGGAGGCTCGCAGTCTGTGYT | | GTGGAGGCTCGACATATGAGCT | |
| | GACGCAGCCG | | GTCTCAGCCACCC | |
| | TCTGGCGGAGGTGGTTCAGGCG | 479 | TCTGGCGGAGGTGGTTCAGGCG | 480 |
| | | | GTGGAGGCTCGTCCTCTGAGCT | |
| | GTGGAGGCTCGCAGCCTGTGCT | | GAGTCAGGAGCCT | |
| | GACTCAGATGACC | | | |
| | TCTGGCGGAGGTGGTTCAGGCG | 481 | TCTGGCGGAGGTGGTTCAGGCG | 482 |
| | GTGGAGGCTCGCAGTCTGCCCT | | GTGGAGGCTCGTCCTCTGGGCC | |
| | GAYTCAGCCTC | | AACTCAGGTGC | |
| | TCTGGCGGAGGTGGTTCAGGCG | 483 | TCTGGCGGAGGTGGTTCAGGCG | 484 |
| | GTGGAGGCTCGCARTCTGCCCT | | GTGGAGGCTCGCTGCCTGTGCT | |
| | GACTCAGCCTS | | GACTCAGCCC | |
| | TCTGGCGGAGGTGGTTCAGGCG | 485 | T | |
| | CTGGCGGAGGTGGTTCAGGCG | 486 | | |
| | GTGGAGGCTCGCAGTCTGTTCT | | GTGGAGGCTCGCAGCCTGTGCT | |
| | GACTCAGCCTCGC | | GACTCAATCATCC | |
| | TCTGGCGGAGGTGGTTCAGGCG | 487 | TCTGGCGGAGGTGGTTCAGGCG | 488 |
| | GTGGAGGCTCGTCCTATGAGCT | | GTGGAGGCTCGCAGCTTGTGCT | |
| | GACTCAGCCACCC | | GACTCAATCGCCC | |
| | TCTGGCGGAGGTGGTTCAGGCG | 489 | TCTGGCGGAGGTGGTTCAGGCG | 490 |
| | GTGGAGGCTCGTCCTCTGAGCT | | GTGGAGGCTCGCAGCCTGTGCT | |
| | GACTCAGCTGCCT | | GACTCAGCCR | |
| | TCTGGCGGAGGTGGTTCAGGCG | 491 | TCTGGCGGAGGTGGTTCAGGCG | 492 |
| | GTGGAGGCTCGTCCTATGAGCT | | GTGGAGGCTCGAATTTTATGCT | |
| | TACACAGCCACCCTC | | GACTCAGCCCCACTCT | |
| | TCTGGCGGAGGTGGTTCAGGCG | 493 | TCTGGCGGAGGTGGTTCAGGCG | 494 |
| | GTGGAGGCTCGTCCTCTGAGCG | | GTGGAGGCTCGCAGRCTGTGGT | |
| | GACTCAGTTGCCT | | GACTCAGGAGCC | |
| | TCTGGCGGAGGTGGTTCAGGCG | 495 | TCTGGCGGAGGTGGTTCAGGCG | 496 |
| | GTGGAGGCTCGTCCTATGAGCT | | GTGGAGGCTCGCAGACTGTGGT | |
| | GACTCAGCCACTCTC | | GACCCAGGAG | |
| | TCTGGCGGAGGTGGTTCAGGCG | 497 | TCTGGCGGAGGTGGTTCAGGCG | 498 |
| | GTGGAGGCTCGTCCTATGAGCT | | GTGGAGGCTCGCAGCCTGTGCT | |
| | GACWCAGCCACMC | | GACTCAGCCA | |
| | TCTGGCGGAGGTGGTTCAGGCG | 499 | TCTGGCGGAGGTGGTTCAGGCG | 500 |
| | GTGGAGGCTCGTCTTCTGAGCT | | GTGGAGGCTCGCAGGCAGGGCT | |
| | GACTCAGGACCCTG | | GACTCAGCCA | |
| | TCTGGCGGAGGTGGTTCAGGCG | 501 | TCTGGCGGAGGTGGTTCAGGCG | 502 |
| | GTGGAGGCTCGTCCTATGTGCT | | GTGGAGGCTCGCGGCCCGTGCT | |
| | GACWCAGCYACCC | | GACTCAG | |
| Jλ | TGCTGGCCGGCCTGGCCGCCTA | 503 | TGCTGGCCGGCCTGGCCGCCTA | 504 |
| Rev | GGACGGTGACCTTGGTCCCAGT | | GGACGGTCAGCTCSGTCCC | |
| | TGCTGGCCGGCCTGGCCGCCTA | 505 | TGCTGGCCGGCCTGGCCGCCGA | 506 |
| | GGACGGTCAGCTTGGTCCCTC | | GGACGGTCACCTTGGTGCCA | |
| | TGCTGGCCGGCCTGGCCGCCTA | 507 | TGCTGGCCGGCCTGGCCGCCGA | 508 |
| | AAATGATCAGCTGGGTTCCTCC | | GGYCGGTCAGCTGGGTGC | |
| | ACCAAATA | | | |

For the amplification of VH fragments, 10 μL of Expand High Fidelity^{PLUS} Reaction Buffer (5×) with 7.5 mM MgCl₂, 1 μL of dNTP (NEB, 10 mM), 2 μL of VH For VH primer (10 μM), 2 μL of JH Rev primer (10 μM), 32.5 μL of water, 0.5 μL of Expand High Fidelity^{PLUS} Enzyme blend, and 2 μL of Heavy chain cDNA were mixed to perform PCR. PCR was performed at 94° C. for 2 minutes, repeated for 30 cycles of 94° C. for 30 seconds, 53° C. for 30 seconds and 72° C. for 1 minute, and then at 72° C. for 7 minutes. PCR for a V_{κ} fragment (primer: V_{κ}/J_{κ}, cDNA: light chain) and a V_{λ} fragment (primer: V_{λ}/J_{λ}, cDNA: light chain) was also performed in the same manner as for the VH fragment.

After PCR was completed, gel extraction was performed. The resulting sample was loaded on 0.7% agarose gel, and a gel having a size of 350 bp was cut. 200 μL of NTI buffer was added per 100 mg of the cut gel, and the resulting gel was located on a column to centrifuge. After removing the flow-through, a washing process of adding 700 μL of Buffer NT3 and performing re-centrifugation was repeated twice, and centrifugation was then performed 1 minute to dry the membrane. Subsequently, the column was transferred to a new tube, 20 μL of Buffer NE was added to the column, and the column was incubated for 1 minute at room temperature and then centrifuged, thereby obtaining amplified fragment DNA.

For VH-V_{κ} scFv overlapping PCR, 10 μL of Expand High Fidelity^{PLUS} Reaction Buffer (5×) with 7.5 mM MgCl₂, 1 μL of dNTP (NEB, 10 mM), 2 μL of VH For VH primer (10 μM), 2 μL of J_{κ} Rev primer (10 μM), 30.5 L of water, 0.5 L of Expand High Fidelity^{PLUS} Enzyme blend, 2 μL of VH fragments, and 2 μL of V_{κ} fragments were mixed to perform PCR. PCR was performed at 94° C. for 2 minutes, repeated for 30 cycles of 94° C. for 30 seconds, 53° C. for 1 minute and 72° C. for 1 minute, and then at 72° C. for 7 minutes. VH-V_{λ} scFv overlapping PCR was also performed in the same manner as above. Here, as the fragments, the VH fragment and the V_{λ} fragment were used, and as the primers, VH For and J_{λ} Rev were used.

After PCR was completed, gel extraction was performed. The resulting sample was loaded on 0.7% agarose gel, and a gel having a size of 750-800 bp was cut. 200 μL of NTI buffer was added per 100 mg of the cut gel, and the resulting gel was located on a column to centrifuge. After removing the flow-through, a washing process of adding 700 μL of Buffer NT3 (MN, 740609.10) and performing re-centrifugation was repeated twice, and centrifugation was then performed 1 minute to dry the membrane. Subsequently, the column was transferred to a new tube, 20 μL of Buffer NE was added to the column, and the column was incubated for 1 minute at room temperature and then centrifuged, thereby obtaining amplified scFv insert DNA.

1.4 μg pcomb3-XTT vector linearized by SfiI (Sigma Aldrich, 11288059001) treatment, 700 ng of scFv insert DNA, 20 μL of 5× ligase reaction buffer, and 5 μL of T4 DNA Ligase (Invitrogen, 15224017) were mixed, autoclaved distilled water was added to a total volume of 100 μL, and then the resulting mixture was incubated at room temperature overnight.

### 3. Selection and Manufacture of Anti-RSV Antibody

3.1. Panning of Phage-Displayed scFv Library

Library panning was performed by an immunotube method. 5 μg/mL of DS-Cav1 (in-house) was put into an immunotube (e. g. Nunc MaxiSorp 444202), and incubated at 4° C. overnight. The next day, an antigen was removed from a DS-Cav1-coated tube, the immunotube was washed twice with PBS-Tween20 (0.1%), fully filled with 2% skim milk and then blocked at room temperature for 2 hours. After blocking, a blocking solution was removed from the immunotube, and 1 mL of the blocked phage library was added to incubate at least 1 hour at 37° C. The phage library was removed from the immunotube and then the immunotube was washed five times with 1 mL of PBS-Tween20 (0.1%).

To recover the phages binding to the immunotube, 1 mL of 10 mM glycine-HCl pH 1.5+1% BSA was added, and the phages were incubated for 10 minutes. The recovered phages were transferred to a 50 ml Falcon tube and immediately neutralized with 150 μL of 1 M Tris-HCl (pH 8.8). XL1-blue was grown to OD-₆₀₀=0.5˜1.0 in 10 mL of a SB medium, and 1.15 mL of the neutralized phages were added for standing incubation at 37° C. for 30 minutes. After 30 minutes, incubation was further performed at 37° C. and 120 rpm.

To calculate the phage output, XL1-blue was diluted 1:10, 1:100, or 1:1000, and 100 μL each of the X11-blue was spread on a SB plate with 100 mg/mL carbenicillin to incubate at 37° C. overnight. The aforementioned XL1-blue was grown with 10 μL carbenicillin (100 mg/mL) and 10 μL tetracycline (50 mg/mL) at 37° C. and 250 rpm for 2 hours. After two hours, a helper phage (10¹² pfu) was added and standing-incubated at 37° C. for 30 minutes. After 30 minutes, incubation was further performed at 37° C. and 120 rpm. In addition, cells grown with 100 μL kanamycin (50 mg/mL) and 100 μL carbenicillin (100 mg/mL) in 90 mL of a SB medium were transferred to a 500 ml flask and grown at 37° C. and 250 rpm overnight. The next day, the cultured cells were centrifuged at 3,500 rpm for 15 minutes, and the supernatant was transferred to a new tube. For phage precipitation, 20 mL of 20% PEG+2.5 M NaCl was added and the cells were incubated on ice for 1 hour. The cells were centrifuged at 8000 rpm for 40 minutes to remove the supernatant. 1 mL of phage PBSB (PBS+1% BSA) on the tube wall was resuspended and transferred to an e-tube and further centrifuged at 13000 rpm for 10 minutes, thereby recovering the supernatant. The above process was repeated by lowering to 3 μg/mL of DS-Cav1 (2nd panning).

### 3.2. Single-Colony Screening

96 individual clones collected from a colony obtained by library panning were seeded in a 96-well cell culture plate (U-bottom) filled with 500 μL SBC (SB+ carbenicillin 100 μg/mL). Negative clones were seeded, and each plate was covered with a breathable sealing film to grow the cells at 250 rpm and 37° C. to OD₆₀₀ 0.8˜ 1.0. After one hour, 50 μL SBC+10 mm IPTG was further added, and incubation was further performed at 250 rpm and 25° C. overnight. The next day, the supernatant was recovered by centrifugation at 3200×g for 20 minutes. The recovered supernatant (scFv) was stored at 4° C.

To select scFv (1st, 2nd panning output) specifically binding to DS-Cav1, screening was performed by ELISA. An ELISA plate was coated with 50 μL of 3 μg/m DS-Cav1 (in-house) at 4° C. overnight. The next day, the DS-Cav1 on the ELISA plate was removed and washed with PBS+Tween 20 0.05% three times. Each antigen-coated well was blocked with 200 μL of PBS+skim milk 5% at 37° C. for 1 hour. The plate was washed four times with PBS+Tween 20 0.05%. 50 μL of scFv was added to each well to allow binding at 37° C. for 1 hour. Again, the plate was washed with PBS+Tween 20 0.05% four times. 50 μL of an HRP-conjugated anti-HA antibody (3000:1) was added to each well and incubated at 37° C. for 1 hour. The plate was washed with PBS+Tween 20 0.05% four times. 50 μL of a TMB solution was added to each well and the cells were incubated for 1 to 15 minutes. 50 μL of a stop solution was added to each well to stop the reaction. After measurement with an ELISA reader, only wells with OD₄₅₀≥ 0.5 were selected. The selected clone was inoculated into 3 mL of SB medium and grown at 250 rpm and 37° C. overnight. The next day, DNA sequencing of the cultured cells was requested.

### 3.3. IgG Expression Vector Cloning

### Insert Production

Insert production was performed by a method using gBlock and a PCR method. The cases using gBlock include a clone in which an open reading frame of scFv is broken and a clone in which a framework different from a germline is confirmed. The clone having a framework different from a germline was manufactured in two formed of a sequence before editing and a sequence after editing. The sequence before editing was prepared by PCR and gBlock was used as the sequence after editing. Since the clone was modified after editing, it was represented as m after the clone numbering name. When there is a clone in which any one of a heavy chain and a light chain is modified, there is an m-attached clone.

In addition, by the PCR method, an insert was obtained using scFv as a template. PCR was performed after 10 μL of Expand High Fidelity^{PLUS} Reaction Buffer (5×) with 7.5 mM MgCl₂, 1 μL of dNTP (NEB, 10 mm), 2 μL of HC/LC/KC forward primer (Heavy chain/Lambda chain/Kappa chain forward primer, 10 μm), 2 μL of HC/LC/KC reverse primer (HC/LC/KC reverse primer, 10 μm), 30.5 μL of water, and 0.5 μL, scFv, and 2 μL of Expand High Fidelity^{PLUS} Enzyme blend were mixed. PCR was performed at 94° C. for 2 minutes, repeated for 30 cycles under conditions of 94° C. for 30 seconds, 53° C. for 1 minute and 72° C. for 1 minute, and at 72° C. for 7 minutes. HC/LC/KC PCR was performed by changing only a template and a primer under the same conditions. A total of 63 clones were performed in the same manner. Additionally, from the NGS sequencing result for the chromium B cell library, 25 types of IgG1s and 6 types of IgG2s were obtained. Inserts for the obtained 31 types of IgGs were manufactured by gBlock synthesis.

TABLE 10
PCR primer sequences for cloning IgG expression vectors

| | | | SEQ |
|---|---|---|---|
| | | | ID |
| | Name | Sequences (5′-3′) | NO: |
| Heavy | HC_F1 | TGCTGTGGGTGAGTGGTACCTGTGGGGAGGTGCAGCTGGTGGAGTCTG | 509 |
| chain | | | |
| | HC_F2 | TGCTGTGGGTGAGTGGTACCTGTGGGCAGGTCACCTTGAAGGAGTCTGGG | 510 |
| | HC_F3 | TGCTGTGGGTGAGTGGTACCTGTGGGCAGGTGCAGCTGGTGCAGTCTG | 511 |
| | HC_R1 | GGGGGAAGACCGATGGGCCCTTGGTGGAGGCTGAGGAGACGGTGACCAGGGTTC | 512 |
| | HC_R2 | GGGGGAAGACCGATGGGCCCTTGGTGGAGGCTGAGGAGACAGTGACCAGGGTTCC | 513 |
| | HC_R3 | GGGGGAAGACCGATGGGCCCTTGGTGGAGGCTGAGGAGACGGTGACCGTGGTTC | 514 |
| | HC_R4 | GGGGGAAGACCGATGGGCCCTTGGTGGAGGCTGAGGAGACGGTGACCGTGGTC | 515 |
| Lambda | LC_F1 | TGCTGTGGGTGAGTGGTACCTGTGGGCAGTCTGTGCTGACGCAGCCG | 516 |
| chain | | | |
| | LC_F2 | TGCTGTGGGTGAGTGGTACCTGTGGGTCCTATGAGCTGACTCAGCCAC | 517 |
| | LC_F3 | TGCTGTGGGTGAGTGGTACCTGTGGGCAGCCTGTGCTGACTCAGCCAC | 518 |
| | LC_F4 | TGCTGTGGGTGAGTGGTACCTGTGGGCAGACTGTGGTGACTCAGGAGCC | 519 |
| | LC_F5 | TGCTGTGGGTGAGTGGTACCTGTGGGCAGTCTGCCCTGACTCAGCCTG | 520 |
| | LC_R1 | GTGGGATTGGCCTTAGGTTGGCCTAGGACGGTGACCTTGGTC | 521 |
| | LC_R2 | GTGGGATTGGCCTTAGGTTGGCCTAGGACGGTCAGCTTGGTC | 522 |
| Kappa | KC_F1 | TGCTGTGGGTGAGTGGTACCTGTGGGGATGTTGTGATGACTCAGTCTCCA | 523 |
| chain | | | |
| | KC_F2 | TGCTGTGGGTGAGTGGTACCTGTGGGGATATTGTGATGACCCAGACTCCA | 524 |
| | KC_F3 | TGCTGTGGGTGAGTGGTACCTGTGGGGACATCCAGATGACCCAGTCTCCA | 525 |
| | KC_F4 | TGCTGTGGGTGAGTGGTACCTGTGGGGAGATTGTGCTGACCCAGTCTCCA | 526 |
| | KC_F5 | TGCTGTGGGTGAGTGGTACCTGTGGGGACATCCAGTTGACCCAGTCTCCA | 527 |
| | KC_R1 | ACGCTTGGAGCGGCCACCGTACGTTTGATCTCCAGCTTGGTCCC | 528 |
| | KC_R2 | ACGCTTGGAGCGGCCACCGTACGTTTAATCTCCAGTCGTGTCCCTTG | 529 |
| | KC_R3 | ACGCTTGGAGCGGCCACCGTACGTTTGATCTCCACCTTGGTCCC | 530 |

TABLE 11
PCR sets for cloning IgG expression vectors

| | | Heavy | Heavy | Light | Light |
|---|---|---|---|---|---|
| # | Clone | chain_F | chain_R | chain_F | chain_R |
| 1 | 2G4 | HC_F1 | HC_R1 | LC_F1 | LC_R1 |
| 2 | 2H3 | HC_F1 | HC_R1 | LC_F1 | LC_R1 |
| 3 | 4H1 | HC_F1 | HC_R1 | LC_F1 | LC_R1 |
| 4 | 4E3 | HC_F1 | HC_R1 | LC_F1 | LC_R1 |
| 5 | 4H5 | HC_F1 | HC_R1 | — | — |
| 6 | 2E7 | HC_F1 | HC_R1 | LC_F2 | LC_R1 |
| 7 | 2H2 | HC_F1 | HC_R1 | LC_F1 | LC_R1 |
| 8 | 12A1 | HC_F1 | HC_R1 | LC_F1 | LC_R1 |
| 9 | 2H1 | HC_F2 | HC_R1 | LC_F1 | LC_R1 |
| 10 | 4G4 | HC_F1 | HC_R1 | LC_F1 | LC_R1 |
| 11 | 12E9 | HC_F1 | HC_R1 | LC_F4 | LC_R1 |
| 12 | 4F11 | HC_F1 | HC_R1 | LC_F3 | LC_R1 |
| 13 | 10E7 | HC_F1 | HC_R1 | LC_F1 | LC_R1 |
| 14 | 2E9 | HC_F2 | HC_R1 | LC_F1 | LC_R1 |
| 15 | 12B11 | HC_F1 | HC_R2 | LC_F2 | LC_R1 |
| 16 | 3H8 | HC_F1 | HC_R3 | LC_F3 | LC_R1 |
| 17 | 4E1 | HC_F1 | HC_R1 | LC_F2 | LC_R1 |
| 18 | 2H9 | HC_F2 | HC_R1 | LC_F2 | LC_R1 |
| 19 | 12C11 | HC_F1 | HC_R1 | LC_F1 | LC_R1 |
| 20 | 3E10 | HC_F1 | HC_R1 | LC_F3 | LC_R1 |
| 21 | 2G1 | HC_F2 | HC_R1 | LC_F1 | LC_R1 |
| 22 | 1E6 | HC_F1 | HC_R2 | LC_F1 | LC_R1 |
| 23 | 2H11 | — | — | — | — |
| 24 | 2F1 | HC_F1 | HC_R1 | LC_F1 | LC_R2 |
| 25 | 13C2 | HC_F1 | HC_R1 | LC_F1 | LC_R1 |
| 26 | 2H8 | HC_F1 | HC_R1 | LC_F3 | LC_R1 |
| 27 | 10A6 | HC_F1 | HC_R1 | LC_F2 | LC_R1 |
| 28 | 4G2 | HC_F1 | HC_R1 | LC_F3 | LC_R1 |
| 29 | 2G11 | HC_F1 | HC_R1 | LC_F1 | LC_R1 |
| 30 | 11H9 | — | — | — | — |
| 31 | 12C9 | — | — | — | — |
| 32 | 7A10 | — | — | — | — |
| 33 | 4G1 | HC_F2 | HC_R1 | LC_F1 | LC_R1 |
| 34 | 7C6 | HC_F1 | HC_R1 | — | — |
| 35 | 7A7 | — | — | — | — |
| 36 | 6A9 | — | — | — | — |
| 37 | 12B8 | — | — | — | — |
| 38 | 12F7 | — | — | — | — |
| 39 | 7A11 | — | — | — | — |
| 40 | 4H11 | HC_F3 | HC_R1 | LC_F2 | LC_R2 |
| 41 | 2G3 | HC_F3 | HC_R1 | LC_F2 | LC_R2 |
| 42 | 12C10 | HC_F3 | HC_R1 | LC_F3 | LC_R2 |
| 43 | 3E8 | HC_F3 | HC_R2 | LC_F3 | LC_R2 |
| 44 | 7D1 | HC_F3 | HC_R1 | LC_F1 | LC_R2 |
| 45 | 4G5 | HC_F3 | HC_R1 | LC_F2 | LC_R2 |
| 46 | 2E8 | HC_F3 | HC_R1 | LC_F2 | LC_R2 |
| 47 | 13H9 | — | — | LC_F2 | LC_R2 |
| 48 | 4E9 | HC_F3 | HC_R1 | LC_F1 | LC_R2 |
| 49 | 4H7 | HC_F3 | HC_R1 | LC_F2 | LC_R2 |
| 50 | 2F8 | HC_F3 | HC_R1 | LC_F2 | LC_R2 |
| 51 | 3E7 | HC_F1 | HC_R2 | — | — |
| 52 | 9H4 | HC_F3 | HC_R4 | KC_F4 | KC_R2 |
| 53 | 8B1 | HC_F3 | HC_R4 | KC_F1 | KC_R1 |
| 54 | 7G12 | — | — | — | — |
| 55 | 12F11 | — | — | KC_F1 | KC_R1 |
| 56 | 5B12 | — | — | — | — |
| 57 | 8C3 | — | — | — | — |
| 58 | 2F4 | HC_F3 | HC_R4 | KC_F2 | KC_R3 |
| 59 | 1C10 | HC_F3 | HC_R1 | KC_F2 | KC_R1 |
| 60 | 5E9 | HC_F3 | HC_R3 | KC_F1 | KC_R2 |
| 61 | 5C9 | HC_F3 | HC_R3 | KC_F1 | KC_R1 |
| 62 | 5C7 | — | — | — | — |
| 63 | 8A11 | HC_F3 | HC_R1 | KC_F1 | KC_R1 |

### Vector

As IgG conversion vectors, a pCIW_heavy chain, a pCIW_lambda chain and a pCIW_kappa chain were prepared. The PCIW_heavy chain was digested with Apa 1 (NEB, R0114L)/Kpn 1 (NEB, R0142L). The pCIW_lambda chain was digested with Bsu36i (NEB, R0524L)/kpn 1 (NEB, R0142L). The pCIW_kappa chain was digested with Bsiwi (NEB, r05531)/kpn 1 (NEB, R0142L). After enzyme digestion, gel extraction was performed. The resulting product was loaded on a 0.7% agarose gel and then a gel with a size of ˜3000 bp was cut. After adding a 3-fold larger amount QG buffer per g of the gel, the cut gel was dissolved at 50° C. After the gel was added to a column and centrifuged, and a washing process of removing the flow-through, adding 750 μL of EB and performing centrifugation again was performed, centrifugation was performed for 1 minute to dry the membrane. Two minutes after the addition of 50 μL of EB to the membrane, the resulting product was recovered by centrifugation.

### Infusion

50 ng each of the linearized pCIW vectors (pCIW_heavy chain, pCIW_lambda, and pCIW_kappa), 25 ng of purified Heavy chain/Lambda chain/Kappa chain insert DNA, and 2 μL of 5× In-Fusion HD Enzyme Premix were mixed, autoclaved distilled water was added to a final volume of 10 μL, and then the resulting product was incubated at 50° C. for 30 minutes.

### Transformation

After addition of 2 μL of a ligation product to 50 μL of XLI-blue and mixing, the resulting mixture was transferred to a cuvette. Electro transformation conditions were set to 2500v, 2000, and 25 μf, followed by application of an electric shock. After 1 mL of SB was resuspended to recover as many cells in the cuvette as possible, incubation was performed at 37° C. for 1 hour at 200 rpm. After incubation, the cells were allowed to settle, the supernatant was discarded, the pellet was resuspended at 100 μL, and then the resulting suspension was spread on a LB-carb plate.

### 3.4. Animal Cell Production

### Midi-Prep

A cell pellet was resuspended with 4 mL of PI buffer. After 4 mL of P2 buffer was added to invert 3 to 4 times, 3 minute-incubation was performed. The resulting product was transferred to a cartridge filter and incubated for 10 minutes. A residue was filtered from the sample in the cartridge filter using a piston, and only the supernatant was obtained. 2 mL of a binding buffer was added, the mixture was added to a column, and the column was washed with 700 μL of ETR buffer and 750 μL of PE buffer. Elution was performed using 200 μL of DDW.

### Transfection (Based on 30 mL)

The day before expression, Expi293 cells were subcultured to 2×10⁶ cells/mL. On the day of expression, it was confirmed whether 3×10⁶ cells/mL or more of the cells were grown. One mixture containing 15 μg of a heavy chain and 15 μg of a light chain prepared in 1.5 mL of an Opti-MEM medium and a mixture containing 80 μL of ExpiFectamine in 1.5 mL of a medium were prepared and incubated for 5 minutes at room temperature. The two mixtures were mixed and incubated for 20 to 30 minutes. The resulting mixture was added to the prepared cells (25.5 mL). After 16 to 20 hours of expression, Enhancers 1 and 2 were added at 150 μL and 1.5 mL, respectively, and then expression was performed in a shaking culture for 5 to 7 days at 37° C.

### IgG (Based on 30 mL)

1 mL of 50% protein A resin washed with PBS was added to the expressed supernatant, and inverted at 4° C. for 2 hours and at room temperature for 30 minutes. After transferring to the purification column, the resin was washed with a 20-fold or more amount of an IgG binding buffer. Elution was performed with 500 μL of an elution buffer six times or more. For each vial, a concentration was measured at a wavelength of 280 nm, and then the measurement results were collected.

A desalting column was centrifuged at 1000 g for 2 minutes to filter a preservative. After adding a PBS buffer to an amount suitable for the column, centrifugation was performed at 1000 g for 2 minutes and then the column was washed repeatedly three times. After adding each sample, centrifugation was performed at 1000 g for 2 minutes, a concentration was measured using a wavelength, and then the measurement results were collected.

### SDS-PAGE Assay

The sample and a 4× non-reducing sample buffer were mixed in a volume ratio of 3:1, heated at 90° C. for 3 minutes and then cooled. The prepared sample was loaded on a 4 to 12% Bis-Tris gel at 3 μg per well, and electrophoresis was performed. The gel was isolated and stained with a PageBlue Protein Staining Solution for 30 minutes or more, followed by destaining using DW and determining the size and purity of a protein.

### 3.5. Analysis of Binding Ability by ELISA

For the analysis of binding ability for 63 types of selected antibodies against DS-Cav1, ELISA was performed. 50 μL of 3 μg/m DS-Cav1 (In-house) was coated on an ELISA plate at 4° C. overnight. The next day, DS-Cav1 on the ELISA plate was removed and washed three times with PBS+Tween 20 0.05%. 200 μL of PBS+skim milk 5% was added to each well coated with an antigen for blocking at 37° C. for 1 hour. After blocking, the plate was washed four times with PBS+Tween 20 0.05%. Subsequently, 63 types of the selected antibodies IgGs were diluted ¼ from 2 μM. The dilution was performed a total of 11 times, making 12 points. 50 μL of the diluted antibodies were put into each well and bound at 37° C. for 1 hour. The plate was washed again with PBS+Tween 20 0.05% four times. 50 μL of an HRP conjugated anti-Fc antibody (3000:1) was added to each well and incubated at 37° C. for 1 hour. The plate was washed with PBS+Tween 20 0.05% four times. 50 μL of a TMB solution was added to each well, and incubation was performed for 1 to 15 minutes. 50 μL of a stop solution was added to each well to stop the reaction. Only clones with OD₄₅₀≥ 0.5 were selected after measurement with an ELISA reader.

### 4. Anti-RSV Neutralizing Antibody Screening: Analysis of Antibody Neutralizing Activity Using Focus Reduction Neutralization Test (FRNT)

The day before the experiment, A549 cells were prepared in a 96 well plate at 4.5×10⁴ cells/well. IgG supernatant dilutions were prepared to make 1:10, 1:100, and 1:1000 dilutions using infection media in a 96 deep well plate to a final volume of 300 μL. Titrated virus RSV A2 (ATCC, Cat #VR-1540) P3 stock was prepared by making a 1:125 dilution in infection media (final titers, first: 1:250, second: 1:500).

To a new 96 deep well plate, each of 200 μL of an IgG supernatant dilution and 200 μL of a virus dilution was mixed, and a neutralization reaction was performed at 37° C. in a 5% CO₂ incubator for 1 hour. A mixture obtained by neutralizing the prepared A549 cells was added at 25 μL/well, and incubated at 37° C. in a 5% CO₂ incubator for 2 hours (the plate was shaken every 15 minutes so as not to dry the cells). 200 μL of 0.8% methylcellulose was added to each well and the cells were cultured at 37° C. in a 5% CO₂ incubator for 3 to 4 days. Subsequently, overlay media were removed by suction and then the plate was washed with 300 μL/well of PBS once. 100% cold methanol was added at 100 μL/well, and the plate was covered with a wrap and stored at 4° C.

After fixation for more than 4 hours, methanol was removed and washed twice with 300 μL of PBS. An RSV fusion protein antibody was diluted 1:1000 in infection media and added at 100 μL/well, followed by incubation for 2 hours at 37° C. After washing three times with PBS, 100 μL of anti-human Fc IgG or anti-mouse IgG-HRP was added per well to be diluted 1:2000 in infection media, and incubated for 1 hour at 37° C. After washing with PBS three times, the washing solution was thoroughly removed, and 100 μL of a TrueBlue substrate (KPL, 5510-0030) was added per well to develop color at room temperature. After color development, the plate was washed and dried, and then Ec50 was calculated by counting spots using an ELISPOT reader.

Sample layouts used in the first and second tests are shown in Table 12 (sample plate layout for first test) and Table 13 (sample plate layout for second test).

TABLE 12

| | 1 | 2 | 3 | 4 | 5 | 6 | 7 | 8 | 9 | 10 | 11 | 12 |
|---|---|---|---|---|---|---|---|---|---|---|---|---|
| A | Blank | 5 | 16 | 24 | 32 | 40 | 48 | 58 | 73 | 81 | 91 | Virus |
| B | Ctrl 1 | 6 | 17 | 25 | 33 | 41 | 51 | 59 | 74 | 83 | 92 | control |
| C | Ctrl 2 | 7 | 18 | 26 | 34 | 42 | 52 | 60 | 75 | 84 | 93 | |
| D | Ctrl 3 | 10 | 19 | 27 | 35 | 43 | 53 | 61 | 76 | 85 | 94 | |
| E | Ctrl 4 | 11 | 20 | 28 | 36 | 44 | 54 | 62 | 77 | 86 | Ctrl 1 | Cell |
| F | 1 | 12 | 21 | 29 | 37 | 45 | 55 | 63 | 78 | 87 | Ctrl 2 | control |
| G | 2 | 14 | 22 | 30 | 38 | 46 | 56 | 68 | 79 | 89 | Ctrl 3 | |
| H | 4 | 15 | 23 | 31 | 39 | 47 | 57 | 69 | 80 | 90 | Ctrl 4 | |

TABLE 13

| | 1 | 2 | 3 | 4 | 5 | 6 | 7 |
|---|---|---|---|---|---|---|---|
| A | 3 | 11 m | 21 m | 42 m | 52 m | Ctrl 2 | Virus |
| B | 8 | 12 m | 27 m | 43 m | 53 m | Ctrl 3 | control |
| C | 9 | 14 m | 30 m | 45 m | 55 m | Ctrl 4 | |
| D | 13 | 15 m | 33 m | 46 m | 62 m | Blank | |
| E | 49 | 16 m | 34 m | 47 m | 71 | X | Cell |
| F | 50 | 17 m | 38 m | 49 m | 82 | X | control |
| G | 6 m | 18 m | 40 m | 50 m | 88 | X | |
| H | 9 m | 20 m | 41 m | 51 m | Ctrl 1 | X | |

### 5. RSV Neutralizing Antibody Measurement (FRNT) of Selected Antibody

Among antibody clones selected from a phage display library or NGS analysis, RSV neutralizing antibodies against the antibodies whose RSV neutralizing activity was primarily confirmed were measured through the above-described procedures. Antibody sample data used in this example is shown in the following tables.

TABLE 14

| # | Sample list |
|---|---|
| 1^{st} | 4, 5, 6, 7, 10, 11, 12, 14, 15, 16, 17, 18, 19, 20, 21, 22, 23, 26, |
| | 33, 38, 40, 42, 43, 45, 46, 47, 48, 51, 52, 53, 61, 62, 63 |
| 2^{nd} | 3, 8, 9, 13, 49, 6m, 11m, 12m, 14m |
| 3^{rd} | 1, 2, 15m, 16m, 17m, 18m, 20m, 21m, 24, 25, 27, 27m, 28, 29, |
| | 33m, 38m, 40m, 41, 41m, 42m, 45m, 46m, 47m, 49m, 50m, |
| | 51m, 52m, 53m, 60, 62m, 84, 88 |
| 4^{th} | 30, 30m, 32, 34, 36, 39, 44, 55, 55m, 56, 57, 58, 66, 68, 69, 71, |
| | 74, 75, 76, 78, 79, 80, 81, 82, 85, 87, 89, 90, 93, 86 |
| 5^{th} | 9m, 35, 37, 50, 59, 91, 94 |
| 6^{th} (3^{rd} | 1, 2, 15m, 16m, 17m, 18m, 20m, 21m, 24, 25, 27, 27m, 28, 29, |
| re-test) | 33m, 38m, 40m, 41, 41m, 42m, 45m, 46m, 47m, 49m, 50m, |
| | 51m, 52m, 53m, 60, 62m, 84, 88 |

TABLE 15

| # | Clone | Name | Library | Expression (mg/L) | Kd (~nM) |
|---|---|---|---|---|---|
| 1 | #1 | 2G4 | C-λ | 32 | 1.01 |
| 2 | #2 | 2H3 (2F5) | C-λ | 66 | 0.98 |
| 3 | #3 | 4H1 | C-λ | 75 | 1.73 |
| 4 | #4 | 4E3 | C-λ | 41 | 0.33 |
| 5 | #5 | 4H5 | C-λ | 297 | 0.23 |
| 6 | #6 | 2E7 | C-λ | 350 | 0.40 |
| 7 | #6m | | | 375 | 2.34 |
| 8 | #7 | 2H2 | C-λ | 127 | 0.44 |
| 9 | #8 | 12A1 | C-λ | 245 | 1.66 |
| 10 | #9 | 2H1 | C-λ | 310 | 1.95 |
| 11 | #9m | | | 424 | |
| 12 | #10 | 4G4 | C-λ | 288 | 0.38 |
| 13 | #11 | 12E9 | C-λ | 437 | 0.57 |
| 14 | #11m | | | 240 | 2.01 |
| 15 | #12 | 4F11 | C-λ | 324 | 0.43 |
| 16 | #12m | | | 235 | 1.82 |
| 17 | #13 | 10E7 | M-λ | 220 | 1.73 |
| 18 | #14 | 2E9 | C-λ | 185 | 0.48 |
| 19 | #14m | | | 225 | 1.34 |
| 20 | #15 | 12B11 | C-λ | 376 | 0.72 |
| 21 | #15m | | | 60 | 1.20 |
| 22 | #16 | 3H8 | M-λ | 359 | 0.51 |
| 23 | #16m | | | 118 | 0.97 |
| 24 | #17 | 4E1 | C-λ | 380 | 0.56 |
| 25 | #17m | | | 32 | 1.13 |
| 26 | #18 | 2H9 | C-λ | 55 | 0.38 |
| 27 | #18m | | | 27 | 1.84 |
| 28 | #19 | 12C11 | C-λ | 220 | 0.72 |
| 29 | #20 | 3E10 | M-λ | 306 | 0.52 |
| 30 | #20m | | | 54 | 0.88 |
| 31 | #21 | 2G1 | C-λ | 140 | 0.55 |
| 32 | #21m | | | 22 | 0.94 |
| 33 | #22 | 1E6 | M-λ | 196 | 0.34 |
| 34 | #23 | 2H11 | C-λ | 96 | 0.45 |
| 35 | #24 | 2F1 | C-λ | 16 | 0.66 |
| 36 | #25 | 13C2 | C-λ | 90 | 1.16 |
| 37 | #26 | 2H8 | C-λ | 248 | 0.42 |
| 38 | #27 | 10A6 | M-λ | 96 | 1.56 |
| 39 | #27m | | | 75 | 1.41 |
| 40 | #28 | 4G2 | C-λ | 128 | 1.05 |
| 41 | #29 | 2G11 (2F12) | C-λ | 64 | 0.85 |
| 42 | #30 | 11H9 | M-λ | | |
| 43 | #30m | | | | |
| 44 | #31 | 12C9 | C-λ | x | |
| 45 | #32 | 7A10 | C-λ | | |
| 46 | #33 | 4G1 | C-λ | 95 | 0.27 |
| 47 | #33m | | | 104 | 0.97 |
| 48 | #34 | 7C6 | C-λ | x | |
| 49 | #34m | | | x | |
| 50 | #35 | 7A7 | C-λ | 369 | |
| 51 | #36 | 6A9 | C-λ | | |
| 52 | #37 | 12B8 | C-λ | 13 | |
| 53 | #38 | 12F7 | C-λ | 584 | 0.27 |
| 54 | #38m | | | 436 | 1.24 |
| 55 | #39 | 7A11 | C-λ | | |
| 56 | #40 | 4H11 | C-λ | 559 | 0.39 |
| 57 | #40m | | | 140 | 1.25 |
| 58 | #41 | 2G3 | C-λ | 134 | 2.11 |
| 59 | #41m | | | 314 | 1.23 |
| 60 | #42 | 12C10 | C-λ | 69 | 0.44 |
| 61 | #42m | | | 88 | 1.25 |
| 62 | #43 | 3E8 | C-λ | 549 | 0.45 |
| 63 | #43m | | | x | |
| 64 | #44 | 7D1 | C-λ | | |
| 65 | #45 | 4G5 | C-λ | 385 | 0.67 |
| 66 | #45m | | | 180 | 0.95 |
| 67 | #46 | 2E8 | C-λ | 584 | 0.47 |
| 68 | #46m | | | 438 | 1.26 |
| 69 | #47 | 13H9 | C-λ | 203 | 0.61 |
| 70 | #47m | | | 480 | 0.94 |
| 71 | #48 | 4E9 | C-λ | 452 | 0.70 |
| 72 | #49 | 4H7 | C-λ | 445 | 2.15 |
| 73 | #49m | | | 186 | 1.11 |
| 74 | #50 | 2F8 | M-λ, C-λ | 4 | |
| 75 | #50m | | | 60 | 1.08 |
| 76 | #51 | 3E7 | C-κ | 541 | 0.37 |
| 77 | #51m | | | 6 | 1.26 |
| 78 | #52 | 9H4 | C-κ | 84 | 0.89 |
| 79 | #52m | | | 170 | 0.89 |
| 80 | #53 | 8B1 | C-κ | 314 | 0.82 |
| 81 | #53m | | | 70 | 0.92 |
| 82 | #54 | 7G12 | C-λ | x | |
| 83 | #55 | 12F11 | C-λ | | |
| 84 | #55m | | | | |
| 85 | #56 | 5B12 | C-κ | | |
| 86 | #57 | 8C3 | C-κ | 132 | |
| 87 | #58 | 2F4 | C-λ | 104 | |
| 88 | #59 | 1C10 (1C8) | C-κ | 32 | |
| 89 | #60 | 5E9 | C-κ | 6 | 1.31 |
| 90 | #61 | 5C9 | C-κ | 187 | 0.80 |
| 91 | #62 | 5C7 | C-κ | 61 | 0.84 |
| 92 | #62m | | | 62 | 1.27 |
| 93 | #63 | 8A11 | C-κ | 66 | 0.69 |
| 94 | #64 | H1 | 10X IgG1 | =#50 | |
| 95 | #65 | H2 | | =#2 | |
| 96 | #66 | H3 | | | |
| 97 | #67 | H4 | | =#29 | |
| 98 | #68 | H5 | | x | |
| 99 | #69 | H6 | | | |
| 100 | #70 | H7 | | =#59 | |
| 101 | #71 | H8 | | | |
| 102 | #72 | H9 | | =#13 | |
| 103 | #73 | H10 | | x | |
| 104 | #74 | H11 | | | |
| 105 | #75 | H12 | | | |
| 106 | #76 | H13 | | | |
| 107 | #77 | H14 | | x | |
| 108 | #78 | H15 | | | |
| 109 | #79 | H16 | | | |
| 110 | #80 | H17 | | | |
| 111 | #81 | H18 | | | |
| 112 | #82 | H19 | | | |
| 113 | #83 | H20 | | x | |
| 114 | #84 | H21 | | 8 | 0.95 |
| 115 | #85 | H22 | | | |
| 116 | #86 | H23 | | | |
| 117 | #87 | H24 | | | |
| 118 | #88 | H25 | | 42 | 1.29 |
| 119 | #89 | G2 H1 | 10X IgG2 | | |
| 120 | #90 | G2 H2 | | | |
| 121 | #91 | G2 H3 | | | |
| 122 | #92 | G2 H4 | | x | |
| 123 | #93 | G2 H5 | | | |
| 124 | #94 | G2 H6 | | | |
| 125 | C1 | AM14 | | 21 | X |
| 126 | C2 | 5C4 | | 142 | 0.57 |
| 127 | C3 | REGN222 | | 59 | 0.19 |
| 128 | C4 | D25 | | 105 | 0.73 |
| 129 | C5 | Synagis | | | |

| | |
|---|---|
| Library | M-λ: insert prepared manually |
| | Γ- λ/C-: chromium library-derived insert |
| | 10x: synthesis after chromium library NGS assay |
| Expression | CEDEX IgG detection |
| | X: no purification |
| Kd | Affinity determination by ELISA |

The day before the experiment, A549 cells were prepared at 4.5×10⁴ cells/well in a 96 well plate. Each sample antibody was prepared to make 4× dilutions (40˜0.009 nM) with infection media from 40 nM (final 20 nM) in 7 steps in a 96 deep well plate to a final volume of 300 μL.

Titrated virus RSV A2 P3 stock was prepared to make a 1:1000 dilution with infection media. 200 μL of an IgG supernatant dilution and 200 μL of a virus dilution were mixed in a new 96 deep well plate, and a neutralization reaction was performed at 37° C. in a 5% CO₂ incubator for 1 hour. A mixture obtained by neutralizing the prepared A549 cells was added at 25 μL/well, and incubated at 37° C. in a 5% CO₂ incubator for 2 hours (the plate was shaken every 15 minutes so as not to dry the cells). 200 μL of 0.8% methylcellulose was added to each well and the cells were cultured at 37° C. in a 5% CO₂ incubator for 3 to 4 days. Overlay media was removed and the plate was washed with 300 μL/well of PBS once. 100% cold methanol was added at 100 μL/well, and the plate was covered with a wrap and stored at 4° C.

After fixation for more than 4 hours, methanol was removed and washed twice with 300 μL of PBS. An RSV fusion protein antibody was diluted 1:1000 with infection media and added at 100 μL/well, followed by incubation for 2 hours at 37° C. After washing three times with PBS, 100 μL of anti-human Fc IgG or anti-mouse IgG-HRP was added per well to be diluted 1:2000 with infection media, and incubated for 1 hour at 37° C. After washing with PBS three times, the washing solution was thoroughly removed and 100 μL of a TrueBlue substrate was added per well to develop color at room temperature. After color development, the plate was washed and dried, and then IC₅₀ was calculated by counting spots using an ELISPOT reader.

### 6. Quantitative Measurement of Binding Ability of Anti-F Antibody to F-Protein

The quantitative binding ability (affinity) to recombinant F-protein of antibodies 8, 9, 12m, 13, 15, 16, 33, 46, and 61, which have the most excellent efficacy in the neutralizing antibody analysis, was measured using Biacore T-200 (GE Healthcare). An antibody to be measured was diluted to a concentration of 0.3 μg/mL in an HBS-EP buffer solution (10 mM HEPES (pH7.4), 150 mM NaCl, 3 mM EDTA, 0.005% surfactant P20) on a protein A chip (CAT. No. BR-1005-XX, GE Healthcare) and captured up to 200 Ru while flowing at a flow rate of 30 μL/min for 1 minute. The purified F-protein was sequentially diluted to a concentration range from 0.04 nM to 10 nM in an HBS-EP buffer solution and subjected to association for 12 seconds and dissociation for 1800 seconds while flowing at a flow rate of 30 μL/min. By flowing 10 mM glycine-HCl pH 2.0 for 30 seconds at a flow rate of 30 μL/min, the dissociation of the captured antibody onto the chip was induced. Affinity was obtained from kinetic rate constants (Kₒₙ and K_{off}) and an equilibrium dissociation constant (K_{D}) using Biacore T-200 evaluation software.

### Example 1. Result of production of RSV F-protein

### 1.1. RSV F-Protein Subtype Production and Purification

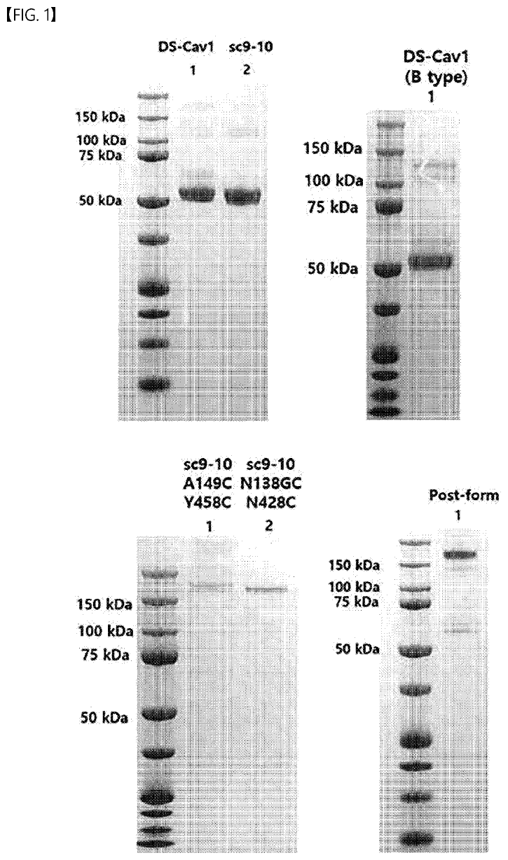

The RSV F-protein is composed of a trimer and its expected size is approximately 180 kDa. The expressed F-protein formed a trimer by a fibritin trimerization domain at the C-terminus. In the cases of the pre-forms of the F protein, DS-Cav1 and DS-Cav1 sc9-10, in SDS-PAGE (a non-covalent bond was broken), it was confirmed that the binding of the fibritin trimerization domain was broken such that most of the F-protein was observed in the form of a monomer. However, in the cases of sc9-10 A149C Y458C and sc9-10 N138GC N428C that have structural stability due to the addition of a covalent bond (disulfide bond), and the post-form F-protein transformed to a more stable structure than a pre-form protein, it was confirmed that even when the binding of the fibritin trimerization domain was broken, the F proteins may be stably present in the form of a trimer (FIG. 1).

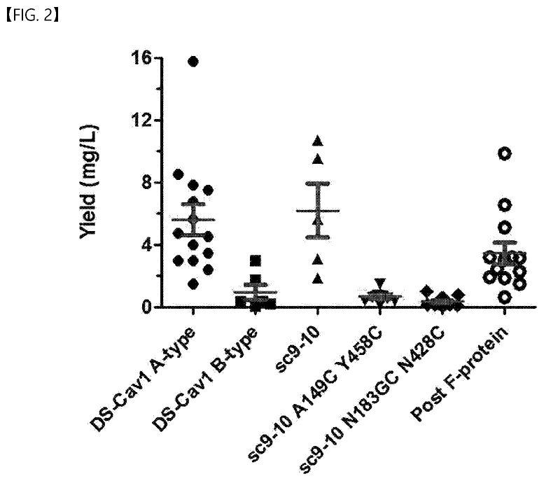

As a result of analyzing the expression rate of the RSV F-protein, it was found that the DS-Cav1 A-type has a better expression rate than the B-type (FIG. 2). In the A-type, it can be seen that the engineered clone sc9-10 has no significant difference in expression level from DS-Cav1, but for a stable trimer form in sc9-10, sc9-10 A149C Y458C and sc9-10 N183GC N428C, which have mutations, exhibit very low expression levels. Overall, depending on the cell passage of Expi293 cells, it was confirmed that there is a difference in expression level. It was confirmed that the longer the cell passage, the lower the expression level, which can eventually affect a yield.

### Example 2. Construction of Human B Cell-Derived Library

### 2.1. Human PBMC Isolation

All PBMCs isolated from the blood collected from a healthy adult had a viability of 99%, and all PBMCs isolated from the blood collected from RSV-convalescent and healthy infants had a viability of 97 to 99%. Each type of cells was dispensed to have a concentration of 1×10⁷ cells per vial and then stored in liquid nitrogen.

### 2.2. Isolation of B Cells Specific for RSV F-Protein (DS-Cav1)

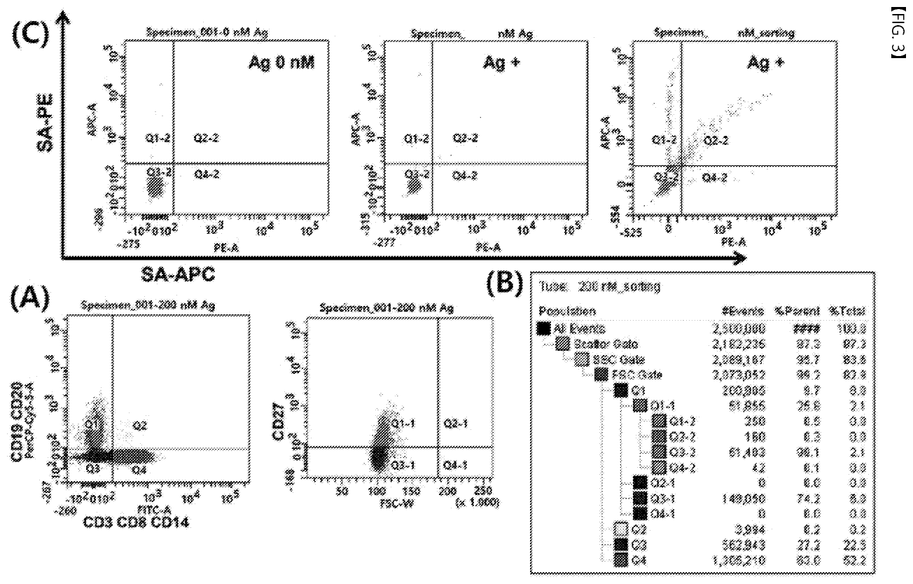

Among lymphocytes of PBMCs, it was confirmed that B cells CD3-CD8 CD14-CD19+CD20+were approximately 8% of the total cells, and memory B cells CD27+were approximately 25% of the B cells. Among memory B cells, cells that are double-positive for APC and PE of the RSV F-protein were approximately 0.3%, and approximately 1000 cells of a total of 3.8×10⁷ cells were collected in 0.5 mL of 2% FBS/PBS (FIGS. 3A-3C).

### 2.3. Construction of B Cell-Derived V(D)J Library

FACS-sorted B cells lysed with Quick extraction buffer were subjected to RT-PCR and cDNA amplification for Ig transcripts using a SMARTer RACE 5′ kit and a RT-IgGKLA primer. The cDNA which had experienced RT-PCR and PCR cleanup was immediately used in a volume of approximately 15 μL to construct a phage-displayed scFv library without gel electrophoresis.

2.4. Construction of chromium single cell V(D)J library

Chromium single cell V(D)J libraries were constructed for memory B cells (Ag⁺CD27⁺ B cells) double-positive for RSV F, memory B cells (Ag⁻CD27⁺ B cells) double-negative for RSV F used as a control, and unsorted PBMCs used as a control. The concentration after cDNA amplification of Ag⁺CD27⁺ B cells (1.82 ng/μL), Ag⁻CD27⁺ B cells (0.383 ng/μL), and PBMC (1.57 ng/μL) was 45.5 μL, and the concentration after a target enrichment step of the Ag⁺CD27⁺ B cells (11.2 ng/μL), the Ag⁻CD27⁺ B cells (1.57 ng/μL), and the PBMC (0.542 ng/μL) was 45.5 μL. The concentration of the final sequencing library after enzymatic fragmentation and cleanup of the Ag⁺CD27⁺ B cells (69.4 ng/μL), the Ag⁻ CD27⁺ B cells (31.2 ng/μL), and the PBMC (15.4 ng/μL) was 35 μL. All concentrations were measured with Qubit.

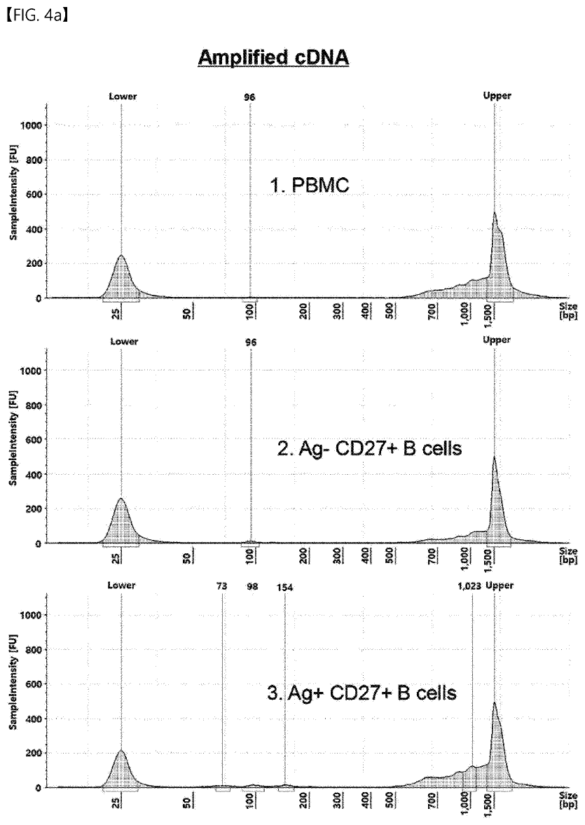

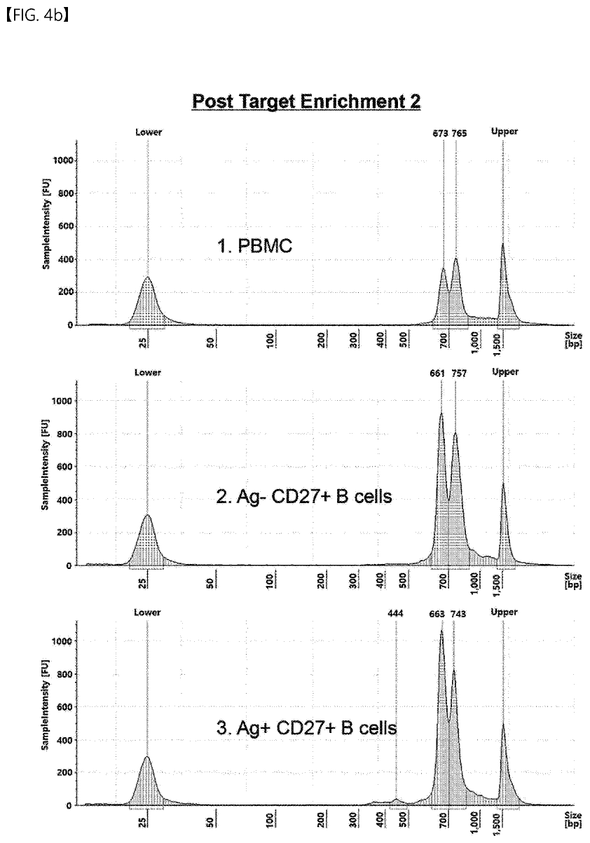

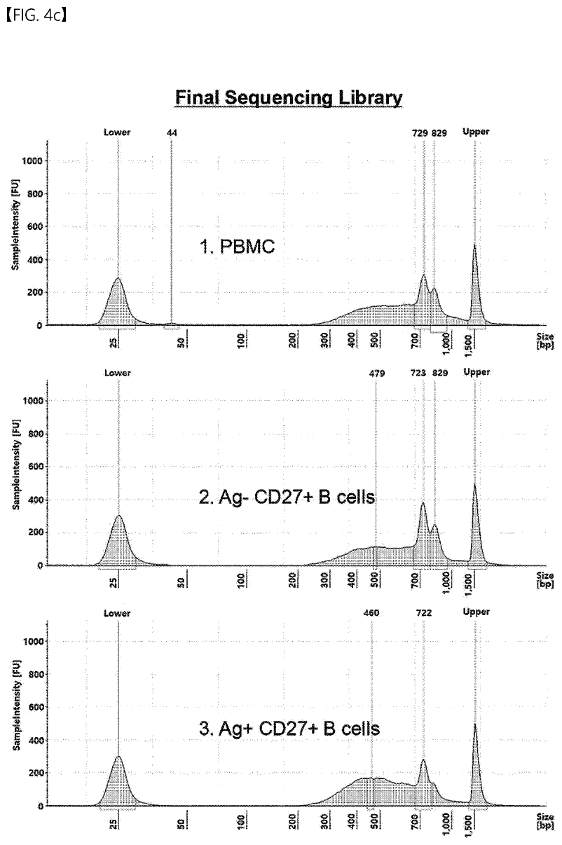

The result of QC performed after each step using Agilent Tapestation is shown in FIGS. 4a-4c. In post target enrichment, Ig transcripts were observed between 600 to 800 bp, and in the final sequencing library step, traces of enzymatic fragmentation were confirmed.

2.5. Construction of Phage Displayed scFv Library

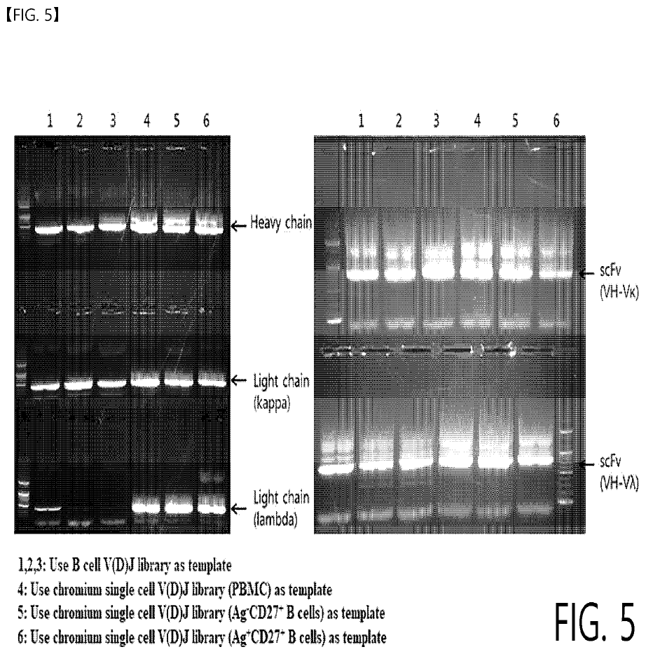

Each phage-displayed scFv library was constructed using the V(D)J library constructed as described above as a template. The results of VH/V_{κ}/V_{λ} PCR and scFv overlapping PCR are shown in FIG. 5.

### Example 3. Results of Selecting and Manufacturing Anti-RSV Antibody

TABLE 16
Result of anti-DS-Cav1 phage display library panning

| | Antigen | | | | |
|---|---|---|---|---|---|
| | (DS-Cav1, | | | | Output |
| Round | In house) | Washing | Insert | Template cDNA | titer |
| 1^{st} | 5 μg/mL | PBST X 5 | VH-Vκ | Ag (+) memory | 3.3 × 10⁷ |
| | | | | B cell pool | |
| | | | | Chromium B | 3.4 × 10⁷ |
| | | | | cell + PBMC | |
| | | | | Chromium Ag (+) | 1.4 × 10⁷ |
| | | | | memory B cell | |
| | | | VH-Vλ | Ag (+) memory | 1.1 × 10⁷ |
| | | | | B cell pool | |
| | | | | Chromium B | 1.7 × 10⁷ |
| | | | | cell + PBMC | |
| | | | | Chromium Ag (+) | 2.0 × 10⁷ |
| | | | | memory B cell | |
| 2^{nd} | 3 μg/mL | PBST X 7 | VH-Vκ | Ag (+) memory | 2.8 × 10⁸ |
| | | | | B cell pool | |
| | | | | Chromium B | 1.2 × 10⁸ |
| | | | | cell + PBMC | |
| | | | | Chromium Ag (+) | 7.4 × 10⁸ |
| | | | | memory B cell | |
| | | | VH-Vλ | Ag (+) memory | 9.3 × 10⁸ |
| | | | | B cell pool | |
| | | | | Chromium B | 2.5 × 10⁷ |
| | | | | cell + PBMC | |
| | | | | Chromium Ag (+) | 3.2 × 10⁹ |
| | | | | memory B cell | |

TABLE 17
Result of single colony screening

| | | 1^{st} | 2^{nd} | | |
|---|---|---|---|---|---|
| | | panning | panning | # of | |
| Insert | Template cDNA | output | output | hit | Remark |
| VH-Vκ | Ag (+) memory | 0/144 | 0/48 | 0/192 | |
| | B cell pool | | | | |
| | Chromium B | 0/48 | 0/48 | 0/96 | Negative |
| | cell + PBMC | | | | control |
| | Chromium Ag (+) | 45/336 | 3/48 | 48/384 | |
| | memory B cell | | | | |
| VH-Vλ | Ag (+) memory | 9/336 | 13/48 | 22/384 | |
| | B cell pool | | | | |
| | Chromium B | 0/48 | 0/48 | 0/96 | Negative |
| | cell + PBMC | | | | control |
| | Chromium Ag (+) | 114/336 | 38/48 | 152/384 | |
| | memory B cell | | | | |

Single colony screening for a total of 1536 samples was performed using ELISA. Among them, only 222 clones with an ELISA binding signal of 0.5 or more were sequenced. As a result of sequencing, the scFv full sequences of 133 out of 222 clones were confirmed, and except a duplicate sequence, a total of 63 unique clones were selected.

TABLE 18
Nucleotide sequences of heavy chain and
light chain variable domains of 63 types
of clones

| | | Heavy chain | | Light chain | |
|---|---|---|---|---|---|
| | | | Editing with | | Editing with |
| | | | SEQ | | SEQ |
| | | | (Nucleotides) | | (Nucleotides) |
| | | | Germline | | Germline |
| | Clone | SEQ | sequence | SEQ | sequence |
| # | name | (Nucleotides) | (Modified) | (Nucleotides) | (Modified) |
| 1 | 2G4 | GAGGTGCAGCTGGT | — | CAGTCTGTGCTGACG | — |
| | | GGAGTCTGGGGGAG | | CAGCCGCCCTCAGTG | |
| | | GCCTGGTCAAGCCT | | TCTGGGGCCCCAGG | |
| | | GGGGGGTCCCTGAG | | GCAGAGGGTCACCA | |
| | | ACTCTCCTGTGCAG | | TCTCCTGCACTGGGA | |
| | | CCTCTGGATTCACG | | GCAGCTCCAACATCG | |
| | | TTCAGTAGCTATAC | | GGGCAGGTTATGAT | |
| | | CATGCACTGGGTCC | | GTACACTGGTACCAG | |
| | | GCCAGGCTCCAGGG | | CAGGTTCCAGGAAC | |
| | | AAGGGGCTGGAGTG | | AGCCCCCAAACTCCT | |
| | | GGTCTCGTCCATAA | | CATCTTTGGTAGCAC | |
| | | CTGGTGGCAGTAGT | | CAATCGGCCCTCAGG | |
| | | TATGTCGACTACTC | | GGTCCCTGACCGATT | |
| | | AGCCTCAGTGAAGG | | CTCTGGCTCCAAGTC | |
| | | GCCGATTCACCATC | | TGGCACCTCAGCCTC | |
| | | TCCAGAGACAACGC | | CCTGGCCATCACTGG | |
| | | TCAGAGCTCACTTT | | CCTCCAGGCTGACGA | |
| | | ATCTGCAAATGAAC | | TGAGGCTGATTATTA | |
| | | AGCCTGAGAGCCGA | | CTGCCAGTCCTATGA | |
| | | GGACACGGCTGTGT | | CCGCAGCCTGAGTCA | |
| | | ATTACTGTGCGAGA | | TGTCTTCGGAACTGG | |
| | | GATGATTATGGTTC | | GACCAAGGTCACCG | |
| | | GGGGAGTTATTCCA | | TCCTAGGC | |
| | | ACTGGTTCGACCCC | | (SEQ ID NO: 50) | |
| | | TGGGGCCAGGGAAC | | | |
| | | CCTGGTCACCGTCT | | | |
| | | CCTCA | | | |
| | | (SEQ ID NO: 49) | | | |
| 2 | 2H3 | GAGGTGCAGCTGGT | | CAGTCTGTCCTGACG | — |
| | | GGAGTCTGGGGGAG | | CAGCCGCCCTCAGTG | |
| | | GCGTGGTCCAGTCT | | TCTGGGGCCCCAGG | |
| | | GGGAGGTCCCTGAG | | GCAGAGGGTCACCA | |
| | | ACTCTCCTGTGCAG | | TCTCCTGCACTGGGA | |
| | | GCTCTGGATTCACC | | GCAGCTCCAACATCG | |
| | | TTCAGTAGCTATAC | | GGGCAGGTTATGAT | |
| | | CATGCACTGGGTCC | | GTACACTGGTACCAG | |
| | | GCCAGGCTCCAGGG | | CAGGTTCCAGGAAC | |
| | | AAGGGGCTGGAGTG | | AGCCCCCAAACTCCT | |
| | | GGTCTCGTCCATAA | | CATCTTTGGTAGCAC | |
| | | CTGGTGGCAGTAGT | | CAATCGGCCCTCAGG | |
| | | TATGTCGACTACTC | | GGTCCCTGACCGATT | |
| | | AGCCTCAGTGAAGG | | CTCTGGCTCCAAGTC | |
| | | GCCGATTCACCATC | | TGGCGCCTCAGCCTC | |
| | | TCCAGAGACAACGC | | CCTGGCCATCACTGG | |
| | | CCAGAGCTCACTTT | | GCTCCAGACTGAGG | |
| | | ATCTGCAAATGAAC | | ATGAGGCTGATTATT | |
| | | AGCCTGAGAGCCGA | | ACTGCCAGTCCTATG | |
| | | GGACACGGCTGTGT | | ACCGCAGCCTGAGTC | |
| | | ATTACTGTGCGAGA | | ATGTCTTCGGAACTG | |
| | | GATGATTATGGTTC | | GGACCAAGGTCACC | |
| | | GGGGAGTTATTCCA | | GTCCTAGGC | |
| | | ACTGGTTCGACCCC | | (SEQ ID NO: 52) | |
| | | TGGGGCCAGGGAAC | | | |
| | | CCTGGTCACCGTCT | | | |
| | | CCTCA | | | |
| | | (SEQ ID NO: 51) | | | |
| 3 | 4H1 | GAGGTGCAGCTGGT | — | CAGTCTGTGCTGACG | |
| | | GGAGTCTGGGGGAG | | CAGCCGCCCTCAGTG | |
| | | GCCTGGTCAAGCCT | | TCTGGGGCCCCAGG | |
| | | GGGGGGTCCCTGAG | | GCAGAGGGTCACCA | |
| | | ACTCTCCTGTGCAG | | TCTCCTGCACTGGGA | |
| | | GCTCTGGATTCACC | | GCAGCTCCAACATCG | |
| | | TTCAGTAGCTATAC | | GGGCAGGTTACGAT | |
| | | CATGCACTGGGTCC | | GTACACTGGTACCAG | |
| | | GCCAGGCTCCAGGG | | CAGGTTCCAGGAAC | |
| | | AAGGGGCTGGAGTG | | AGCCCCCAAACTCCT | |
| | | GGTCTCGTCCATAA | | CATCTTTGGTAGCAC | |
| | | CTGGTGGCAGTAGT | | CAATCGGCCCTCAGG | |
| | | TATGTCGACTACTC | | GGTCCCTGACCGATT | |
| | | AGCCTCAGTGAAGG | | CTCTGGTTCCAAGTC | |
| | | GCCGATTCACCATC | | TGGCGCCTCTGCCTC | |
| | | TCCAGAGACAACGC | | CCTGGCCATCACTGG | |
| | | CCAGAGCTCACTTT | | CCTCCAGGCTGACGA | |
| | | ATCTGCAAATGAAC | | TGAGGCTGATTATTA | |
| | | AGCCTGAGAGCCGA | | CTGCCAGCCCTATGA | |
| | | GGACACGGCTGTGT | | CCGCAGCCTGAGTCA | |
| | | ATTACTGTGCGAGA | | TGTCTTCGGAACTGG | |
| | | GATGATTATGGTTC | | GACCAAGGTCACCG | |
| | | GGGGAGTTATTCCA | | TCCTACGC | |
| | | ACTGGTTCGACCCC | | (SEQ ID NO: 54) | |
| | | TGGGGCCAGGGAAC | | | |
| | | CCTGGTCACCGTCT | | | |
| | | CCTCA | | | |
| | | (SEQ ID NO: 53) | | | |
| 4 | 4E3 | GAGGTGCAGCTGGT | — | CAGTCTGTGCTGACG | — |
| | | GGAGTCTGGGGGAG | | CAGCCGCCCTCAGTG | |
| | | GCCTGGTCAAGCCT | | TCTGGGGCCCCAGG | |
| | | GGGGGGTCCCTGAG | | GCAGAGGGTCACCA | |
| | | ACTCTCCTGTGCAG | | TCTCCTGCACTGGGA | |
| | | GCTCTGGATTCACC | | GCAGCTCCAACATCG | |
| | | TTCAGTAGCTATAC | | GGGCAGGTTACGAT | |
| | | CATGCACTGGGTCC | | GTACACTGGTACCAG | |
| | | GCCAGGCTCCAGGG | | CAGGTTCCAGGAAC | |
| | | AAGGGGCTGGAGTG | | AGCCCCCAAACTCCT | |
| | | GGTCTCGTCCATAA | | CATCTTTGGTAGCAC | |
| | | CTGGTGGCAGTAGT | | CAATCGGCCCTCAGG | |
| | | TATGTCGACTACTC | | GGTCCCTGACCGATT | |
| | | AGCCTCAGTGAAGG | | CTCTGGCTCCAAGTC | |
| | | GCCGATTCACCATC | | TGGCGCCTCTGCCTC | |
| | | TCCAGAGACAACGC | | CCTGGCCATCACTGG | |
| | | CCAGAGCTCACTTT | | CCTCCAGGCTGACGA | |
| | | ATCTGCAAATGAAC | | TGAGGCTGATTATTA | |
| | | AGCCTGAGAGCCGA | | CTGCCAGTCCTATGA | |
| | | GGACACGGCTGTGT | | CCGCAGCCTGAGTCA | |
| | | ATTACTGTGCGAGA | | TGTCTTCGGAACTGG | |
| | | GATGATTATGGTTC | | GACCAAGGTCACCG | |
| | | GGGGAGTTATTCCA | | TCCTAGGC | |
| | | ACTGGTTCGACCCC | | (SEQ ID NO: 56) | |
| | | TGGGGCCAGGGAAC | | | |
| | | CCTGGTCACCGTCT | | | |
| | | CCTCA | | | |
| | | (SEQ ID NO: 55) | | | |
| 5 | 4H5 | GAGGTGCAGCTGGT | — | CAGTCTGTGCTGACG | — |
| | | GGAGTCTGGGGGAG | | CAGCCGCCCTCAGTG | |
| | | GCCTGGTCAAGCCT | | TCTGGGGCCCCAGG | |
| | | GGGGGGTCCCTGAG | | GCAGAGGGTCACCA | |
| | | ACTCTCCTGTGCAG | | TCTCCTGCACTGGGA | |
| | | GCTCTGGATTCACC | | GCAGCTCCAACATCG | |
| | | TTCAGTAGCTATAC | | GGGCAGGTTATGAT | |
| | | CATGCACTGGGTCC | | GTACACTGGTATCAG | |
| | | GCCAGGCTCCAGGG | | CAGCTTCCGGGAGC | |
| | | AAGGGGCTGGAGTG | | AGCCCCCAGACTCCT | |
| | | GGTCTCGTCCATAA | | CATGTTTGGTAACAG | |
| | | CTGGTGGCAGTAGT | | CAATCGGCCCTCGGG | |
| | | TATGTCGACTACTC | | GGTACCTGACCGCTT | |
| | | AGCCTCAGTGAAGG | | CTCTGGCTCCAAGTC | |
| | | GCCGATTCACCATC | | TGGCACCTCCGCCTC | |
| | | TCCAGAGACAACGC | | CCTGGCCATCACTGG | |
| | | CCAGAGCTCACTTT | | TCTCCAGGCTGAGGA | |
| | | ATCTGCAAATGAAC | | TGAGGCTGATTATTA | |
| | | AGCCTGAGAGCCGA | | CTGCCAGTCCTATGA | |
| | | GGACACGGCTGTGT | | CCGCAGCCTGAGTCA | |
| | | ATTACTGTGCGAGA | | TGTCTTCGGAACTGG | |
| | | GATGATTATGGTTC | | CACCAAGGTGACCG | |
| | | GGGGAGTTATTCCA | | TCCTCGGC | |
| | | ACTGGTTCGACCCC | | (SEQ ID NO: 58) | |
| | | TGGGGCCAGGGAAC | | | |
| | | CCTGGTCACCGTCT | | | |
| | | CCTCA | | | |
| | | (SEQ ID NO: 57) | | | |
| 6 | 2E7 | GAGGTGCAGCTGGT | | TCCTATGAGCTGACT | CAGTCTGTG |
| | | GGAGTCTGGGGGAG | | CAGCCACCCTCAGCG | CTGACGCAG |
| | | GCCTGGTCAAGCCT | | TCTGGGACCCCCGGG | CCGCCCTCA |
| | | GGGGGGTCCCTGAG | | CAGAGGGTCACCAT | GCGTCTGGG |
| | | ACTCTCCTGTGCAG | | CTCCTGCACTGGGAG | ACCCCCGGG |
| | | GCTCTGGATTCACC | | CAGCTCCAACATCGG | CAGAGGGTC |
| | | TTCAGTAGCTATAC | | GGCAGGTTATGATGT | ACCATCTCC |
| | | CATGCACTGGGTCC | | ACACTGGTATCAGCA | TGCACTGGG |
| | | GCCAGGCTCCAGGG | | GCTTCCGGGAGCAG | AGCAGCTCC |
| | | AAGGGGCTGGAGTG | | CCCCCAGACTCCTCA | AACATCGGG |
| | | GGTCTCGTCCATAA | | TGTTTGGTAACAGCA | GCAGGTTAT |
| | | CTGGTGGCAGTAGT | | ATCGGCCCTCGGGG | GATGTACAC |
| | | TATGTCGACTACTC | | GTACCTGACCGCTTC | TGGTATCAG |
| | | AGCCTCAGTGAAGG | | TCTGGCTCCAAGTCT | CAGCTTCCG |
| | | GCCGATTCACCATC | | GGCACCTCCGCCTCC | GGAGCAGCC |
| | | TCCAGAGACAACGC | | CTGGCCATCACTGGC | CCCAGACTC |
| | | CCAGAGCTCACTTT | | CTCCAGGCTGACGAT | CTCATGTTT |
| | | ATCTGCAAATGAAC | | GAGGCTGATTATTAC | GGTAACAGC |
| | | AGCCTGAGAGCCGA | | TGCCAGTCCTATGAC | AATCGGCCC |
| | | GGACACGGCTGTAT | | CGCAGCCTGAGTCAT | TCGGGGGTA |
| | | ATTACTGTGCGAGA | | GTCTTCGGAACTGGG | CCTGACCGC |
| | | GATGATTATGGTTC | | ACCAAGGTCACCGTC | TTCTCTGGC |
| | | GGGGAGTTATTCCA | | CTAGGC | TCCAAGTCT |
| | | ACTGGTTCGACCCC | | (SEQ ID NO: 59) | GGCACCTCC |
| | | TGGGGCCAGGGAAC | | | GCCTCCCTG |
| | | CCTGGTCACCGTCT | | | GCCATCACT |
| | | CCTCA | | | GGCCTCCAG |
| | | (SEQ ID NO: 44) | | | GCTGACGAT |
| | | | | | GAGGCTGAT |
| | | | | | TATTACTGC |
| | | | | | CAGTCCTAT |
| | | | | | GACCGCAGC |
| | | | | | CTGAGTCAT |
| | | | | | GTCTTCGGA |
| | | | | | ACTGGG |
| | | | | | ACCAAGGTC |
| | | | | | ACCGTCCTA |
| | | | | | GGC |
| | | | | | (SEQ ID NO: |
| | | | | | 45) |
| 7 | 2H2 | CAGGTGCAGCTGGT | — | CAGTCTGTGCTGACG | — |
| | | GCAGTCTGGGGGAG | | CAGCCGCCCTCAGTG | |
| | | GCTTGATACAGCCT | | TCTGGGGCCCCAGG | |
| | | GGGGGGTCCCTGAG | | GCAGAGGGTCACCA | |
| | | ACTCTCCTGTGCAG | | TCTCCTGCACTGGGA | |
| | | GCTCTGGATTCACC | | GCAGCTCCAACATCG | |
| | | TTCAGTAGCTATAC | | GGGCAGGTTATGAT | |
| | | CATGCACTGGGTCC | | GTACACTGGTACCAG | |
| | | GCCAGGCTCCAGGG | | CAACTTCCAGGAAC | |
| | | AAGGGGCTGGAGTG | | AGCCCCCAAACTCCT | |
| | | GGTCTCGTCCATAA | | CATCTTTGCTAACAC | |
| | | CTGGTGGCAGTAGT | | CAATCGGCCCTCAGG | |
| | | TATGTCGACTACTC | | GGTCCCTGATCGATT | |
| | | AGCCTCAGTGAAGG | | CTCTGGCTCCAAGTC | |
| | | GCCGATTCACCATC | | TGGCGCCTCTGCCTC | |
| | | TCCAGAGACAACGC | | CCTGGCCATCACTGG | |
| | | CCAGAGCTCACTTT | | CCTCCAGGCTGACGA | |
| | | ATCTGCAAATGAAC | | TGAGGCTGATTATTA | |
| | | AGCCTGAGAGCCGA | | CTGCCAGTCCTATGA | |
| | | GGACACGGCCGTGT | | CCGCAGCCTGAGTCA | |
| | | ATTACTGTGCGAGA | | TGTCTTCGGAACTGG | |
| | | GATGATTATGGTTC | | GACCAAGGTCACCG | |
| | | GGGGAGTTATTCCA | | TCCTAGGC | |
| | | ACTGGTTCGACCCC | | (SEQ ID NO: 61) | |
| | | TGGGGCCAGGGAAC | | | |
| | | CCTGGTCACCGTCT | | | |
| | | CCTCA | | | |
| | | (SEQ ID NO: 60) | | | |
| 8 | 12A1 | GAGGTGCAGCTGGT | — | CAGTCTGTCCTGACG | — |
| | | GGAGTCTGGGGGAG | | CAGCCGCCCTCAGTG | |
| | | GCCTGATACAGCCT | | TCTGGGGCCCCAGG | |
| | | GGGGGGTCCCTGAG | | GCAGAGGGTCACCA | |
| | | ACTCTCCTGTGCAG | | TCTCCTGCACTGGGA | |
| | | GCTCTGGATTCACC | | GCAGCTCCGACATCG | |
| | | TTCAGTAGCTATAC | | GGGCAGGTTATGAT | |
| | | CATGCACTGGGTCC | | GTACACTGGTACCAG | |
| | | GCCAGGCTCCAGGG | | CAACTTCCAGGAAC | |
| | | AAGGGGCTGGAGTG | | AGCCCCCAAACTCCT | |
| | | GGTCTCGTCCATAA | | CATCTTTGCTAACAC | |
| | | CTGGTGGCAGTAGT | | CAATCGGCCCTCAGG | |
| | | TATGTCGACTACTC | | GGTCCCTGATCGATT | |
| | | AGCCTCAGTGAAGG | | CTCTGGCTCCAAGTC | |
| | | GCCGATTCACCATC | | TGGCGCCTCTGCCTC | |
| | | TCCAGAGACAACGC | | CCTGGCCATCACTGG | |
| | | CCAGAGCTCACTTT | | CCTCCAGGCTGACGA | |
| | | ATCTGCAAATGAAC | | TGAGGCTGATTATTA | |
| | | AGCCTGAGAGCCGA | | CTGCCAGTCCTATGA | |
| | | GGACACGGCCGTGT | | CCGCAGCCTGAGTCA | |
| | | ATTACTGTGCGAGA | | TGTCTTCGGAACTGG | |
| | | GATGATTATGGTTC | | GACCAAGGTCACCG | |
| | | GGGGAGTTATTCCA | | TCCTAGGC | |
| | | ACTGGTTCGACCCC | | (SEQ ID NO: 35) | |
| | | TGGGGCCAGGGAAC | | | |
| | | CCTGGTCACCGTCT | | | |
| | | CCTCA | | | |
| | | (SEQ ID NO: 34) | | | |
| 9 | 2H1 | CAGGTCACCTTGAA | TGCTGTGGGT | CAGTCTGTGCTGACG | — |
| | | GGAGTCTGGGGGAG | GAGTGGTAC | CAGCCGCCCTCAGTG | |
| | | GCGTGGTCCAGTCT | CTGTGGGGA | TCTGGGGCCCCAGG | |
| | | GGGAGGTCCCTGAG | GGTGCAGCT | GCAGAGGGTCACCA | |
| | | ACTCTCCTGTGCAG | GGTGGAGTC | TCTCCTGCACTGGGA | |
| | | CCTCTGGATTCACCT | TGGGGGAGG | GCAGCTCCAACATCG | |
| | | TCAGTAGCTATACC | CGTGGTCCA | GGGCAGGTTATGAT | |
| | | ATGCACTGGGTCCG | GTCTGGGAG | GTACACTGGTACCAG | |
| | | CCAGGCTCCAGGGA | GTCCCTGAG | CAGCTTCCAGGAAG | |
| | | AGGGGCTGGAGTGG | ACTCTCCTGT | AGCCCCCAAACTCCT | |
| | | GTCTCGTCCATAAC | GCAGCCTCT | CATCTATGCTAACAC | |
| | | TGGTGGCAGTAGTT | GGATTCACCT | CAATCGGCCCTCAGG | |
| | | ATGTCGACTACTCA | TCAGTAGCT | GGTCGCTGACCGATT | |
| | | GCCTCAGTGAAGGG | ATACCATGC | CTCTGGCTCCAAGTC | |
| | | CCGATTCACCATCT | ACTGGGTCC | TGGCGCCTCTGCCTC | |
| | | CCAGAGACAACGCC | GCCAGGCTC | CCTGGCCATCACTGG | |
| | | CAGAGCTCACTTTA | CAGGGAAGG | CCTCCAGGCTGACGA | |
| | | TCTGCAAATGAACA | GGCTGGAGT | TGAGGCTGATTATTA | |
| | | GCCTGAGAGCCGAG | GGGTCTCGTC | CTGCCAGTCCTATGA | |
| | | GACACGGCTGTGTA | CATAACTGG | CCGCAGCCTGAGTCA | |
| | | TTACTGTGCGAGAG | TGGCAGTAG | TGTCTTCGGAACTGG | |
| | | ATGATTATGGTTCG | TTATGTCGAC | GACCAAGGTCACCG | |
| | | GGGAGTTATTCCAA | TACTCAGCCT | TCCTAGGC | |
| | | CTGGTTCGACCCCT | CAGTGAAGG | (SEQ ID NO: 37) | |
| | | GGGGCCAGGGAACC | GCCGATTCA | | |
| | | CTGGTCACCGTCTC | CCATCTCCA | | |
| | | CTCA | GAGACAACG | | |
| | | (SEQ ID NO: 36) | CCCAGAGCT | | |
| | | | CACTTTATCT | | |
| | | | GCAAATGAA | | |
| | | | CAGCCTGAG | | |
| | | | AGCCGAGGA | | |
| | | | CACGGCTGT | | |
| | | | GTATTACTGT | | |
| | | | GCGAGAGAT | | |
| | | | GATTATGGTT | | |
| | | | CGGGGAGTT | | |
| | | | ATTCCAACTG | | |
| | | | GTTCGACCC | | |
| | | | CTGGGGCCA | | |
| | | | GGGAACCCT | | |
| | | | GGTCACCGT | | |
| | | | CTCCTCAGCC | | |
| | | | TCCACCAAG | | |
| | | | GGCCCATCG | | |
| | | | GTCTTCCCCC | | |
| | | | (SEQ ID NO: | | |
| | | | 62) | | |
| 10 | 4G4 | GAGGTGCAGCTGGT | — | CAGTCTGTGCTGACG | — |
| | | GGAGTCTGGGGGAG | | CAGCCGCCCTCAGTG | |
| | | GCCTGGTCAAGCCT | | TCTGGGGCCCCAGG | |
| | | GGGGGGTCCCTGAG | | GCAGAGGGTCACCA | |
| | | ACTCTCCTGTGCAG | | TTTCCTGCACTGGGA | |
| | | GCTCTGGATTCACC | | ACAGCTCCAACCTCG | |
| | | TTCAGTAGCTATAC | | GGGCAGGTTATGAT | |
| | | CATGCACTGGGTCC | | GTACACTGGTACCAG | |
| | | GCCAGGCTCCAGGG | | CAACTTCCAGGAAC | |
| | | AAGGGGCTGGAGTG | | AGCCCCCAAACTCCT | |
| | | GGTCTCGTCCATAA | | CATCTATGCTAACAC | |
| | | CTGGTGGCAGTAGT | | CAATCGGCCCTCAGG | |
| | | TATGTCGACTACTC | | GGTCCCTGACCGATT | |
| | | AGCCTCAGTGAAGG | | CTCTGGCTCCAAGTC | |
| | | GCCGATTCACCATC | | TGGCGCCTCAGCCTC | |
| | | TCCAGAGACAACGC | | CCTGGCCATCACTGG | |
| | | CCAGAGCTCACTTT | | GCTCCAGACTGAGG | |
| | | ATCTGCAAATGAAC | | ATGAGGCTGATTATT | |
| | | AGCCTGAGAGCCGA | | ACTGCCAGTCCTATG | |
| | | GGACACGGCTGTGT | | ACCGCAGCCTGAGTC | |
| | | ATTACTGTGCGAGA | | ATGTCTTCGGAACTG | |
| | | GATGATTATGGTTC | | GGACCAAGGTCACC | |
| | | GGGGAGTTATTCTA | | GTCCTAGGC | |
| | | ACTGGTTCGACCCC | | (SEQ ID NO: 64) | |
| | | TGGGGCCAGGGAAC | | | |
| | | CCTGGTCACCGTCT | | | |
| | | CCTCA | | | |
| | | (SEQ ID NO: 63) | | | |
| 11 | 12E9 | GAGGTGCAGCTGGT | — | CAGACTGTGGTGACT | CAGTCTGTG |
| | | GGAGTCTGGGGGAG | | CAGGAGCCCTCAGT | CTGACGCAG |
| | | GCCTGGTCAAGCCT | | GTCTGGGGCCCCAG | CCGCCCTCA |
| | | GGGGGGTCCCTGAG | | GGCAGAGGGTCACC | GTGTCTGGG |
| | | ACTCTCCTGTGCAG | | ATCTCCTGCACTGGG | GCCCCAGGG |
| | | CCTCTGGATTCACCT | | AGCAGCTCCAACATC | CAGAGGGTC |
| | | TCACTAGTTATAGG | | GGGGCAGGTTATGA | ACCATCTCC |
| | | ATGCATTGGGTCCG | | TGTACACTGGTACCA | TGCACTGGG |
| | | CCAGGCTCCAGGGA | | GCAGCTTCCAGGAA | AGCAGCTCC |
| | | AGGGGCTGGAGTGG | | GAGCCCCCAAACTCC | AACATCGGG |
| | | GTCTCATCAATTACT | | TCATCTTTGCTAACA | GCAGGTTAT |
| | | GGTGGTGGTAATTA | | CCAATCGGCCCTCAG | GATGTACAC |
| | | TATAGAGTACGCAG | | GGGTCCCTGACCGAT | TGGTACCAG |
| | | ACTCAGTGAAGGGC | | TCTCTGGCTCCAAGT | CAGCTTCCA |
| | | CGATTCACCATCTC | | CTGGCGCCTCTGCCT | GGAAGAGCC |
| | | CAGAGACAACGCCC | | CCCTGGCCATCACTG | CCCAAACTC |
| | | AGAGCTCACTTTAT | | GCCTCCAGGCTGACG | CTCATCTTT |
| | | CTGCAAATGAACAG | | ATGAGGCTGATTATT | GCTAACACC |
| | | CCTGAGAGCCGAGG | | ACTGCCAGTCCTATG | AATCGGCCC |
| | | ACACGGCTGTGTAT | | ACCGCAGCCTGAGTC | TCAGGGGTC |
| | | TACTGTGCGAGAGA | | ATGTCTTCGGAACTG | CCTGACCGA |
| | | TGATTATGGTTCGG | | GGACCAAGGTCACC | TTCTCTGGC |
| | | GGAGTTATTCCAAC | | GTCCTAGGC | TCCAAGTCT |
| | | TGGTTCGACCCCTG | | (SEQ ID NO: 65) | GGCGCCTCT |
| | | GGGCCAGGGAACCC | | | GCCTCCCTG |
| | | TGGTCACCGTCTCCT | | | GCCATCACT |
| | | CA | | | GGCCTCCAG |
| | | (SEQ ID NO: 46) | | | GCTGACGAT |
| | | | | | GAGGCTGAT |
| | | | | | TATTACTGC |
| | | | | | CAGTCCTAT |
| | | | | | GACCGCAGC |
| | | | | | CTGAGTCAT |
| | | | | | GTCTTCGGA |
| | | | | | ACTGGGACC |
| | | | | | AAGGTCACC |
| | | | | | GTCCTAGGC |
| | | | | | (SEQ ID NO: |
| | | | | | 39) |
| 12 | 4F11 | GAGGTGCAGCTGGT | — | CAGCCTGTGCTGACT | CAGTCTGTG |
| | | GGAGTCTGGGGGAG | | CAGCCACCCTCAGTG | CTGACGCAG |
| | | GCCTGGTCAAGCCT | | TCTGGGGCCCCAGG | CCGCCCTCA |
| | | GGGGGGTCCCTGAG | | GCAGAGGGTCACCA | GTGTCTGGG |
| | | ACTTTCCTGTGCTGG | | TCTCCTGCACTGGGA | GCCCCAGGG |
| | | CTCTGGATTCGCCTT | | GCAGCTCCAACATCG | CAGAGGGTC |
| | | CAGTAGTTACACTA | | GGGCAGGTTATGAT | ACCATCTCC |
| | | TGCACTGGGTGCGC | | GTACACTGGTACCAG | TGCACTGGG |
| | | CAGGCTCCAGGGAA | | CAGGTTCCAGGAAC | AGCAGCTCC |
| | | GGGGCTGGAGTGGG | | GGCCCCCAAACTCCT | AACATCGGG |
| | | TCTCATCCATCACTG | | CATCTTTGGTAGCAC | GCAGGTTAT |
| | | GCGGCAGTAGTTAC | | CAATCGGCCCTCAGG | GATGTACAC |
| | | CTAGACTACGCACA | | GGTCCCTGACCGATT | TGGTACCAG |
| | | CTCAGTGAAGGGCC | | CTCTGGCTCCAAGTC | CAGGTTCCA |
| | | GATTCACCATCTCC | | TGGCGCCTCAGCCTC | GGAACGGCC |
| | | AGAGACAATGGCCA | | CCTGGCCATCACTGG | CCCAAACTC |
| | | GAACTCACTGTTTCT | | GCTCCAGACTGAGG | CTCATCTTT |
| | | GCAAATGAACAGCC | | ATGAGGCTGATTATT | GGTAGCACC |
| | | TGAGGACCGAGGAC | | ACTGCCAGTCCTATG | AATCGGCCC |
| | | ACGGCTGTATATTA | | ACCGCAGCCTGAGTC | TCAGGGGTC |
| | | CTGTGCGAGAGATG | | ATGTCTTCGGAACTG | CCTGACCGA |
| | | ACTATGGTTCGGGG | | GGACCAAGGTCACC | TTCTCTGGC |
| | | AGTTATTCCAACTA | | GTCCTAGGC | TCCAAGTCT |
| | | CTTCGACCCCTGGG | | (SEQ ID NO: 66) | GGCGCCTCA |
| | | GCCAGGGAACCCTG | | | GCCTCCCTG |
| | | GTCACCGTCTCCTC | | | GCCATCACT |
| | | A | | | GGGCTCCAG |
| | | (SEQ ID NO: 47) | | | ACTGAGGAT |
| | | | | | GAGGCTGAT |
| | | | | | TATTACTGC |
| | | | | | CAGTCCTAT |
| | | | | | GACCGCAGC |
| | | | | | CTGAGTCAT |
| | | | | | GTCTTCGGA |
| | | | | | ACTGGGACC |
| | | | | | AAGGTCACC |
| | | | | | GTCGCTAGG |
| | | | | | C |
| | | | | | (SEQ ID NO: |
| | | | | | 48) |
| 13 | 10E7 | GAGGTGCAGCTGGT | — | CAGTCTGTGCTGACG | — |
| | | GGAGTCTGGGGGAG | | CAGCCGCCCTCAGTG | |
| | | GCCTGGTCAAGCCT | | TCTGGGGCCCCAGG | |
| | | GGGGGGTCCCTGAG | | GCAGAGGGTCACCA | |
| | | ACTCTCCTGTGCAG | | TCTCCTGCACTGGGA | |
| | | CCTCTGGATTCACCT | | GCAGCTCCAACATCG | |
| | | TCACTAGTTATAGG | | GGGCAGGTTATGAT | |
| | | ATGCATTGGGTCCG | | GTACACTGGTACCAG | |
| | | CCAGGCTCCAGGGA | | CAGCTTCCAGGAAG | |
| | | AGGGGCTGGAGTGG | | AGCCCCCAAACTCCT | |
| | | GTCTCATCAATTACT | | CATCTTTGCTAACAC | |
| | | GGTGGTGGTAATTA | | CAATCGGCCCTCAGG | |
| | | TATAGAGTACGCAG | | GGTCCCTGACCGATT | |
| | | ACTCAGTGAAGGGC | | CTCTGGCTCCAAGTC | |
| | | CGGTTCACCATCTC | | TGGCGCCTCTGCCTC | |
| | | CAGAGACAACGCCA | | CCTGGCCATCACTGG | |
| | | AGAACTCACTGGAT | | CCTCCAGGCTGACGA | |
| | | CTGCAAATGAACAG | | TGAGGCTGATTATTA | |
| | | CCTGAGAGCCGAGG | | CTGCCAGTCCTATGA | |
| | | ACACGGCTATTTAT | | CCGCAGCCTGAGTCA | |
| | | TACTGTGCGAGAGA | | TGTCTTCGGAACTGG | |
| | | TATGTATGGTTTGG | | GACCAAGGTCACCG | |
| | | GGAGTTATTATTCG | | TCCTAGGC | |
| | | CCTAACTACTTCGA | | (SEQ ID NO: 39) | |
| | | CTCCTGGGGCCAGG | | | |
| | | GAACCCTGGTCACC | | | |
| | | GTCTCCTCA | | | |
| | | (SEQ ID NO: 38) | | | |
| 14 | 2E9 | CAGGTCACCTTGAA | AGGTGCAGC | GTACACTGGTACCAG | — |
| | | GGAGTCTGGGGGAG | TGGTGGAGT | CAGTCTGTGCTGACG | |
| | | GCCTGGTCAAGCCT | CTGGGGGAG | CAGCCGCCCTCAGTT | |
| | | GGGGGGTCCCTGAG | GCCTGGTCA | TCTGGGGCCCCAGG | |
| | | ACTCTCCTGTGCAG | AGCCTGGGG | GCAGAGGGTCACCA | |
| | | GCTCTGGATTCACC | GGTCCCTGA | TCTCCTGCACTGGGA | |
| | | TTCAGTAGCTATAC | GACTCTCCTG | GCAGCTCCAACATCG | |
| | | CATGCACTGGGTCC | TGCAGGCTCT | GGGCAGGTTATGAT | |
| | | GCCAGGCTCCAGGG | GGATTCACCT | CAGCTTCCAGGAAG | |
| | | AAGGGGCTGGAGTG | TCAGTAGCT | AGCCCCCAAACTCCT | |
| | | GGTCTCGTCCATAA | ATACCATGC | CATCTTTGCTAACAC | |
| | | CTGGTGGCAGTAGT | ACTGGGTCC | CAATCGGCCCTCAGG | |
| | | TATGTCGACTACTC | GCCAGGCTC | GGTCCCTGACCGATT | |
| | | AGCCTCAGTGAAGG | CAGGGAAGG | CTCTGGCTCCAAGTC | |
| | | GCCGATTCACCATC | GGCTGGAGT | TGGCGCCTCTGCCTC | |
| | | TCCAGAGACAACGC | GGGTCTCGTC | CCTGGCCATCACTGG | |
| | | CAAGAACTCACTGT | CATAACTGG | CCTCCAGGCTGACGA | |
| | | TTCTGCAAATGAAC | TGGCAGTAG | TGAGGCTGATTATTA | |
| | | AGCCTGAGAGCCGA | TTATGTCGAC | CTGCCAGTCCTATGA | |
| | | GGACACGGCTGTGT | TACTCAGCCT | CCGCAGCCTGAGTCA | |
| | | ATTACTGTGCGAGA | CAGTGAAGG | TGTCTTCGGAACTGG | |
| | | GATGATTATGGTTC | GCCGATTCA | GACCAAGGTCACCG | |
| | | GGGGAGTTATTCCA | CCATCTCCA | TCCTAGGC | |
| | | ACTGGTTCGACCCC | GAGACAACG | (SEQ ID NO: 69) | |
| | | TGGGGCCAGGGAAC | CCAAGAACT | | |
| | | CCTGGTCACCGTCT | CACTGTTTCT | | |
| | | CCTCA | GCAAATGAA | | |
| | | (SEQ ID NO: 67) | CAGCCTGAG | | |
| | | | AGCCGAGGA | | |
| | | | CACGGCTGT | | |
| | | | GTATTACTGT | | |
| | | | GCGAGAGAT | | |
| | | | GATTATGGTT | | |
| | | | CGGGGAGTT | | |
| | | | ATTCCAACTG | | |
| | | | GTTCGACCC | | |
| | | | CTGGGGCCA | | |
| | | | GGGAACCCT | | |
| | | | GGTCACCGT | | |
| | | | CTCCTCA | | |
| | | | (SEQ ID NO: | | |
| | | | 68) | | |
| 15 | 12B11 | GAGGTGCAGCTGGT | — | TCCTATGAGCTGACT | CAGTCTGTG |
| | | GGAGTCTGGGGGAG | | CAGCCACCCTCAGTG | CTGACGCAG |
| | | GCGTGGTCCAGCCT | | TCTGGGGCCCCAGG | CCGCCCTCA |
| | | GGGAGGTCCCTGAG | | GCAGAGGGTCACCA | GTGTCTGGG |
| | | ACTCGCCTGTGCAG | | TCTCCTGCACTGGGA | GCCCCAGGG |
| | | CCTCTGGATTCACCT | | GCAGCTCCAACATCG | CAGAGGGTC |
| | | TCAATAGCTATGCT | - | GGGCAGGTTATGAT | ACCATCTCC |
| | | ATGCACTGGGTCCG | | GTACACTGGTACCAG | TGCACTGGG |
| | | CCAGGCTCCAGGGA | | CAGCTTCCAGGAAG | AGCAGCTCC |
| | | AGGGGCTGGAGTGG | | AGCCCCCAAACTCCT | AACATCGGG |
| | | GTCTCGTCCATAAC | | CATCTTTGCTAACAC | GCAGGTTAT |
| | | TGGTGGCAGTAGTT | | CAATCGGCCCTCAGG | GATGTACAC |
| | | ATGTCGACTACTCA | | GGTCCCTGACCGATT | TGGTACCAG |
| | | GCCTCAGTGAAGGG | | CTCTGGCTCCAAGTC | CAGCTTCCA |
| | | CCGATTCACCATCT | | TGGCACCTCCGCCTC | GGAAGAGCC |
| | | CCAGAGACAACGCC | | CCTGGCCATCACTGG | CCCAAACTC |
| | | CAGAGCTCACTTTA | | TCTCCAGGCTGAGGA | CTCATCTTT |
| | | TCTGCAAATGAACA | | TGAGGCTGATTATTA | GCTAACACC |
| | | GCCTGAGAGCCGAG | | CTGCCAGTCCTATGA | AATCGGCCC |
| | | GACACGGCTGTGTA | | CAGTAGCCTGAGTTA | TCAGGGGTC |
| | | TTACTGTGCGAGAG | | TGTCTTCGGAACTGG | CCTGACCGA |
| | | ATATGTATGGTTTG | | GACCAAGGTCACCG | TTCTCTGGC |
| | | GGGAGTTATTATTC | | TCCTAGGC | TCCAAGTCT |
| | | GCCTAACTACTTCG | | (SEQ ID NO: 71) | GGCACCTCC |
| | | ACCCCTGGGGCCAG | | | GCCTCCCTG |
| | | GGAACCCTGGTCAC | | | GCCATCACT |
| | | TGTCTCCTCA(SEQ | | | GGTCTCCAG |
| | | ID NO: 70) | | | GCTGAGGAT |
| | | | | | GAGGCTGAT |
| | | | | | TATTACTGC |
| | | | | | CAGTCCTAT |
| | | | | | GACAGTAGC |
| | | | | | CTGAGTTAT |
| | | | | | GTCTTCGGA |
| | | | | | ACTGGG |
| | | | | | ACCAAGGTC |
| | | | | | ACCGTCCTA |
| | | | | | GGC |
| | | | | | (SEQ ID NO: |
| | | | | | 72) |
| 16 | 3H8 | GAGGTGCAGCTGGT | — | CAGCCTGTGCTGACT | CAGTCTGTG |
| | | GGAGTCTGGGGGAG | | CAGCCA C | CTGACGCAG |
| | | GCCTGGTCAAGCCT | | TCTCAGCGTCTGGGA | CCG CTC |
| | | GGGGGGTCCCTGAG | | CCCCCGGGCAGAGG | TCA |
| | | ACTCTCCTGTGCAG | | GTCACCATCTCCTGC | GCGTCTGGG |
| | | GCTCTGGATTCACC | | ACTGGGAGCAGCTC | ACCCCCGGG |
| | | TTCAGTAGCTATAC | | CAACATCGGGGCAG | CAGAGGGTC |
| | | CATGCACTGGGTCC | | GTTATGATGTACACT | ACCATCTCC |
| | | GCCAGGCTCCAGGG | | GGTACCAGCAGCTTC | TGCACTGGG |
| | | AAGGGGCTGGAGTG | | CAGGAAGAGCCCCC | AGCAGCTCC |
| | | GGTCTCGTCCATAA | | AAACTCCTCATCTTT | AACATCGGG |
| | | CTGGTGGCAGTAGT | | GCTAACACCAATCG | GCAGGTTAT |
| | | TATGTCGACTACTC | | GCCCTCAGGGGTCCC | GATGTACAC |
| | | AGCCTCAGTGAAGG | | TGACCGATTCTCTGG | TGGTACCAG |
| | | GCCGATTCACCATC | | CTCCAAGTCGGGCAC | CAGCTTCCA |
| | | TCCAGAGATAACGC | | CTCAGCCTCCCTGGC | GGAAGAGCC |
| | | CCAGAGCTCACTTT | | CATCACTGGGCTCCA | CCCAAACTC |
| | | ATCTGCAAATGAAC | | GGCTGAGGATGAGG | CTCATCTTT |
| | | AGCCTGAGAGCCGA | | CTGATTATTACTGCC | GCTAACACC |
| | | GGACACGGCTGTGT | | AGTCCTATGACCGCA | AATCGGCCC |
| | | ATTACTGTGCGAGA | | GCCTGAGTCATGTCT | TCAGGGGTC |
| | | GATGATTATGGTTC | | TCGGAACTGG | CCTGACCGA |
| | | GGGGAGTTATTCCA | | GACCAAGGTCACCG | TTCTCTGGC |
| | | ACTGGTTCGACCCC | | TCCTAGGC | TCCAAGTCG |
| | | TGGGGCCGGGGAAC | | (SEQ ID NO: 41) | GGCACCTCA |
| | | CACGGTCACCGTCT | | | GCCTCCCTG |
| | | CCTCA | | | GCCATCACT |
| | | (SEQ ID NO: 40) | | | GGGCTCCAG |
| | | | | | GCTGAGGAT |
| | | | | | GAGGCTGAT |
| | | | | | TATTACTGC |
| | | | | | CAGTCCTAT |
| | | | | | GACCGCAGC |
| | | | | | CTGAGTCAT |
| | | | | | GTCTTCGGA |
| | | | | | ACTGGG |
| | | | | | ACCAAGGTC |
| | | | | | ACCGTCCTA |
| | | | | | GGC |
| | | | | | (SEQ ID NO: |
| | | | | | 73) |
| 17 | 4E1 | GAGGTGCAGCTGGT | — | TCCTATGAGCTGACT | CAG TCT |
| | | GGAGTCTGGGGGAG | | CAGCCAC | GTG CTG |
| | | GCTTGGTACAGCCT | | CCTCAGTGTCTGGGG | ACG CAG |
| | | GGAGGGTCCCTGAG | | CCCCAGGGCAGAGG | CCG CCC |
| | | ACTCGCCTGTGCAG | | GTCACCATCTCCTGC | TCAGTGTCT |
| | | CCTCTGGATTCACCT | | ACTGGGAGCAGCTC | GGGGCCCCA |
| | | TCAGTAGCTATACC | | CAACATCGGGGCAG | GGGCAGAGG |
| | | ATGCACTGGGTCCG | | GTTATGATGTACACT | GTCACCATC |
| | | CCAGGCTCCAGGGA | | GGTACCAGCAGCTTC | TCCTGCACT |
| | | AGGGGCTGGAGTGG | | CAGGAAGAGCCCCC | GGGAGCAGC |
| | | GTCTCGTCCATAAC | | AAACTCCTCATCTTT | TCCAACATC |
| | | TGGTGGCAGTAGTT | | GCTAACACCAATCG | GGGGCAGGT |
| | | ATGTCGACTACTCA | | GCCCTCAGGGGTCCC | TATGATGTA |
| | | GCCTCAGTGAAGGG | | TGACCGATTCTCTGG | CACTGGTAC |
| | | CCGATTCACCATCT | | CTCCAAGTCTGGCGC | CAGCAGCTT |
| | | CCAGAGACAACGCC | | CTCTGCCTCCCTGGC | CCAGGAAGA |
| | | CAGAGCTCACTTTA | | CATCACTGGCCTCCA | GCCCCCAAA |
| | | TCTGCAAATGAACA | | GGCTGAGGATGAGG | CTCCTCATC |
| | | GCCTAAGAGCCGAG | | CTGATTATTACTGCC | TTTGCTAAC |
| | | GACACGGCTGTGTA | | AGTCCTATGACCGCA | ACCAATCGG |
| | | TTACTGTGCGAGAG | | GCCTGAGTCATGTCT | CCCTCAGGG |
| | | ATGATTATGGTTCG | | TCGGAACTGGGACC | GTCCCTGAC |
| | | GGGAGTTATTCCAA | | AAGGTCACCGTCCTA | CGATTCTCT |
| | | CTGGTTCGACCCCT | | GGC | GGCTCCAAG |
| | | GGGGCCAGGGAACC | | (SEQ ID NO: 75) | TCTGGCGCC |
| | | CTGGTCACCGTCTC | | | TCTGCCTCC |
| | | CTCA | | | CTGGCCATC |
| | | (SEQ ID NO: 74) | | | ACTGGCCTC |
| | | | | | CAGGCTGAG |
| | | | | | GATGAGGCT |
| | | | | | GATTATTAC |
| | | | | | TGCCAGTCC |
| | | | | | TATGACCGC |
| | | | | | AGCCTGAGT |
| | | | | | CATGTCTTC |
| | | | | | GGAACTGGG |
| | | | | | ACCAAGGTC |
| | | | | | ACCGTCCTA |
| | | | | | GGC |
| | | | | | (SEQ ID NO: |
| | | | | | 76) |
| 18 | 2H9 | CAGGTCACCTTGAA | GAGGTGCAG | TCCTATGAGCTGACT | CAGTCTGTG |
| | | GGAGTCTGGGGGAG | CTGGTGGAG | CAGCCACTCTCAGTG | CTGACGCAG |
| | | GCCTGGTCAAGCCT | TCTGGGGGA | TCTGGGGCCCCAGG | CCG |
| | | GGGGGGTCCCTGAG | GGCCTGGTC | GCAGAGGGTCACCA | CTCTCAGTG |
| | | ACTCTCCTGTGCAG | AAGCCTGGG | TTTCCTGCACTGGGA | TCTGGGGCC |
| | | CCTCTGGATTCACCT | GGGTCCCTG | GCAGCTCCAACATCG | CCAGGGCAG |
| | | TCACTAGTTATAGG | AGACTCTCCT | GGGCAGGTTATGAT | AGGGTCACC |
| | | ATGCATTGGGTCCG | GTGCAGCCT | GTACACTGGTACCAG | ATTTCCTGC |
| | | CCAGGCTCCAGGGA | CTGGATTCAC | CAGGTTCCAGGAAC | ACTGGGAGC |
| | | AGGGGCTGGAGTGG | CTTCACTAGT | AGCCCCCAAACTCCT | AGCTCCAAC |
| | | GTCTCATCAATTACT | TATAGGATG | CATCTTTGGTAGCAC | ATCGGGGCA |
| | | GGTGGTGGTAATTA | CATTGGGTCC | CAATCGGCCCTCAGG | GGTTATGAT |
| | | TATAGAGTACGCAG | GCCAGGCTC | GGTCCCTGACCGATT | GTACACTGG |
| | | ACTCAGTGAAGGGC | CAGGGAAGG | CTCTGGCTCCAAGTC | TACCAGCAG |
| | | CGGTTCACCATCTC | GGCTGGAGT | TGGCGCCTCTGCCTC | GTTCCAGGA |
| | | CAGAGACAACGCCA | GGGTCTCATC | CCTGGCCATCACTGG | ACAGCCCCC |
| | | AGAACTCACTGGAT | AATTACTGGT | CCTCCAGGCTGACGA | AAACTCCTC |
| | | CTGCAAATGAACAG | GGTGGTAAT | TGAGGCTGATTATTA | ATCTTTGGT |
| | | CCTGAGAGCCGAGG | TATATAGAG | CTGCCAGTCCTATGA | AGCACCAAT |
| | | ACACGGCTATTTAT | TACGCAGAC | CCGCAGCCTGAGTCA | CGGCCCTCA |
| | | TACTGTGCGAGAGA | TCAGTGAAG | TGTCTTCGGAACTGG | GGGGTCCCT |
| | | TATGTATGGTTTGG | GGCCGGTTC | GACCAAGGTCACCG | GACCGATTC |
| | | GGAGTTATTATTCG | ACCATCTCC | TCCTAGGC | TCTGGCTCC |
| | | CCTAACTACTTCGA | AGAGACAAC | (SEQ ID NO: 79) | AAGTCTGGC |
| | | CCCCTGGGGCCAGG | GCCAAGAAC | | GCCTCTGCC |
| | | GAACCCTGGTCACC | TCACTGGATC | | TCCCTGGCC |
| | | GTCTCCTCA | TGCAAATGA | | ATCACTGGC |
| | | (SEQ ID NO: 77) | ACAGCCTGA | | CTCCAGGCT |
| | | | GAGCCGAGG | | GACGATGAG |
| | | | ACACGGCTA | | GCTGATTAT |
| | | | TTTATTACTG | | TACTGCCAG |
| | | | TGCGAGAGA | | TCCTATGAC |
| | | | TATGTATGGT | | CGCAGCCTG |
| | | | TTGGGGAGT | | AGTCATGTC |
| | | | TATTATTCGC | | TTCGGAACT |
| | | | CTAACTACTT | | GGG |
| | | | CGACCCCTG | | ACCAAGGTC |
| | | | GGGCCAGGG | | ACCGTCCTA |
| | | | AACCCTGGT | | GGC |
| | | | CACCGTCTCC | | (SEQ ID NO: |
| | | | TCA | | 80) |
| | | | (SEQ ID NO: | | |
| | | | 78) | | |
| 19 | 12C11 | GAGGTGCAGCTGGT | — | CAGTCTGTGCTGACG | — |
| | | GGAGTCTGGGGGAG | | CAGCCGCCCTCAGTG | |
| | | GCCTGGTCAAGCCT | | TCTGGGGCTCCAGGG | |
| | | GGGGGGTCCCTGAG | | CAGAGGGTCACCAT | |
| | | ACTCTCCTGTGCAG | | CTCCTGCACTGGGAG | |
| | | CCTCTGGATTCACCT | | CAGCTCCAACATCGG | |
| | | TCACTAGTTATAGG | | GGCAGGTTATGATGT | |
| | | ATGCATTGGGTCCG | | ACACTGGTACCAGC | |
| | | CCAGGCTCCAGGGA | | AGGTTCCAGGAACA | |
| | | AGAGGCTGGAGTGG | | GCCCCCAAACTCCTC | |
| | | GTCTCATCAATTACT | | ATCTTTGGTAGCACC | |
| | | GGTGGTGGTAATTA | | AATCGGCCCTCAGG | |
| | | TATAGAGTACGCAG | | GGTCCCTGACCGATT | |
| | | ACTCAGTGAAGGGC | | CTCTGGCTCCAAGTC | |
| | | CGGTTCACCATCTC | | TGGCGCCTCAGCCTC | |
| | | CAGAGACAACGCCA | | CCTGGCCATCACTGG | |
| | | AGAACTCACTGGAT | | GCTCCAGACTGAGG | |
| | | CTGCAAATGAACAG | | ATGAGGCTGATTATT | |
| | | CCTGAGAGCCGAGG | | ACTGCCAGTCCTATG | |
| | | ACACGGCTATTTAT | | ACCGCAGCCTGAGTC | |
| | | TACTGTGCGAGAGA | | ATGTCTTCGGAACTG | |
| | | TATGTATGGTTTGG | | GG | |
| | | GGAGTTATTATTCG | | ACCAAGGTCACCGTC | |
| | | CCTAACTACTTCGA | | CTAGGC | |
| | | CCCCTGGGGCCAGG | | (SEQ ID NO: 82) | |
| | | GAACCCTGGTCACC | | | |
| | | GTCTCCTCA | | | |
| | | (SEQ ID NO: 81) | | | |
| 20 | 3E10 | GAGGTGCAGCTGGT | — | CAGCCTGTGCTGACT | CAGTCTGTG |
| | | GGAGTCTGGGGGAG | | CAGCCACCCTCAGTG | CTGACGCAG |
| | | GCCTGGTCAAGCCT | | TCTGGGGCCCCAGG | CCGCCCTCA |
| | | GGGGGGTCCCTGAG | | GCAGAGGGTCACCA | GTGTCTGGG |
| | | ACTCTCCTGTGCAG | | TCTCCTGCACTGGGA | GCCCCAGGG |
| | | CCTCTGGATTCACCT | | GCAGCTCCAACATCG | CAGAGGGTC |
| | | TCACTAGTTATAGG | | GGGCAGGTTATGAT | ACCATCTCC |
| | | ATGCATTGGGTCCG | | GTACACTGGTACCAG | TGCACTGGG |
| | | CCAGGCTCCAGGGA | | CAGGTTCCAGGAAC | AGCAGCTCC |
| | | AGGGGCTGGAGTGG | | AGCCCCCAAACTCCT | AACATCGGG |
| | | GTCTCATCAATTACT | | CATCTTTGGTAGCAC | GCAGGTTAT |
| | | GGTGGTGGTAATTA | | CAATCGGCCCTCAGG | GATGTACAC |
| | | TATAGAGTACGCAG | | GGTCCCTGACCGATT | TGGTACCAG |
| | | ACTCAGTGAAGGGC | | CTCTGGCTCCAAGTC | CAGGTTCCA |
| | | CGGTTCACCATCTC | | TGGCGCCTCAGCCTC | GGAACAGCC |
| | | CAGAGACAACGCCA | | CCTGGCCATCACTGG | CCCAAACTC |
| | | AGAACTCACTGGAT | | GCTCCAGACTGAGG | CTCATCTTT |
| | | CTGCAAATGAACAG | | ATGAGGCTGATTATT | GGTAGCACC |
| | | CCTGAGAGCCGAGG | | ACTGCCAGTCCTATG | AATCGGCCC |
| | | ACACGGCTATTTAT | | ACCGCAGCCTGAGTC | TCAGGGGTC |
| | | TACTGTGCGAGAGA | | ATGTCTTCGGAACTG | CCTGACCGA |
| | | TATGTATGGTTTGG | | GGACCAAGGTCACC | TTCTCTGGC |
| | | GGAGTTATTATTCG | | GTCCTAGGC | TCCAAGTCT |
| | | CCTAACTACTTCGA | | (SEQ ID NO: 84) | GGCGCCTCA |
| | | CCCCTGGGGCCAGG | | | GCCTCCCTG |
| | | GAACCCTGGTCACC | | | GCCATCACT |
| | | GTCTCCTCA | | | GGGCTCCAG |
| | | (SEQ ID NO: 83) | | | ACTGAGGAT |
| | | | | | GAGGCTGAT |
| | | | | | TATTACTGC |
| | | | | | CAGTCCTAT |
| | | | | | GACCGCAGC |
| | | | | | CTGAGTCAT |
| | | | | | GTCTTCGGA |
| | | | | | ACTGGG |
| | | | | | ACCAAGGTC |
| | | | | | ACCGTCCTA |
| | | | | | GGC |
| | | | | | (SEQ ID NO: |
| | | | | | 85) |
| 21 | 2G1 | CAGGTCACCTTGAA | GGGTCTCATC | CAGTCTGTGCTGACG | — |
| | | GGAGTCTGGGGGAG | AATTACTGGT | CAGCCGCCCTCAGTG | |
| | | GCCTGGTCAAGCCT | GAGGTGCAG | TCTGGGGCCCCAGG | |
| | | GGGGGGTCCCTGAG | CTGGTGGAG | GCAGAGGGTCACCA | |
| | | ACTCTCCTGTGCAG | TCTGGGGGA | TCTCCTGCACTGGGA | |
| | | CCTCTGGATTCACCT | GGCCTGGTC | GCAGCTCCAACATCG | |
| | | TCACTAGTTATAGG | AAGCCTGGG | GGGCAGGTTATGAT | |
| | | ATGCATTGGGTCCG | GGGTCCCTG | GTACACTGGTATCAG | |
| | | CCAGGCTCCAGGGA | AGACTCTCCT | CAGCTTCCAGGAAC | |
| | | AGGGGCTGGAGTGG | GTGCAGCCT | AGCCCCCAAACTCCT | |
| | | GTCTCATCAATTACT | CTGGATTCAC | CATCTTTGGTAGCAC | |
| | | GGTGGTGGTAATTA | CTTCACTAGT | CAATCGGCCCTCAGG | |
| | | TATAGAGTACGCAG | TATAGGATG | GGTCCCTGACCGATT | |
| | | ACTCAGTGAAGGGC | CATTGGGTCC | CTCTGGCTCCAAGTC | |
| | | CGGTTCACCATCTC | GCCAGGCTC | TGGCGCCTCAGCCTC | |
| | | CAGAGACAACGCCA | CAGGGAAGG | CCTGGCCATCACTGG | |
| | | AGAACTCACTGGAT | GGCTGGAGT | GCTCCAGACTGAGG | |
| | | CTGCAAATGAACAG | GGTGGTAAT | ATGAGGCTGATTATT | |
| | | CCTGAGAGCCGAGG | TATATAGAG | ACTGCCAGTCCTATG | |
| | | ACACGGCTATTTAT | TACGCAGAC | ACCGCAGCCTGAGTC | |
| | | TACTGTGCGAGAGA | TCAGTGAAG | ATGTCTTCGGAACTG | |
| | | TATGTATGGTTTGG | GGCCGGTTC | GG | |
| | | GGAGTTATTATTCG | ACCATCTCC | ACCAAGGTCACCGTC | |
| | | CCTAACTACTTCGA | AGAGACAAC | CTAGGC | |
| | | CCCCTGGGGCCAGG | GCCAAGAAC | (SEQ ID NO: 88) | |
| | | GAACCCTGGTCACC | TCACTGGATC | | |
| | | GTCTCCTCA | TGCAAATGA | | |
| | | (SEQ ID NO: 86) | ACAGCCTGA | | |
| | | | GAGCCGAGG | | |
| | | | ACACGGCTA | | |
| | | | TTTATTACTG | | |
| | | | TGCGAGAGA | | |
| | | | TATGTATGGT | | |
| | | | TTGGGGAGT | | |
| | | | TATTATTCGC | | |
| | | | CTAACTACTT | | |
| | | | CGACCCCTG | | |
| | | | GGGCCAGGG | | |
| | | | AACCCTGGT | | |
| | | | CACCGTCTCC | | |
| | | | TCA | | |
| | | | (SEQ ID NO: | | |
| | | | 87) | | |
| 22 | 1E6 | GAGGTGCAGCTGGT | — | CAGTCTGTGCTGACG | — |
| | | GGAGTCTGGGGGAG | | CAGCCGCCC TCA | |
| | | GCCTGGTCAAGCCT | | GTTTCTGGGGCCCCA | |
| | | GGGGGGTCCCTGAG | | GGGCAGAGGGTCAC | |
| | | ACTCTCCTGTGCAG | | CATCTCCTGCACTGG | |
| | | CCTCTGGATTCACCT | | GAGCAGCTCCAACA | |
| | | TCACTAGTTATAGG | | TCGGGGCAGGTTATG | |
| | | ATGCATTGGGTCCG | | ATGTACACTGGTACC | |
| | | CCAGGCTCCAGGGA | | AGCAGCTTCCAGGA | |
| | | AGGGGCTGGAGTGG | | AGAGCCCCCAAACT | |
| | | GTCTCATCAATTACT | | CCTCATCTTTGCTAA | |
| | | GGTGGTGGTAATTA | | CACCAATCGGCCCTC | |
| | | TATAGAGTACGCAG | | AGGGGTCCCTGACC | |
| | | ACTCAGTGAAGGGC | | GATTCTCTGGCTCCA | |
| | | CGGTTCACCATCTC | | AGTCTGGCGCCTCTG | |
| | | CAGAGACAACGCCA | | CCTCCCTGGCCATCA | |
| | | AGAACTCACTGGAC | | CTGGCCTCCAGGCTG | |
| | | CTGCAAATGAACAG | | ACGATGAGGCTGATT | |
| | | CCTGAGAGCCGAGG | | ATTACTGCCAGTCCT | |
| | | ACACGGCTATTTAT | | ATGACCGCAGCCTG | |
| | | TACTGTGCGAGAGA | | AGTCATGTCTTCGGA | |
| | | TATGTATGGTTTGG | | ACTGGG | |
| | | GGAGTTATTATTCG | | ACCAAGGTCACCGTC | |
| | | CCTAACTACTTCGA | | CTAGGC | |
| | | CCCCTGGGGCCAGG | | (SEQ ID NO: 90) | |
| | | GAACCCTGGTCACT | | | |
| | | GTCTCCTCA | | | |
| | | (SEQ ID NO: 89) | | | |
| 23 | 2H11 | CAGGTCCAGCTGGT | — | CAGTCTGTGTTGACG | — |
| | | ACAGTCTGGGGGAG | | CAGCCGCCCTCAGTG | |
| | | GCCTGGTCAAGCCT | | TCTGGGGCCCCAGG | |
| | | GGGGGGTCCCTGAG | | GCAGAGGGTCACCA | |
| | | ACTCTCCTGTGCAG | | TCTCCTGCACTGGGA | |
| | | CCTCTGGATTCACCT | | GCAGCTCCAACATCG | |
| | | TCACTAGTTATAGG | | GGGCAGGTTATGAT | |
| | | ATGCATTGGGTCCG | | GTACACTGGTACCAG | |
| | | CCAGGCTCCAGGGA | | CAGCTTCCAGGAAG | |
| | | AGGGGCTGGAGTGG | | AGCCCCCAAACTCCT | |
| | | GTCTCATCAATTACT | | CATCTTTGCTAACAC | |
| | | GGTGGTGGTAATTA | | CAATCGGCCCTCAGG | |
| | | TATAGAGTACGCAG | | GGTCCCTGACCGATT | |
| | | ACTCAGTGAAGGGC | | CTCTGGCTCCAAGTC | |
| | | CGGTTCACCATCTC | | TGGCGCCTCTGCCTC | |
| | | CAGAGACAACGCCA | | CCTGGCCATCTCTGG | |
| | | AGAACTCACTGGAT | | GCTCCAGGCTGAGG | |
| | | CTGCAAATGAACAG | | ATGAGGCTCATTATT | |
| | | CCTGAGAGCCGAGG | | ACTGCCAGTCCTATG | |
| | | ACACGGCTATTTAT | | ACAGGAGCCTGAAT | |
| | | TACTGTGCGAGAGA | | GTGGTTTTCGGCGGA | |
| | | TATGTATGGTTTGG | | GGGACCGAGCTGAC | |
| | | GGAGTTATTATTCG | | CGTCCTAGGC | |
| | | CCTAACTACTTCGA | | (SEQ ID NO: 92) | |
| | | CCCCTGGGGCCAGG | | | |
| | | GAACCACGGTCACC | | | |
| | | GTCTCCTCA | | | |
| | | (SEQ ID NO: 91) | | | |
| 24 | 2F1 | GAGGTGCAGCTGGT | — | CAGTCTGTGCTGACG | — |
| | | GGAGTCTGGGGGAG | | CAGCCGCCCTCAGTG | |
| | | GCCTGGTCAAGCCT | | TCTGGGGCCCCAGG | |
| | | GGGGGGTCCCTGAG | | GCAGAGGGTCACCA | |
| | | ACTCTCCTGTGCAG | | TCTCCTGCACTGGGA | |
| | | CCTCTGGATTCACCT | | GCAGCTCCAACATCG | |
| | | TCACTAGTTATAGG | | GGGCAGGTTATGAT | |
| | | ATGCATTGGGTCCG | | GTACACTGGTACCAG | |
| | | CCAGGCTCCAGGGA | | CAGGTTCCAGGAAC | |
| | | AGGGGCTGGAGTGG | | AGCCCCCAAACTCCT | |
| | | GTCTCATCAATTACT | | CATCTTTGGTAGCAC | |
| | | GGTGGTGGTAATTA | | CAATCGGCCCTCAGG | |
| | | TATAGAGTACGCAG | | GGTCCCTGACCGATT | |
| | | ACTCAGTGAAGGGC | | CTCTGGCTCCAAGTC | |
| | | CGGTTCACCATCTC | | TGGCGCCTCAGCCTC | |
| | | CAGAGACAACGCCA | | CCTGGCCATCACTGG | |
| | | AGAACTCACTGGAT | | GCTCCAGACTGAGG | |
| | | CTGCAAATGAACAG | | ATGAGGCTGATTATT | |
| | | CCTGAGAGCCGAGG | | ATTGCCAGTCCTATG | |
| | | ACACGGCTATTTAT | | ACAGAAGCCTGAGT | |
| | | TACTGTGCGAGAGA | | GCTTGGGTGTTCGGC | |
| | | TATGTATGGTTTGG | | GGAGGG | |
| | | GGAGTTATTATTCG | | ACCAAGCTGACCGTC | |
| | | CCTAACTACTTCGA | | CTAGGC | |
| | | CCCCTGGGGCCAGG | | (SEQ ID NO: 94) | |
| | | GAACCCTGGTCACC | | | |
| | | GTCTCCTCA | | | |
| | | (SEQ ID NO: 93) | | | |
| 25 | 13C2 | GAGGTGCAGCTGGT | — | CAGTCTGTCCTGACG | — |
| | | GGAGTCTGGGGGAG | | CAGCCGCCCTCAGTG | |
| | | GCCTGGTCAAGCCT | | TCTGGGGCCCCAGG | |
| | | GGGGGGTCCCTGAG | | GCAGAGGGTCACCA | |
| | | ACTCTCCTGTGCAG | | TCTCCTGCACTGGGA | |
| | | CCTCTGGATTCACCT | | GCAGCTCCAACATCG | |
| | | TCACTAGTTATAGG | | GGGCAGGTTATGAT | |
| | | ATGCATTGGGTCCG | | GTACACTGGTACCAG | |
| | | CCAGGCTCCAGGGA | | CAGGTTCCAGGAAC | |
| | | AGGGGCTGGAGTGG | | AGCCCCCAAACTCCT | |
| | | GTCTCATCAATTACT | | CATCTTTGGTAGCAC | |
| | | GGTGGTGGTAATTA | | CGATCGGCCCTCAGG | |
| | | TATAGAGTACGCAG | | GGTCCCTGATCGCTT | |
| | | ACTCAGTGAAGGGC | | CTCTGGCTCCAAGTC | |
| | | CGGTTCACCATCTC | | TGGCAACACGGCCTC | |
| | | CAGAGACAACGCCA | | CCTGACCATCTCTGG | |
| | | AGAACTCACTGGAT | | GCTCCAGGCTGAGG | |
| | | CTGCAAATGAACAG | | ATGAGGCTGATTATT | |
| | | CCTGAGAGCCGAGG | | ATTGCCAGTCCTATG | |
| | | ACACGGCTATTTAT | | ACCGCAGCCTGAGTC | |
| | | TACTGTGCGAGAGA | | ATGTCTTCGGAACTG | |
| | | TATGTATGGTTTGG | | GG | |
| | | GGAGTTATTATTCG | | ACCAAGGTCACCGTC | |
| | | CCTAACTACTTCGA | | CTAGGC | |
| | | CCCCTGGGGCCAGG | | (SEQ ID NO: 96) | |
| | | GAACCCTGGTCACC | | | |
| | | GTCTCCTCA | | | |
| | | (SEQ ID NO: 95) | | | |
| 26 | 2H8 | GAGGTGCAGCTGGT | — | CAGTCTGTGCTGACG | — |
| | | GGAGTCTGGGGGAG | | CAGCCGCCCTCAGTG | |
| | | GCCTGGCCAAGCCT | | TCTGGGGCCCCAGG | |
| | | GGGGGGTCCCTGAG | | GCAGAGGGTCACCA | |
| | | ACTCTCCTGTGCAG | | TCTCCTGCACTGGGA | |
| | | CCTCTGGATTCACCT | | GCAGCTCCAACATCG | |
| | | TCACTAGTTATAGG | | GGGCAGGTTATGAT | |
| | | ATGCATTGGGTCCG | | GTACACTGGTATCAG | |
| | | CCAGGCTCCAGGGA | | CAGCTTCCGGGAGC | |
| | | AGGGGCTGGAGTGG | | AGCCCCCAGACTCCT | |
| | | GTCTCATCAATTACT | | CATGTTTGGTAACAG | |
| | | GGTGGTGGTAATTA | | CAATCGGCCCTCGGG | |
| | | TATAGAGTACGCAG | | GGTACCTGACCGCTT | |
| | | ACTCAGTGAAGGGC | | CTCTGGCTCCAAGTC | |
| | | CGGTTCACCATCTC | | TGGCACCTCCGCCTC | |
| | | CAGAGACAACGCCA | | CCTGGCCATCACTGG | |
| | | AGAACTCACTGGAT | | TCTCCAGGCTGAGGA | |
| | | CTGCAAATGAACAG | | TGAGGCTGATTATTA | |
| | | CCTGAGAGCCGAGG | | CTGCCAGTCCTATGA | |
| | | ACACGGCTATTTAT | | CCGCAGCCTGAGTCA | |
| | | TACTGTGCGAGAGA | | TGTCTTCGGAACTGG | |
| | | TATGTATGGTTTGG | | GACCAAGGTCACCG | |
| | | GGAGTTATTATTCG | | TCCTAGGC | |
| | | CCTAACTACTTCGA | | (SEQ ID NO: 98) | |
| | | CCCCTGGGGCCAGG | | | |
| | | GAACCCTGGTCACC | | | |
| | | GTCTCCTCA | | | |
| | | (SEQ ID | | | |
| | | NO: 97) | | | |
| 27 | 10A6 | GAGGTGCAGCTGGT | — | TCCTATGAGCTGACT | CAGTCTGTG |
| | | GGAGTCTGGGGGAG | | CAGCCACCCTCAGTG | CTGACGCAG |
| | | GCCTGGTCAAGCCT | | TCTGGGGCCCCAGG | CCGCCC TCA |
| | | GGGGGGTCCCTGAG | | GCAGAGGGTCACCA | GTGTCTGGG |
| | | ACTCTCCTGTGCAG | | TCTCCTGCACTGGGA | GCCCCAGGG |
| | | CCTCTGGATTCACCT | | GCAGCTCCAACATCG | CAGAGGGTC |
| | | TCACTAGTTATAGG | | GGGCAGGTTATGAT | ACCATCTCC |
| | | ATGCATTGGGTCCG | | GTACACTGGTATCAG | TGCACTGGG |
| | | CCAGGCTCCAGGGA | | CAGCTTCCGGGAGC | AGCAGCTCC |
| | | AGGGGCTGGAGTGG | | AGCCCCCAGACTCCT | AACATCGGG |
| | | GTCTCATCAATTACT | | CATGTTTGGTAACAG | GCAGGTTAT |
| | | GGTGGTGGTAATTA | | CAATCGGCCCTCGGG | GATGTACAC |
| | | TATAGAGTACGCAG | | GGTACCTGACCGCTT | TGGTATCAG |
| | | ACTCAGTGAAGGGC | | CTCTGGCTCCAAGTC | CAGCTTCCG |
| | | CGGTTCACCATCTC | | TGGCACCTCCGCCTC | GGAGCAGCC |
| | | CAGAGACAACGCCA | | CCTGGCCATCACTGG | CCCAGACTC |
| | | AGAACTCACTGGAT | | TCTCCAGGCTGAGGA | CTCATGTTT |
| | | CTGCAAATGAACAG | | TGAGGCTGATTATTA | GGTAACAGC |
| | | CCTGAGAGCCGAGG | | CTGCCAGTCCTATGA | AATCGGCCC |
| | | ACACGGCTATTTAT | | CAGTAGCCTGAGTTA | TCGGGGGTA |
| | | TACTGTGCGAGAGA | | TGTCTTCGGAACTGG | CCTGACCGC |
| | | TATGTATGGTTTGG | | GACCAAGGTCACCG | TTCTCTGGC |
| | | GGAGTTATTATTCG | | TCCTAGGC | TCCAAGTCT |
| | | CCTAACTACTTCGA | | (SEQ ID NO: 100) | GGCACCTCC |
| | | CTCCTGGGGCCAGG | | | GCCTCCCTG |
| | | GAACCCTGGTCACC | | | GCCATCACT |
| | | GTCTCCTCA | | | GGTCTCCAG |
| | | (SEQ ID | | | GCTGAGGAT |
| | | NO: 99) | | | GAGGCTGAT |
| | | | | | TATTACTGC |
| | | | | | CAGTCCTAT |
| | | | | | GACAGTAGC |
| | | | | | CTGAGTTAT |
| | | | | | GTCTTCGGA |
| | | | | | ACTGGGACC |
| | | | | | AAGGTCACC |
| | | | | | GTCCTAGGC |
| | | | | | (SEQ ID NO: |
| | | | | | 101) |
| 28 | 4G2 | GAGGTGCAGCTGGT | — | CAGTCTGTGCTGACG | — |
| | | GGAGTCTGGGGGAG | | CAGCCGCCC TCA | |
| | | GCCTGGTCAAGCCT | | GTGTCTGGGGCCCCA | |
| | | GGGGGGTCCCTGAG | | GGGCAGAGGGTCAC | |
| | | ACTCTCCTGTGCAG | | CATCTCCTGCACTGG | |
| | | CCTCTGGATTCACCT | | GAGCAGCTCCAACA | |
| | | TCACTAGTTATAGG | | TCGGGGCAGGTTATG | |
| | | ATGCATTGGGTCCG | | ATGTACACTGGTATC | |
| | | CCAGGCTCCAGGGA | | AGCAGCTTCCGGGA | |
| | | AGGGGCTGGAGTGG | | GCAGCCCCCAGACTC | |
| | | GTCTCATCAATTACT | | CTCATGTTTGGTAAC | |
| | | GGTGGTGGTAATTA | | AGCAATCGGCCCTCG | |
| | | TATAGAGTACGCAG | | GGGGTACCTGACCG | |
| | | ACTCAGTGAAGGGC | | CTTCTCTGGCTCCAA | |
| | | CGGTTCACCATCTC | | GTCTGGCACCTCCGC | |
| | | CAGAGACAACGCCA | | CTCCCTGGCCATCAC | |
| | | AGAACTCACTGGAT | | TGGTCTCCAGGCTGA | |
| | | CTGCAAATGAACAG | | GGATGAGGCTGATT | |
| | | CCTGAGAGCCGAGG | | ATTACTGCCAGTCCT | |
| | | ACACGGCTATTTAT | | ATGACAGTAGCCTG | |
| | | TACTGTGCGAGAGA | | AGTCATGTCTTCGGA | |
| | | TATGTATGGTTTGG | | ACTGGGACCAAGGT | |
| | | GGAGTTATTATTCG | | CACCGTCCTAGGC | |
| | | CCTAACTACTTCGA | | (SEQ ID NO: 103) | |
| | | CCCCTGGGGCCAGG | | | |
| | | GAACCCTGGTCACC | | | |
| | | GTCTCCTCA(SEQ ID | | | |
| | | NO: 102) | | | |
| 29 | 2G11 | GAGGTGCAGCTGGT | — | CAGTCTGTGCTGACG | — |
| | | GGAGTCTGGGGGAG | | CAGCCGCCCTCAGTG | |
| | | GCTTGGTCCAGCCT | | TCTGGGGCCCCAGG | |
| | | GGGGGGTCCCTGAG | | GCAGAGGGTCACCA | |
| | | ACTCTCCTGTGCAG | | TCTCCTGCACTGGGA | |
| | | CCTCTGGATTCACCT | | GCAGCTCCAACATCG | |
| | | TCACTAGTTATAGG | | GGGCAGGTTATGAT | |
| | | ATGCATTGGGTCCG | | GTACACTGGTATCAG | |
| | | CCAGGCTCCAGGGA | | CAGCTTCCGGGAGC | |
| | | AGGGGCTGGAGTGG | | AGCCCCCAGACTCCT | |
| | | GTCTCATCAATTACT | | CATGTTTGGTAACAG | |
| | | GGTGGTGGTAATTA | | CAATCGGCCCTCGGG | |
| | | TATAGAGTACGCAG | | GGTACCTGACCGCTT | |
| | | ACTCAGTGAAGGGC | | CTCTGGCTCCAAGTC | |
| | | CGGTTCACCATCTC | | TGGCACCTCCGCCTC | |
| | | CAGAGACAACGCCA | | CCTGGCCATCACTGG | |
| | | AGAACTCACTGGAT | | TCTCCAGGCTGAGGA | |
| | | CTGCAAATGAACAG | | TGAGGCTGATTATTA | |
| | | CCTGAGAGCCGAGG | | CTGCCAGTCCTATGA | |
| | | ACACGGCTATTTAT | | CAGTAGCCTGAGTTA | |
| | | TACTGTGCGAGAGA | | TGTCTTCGGAACTGG | |
| | | TATGTATGGTTTGG | | GACCAAGGTCACCG | |
| | | GGAGTTATTATTCG | | TCCTAGGC | |
| | | CCTAACTACTTCGA | | (SEQ ID NO: 105) | |
| | | CCCCTGGGGCCAGG | | | |
| | | GAACCCTGGTCACC | | | |
| | | GTCTCCTCA(SEQ ID | | | |
| | | NO: 104) | | | |
| 30 | 11H9 | GAGGTGCAGCTGGT | — | TGCTGTGGGTGAGTG | CAGTCTGTG |
| | | GGAGTCTGGGGGAG | | GTACCTGTGGGCAGT | CTGACGCAG |
| | | GCTTGGTACAGCCT | | CTGTGCTGACGCAGC | CCGCCCTCA |
| | | GGGGGGTCCCTGAG | | CGCCCTCAGTGTCTG | GTGTCTGGG |
| | | ACTCTCCTGTGCAG | | GGGCCCCAGGGCAG | GCCCCAGGG |
| | | CATCTGAATTCACC | | AGGGTCACCATCTCC | CAGAGGGTC |
| | | TTTAGCATGAACTG | | TGCACTGGGAGCAG | ACCATCTCC |
| | | GGTCCGACAGGCTC | | CTCCAACATCGGGGC | TGCACTGGG |
| | | CAGGGAAGGGGCTG | | AGGTTATGATGTACA | AGCAGCTCC |
| | | GAGTGGGTCTCATC | | CTGGTACCAGCAGCT | AACATCGGG |
| | | AGTTCGAGGTGGCG | | TCCAGGAAGAGCCC | GCAGGTTAT |
| | | GTACTGAAACATAC | | CCAAACTCCTCATCT | GATGTACAC |
| | | TATGCAGACTCCGT | | TTGCTAACACCAATC | TGGTACCAG |
| | | GAAGGGCCGGTTCA | | GGCCCTCAGGGGTCC | CAGCTTCCA |
| | | CCGTCTCCAGAGAC | | CTGACCGATTCTCTG | GGAAGAGCC |
| | | AATTCCAAGAACAC | | GCTCCAAGTCTGGCG | CCCAAACTC |
| | | CCTGCATCTGCAAA | | CCTCTGCCTCCCTGG | CTCATCTTT |
| | | TGAACAGCCTGAGA | | CCATCACTGGCCTCC | GCTAACACC |
| | | GCCGAGGACACGGC | | AGGCTGACGATGAG | AATCGGCCC |
| | | CCTTTATTACTGTGC | | GCTGATTATTACTGC | TCAGGGGTC |
| | | GGGCGGCCCTATAG | | CAGTCTTATGACAGC | CCTGACCGA |
| | | TGGAACCCAACATT | | AAACATCATGTGGTC | TTCTCTGGC |
| | | GACTACTTTAACTC | | TTTGGCACAGGGACC | TCCAAGTCT |
| | | CTGGGGCCAGGGAA | | AAGCTGACCGTCCTA | GGCGCCTCT |
| | | CCCTGGTCACCGTC | | GGCCAACCTAAGGC | GCCTCCCTG |
| | | TCCTCA | | CAATCCCAC | GCCATCACT |
| | | (SEQ ID NO: 106) | | (SEQ ID NO: 107) | GGCCTCCAG |
| | | | | | GCTGACGAT |
| | | | | | GAGGCTGAT |
| | | | | | TATTACTGC |
| | | | | | CAGTCTTAT |
| | | | | | GACAGCAAA |
| | | | | | CATCATGTG |
| | | | | | GTCTTTGGC |
| | | | | | ACAGGG |
| | | | | | ACCAAGCTG |
| | | | | | ACCGTCCTA |
| | | | | | GGC |
| | | | | | (SEQ ID NO: |
| | | | | | 108) |
| 31 | 12C9 | ″GAGGTGCAGCTGG | — | CAGTCTGTGTTGACG | — |
| | | TGGAGTCTGGGGGA | | CAGCCGCCCTCAGTG | |
| | | GGCTTGGTCCAGCC | | TCTGGGGCCCCAGG | |
| | | TGGGGGGTCCCTGA | | GCAGAGGGTCACCA | |
| | | GACTCTCCTGTGCA | | TCTCCTGCACTGGGA | |
| | | GCCTCTGGGTTCTCC | | GCAGCTCCAACATCG | |
| | | TTTAGGAGTTATTG | | GGGCAGGTTATGAT | |
| | | GATGAGCTGGCTCC | | GTACACTGGTACCAG | |
| | | GCCAGGCCCCAGGG | | CAGGTTCCAGGAAC | |
| | | AAGGGGCTGCAGTG | | AGCCCCCAAACTCCT | |
| | | GGTGGCCAACATAA | | CATCTTTGGTAGCAC | |
| | | AGCCAGACGGAAGT | | CAATCGGCCCTCAGG | |
| | | GTGGAAAGTTATGT | | GGTCCCTGACCGATT | |
| | | GGACTCCGTGGAGG | | CTCTGGCTCCAAGTC | |
| | | GCCGATTCACCATC | | TGGCGCCTCAGCCTC | |
| | | TCCAGAGACAACGC | | CCTGGCCATCATTGG | |
| | | CAAGAATTCACTGT | | GCTCCAGACTGAGG | |
| | | TTCTGCAAATGAAC | | ATGAGGCTGATTATT | |
| | | AGCCTGTCAGCCGA | | ACTGCCAGTCCTATG | |
| | | GGACATGGCTGTTT | | ACCGCAGCCTGAGTC | |
| | | ATTACTGTGCGAGG | | ATGTCTTCGGAACTG | |
| | | ACGGACGACGGCAG | | GCACCCAGCTGACC | |
| | | CAGCTGGTTCGTGT | | GACCTCGGC | |
| | | CCACCAGTAGTTTC | | (SEQ ID NO: 110) | |
| | | TACGGTATGGACGT | | | |
| | | CTGGGGCCAAGGGA | | | |
| | | CCACGGTCACCGTC | | | |
| | | TCCTCA | | | |
| | | (SEQ ID NO: 109) | | | |
| 32 | 7A10 | GAGGTGCAGCTGGT | — | CAGTCTGTGTTGACG | — |
| | | GGAGTCTGGGGGAG | | CAGCCGCCCTCAGTG | |
| | | GCCTGGTCAAGCCT | | TCTGGGGCCCCAGG | |
| | | GGGGGGTCCCTGAG | | GCAGAGGGTCACCA | |
| | | ACTCTCCTGTGCAG | | TTTCCTGCACTGGGA | |
| | | CCTCTGGATTCACCT | | ACAGCTCCAACCTCG | |
| | | TCAGTAGCTATACC | | GGGCAGGTTATGAT | |
| | | ATGCACTGGGTCCG | | GTACACTGGTACCAG | |
| | | CCAGGCTCCAGGGA | | CAGGTTCCAGGAAC | |
| | | AGGGGCTGGAGTGG | | AGCCCCCAAACTCCT | |
| | | GTCTCGTCCATAAC | | CATCTTTGGTAGCAC | |
| | | TGGTGGCAGTAGTT | | CAATCGGCCCTCAGG | |
| | | ATGTCGACTACTCA | | GGTCCCTGACCGATT | |
| | | GCCTCAGTGAAGGG | | CTCTGGCTCCAAGTC | |
| | | CCGATTCACCATCT | | TGGCGCCTCAGCCTC | |
| | | CCCGAGACAATTCC | | CCTGACAATCTCTGG | |
| | | AAGAACACGCTGTA | | GCTCCAGGCTGAGG | |
| | | TCTGCAAATGAGCA | | ACGAGGCTGATTATT | |
| | | GCCTGAGAACTGAC | | ACTGCTGTTCGTATG | |
| | | GACACGGCCGTATA | | CAGCTAGTAGCTCTA | |
| | | TTTCTGTGCGAAAG | | GGGTCTTCGGAACTG | |
| | | ACCAACGTGGAGAC | | GCACCAAGGTGACC | |
| | | AGCTATGACTCGGT | | GTCCTCGGC | |
| | | TATGAACTGGTACT | | (SEQ ID NO: 112) | |
| | | TCGATCTCTGGGGC | | | |
| | | CGTGGCACCACGGT | | | |
| | | CACCGTCTCCTCA(S | | | |
| | | EQ ID NO: 111) | | | |
| 33 | 4G1 | CAGGTCACCTTGAA | TGCTGTGGGT | CAGTCTGTGCTGACG | - |
| | | GGAGTCTGGGGGCG | GAGTGGTAC | CAGCCGCCCTCAGTG | |
| | | GCCTGGTTAAGCCT | CTGTGGGGA | TCTGGGGCCCCAGG | |
| | | GGGGGGTCCCTGAG | GGTGCAGCT | GCAGAGGGTCACCA | |
| | | ACTCTCGTGTGCAG | GGTGGAGTC | TTTCCTGCACTGGGA | |
| | | CCTCTGGGTTCAGG | TGGGGGCGG | ACAGCTCCAACCTCG | |
| | | CTCAGTAGCTATGG | CCTGGTTAA | GGGCAGGTTATGAT | |
| | | CATGAACTGGGTCC | GCCTGGGGG | GTACACTGGTACCAG | |
| | | GCCAGGCTCCAGGG | GTCCCTGAG | CAGCTTCCAGGAAG | |
| | | AAGGGGCTGGAGTG | ACTCTCGTGT | AGCCCCCAAACTCCT | |
| | | GGTCTCATCCATTTC | GCAGCCTCT | CATCTTTGCTAACAC | |
| | | TGCTAGTAGTAGTT | GGGTTCAGG | CAATCGGCCCTCAGG | |
| | | TTATAAACTATGCA | CTCAGTAGCT | GGTCCCTGACCGATT | |
| | | GACTCAGTGAGGGA | ATGGCATGA | CTCTGGCTCCAAGTC | |
| | | CCGATTCACCATCT | ACTGGGTCC | TGGCGCCTCTGCCTC | |
| | | CCAGAGACAACGCC | GCCAGGCTC | CCTGGCCATCACTAG | |
| | | AAGAACTCACTGTA | CAGGGAAGG | CCTCCAGGCTGACGA | |
| | | TCTGCAAATGAACA | GGCTGGAGT | TGAGGCTGATTATTA | |
| | | GCCTGAGAGCCGAG | GGGTCTCATC | CTGCCAGTCCTATGA | |
| | | GACACGGCTGTGTA | CATTTCTGCT | CCGCAGCCTGAGTCA | |
| | | TTACTGTGCGAGAG | AGTAGTAGT | TGTCTTCGGAACTGG | |
| | | ATGATTATGGTTCG | TTTATAAACT | GACCAAGGTCACCG | |
| | | GGGAGTTATTCCAA | ATGCAGACT | TCCTAGGC | |
| | | CTGGTTCGACCCCT | CAGTGAGGG | (SEQ ID NO: 43) | |
| | | GGGGCCAGGGAACC | ACCGATTCA | | |
| | | CTGGTCACCGTCTC | CCATCTCCA | | |
| | | CTCA | GAGACAACG | | |
| | | (SEQ ID NO: 42) | CCAAGAACT | | |
| | | | CACTGTATCT | | |
| | | | GCAAATGAA | | |
| | | | CAGCCTGAG | | |
| | | | AGCCGAGGA | | |
| | | | CACGGCTGT | | |
| | | | GTATTACTGT | | |
| | | | GCGAGAGAT | | |
| | | | GATTATGGTT | | |
| | | | CGGGGAGTT | | |
| | | | ATTCCAACTG | | |
| | | | GTTCGACCC | | |
| | | | CTGGGGCCA | | |
| | | | GGGAACCCT | | |
| | | | GGTCACCGT | | |
| | | | CTCCTCAGCC | | |
| | | | TCCACCAAG | | |
| | | | GGCCCATCG | | |
| | | | GTCTTCCCCC | | |
| | | | (SEQ ID NO: | | |
| | | | 113) | | |
| 34 | 7C6 | GAGGTGCAGCTGGT | TGCTGTGGGT | CAGTCTGTGTTGACG | — |
| | | GGAGTCTGGGGGAG | GAGTGGTAC | CAGCCGCCCTCAGTG | |
| | | GCCTGGTCAAGCCT | CTGTGGGCA | TCTGGGGCCCCAGG | |
| | | GGGGGGTCCCTGAG | GGTTCAGCT | GCAGAGGGTCACCA | |
| | | ACTCTCCTGTGCAG | GGTGCAGTC | TCTCCTGCACTGGGA | |
| | | CCTCTGGATTCACCT | TGGAGCTGA | GCAGCTCCAACATCG | |
| | | TCAGTCATTATGGT | GGTGAAGAA | GGGCAGGTTATGAT | |
| | | ATTAGTTGGGTGCG | GCCTGGGGC | GTACACTGGTACCAG | |
| | | CCAGGCCCCTGGAC | CTCAGTGAA | CAGGTTCCAGGAAC | |
| | | AAGGCCTTGAGTGG | GGTC | AGCCCCCAAACTCCT | |
| | | ATGGCCTGGATCAG | TCCTGCAAG | CATCTTTGGTAGCAC | |
| | | CGCTTACAATGGTA | GCTTCTGGAT | CAATCGGCCCTCAGG | |
| | | ACACAGACTCCATA | TCACCTTCAG | GGTCCCTGACCGATT | |
| | | CAGAAGGTCCAGGG | TCATTATGGT | CTCTGGCTCCAAGTC | |
| | | CAGAGTCACCATGA | ATTAGTTGG | TGGCGCCTCAGCCTC | |
| | | CCACAGACACATCC | GTGCGCCAG | CCTGGCCATCACTGG | |
| | | ACGAACACAGCCTA | GCCCCTGGA | GCTCCAGGCTGAGG | |
| | | CTTGGAATTGAGGA | CAAGGCCTT | ACGAGGCTGATTATT | |
| | | GCCTGAGATCTGAC | GAGTGGATG | ACTGCAGCTCATATA | |
| | | GACACGGCCGTGTA | GCCTGGATC | CAAGCAGCAGCACT | |
| | | TTACTGTGTAAGAG | AGCGCTTAC | TTCGTGGTATTTGGT | |
| | | ATGTCCCGGCCACA | AATGGTAAC | GGAGGAACCCAGCT | |
| | | GGAGGAGCTGCCAC | ACAGACTCC | GATCATTTTAGGC | |
| | | GGCTGACTACTGGG | ATACAGAAG | (SEQ ID NO: 116) | |
| | | GCCAAGGAACCCTG | GTCCAGGGC | | |
| | | GTCACCGTCTCCTC | AGAGTCACC | | |
| | | A | ATGACCACA | | |
| | | (SEQ ID NO: 114) | GACACATCC | | |
| | | | ACGAACACA | | |
| | | | GCCTACTTGG | | |
| | | | AATTGAGGA | | |
| | | | GCCTGAGAT | | |
| | | | CTGACGACA | | |
| | | | CGGCCGTGT | | |
| | | | ATTACTGTGT | | |
| | | | AAGAGATGT | | |
| | | | CCCGGCCAC | | |
| | | | AGGAGGAGC | | |
| | | | TGCCACGGC | | |
| | | | TGACTACTG | | |
| | | | GGGCCAAGG | | |
| | | | AACCCTGGT | | |
| | | | CACCGTCTCC | | |
| | | | TCAGCCTCC | | |
| | | | ACCAAGGGC | | |
| | | | CCATCGGTCT | | |
| | | | TCCCCC | | |
| | | | (SEQ ID NO: | | |
| | | | 115) | | |
| 35 | 7A7 | CAGGTGCAGCTGGT | — | CAGTCTGCCCTGACT | — |
| | | GGAGTCTGGGGGAG | | CAGCCTGCCTCCGTG | |
| | | GCGTGGTCCAGCCT | | TCTGGGTCTCCTGGA | |
| | | GGGAGGTCCCTGAG | | CAGTCGATCACCATC | |
| | | ACTCTCCTGTGCAG | | TCCTGCACTGGAACC | |
| | | CCTCT | | AGCAGTGACGTTGGT | |
| | | GGATTCACCTTCAA | | GGTTATAACTATGTC | |
| | | TAGCTATGCTATGC | | TCCTGGTTCCAACAG | |
| | | ACTGGGTCCGCCAG | | TACCCAGGCAAAGC | |
| | | GCTCCAGGCAACGG | | CCCCAAACTCATGAT | |
| | | GCTGGACTGGGTGG | | TTATGATGTCAGTAA | |
| | | CAGGTATATCATAT | | GCGGCCCTCAGGGG | |
| | | GATGGAGGCAATAA | | TTTCTAATCGCTTCT | |
| | | ATATTACGCAGACT | | CTGGCTCCAAGTCTG | |
| | | CCGTGAAGGACCGA | | GCAACACGGCCTCCC | |
| | | TTCACCATCTCCCG | | TGACCATCTCTGAGC | |
| | | AGACAATTCCAAGA | | TCCAGGCTGAGGAT | |
| | | ACACGCTGTATCTG | | GAGGCTGATTATTAC | |
| | | CAAATGAGCAGCCT | | TGCAGCTCATATACA | |
| | | GAGAACTGACGACA | | AGCAGCAGCACTGG | |
| | | CGGCTGTATATTAC | | CGTATTCGGCGGAG | |
| | | TGTGCGAAAGATGC | | GGACCAAGCTGACC | |
| | | CGGATGGGAGGCTT | | GTCCTAGGC | |
| | | GGTGGTACTTCGAC | | (SEQ ID NO: 118) | |
| | | CTCTGGGGCCGTGG | | | |
| | | CACCCTGGTCACTG | | | |
| | | TCTCCTCA | | | |
| | | (SEQ ID NO: 117) | | | |
| 36 | 6A9 | GAGGTGCAGCTGGT | — | TGCTGTGGGTGAGTG | — |
| | | GGAGTCTGGGGGAG | | GTACCTGTGGGCAGT | |
| | | GCTTGGTACAGCCT | | CTGCCCTGACTCAGC | |
| | | GGGGGGTCCCTGAG | | CTCGCTCAGTGTCCG | |
| | | ACTCTCCTGTGCAG | | GGTCTCCTGGACAGT | |
| | | CCTCTGGAACCTTT | | CAGTCACCATCTCCT | |
| | | ACCAAATATGCCAT | | GCACTGGAACCAGC | |
| | | GACCTGGGTCCGCC | | AGTGATGTTGGGAGT | |
| | | AGGCTCTAGGGAAG | | TATAACCTTGTCTCC | |
| | | GGGCTGGAGTGGGT | | TGGTACCAACAGCA | |
| | | CTCAACTATTGGCA | | CCCAGGCAAAGCCC | |
| | | GTGGTAGTGACACT | | CCAAACTCATGATTT | |
| | | CACTACGCAGACTC | | ATGATGTCAGTAAGC | |
| | | CGTGAAGGGCCGCT | | GGCCCTCAGGGGTTT | |
| | | TCACCATCTCCAGA | | CTAATCGCTTCTCTG | |
| | | GACAATTCCAGGAA | | GCTCCAAGTCTGGCA | |
| | | CACTCTGTCTCTACA | | ACACGGCCTCCCTGA | |
| | | AATGAACAGCCTGA | | CCATCTCCGGGCTCC | |
| | | GAGCCGAGGACACG | | AGGCTGAGGATGAG | |
| | | GCCGTATATTACTG | | GCTGATTATTACTGC | |
| | | TGCCAAATATCTGG | | AGCTCATATGCAGGC | |
| | | GAATAACAGTGGGT | | AGCGGGAATGTGGT | |
| | | GATACGGGTTTTCG | | ATTTGGTGGAGGAA | |
| | | GACCTTTGACTACT | | CCCAGCTGATCATTT | |
| | | GGGGCCAGGGAACC | | TAGGCCAACCTAAG | |
| | | ACGGTCACCGTCTC | | GCCAATCCCAC | |
| | | TTCA | | (SEQ ID NO: 120) | |
| | | (SEQ ID NO: 119) | | | |
| 37 | 12B8 | CAGGTGCAGCTGGT | — | CAGTCTGCCCTGACT | — |
| | | GGAGTCTGGGGGAG | | CAGCCTCCCTCCGCG | |
| | | GCGTGGTCCAGCCT | | TCCGGGTCTCCTGGA | |
| | | GGGAGGTCCCTGAG | | CAGTCAGTCACCATC | |
| | | ACTCTCCTGTGCAG | | TCCTGCACTGGAACC | |
| | | CCTCTGGATTCACCT | | AGCAGTGACGTTGGT | |
| | | TCAGTATCTATGCT | | GGTTATAACTATGTC | |
| | | ATGCACTGGGTCCG | | TCCTGGTACCGACAG | |
| | | CCAGGCTCCAGGCA | | CACCCAGGCAAAGC | |
| | | AGGGGCTAGAGTGG | | CCCCAAACTCCTGAT | |
| | | GTGGCAGTTGTTTC | | TTATGAGGTCAATAA | |
| | | ATATGATGGAAGCG | | CCGGCCCTCAGGGGT | |
| | | AGAAATACTACGCA | | CCCTAGTCGCTTCTC | |
| | | GACTCCGTGCAGGG | | TGGCTCCAAGTCTGG | |
| | | CCGATTCACCATCT | | CAACACGGCCTCCCT | |
| | | CCAGAGACAAGTCC | | GACCATCTCTGGGCT | |
| | | AAGAACACCCTGTA | | CCAGGCTGAGGATG | |
| | | TCTGCAAATGAACA | | AGGCTGATTATTACT | |
| | | GCCTGACAGCTGAG | | GCAGCTCATATACAA | |
| | | GACACGGCTGTCTA | | GCAGCAGCACTTTCG | |
| | | TTACTGTGCGAGAG | | TGGTACTTGGTGGAG | |
| | | AGCCCTGGGTGGGG | | GAACCCAGCTGATC | |
| | | ACAATTGGCTACTG | | ATTTTAGGC | |
| | | GGGCCAGGGAACCC | | (SEQ ID NO: 122) | |
| | | TGGTCACCGTCTCCT | | | |
| | | CA | | | |
| | | (SEQ ID NO: 121) | | | |
| 38 | 12F7 | CAAATGCAGCTGGT | TGCTGTGGGT | TGCTGTGGGTGAGTG | CAGTCTGTG |
| | | GCAGTCTGGGCCTG | GAGTGGTAC | GTACCTGTGGGCAGT | CTGACTCAG |
| | | AGGTGAAGAAGCCT | CTGTGGGCA | CTGTGCTGACTCAGC | CCACCCTCA |
| | | GGGTCCTCGGTGAA | GGTTCAGCT | CACCCTCAGCGTCTG | GCGTCTGGG |
| | | GGTCTCCTGCAAGG | GGTGCAGTC | GGACCCCCGGGCAG | GCCCCCGGG |
| | | CTTCGGGTTACACC | TGGAGCTGA | AGGGTCACCATCTCT | CAGCGGGTC |
| | | TTTACCGACTACGG | GGTGAAGAA | TGTTCTGGAAGCAGC | ATCATCTCT |
| | | TCTCAGCTGGGTCC | GCCTGGGGC | TCCAACGTCGGAAGT | TGTTCTGGA |
| | | GGCAGGCCCCCGGC | CTCAGTGAA | AATACTGTCAACTGG | AGCAGCTCC |
| | | CACGGCCTTGAGTG | GGTCTCCTGC | TACCAGCAGCTCCCA | AACGTCGGA |
| | | GATGGGATGGATCA | AAGGCTTCG | GGAACGGCCCCCAA | AGTAATACT |
| | | CCGCTTACAATGGC | GGTTACACCT | ACTCCTCATCTACGA | GTCAACTGG |
| | | GACACAAACTATGC | TTACCGACTA | TAAGAATGAGCGGC | TACCAGCAG |
| | | ACAGAAGTTCCAGG | CGGTCTCAG | CCTCAGGGGTCCCGG | CTCCCAGGA |
| | | ACAGACTGTCCGTG | CTGGGTCCG | ACCGATTCTCTGCCT | ACGGCCCCC |
| | | ACCACTGACATATC | GCAGGCCCC | CCAAGTCTGGCACCT | AAACTCCTC |
| | | CACGAGCACAGCCT | CGGCCACGG | CAGCCTCCCTGGCCA | ATCTACGAT |
| | | ACATGGAATTACGG | CCTTGAGTG | TCAGTGGCCTCCAGT | AAGAATGAG |
| | | AGCCTGAAATCTGA | GATGGGATG | CTGAAGATGAGGCT | CGGCCCTCA |
| | | CGACACGGCCGTTT | GATCACCGC | GATTATTACTGTGCA | GGGGTCCCG |
| | | ATTATTGTGCGAGA | TTACAATGG | GCATGGGATGACAG | GACCGATTC |
| | | CACGTCTTTTGCAGT | CGACACAAA | CCTGAATGGTTGGGT | TCTGCCTCC |
| | | GGTGATGGGTGTTA | CTATGCACA | GTTCGGTGGAGGGA | AAGTCTGGC |
| | | CTCCGGCCTTGGGT | GAAGTTCCA | CCGAGCTGACCGTCC | ACCTCAGCC |
| | | CCTGGGGCCAGGGA | GGACAGACT | TAGGCCAACCTAAG | TCCCTGGCC |
| | | ACCCTGGTCACCGT | GTCCGTGAC | GCCAATCCCAC | ATCAGTGGC |
| | | CTCCTCA | CACTGACAT | (SEQ ID NO: 125) | CTCCAGTCT |
| | | (SEQ ID NO: 123) | ATCCACGAG | | GAAGATGAG |
| | | | CACAGCCTA | | GCTGATTAT |
| | | | CATGGAATT | | TACTGTGCA |
| | | | ACGGAGCCT | | GCATGGGAT |
| | | | GAAATCTGA | | GACAGCCTG |
| | | | CGACACGGC | | AATGGTTGG |
| | | | CGTTTATTAT | | GTGTTCGGT |
| | | | TGTGCGAGA | | GGAGGGACC |
| | | | CACGTCTTTT | | AAGCTGACC |
| | | | GCAGTGGTG | | GTCCTAGGC |
| | | | ATGGGTGTT | | (SEQ ID NO: |
| | | | ACTCCGGCC | | 126) |
| | | | TTGGGTCCTG | | |
| | | | GGGCCAGGG | | |
| | | | AACCCTGGT | | |
| | | | CACCGTCTCC | | |
| | | | TCAGCCTCC | | |
| | | | ACCAAGGGC | | |
| | | | CCATCGGTCT | | |
| | | | TCCCCC | | |
| | | | (SEQ ID NO: | | |
| | | | 124) | | |
| 39 | 7A11 | GAGGTGCAGCTGGT | — | CAGTCTGCCCTGACT | — |
| | | GGAGTCTGGGGGAG | | CAGCCTGCCTCCGTG | |
| | | GCCTGGTCAAGCCT | | TCTGGGTCTCCTGGA | |
| | | GGGGGGTCCCTGAG | | CAGTCGATCACCATC | |
| | | ACTCTCCTGTGCAG | | TCCTGCACTGGAACC | |
| | | CCTCTGGGTTCAGG | | AGCAGTGATGTTGG | |
| | | CTCAGTAGCTATGG | | GAGTTATAACCTTGT | |
| | | CATGAACTGGGTCC | | CTCCTGGTACCAACA | |
| | | GCCAGGCTCCAGGG | | GCACCCAGGCAAAG | |
| | | AAGGGGCTGGAGTG | | CCCCCAAACTCCTGA | |
| | | GGTCTCATCCATTTC | | TTTATGAGGACACTA | |
| | | TGCTAGTAGTAGTT | | AGCGGCCCTCAGGG | |
| | | TTATAAACTATGCA | | ATCCCAGACCGATTC | |
| | | GACTCAGTGAGGGG | | TCTGGCTCCAGCTCA | |
| | | CCGATTCACCATCT | | GGAAACACAGCTTC | |
| | | CCAGAGACAAGTCC | | CTTGACCATCACTGG | |
| | | AAGAACACCCTGTA | | GGCTCAGGCGGAAG | |
| | | TCTGCAAATGAACA | | ATGAGGCTGAGTATT | |
| | | GCCTGACAGCTGAG | | ACTGTAGTTCCCGGG | |
| | | GACACGGCTGTCTA | | ACAGCAGTGGTAAC | |
| | | TTACTGTGCGAGAG | | CATCTGGTGTTCGGC | |
| | | AGCCCTGGGTGGGG | | GGAGGGACCAAGCT | |
| | | ACAATTGACTACTG | | GACCGTCCTAGGC | |
| | | GGGCCAGGGAACCC | | (SEQ ID NO: 128) | |
| | | TGGTCACCGTCTCCT | | | |
| | | CA | | | |
| | | (SEQ ID NO: 127) | | | |
| 40 | 4H11 | CAGGTGCAGCTGGT | — | TCCTATGAGCTGACT | CAGTCTGTG |
| | | GCAGTCTGGAGCTG | | CAGCCACCCTCAGCG | CTGACTCAG |
| | | AGGTGAAGAAGCCT | | TCTGGGGCCCCCGGG | CCACCCTCA |
| | | GGGGCCTCAGTGAA | | CAGCGGGTCATCATC | GCGTCTGGG |
| | | GGTCTCCTGCAAGG | | TCTTGTTCTGGAAGC | GCCCCCGGG |
| | | CTTCGGGTTACACC | | AGCTCCAACGTCGG | CAGCGGGTC |
| | | TTTACCGACTACGG | | AAGTAATACTGTCAA | ATCATCTCT |
| | | TCTCAGCTGGGTCC | | CTGGTACCAGCAGCT | TGTTCTGGA |
| | | GGCAGGCCCCCGGC | | TCCAGGAAGAGCCC | AGCAGCTCC |
| | | CACGGCCTTGAGTG | | CCAAACTCCTCATCT | AACGTCGGA |
| | | GATGGGATGGATCA | | TTGCTAACACCAATC | AGTAATACT |
| | | CCGCTTACAATGGC | | GGCCCTCAGGGGTCC | GTCAACTGG |
| | | GACACAAACTATGC | | CTGACCGATTCTCTG | TACCAGCAG |
| | | ACAGAAGTTCCAGG | | GCTCCAAGTCTGGCA | CTTCCAGGA |
| | | ACAGACTGTCCGTG | | CCTCAGCCTCCCTGG | AGAGCCCCC |
| | | ACCACTGACATATC | | CCATCAGTGGCCTCC | AAACTCCTC |
| | | CACGAGCACAGCCT | | AGTCTGAAGATGAG | ATCTTTGCT |
| | | ACATGGAATTACGG | | GCTGATTATTACTGT | AACACCAAT |
| | | AGCCTGAAATCTGA | | GCAGCATGGGATGA | CGGCCCTCA |
| | | CGACACGGCCGTTT | | CAGCCTGAATGGTTG | GGGGTCCCT |
| | | ATTATTGTGCGAGA | | GGTGTTCGGTGGAG | GACCGATTC |
| | | CACGTCTTTTGCAGT | | GGACCAAGCTGACC | TCTGGCTCC |
| | | GGTGATGGGTGTTA | | GTCCTAGGC | AAGTCTGGC |
| | | CTCCGGCCTTGGGT | | (SEQ ID NO: 130) | ACCTCAGCC |
| | | CCTGGGGCCAGGGA | | | TCCCTGGCC |
| | | ACCCTGGTCACCGT | | | ATCAGTGGC |
| | | CTCCTCA | | | CTCCAGTCT |
| | | (SEQ ID NO: 129) | | | GAAGATGAG |
| | | | | | GCTGATTAT |
| | | | | | TACTGTGCA |
| | | | | | GCATGGGAT |
| | | | | | GACAGCCTG |
| | | | | | AATGGTTGG |
| | | | | | GTGTTCGGT |
| | | | | | GGAGGGACC |
| | | | | | AAGCTGACC |
| | | | | | GTCCTAGGC |
| | | | | | (SEQ ID NO: |
| | | | | | 131) |
| 41 | 2G3 | CAGGTGCAGCTGGT | — | TCCTATGAGCTGACT | CAGTCTGTG |
| | | GCAGTCTGGGGCTG | | CAGCCACTCTCAGCG | CTGACTCAG |
| | | AGGTGAAGAAGCCG | | TCTGGGACCCCCGGG | CCACTCTCA |
| | | GGGGCCTCAGTGAA | | CAGAGGGTCACCAT | GCGTCTGGG |
| | | GGTCTCCTGCAAGG | | CTCTTGTTCTGGAAG | ACCCCCGGG |
| | | CTTCGGGTTACACC | | CAGCTCCAACATCGG | CAGAGGGTC |
| | | TTTACCGACTACGG | | AAGTGGTACTGTAA | ACCATCTCT |
| | | TCTCAGCTGGGTCC | | ACTGGTACCAGCAG | TGTTCTGGA |
| | | GGCAGGCCCCCGGC | | CTCTCAGGAACGGCC | AGCAGCTCC |
| | | CACGGCCTTGAGTG | | CCCAAACTCCTCATG | AACATCGGA |
| | | GATGGGATGGATCA | | CATAGTGATAATCAG | AGTGGTACT |
| | | CCGCTTACAATGGC | | CGCCCCTCAGGGGTC | GTAAACTGG |
| | | GACACAAACTATGC | | CCTGACCGATTCTCT | TACCAGCAG |
| | | ACAGAAGTTCCAGG | | GGCTCCAAGTCTGGC | CTCTCAGGA |
| | | ACAGACTGTCCGTG | | ACCTCAGCCTCCCTG | ACGGCCCCC |
| | | ACCACTGACATATC | | GCCATCAGTGGCCTC | AAACTCCTC |
| | | CACGAGCACAGCCT | | CAGTCTGAAGATGA | ATGCATAGT |
| | | ACATGGAATTACGG | | GGCTGATTATTACTG | GATAATCAG |
| | | AGCCTGAAATCTGA | | TGCAGCATGGGATG | CGCCCCTCA |
| | | CGACACGGCCGTTT | | ACAGCCTGAATGGTT | GGGGTCCCT |
| | | ATTATTGTGCGAGA | | GGGTGTTCGGTGGA | GACCGATTC |
| | | CACGTCTTTTGCAGT | | GGGACCAAGCTGAC | TCTGGCTCC |
| | | GGTGATGGGTGTTA | | CGTCCTAGGC | AAGTCTGGC |
| | | CTCCGGCCTTGGGT | | (SEQ ID NO: 133) | ACCTCAGCC |
| | | CCTGGGGCCAGGGA | | | TCCCTGGCC |
| | | ACCCTGGTCACCGT | | | ATCAGTGGC |
| | | CTCCTCA | | | CTCCAGTCT |
| | | (SEQ ID NO: 132) | | | GAAGATGAG |
| | | | | | GCTGATTAT |
| | | | | | TACTGTGCA |
| | | | | | GCATGGGAT |
| | | | | | GACAGCCTG |
| | | | | | AATGGTTGG |
| | | | | | GTGTTCGGT |
| | | | | | GGAGGGACC |
| | | | | | AAGCTGACC |
| | | | | | GTCCTAGGC |
| | | | | | (SEQ ID NO: |
| | | | | | 134) |
| 42 | 12C10 | CAGGTGCAGCTGGT | — | CAGCCTGTGCTGACT | CAGTCTGTG |
| | | GCAGTCTGGAAGTG | | CAGCCACCCTCAGTG | CTGACTCAG |
| | | AGGTGAAGAAGCCT | | TCTGGGACCCCCGGG | CCACCCTCA |
| | | GGGGCCTCAGTGAA | | CAGAGGGTCACCAT | GTGTCTGGG |
| | | GGTCTCCTGCAAGG | | CTCTTGTTCTGGAAG | ACCCCCGGG |
| | | CTTCGGGTTACACC | | CAGCTCCAACATCGG | CAGAGGGTC |
| | | TTTACCGACTACGG | | AAGTGATACTGTAA | ACCATCTCT |
| | | TCTCAGCTGGGTCC | | ACTGGTACCAGCAG | TGTTCTGGA |
| | | GGCAGGCCCCCGGC | | CTCTCAGGAACGGCC | AGCAGCTCC |
| | | CACGGCCTTGAGTG | | CCCAAACTCCTCATG | AACATCGGA |
| | | GATGGGATGGATCA | | CATAGTGATAATCAG | AGTGATACT |
| | | CCGCTTACAATGGT | | CGCCCCTCAGGGGTC | GTAAACTGG |
| | | GACACAAACTATGC | | CCTGACCGATTCTCT | TACCAGCAG |
| | | ACAGAAGTTCCAGG | | GGCTCCAAGTCTGGC | CTCTCAGGA |
| | | ACAGACTGTCCGTG | | ACCTCAGCCTCCCTG | ACGGCCCCC |
| | | ACCACTGACATATC | | GCCATCAGTGGCCTC | AAACTCCTC |
| | | CACGAGCACAGCCT | | CAGTCTGAAGATGA | ATGCATAGT |
| | | ACATGGAATTACGG | | GGCTGATTATTACTG | GATAATCAG |
| | | AGCCTGAAATCTGA | | TGCAGCATGGGATG | CGCCCCTCA |
| | | CGACACGGCCGTTT | | ACAGCCTGAATGGTT | GGGGTCCCT |
| | | ATTATTGTGCGAGA | | GGGTGTTCGGTGGA | GACCGATTC |
| | | CACGTCTTTTGCAGT | | GG | TCTGGCTCC |
| | | GGTGATGGGTGTTA | | GACCAAGCTGACCG | AAGTCTGGC |
| | | CTCCGGCCTTGGGT | | TCCTAGGC | ACCTCAGCC |
| | | CCTGGGGCCAGGGA | | (SEQ ID NO: 136) | TCCCTGGCC |
| | | ACCCTGGTCACCGT | | | ATCAGTGGC |
| | | CTCCTCA | | | CTCCAGTCT |
| | | (SEQ ID NO: 135) | | | GAAGATGAG |
| | | | | | GCTGATTAT |
| | | | | | TACTGTGCA |
| | | | | | GCATGGGAT |
| | | | | | GACAGCCTG |
| | | | | | AATGGTTGG |
| | | | | | GTGTTCGGT |
| | | | | | GGAGGG |
| | | | | | ACCAAGCTG |
| | | | | | ACCGTCCTA |
| | | | | | GGC |
| | | | | | (SEQ ID NO: |
| | | | | | 137) |
| 43 | 3E8 | CAGGTTCAGCTGGT | CAGGTTCAG | CAGCCTGTGCTGACT | CAGTCTGTG |
| | | GCAGTCTGGAAGTG | CTGGTGCAG | CAGCCACCCTCAGCG | CTGACTCAG |
| | | AGGTGAAGAAGCCG | TCTGGAAGT | TCTGGGACCCCCGGG | CCACCCTCA |
| | | GGGGCCTCAGTGAA | GAGGTGAAG | CAGAGGGTCACCAT | GCGTCTGGG |
| | | GGTCTCCTGCAAGG | AAGCCGGGG | CTCTTGTTCTGGAAG | ACCCCCGGG |
| | | CTTCGGGTTACACC | GCCTCAGTG | CAGCTCCAACATCGG | CAGAGGGTC |
| | | TTTACCGACTACGG | AAGGTCTCCT | AAGTGATACTGTAA | ACCATCTCT |
| | | TCTCAGCTGGGTCC | GCAAGGCTT | ACTGGTACCAGCAG | TGTTCTGGA |
| | | GGCAGGCCCCCGGC | CGGGTTACA | CTCTCAGGAACGGCC | AGCAGCTCC |
| | | CACGGCCTTGAGTG | CCTTTACCGA | CCCAAACTCCTCATG | AACATCGGA |
| | | GATGGGATGGATCA | CTACGGTCTC | CATAGTGATAATCAG | AGTGATACT |
| | | CCGCTTACAATGGC | AGCTGGGTC | CGCCCCTCAGGGGTC | GTAAACTGG |
| | | GACACAAACTATGC | CGGCAGGCC | CCTGACCGATTCTCT | TACCAGCAG |
| | | ACAGAAGTTCCAGG | CCCGGCCAC | GGCTCCAAGTCTGGC | CTCTCAGGA |
| | | ACAGACTGTCCGTG | GGCCTTGAG | ACCTCAGCCTCCCTG | ACGGCCCCC |
| | | ACCACTGACATATC | TGGATGGGA | GCCATCAGTGGCCTC | AAACTCCTC |
| | | CACGAGCACAGCCT | TGGATCACC | CAGTCTGAAGATGA | ATGCATAGT |
| | | ACATGGAATTACGG | GCTTACAAT | GGCTGATTATTACTG | GATAATCAG |
| | | AGCCTGAAATCTGA | GGCGACACA | TGCAGCATGGGATG | CGCCCCTCA |
| | | CGACACGGCCGTTT | AACTATGCA | ACAGCCTGAATGGTT | GGGGTCCCT |
| | | ATTATTGTGCGAGA | CAGAAGTTC | GGGTGTTCGGTGGA | GACCGATTC |
| | | CACGTCTTTTGCAGT | CAGGACAGA | GGGACCAAGCTGAC | TCTGGCTCC |
| | | GGTGATGGGTGTTA | CTGTCCGTGA | CGTCCTAGGC | AAGTCTGGC |
| | | CTCCGGCCTTGGGT | CCACTGACA | (SEQ ID NO: 140) | ACCTCAGCC |
| | | CCTGGGGCCAGGGA | TATCCACGA | | TCCCTGGCC |
| | | ACCCTGGTCACCGT | GCACAGCCT | | ATCAGTGGC |
| | | CTCCTCA | ACATGGAAT | | CTCCAGTCT |
| | | (SEQ ID NO: 138) | TACGGAGCC | | GAAGATGAG |
| | | | TGAAATCTG | | GCTGATTAT |
| | | | ACGACACGG | | TACTGTGCA |
| | | | CCGTTTATTA | | GCATGGGAT |
| | | | TTGTGCGAG | | GACAGCCTG |
| | | | ACACGTCTTT | | AATGGTTGG |
| | | | TGCAGTGGT | | GTGTTCGGT |
| | | | GATGGGTGT | | GGAGGGACC |
| | | | TACTCCGGC | | AAGCTGACC |
| | | | CTTGGGTCCT | | GTCCTAGGC |
| | | | GGGGCCAGG | | (SEQ ID NO: |
| | | | GAACCCTGG | | 141) |
| | | | TCACCGTCTC | | |
| | | | CTCA | | |
| | | | (SEQ ID NO: | | |
| | | | 139) | | |
| 44 | 7D1 | CAGGTGCAGCTGGT | — | CAGTCTGTGCTGACT | — |
| | | GCAGTCTGGAGCTG | | CAGCCACCCTCAGCG | |
| | | AGGTGAAGAAGCCG | | TCTGGGACCCCCGGG | |
| | | GGGGCCTCAGTGAA | | CAGAGGGTCACCAT | |
| | | AGTCTCCTGCAAGG | | CTCTTGTTCTGGAAG | |
| | | CTTCGGGTTACACC | | CAGCTCCAACATCGG | |
| | | TTTACCGACTACGG | | AAGTAATACTGTAA | |
| | | TCTCAGCTGGGTCC | | ACTGGTACCAGCAG | |
| | | GGCAGGCCCCCGGC | | CTCCCAGGAACGGC | |
| | | CACGGCCTTGAGTG | | CCCCAAACTCCTCAT | |
| | | GATGGGATGGATCA | | CTATAGTAATAATCA | |
| | | CCGCTTACAATGGC | | GCGGCCCTCAGGGG | |
| | | GACACAAACTATGC | | TCCCTGACCGATTCT | |
| | | ACAGAAGTTCCAGG | | CTGGCTCCACGTCTG | |
| | | ACAGACTGTCCGTG | | GCACCTCAGCCTCCC | |
| | | ACCACTGACATATC | | TGGCCATCAGTGGAC | |
| | | CACGAGCACAGCCT | | TCCAGTCTGAAGATG | |
| | | ACATGGAATTACGG | | AGGCTGATTATTACT | |
| | | AGCCTGAAATCTGA | | GTGCAGCATGGGAT | |
| | | CGACACGGCCGTTT | | GACAGCCTGAATGG | |
| | | ATTATTGTGCGAGA | | TTGGGTGTTCGGTGG | |
| | | CACGTCTTTTGCAGT | | AGGGACCAAGCTGA | |
| | | GGTGATGGGTGTTA | | CCGTCCTAGGC | |
| | | CTCCGGCCTTGGGT | | (SEQ ID NO: 143) | |
| | | CCTGGGGCCAGGGA | | | |
| | | ACCCTGGTCACCGT | | | |
| | | CTCCTCA | | | |
| | | (SEQ ID NO: 142) | | | |
| 45 | 4G5 | CAGGTGCAGCTGGT | — | TCCTATGAGCTGACT | CAGTCTGTG |
| | | GCAGTCTGGAAGTG | | CAGCCACTCTCAGCG | CTGACTCAG |
| | | AGGTGAAGAAGCCT | | TCTGGGACCCCCGGG | CCACTCTCA |
| | | GGGGCCTCAGTGAA | | CAGAGGGTCACCAT | GCGTCTGGG |
| | | GGTCTCCTGCAAGG | | CTCTTGTTCTGGAAG | ACCCCCGGG |
| | | CTTCGGGTTACACC | | CAGCTCCAACATCGG | CAGAGGGTC |
| | | TTTACCGACTACGG | | AAGTAATACTGTAA | ACCATCTCT |
| | | TCTCAGCTGGGTCC | | ACTGGTACCAGCAG | TGTTCTGGA |
| | | GGCAGGCCCCCGGC | | CTCCCAGGAACGGC | AGCAGCTCC |
| | | CACGGCCTTGAGTG | | CCCCAAACTCCTCAT | AACATCGGA |
| | | GATGGGATGGATCA | | CTATAGTAATAATCA | AGTAATACT |
| | | CCGCTTACAATGGC | | GCGGCCCTCAGGGG | GTAAACTGG |
| | | GACACAAACTATGC | | TCCCTGACCGATTCT | TACCAGCAG |
| | | ACAGAAGTTCCAGG | | CTGGCTCCAAGTCTG | CTCCCAGGA |
| | | ACAGACTGTCCGTG | | GCACCTCAGCCTCCC | ACGGCCCCC |
| | | ACCACTGACATATC | | TGGCCATCAGTGGGC | AAACTCCTC |
| | | CACGAGCACAGCCT | | TCCAGTCTGAGGATG | ATCTATAGT |
| | | ACATGGAATTACGG | | AGGCTGATTATTACT | AATAATCAG |
| | | AGCCTGAAATCTGA | | GTGCAGCATGGGAT | CGGCCCTCA |
| | | CGACACGGCCGTTT | | GACAGCCTGAATGG | GGGGTCCCT |
| | | ATTATTGTGCGAGA | | TTGGGTGTTCGGTGG | GACCGATTC |
| | | CACGTCTTTTGCAGT | | AGGGACCAAGCTGA | TCTGGCTCC |
| | | GGTGATGGGTGTTA | | CCGTCCTAGGC | AAGTCTGGC |
| | | CTCCGGCCTTGGGT | | (SEQ ID NO: 145) | ACCTCAGCC |
| | | CCTGGGGCCAGGGA | | | TCCCTGGCC |
| | | ACCCTGGTCACCGT | | | ATCAGTGGG |
| | | CTCCTCA(SEQ ID | | | CTCCAGTCT |
| | | NO: 144) | | | GAGGATGAG |
| | | | | | GCTGATTAT |
| | | | | | TACTGTGCA |
| | | | | | GCATGGGAT |
| | | | | | GACAGCCTG |
| | | | | | AATGGTTGG |
| | | | | | GTGTTCGGT |
| | | | | | GGAGGGACC |
| | | | | | AAGCTGACC |
| | | | | | GTCCTAGGC |
| | | | | | (SEQ ID NO: |
| | | | | | 146) |
| 46 | 2E8 | CAGGTGCAGCTGGT | — | TCCTATGAGCTGACT | CAGTCTGTG |
| | | GCAGTCTGGGCCTG | | CAGCCACCCTCAGCG | CTGACTCAG |
| | | AGGTGAAGAAGCCT | | TCTGGGGCCCCCGGG | CCACCCTCA |
| | | GGGTCCTCGGTGAA | | CAGCGGGTCATCATC | GCGTCTGGG |
| | | GGTCTCCTGCAAGG | | TCTTGTTCTGGAAGC | GCCCCCGGG |
| | | CTTCGGGTTACACC | | AGCTCCAACGTCGG | CAGCGGGTC |
| | | TTTACCGACTACGG | | AAGTAATACTGTCAA | ATCATCTCT |
| | | TCTCAGCTGGGTCC | | CTGGTACCAGCAGCT | TGTTCTGGA |
| | | GGCAGGCCCCCGGC | | CCCAGGAACGGCCC | AGCAGCTCC |
| | | CACGGCCTTGAGTG | | CCAAACTCCTCATCT | AACGTCGGA |
| | | GATGGGATGGATCA | | ACGATAAGAATGAG | AGTAATACT |
| | | CCGCTTACAATGGC | | CGGCCCTCAGGGGTC | GTCAACTGG |
| | | GACACAAACTATGC | | CCGGACCGATTCTCT | TACCAGCAG |
| | | ACAGAAGTTCCAGG | | GCCTCCAAGTCTGGC | CTCCCAGGA |
| | | ACAGACTGTCCGTG | | ACCTCAGCCTCCCTG | ACGGCCCCC |
| | | ACCACTGACATATC | | GCCATCAGTGGCCTC | AAACTCCTC |
| | | CACGAGCACAGCCT | | CAGTCTGAAGATGA | ATCTACGAT |
| | | ACATGGAATTACGG | | GGCTGATTATTACTG | AAGAATGAG |
| | | AGCCTGAAATCTGA | | TGCAGCATGGGATG | CGGCCCTCA |
| | | CGACACGGCCGTTT | | ACAGCCTGAATGGTT | GGGGTCCCG |
| | | ATTATTGTGCGAGA | | GGGTGTTCGGTGGA | GACCGATTC |
| | | CACGTCTTTTGCAGT | | GGGACCAAGCTGAC | TCTGCCTCC |
| | | GGTGATGGGTGTTA | | CGTCCTAGGC | AAGTCTGGC |
| | | CTCCGGCCTTGGGT | | (SEQ ID NO: 148) | ACCTCAGCC |
| | | CCTGGGGCCAGGGA | | | TCCCTGGCC |
| | | ACCCTGGTCACCGT | | | ATCAGTGGC |
| | | CTCCTCA(SEQ ID | | | CTCCAGTCT |
| | | NO: 147) | | | GAAGATGAG |
| | | | | | GCTGATTAT |
| | | | | | TACTGTGCA |
| | | | | | GCATGGGAT |
| | | | | | GACAGCCTG |
| | | | | | AATGGTTGG |
| | | | | | GTGTTCGGT |
| | | | | | GGAGGG |
| | | | | | ACCAAGCTG |
| | | | | | ACCGTCCTA |
| | | | | | GGC |
| | | | | | (SEQ ID NO: |
| | | | | | 149) |
| 47 | 13H9 | CAGGTTCAGCTGGT | — | TCCTATGAGCTGATA | CAGTCTGTG |
| | | GCAGTCTGGAGCTG | | CAGCCACCCTCAGCG | CTGACTCAG |
| | | AGGTGAAGAAGCCT | | TCTGGGGCCCCCGGG | CCACCCTCA |
| | | GGGGCCTCAGTGAA | | CAGCGGGTCATCATC | GCGTCTGGG |
| | | GGTCTCCTGCAAGG | | TCTTGTTCTGGAAGC | GCCCCCGGG |
| | | CTTCTGGTTACACC | | AGCTCCAACGTCGG | CAGCGGGTC |
| | | GGTTACACCTTTAC | | AAGTAATACTGTCAA | ATCATCTCT |
| | | CGACTACGGTCTCA | | CTGGTACCAGCAGCT | TGTTCTGGA |
| | | GCTGGGTCCGGCAG | | CCCAGGAACGGCCC | AGCAGCTCC |
| | | GCCCCCGGCCACGG | | CCAAACTCCTCATCT | AACGTCGGA |
| | | CCTTGAGTGGATGG | | ACGATAAGAATGAG | AGTAATACT |
| | | GATGGATCACCGCT | | CGGCCCTCAGGGGTC | GTCAACTGG |
| | | TACAATGGCGACAC | | CCGGACCGATTCTCT | TACCAGCAG |
| | | AAACTATGCACAGA | | GCCTCCAAGTCTGGC | CTCCCAGGA |
| | | AGTTCCAGGACAGA | | ACCTCAGCCTCCCTG | ACGGCCCCC |
| | | CTGTCCGTGACCAC | | GCCATCAGTGGCCTC | AAACTCCTC |
| | | TGACATATCCACGA | | CAGTCTGAAGATGA | ATCTACGAT |
| | | GCACAGCCTACATG | | GGCTGATTATTACTG | AAGAATGAG |
| | | GAATTACGGAGCCT | | TGCAGCATGGGATG | CGGCCCTCA |
| | | GAAATCTGACGACA | | ACAGCCTGAATGGTT | GGGGTCCCG |
| | | CGGCCGTTTATTATT | | GGGTGTTCGGTGGA | GACCGATTC |
| | | GTGCGAGACACGTC | | GGGACCGAGCTGAC | TCTGCCTCC |
| | | TTTTGCAGTGGTGA | | CGTCCTAGGC | AAGTCTGGC |
| | | TGGGTGTTACTCCG | | (SEQ ID NO: 151) | ACCTCAGCC |
| | | GCCTTGGGTCCTGG | | | TCCCTGGCC |
| | | GGCCAGGGAACCCT | | | ATCAGTGGC |
| | | GGTCACCGTCTCCT | | | CTCCAGTCT |
| | | CA(SEQ ID NO: 150) | | | GAAGATGAG |
| | | | | | GCTGATTAT |
| | | | | | TACTGTGCA |
| | | | | | GCATGGGAT |
| | | | | | GACAGCCTG |
| | | | | | AATGGTTGG |
| | | | | | GTGTTCGGT |
| | | | | | GGAGGGACC |
| | | | | | AAGCTGACC |
| | | | | | GTCCTAGGC |
| | | | | | (SEQ ID NO: |
| | | | | | 152) |
| 48 | 4E9 | CAGGTGCAGCTGGT | — | CAGTCTGTGCTGACG | — |
| | | GCAGTCTGGAGCTG | | CAGCCGCCCTCAGTG | |
| | | AGGTGAAGAAGCCT | | TCTGGGGCCCCAGG | |
| | | GGGTCCTCGGTGAA | | GCAGAGGGTCACCA | |
| | | GGTCTCCTGCAAGG | | TCTCTTGTTCTGGAA | |
| | | CTTCGGGTTACACC | | GCAGCTCCAACGTCG | |
| | | TTTACCGACTACGG | | GAAGTAATACTGTCA | |
| | | TCTCAGCTGGGTCC | | ACTGGTACCAGCAG | |
| | | GGCAGGCCCCCGGC | | CTCCCAGGAACGGC | |
| | | CACGGCCTTGAGTG | | CCCCAAACTCCTCAT | |
| | | GATGGGATGGATCA | | CTACGATAAGAATG | |
| | | CCGCTTACAATGGC | | AGCGGCCCTCAGGG | |
| | | GACACAAACTATGC | | GTCCCGGACCGATTC | |
| | | ACAGAAGTTCCAGG | | TCTGCCTCCAAGTCT | |
| | | ACAGACTGTCCGTG | | GGCACCTCATCCTCC | |
| | | ACCACTGACATATC | | CTGGCCATCGGTGGG | |
| | | CACGAGCACAGCCT | | CTCCGGTCTGAAGAT | |
| | | ACATGGAATTACGG | | GAGGCTGATTATTAC | |
| | | AGCCTGAAATCTGA | | TGTGGGACATGGGA | |
| | | CGACACGGCCGTTT | | TGACAACCTGAATG | |
| | | ATTATTGTGCGAGA | | GTTGGGTGTTCGGCG | |
| | | CACGTCTTTTGCAGT | | GAGGGACCAAGCTG | |
| | | GGTGATGGGTGTTA | | ACCGTCCTAGGC | |
| | | CTCCGGCCTTGGGT | | (SEQ ID NO: 154) | |
| | | CCTGGGGCCAGGGA | | | |
| | | ACCCTGGTCACCGT | | | |
| | | CTCCTCA(SEQ ID | | | |
| | | NO: 153) | | | |
| 49 | 4H7 | CAGGTGCAGCTGGT | — | TCCTATGAGCTGACT | CAGTCTGTG |
| | | GCAGTCTGGAGCTG | | CAGCCACCCTCAGCG | CTGACTCAG |
| | | AGGTGAAGAAGCCT | | TCTGGGGCCCCCGGG | CCACCCTCA |
| | | GGGGCCTCAGTGAA | | CAGCGGGTCATCATC | GCGTCTGGG |
| | | GGTCTCCTGCAAGG | | TCTTGTTCTGGAAGC | GCCCCCGGG |
| | | CTTCGGGTTACACC | | AGCTCCAACGTCGG | CAGCGGGTC |
| | | TTTACCGACTACGG | | AAGTAATACTGTCAA | ATCATCTCT |
| | | TCTCAGCTGGGTCC | | CTGGTACCAGCAGCT | TGTTCTGGA |
| | | GGCAGGCCCCCGGC | | CCCAGGAACGGCCC | AGCAGCTCC |
| | | CACGGCCTTGAGTG | | CCAAACTCCTCATCT | AACGTCGGA |
| | | GATGGGATGGATCA | | ACGATAAGAATGAG | AGTAATACT |
| | | CCGCTTACAATGGC | | CGGCCCTCAGGGGTC | GTCAACTGG |
| | | GACACAAACTATGC | | CCGGACCGATTCTCT | TACCAGCAG |
| | | ACAGAAGTTCCAGG | | GCCTCCAAGTCTGGC | CTCCCAGGA |
| | | ACAGACTGTCCGTG | | ACCTCATCCTCCCTG | ACGGCCCCC |
| | | ACCACTGACATATC | | GCCATCGGTGGGCTC | AAACTCCTC |
| | | CACGAGCACAGCCT | | CGGTCTGAAGATGA | ATCTACGAT |
| | | ACATGGAATTACGG | | GGCTGATTATTACTG | AAGAATGAG |
| | | AGCCTGAAATCTGA | | TGCGACATGGGATG | CGGCCCTCA |
| | | CGACACGGCCGTTT | | ACAACCTGAATGGTT | GGGGTCCCG |
| | | ATTATTGTGCGAGA | | GGGTGTTCGGCGGA | GACCGATTC |
| | | CACGTCTTTTGCAGT | | GGGACCAAGCTGAC | TCTGCCTCC |
| | | GGTGATGGGTGTTA | | CGTCCTAGGC | AAGTCTGGC |
| | | CTCCGGCCTTGGGT | | (SEQ ID NO: 156) | ACCTCATCC |
| | | CCTGGGGCCAGGGA | | | TCCCTGGCC |
| | | ACCCTGGTCACCGT | | | ATCGGTGGG |
| | | CTCCTCA(SEQ ID | | | CTCCGGTCT |
| | | NO: 155) | | | GAAGATGAG |
| | | | | | GCTGATTAT |
| | | | | | TACTGTGCG |
| | | | | | ACATGGGAT |
| | | | | | GACAACCTG |
| | | | | | AATGGTTGG |
| | | | | | GTGTTCGGC |
| | | | | | GGAGGGACC |
| | | | | | AAGCTGACC |
| | | | | | GTCCTAGGC |
| | | | | | (SEQ ID NO: |
| | | | | | 157) |
| 50 | 2F8 | CAGGTGCAGCTGGT | — | TCCTATGAGCTGACT | CAGTCTGTG |
| | | GCAGTCTGGAGCTG | | CAGCCACCCTCAGCG | CTGACTCAG |
| | | AGGTGAAGAAGCCT | | TCTGGGGCCCCCGGG | CCACCCTCA |
| | | GGGGCCTCAGTGAA | | CAGCGGGTCATCATC | GCGTCTGGG |
| | | GGTCTCCTGCAAGG | | TCTTGTTCTGGAAGC | GCCCCCGGG |
| | | CTTCGGGTTACACC | | AGCTCCAACGTCGG | CAGCGGGTC |
| | | TTTACCGACTACGG | | AAGTAATACTGTCAA | ATCATCTCT |
| | | TCTCAGCTGGGTCC | | CTGGTACCAGCAGCT | TGTTCTGGA |
| | | GGCAGGCCCCCGGC | | CCCAGGAACGGCCC | AGCAGCTCC |
| | | CACGGCCTTGAGTG | | CCAAACTCCTCATCT | AACGTCGGA |
| | | GATGGGATGGATCA | | ACGATAAGAATGAG | AGTAATACT |
| | | CCGCTTACAATGGC | | CGGCCCTCAGGGGTC | GTCAACTGG |
| | | GACACAAACTATGC | | CCGGACCGATTCTCT | TACCAGCAG |
| | | ACAGAAGTTCCAGG | | GCCTCCAAGTCTGGC | CTCCCAGGA |
| | | ACAGACTGTCCGTG | | ACCTCATCCTCCCTG | ACGGCCCCC |
| | | ACCACTGACATATC | | GCCATCGGTGGGCTC | AAACTCCTC |
| | | CACGAGCACAGCCT | | CGGTCTGAAGATGA | ATCTACGAT |
| | | ACATGGAATTACGG | | GGCTGATTATTACTG | AAGAATGAG |
| | | AGCCTGAAATCTGA | | TGGGACATGGGATG | CGGCCCTCA |
| | | CGACACGGCCGTTT | | ACAACCTGAATGGTT | GGGGTCCCG |
| | | ATTATTGTGCGAGA | | GGGTGTTCGGCGGA | GACCGATTC |
| | | CACGTCTTTTGCAGT | | GGGACCAAGCTGAC | TCTGCCTCC |
| | | GGTGATGGGTGTTA | | CGTCCTAGGC | AAGTCTGGC |
| | | CTCCGGCCTTGGGT | | (SEQ ID NO: 159) | ACCTCATCC |
| | | CCTGGGGCCAGGGA | | | TCCCTGGCC |
| | | ACCCTGGTCACCGT | | | ATCGGTGGG |
| | | CTCCTCA(SEQ ID | | | CTCCGGTCT |
| | | NO: 158) | | | GAAGATGAG |
| | | | | | GCTGATTAT |
| | | | | | TACTGTGGG |
| | | | | | ACATGGGAT |
| | | | | | GACAACCTG |
| | | | | | AATGGTTGG |
| | | | | | GTGTTCGGC |
| | | | | | GGAGGG |
| | | | | | ACCAAGCTG |
| | | | | | ACCGTCCTA |
| | | | | | GGC |
| | | | | | (SEQ ID NO: |
| | | | | | 160) |
| 51 | 3E7 | GAGGTGCAGCTGGT | TGCTGTGGGT | CAGTCTGTGCTGACT | — |
| | | GGAGTCTGGGACTG | GAGTGGTAC | CAGCCACCCTCAGCG | |
| | | AGGTGAAGAAGCCT | CTGTGGGGA | TCTGGGGCCCCCGGG | |
| | | GGGGCCTCAGTGAA | GGTGCAGCT | CAGCGGGTCATCATC | |
| | | GGTCTCCTGCAAGG | GGTGCAGTC | TCTTGTTCTGGAAGC | |
| | | CTTCGGGTTACACC | TGGGGCTGA | AGCTCCAACGTCGG | |
| | | TTTACCGACTACGG | GGTGAAGAA | AAGTAATACTGTCAA | |
| | | TCTCAGCTGGGTCC | GCCTGGGGC | CTGGTACCAGCAGCT | |
| | | GGCAGGCCCCCGGC | CTCAGTGAA | CCCAGGAACGGCCC | |
| | | CACGGCCTTGAGTG | GGTCTCCTGC | CCAAACTCCTCATCT | |
| | | GATGGGATGGATCA | AAGGCTTCG | ACGATAAGAATGAG | |
| | | CCGCTTACAATGGC | GGTTACACCT | CGGCCCTCAGGGGTC | |
| | | GACACAAACTATGC | TTACCGACTA | CCGGACCGATTCTCT | |
| | | ACAGAAGTTCCAGG | CGGTCTCAG | GCCTCCAAGTCTGGC | |
| | | ACAGACTGTCCGTG | CTGGGTCCG | ACCTCATCCTCCCTG | |
| | | ACCACTGACATATC | GCAGGCCCC | GCCATCGGTGGGCTC | |
| | | CACGAGCACAGCCT | CGGCCACGG | CGGTCTGAAGATGA | |
| | | ACATGGAATTACGG | CCTTGAGTG | GGCTGATTATTACTG | |
| | | AGCCTGAAATCTGA | GATGGGATG | TGGGACATGGGATG | |
| | | CGACACGGCCGTTT | GATCACCGC | ACAACCTGAATGGTT | |
| | | ATTATTGTGCGAGA | TTACAATGG | GGGTGTTCGGCGGA | |
| | | CACGTCTTTTGCAGT | CGACACAAA | GGGACCAAGCTGAC | |
| | | GGTGATGGGTGTTA | CTATGCACA | CGTCCTAGGC | |
| | | CTCCGGCCTTGGGT | GAAGTTCCA | (SEQ ID NO: 163) | |
| | | CCTGGGGCCAGGGA | GGACAGACT | | |
| | | ACCCTGGTCACTGT | GTCCGTGAC | | |
| | | CTCCTCA | CACTGACAT | | |
| | | (SEQ ID NO: 161) | ATCCACGAG | | |
| | | | CACAGCCTA | | |
| | | | CATGGAATT | | |
| | | | ACGGAGCCT | | |
| | | | GAAATCTGA | | |
| | | | CGACACGGC | | |
| | | | CGTTTATTAT | | |
| | | | TGTGCGAGA | | |
| | | | CACGTCTTTT | | |
| | | | GCAGTGGTG | | |
| | | | ATGGGTGTT | | |
| | | | ACTCCGGCC | | |
| | | | TTGGGTCCTG | | |
| | | | GGGCCAGGG | | |
| | | | AACCCTGGT | | |
| | | | CACTGTCTCC | | |
| | | | TCAGCCTCC | | |
| | | | ACCAAGGGC | | |
| | | | CCATCGGTCT | | |
| | | | TCCCCC | | |
| | | | (SEQ ID NO: | | |
| | | | 162) | | |
| 52 | 9H4 | CAGGTGCAGCTGGT | GAGAGGTGC | GAAATTGTGCTGACC | — |
| | | GCAGTCTGGGGGAG | AGCTGGTGG | CAGTCTCCAGGCATC | |
| | | GCTTGGTACAGCCT | AGTCTGGGG | CAGTCTTTGTCTCCA | |
| | | GGAGGGTCCCTGAG | GAGGCTTGG | GGGGAAACAGCCAC | |
| | | AATCTCCTGTACAG | TACAGCCTG | CCTCTCCTGCAGGGC | |
| | | CCTCTGGATTCACCT | GAGGGTCCC | CAGTGAGAGTATTA | |
| | | TCAGGAATTATGAA | TGAGAATCT | GCAGCAGTTACTTCG | |
| | | ATGAATTGGGTCCG | CCTGTACAG | CCTGGTACCAGCAG | |
| | | CCAGGCTCCAGGGA | CCTCTGGATT | AAACCTGGCCAGGC | |
| | | AGGGGCTGGAGTGG | CACCTTCAG | TCCCAGGCTCCTCAT | |
| | | GTTGCATACATTAG | GAATTATGA | CTATGGTGCATCCAG | |
| | | TAGTAGTGGTAGTT | AATGAATTG | CAGGGCCTCTGGCAT | |
| | | CCAGATACTACGCA | GGTCCGCCA | CCCAGACAGGTTCA | |
| | | GACTCTGTGAAGGG | GGCTCCAGG | GTGGCAGTGGGTCTG | |
| | | CCGATTCACCATCT | GAAGGGGCT | GGACAGACTTCACTC | |
| | | CCAGAGACAACGCC | GGAGTGGGT | TCACCATCAGCAGAC | |
| | | AAGAACTCACTGTT | TGCATACATT | TGGAGCCTGAAGATT | |
| | | TCTGCAAATGAACA | AGTAGTAGT | TTGCAGTGTATTACT | |
| | | GCCTGAGAGCCGAG | GGTAGTTCC | GTCAGCAGTATGGTA | |
| | | GACATGGCTGTTTA | AGATACTAC | GCTCACCTCCCATCA | |
| | | TTACTGTGCGAGGA | GCAGACTCT | CCTTCGGC | |
| | | CGGACGACGGCAGC | GTGAAGGGC | CAAGGGACACGACT | |
| | | AGCTGGTTCGTGTC | CGATTCACC | GGAGATTAAACGA | |
| | | CACCAGTAGTTTCT | ATCTCCAGA | (SEQ ID NO: 166) | |
| | | ACGGTATGGACGTC | GACAACGCC | | |
| | | TGGGGCCAAGGGAC | AAGAACTCA | | |
| | | CACGGTCACCGTCT | CTGTTTCTGC | | |
| | | CCTCA | AAATGAACA | | |
| | | (SEQ ID NO: 164) | GCCTGAGAG | | |
| | | | CCGAGGACA | | |
| | | | TGGCTGTTTA | | |
| | | | TTACTGTGCG | | |
| | | | AGGACGGAC | | |
| | | | GACGGCAGC | | |
| | | | AGCTGGTTC | | |
| | | | GTGTCCACC | | |
| | | | AGTAGTTTCT | | |
| | | | ACGGTATGG | | |
| | | | ACGTCTGGG | | |
| | | | GCCAAGGGA | | |
| | | | CCACGGTCA | | |
| | | | CCGTCTCCTC | | |
| | | | A | | |
| | | | (SEQ ID NO: | | |
| | | | 165) | | |
| 53 | 8B1 | CAGGTGCAGCTGGT | GAGAGGTGC | GATGTTGTGATGACT | GAGATTGTG |
| | | GCAGTCT | AGCTGGTGG | CAGTCTCCA | CTGACCCAG |
| | | GGGGGAGGCTTGGT | AGTCTGGGG | GGCACCCTGTCTTTG | TCTCCAGGC |
| | | ACAGCCTGGAGGGT | GAGGCTTGG | TCTCCAGGGGAAAG | ACCCTGTCT |
| | | CCCTGAGAATCTCC | TACAGCCTG | AGCCACCCTCTCCTG | TTGTCTCCA |
| | | TGTACAGCCTCTGG | GAGGGTCCC | CAGGGCCAGTCAGA | GGGGAAAG |
| | | ATTCACCTTCAGGA | TGAGAATCT | GTGTTAGTAGCAGCT | AGCCACCCT |
| | | ATTATGAAATGAAT | CCTGTACAG | ACTTAGCCTGGTACC | CTCCTGCAG |
| | | TGGGTCCGCCAGGC | CCTCTGGATT | AGCAGAAACCTGGC | GGCCAGTCA |
| | | TCCAGGGAAGGGGC | CACCTTCAG | CAGGCTCCCAGGCTC | GAGTGTTAG |
| | | TGGAGTGGGTTGCA | GAATTATGA | CTCATCTACGGTGTG | TAGCAGCTA |
| | | TACATTAGTAGTAG | AATGAATTG | TCCAGCAGGGCCACT | CTTAGCCTG |
| | | TGGTAGTTCCAGAT | GGTCCGCCA | GGCATCCCAGACAG | GTACCAGCA |
| | | ACTACGCAGACTCT | GGCTCCAGG | GTTCAGTGGCAGTGG | GAAACCTGG |
| | | GTGAAGGGCCGATT | GAAGGGGCT | GTCTGGGACAGACTT | CCAGGCTCC |
| | | CACCATCTCCAGAG | GGAGTGGGT | CACTCTCACCATCAG | CAGGCTCCT |
| | | ACAACGCCAAGAAC | TGCATACATT | CAGACTGGAGCCTG | CATCTACGG |
| | | ACGCTGTATCTGCA | AGTAGTAGT | AAGATTTTGCAGTGT | TGTGTCCAG |
| | | AATGAGCAGCCTGA | GGTAGTTCC | ATTACTGTCAGCAGT | CAGGGCCAC |
| | | GATCTGACGACACG | AGATACTAC | ATGGTAGCTCACCTC | TGGCATCCC |
| | | GCCGTGTATTACTG | GCAGACTCT | CCATCACCTTCGGCC | AGACAGGTT |
| | | TGCGAGGACGGACG | GTGAAGGGC | AGGGGACCAAGCTG | CAGTGGCAG |
| | | ACGGCAGCAGCTGG | CGATTCACC | GAGATCAAACGA | TGGGTCTGG |
| | | TTCGTGTCCACCAG | ATCTCCAGA | (SEQ ID NO: 169) | GACAGACTT |
| | | TAGTTTCTACGGTAT | GACAACGCC | | CACTCTCAC |
| | | GGACGTCTGGGGCC | AAGAACACG | | CATCAGCAG |
| | | AAGGGACCACGGTC | CTGTATCTGC | | ACTGGAGCC |
| | | ACCGTCTCCTCA | AAATGAGCA | | TGAAGATTT |
| | | (SEQ ID NO: 167) | GCCTGAGAT | | TGCAGTGTA |
| | | | CTGACGACA | | TTACTGTCA |
| | | | CGGCCGTGT | | GCAGTATGG |
| | | | ATTACTGTGC | | TAGCTCACC |
| | | | GAGGACGGA | | TCCCATCAC |
| | | | CGACGGCAG | | CTTCGGCCA |
| | | | CAGCTGGTTC | | G GGG ACC |
| | | | GTGTCCACC | | AAG CTG |
| | | | AGTAGTTTCT | | GAG ATC |
| | | | ACGGTATGG | | AAA CGT |
| | | | ACGTCTGGG | | (SEQ ID NO: |
| | | | GCCAAGGGA | | 170) |
| | | | CCACGGTCA | | |
| | | | CCGTCTCCTC | | |
| | | | A | | |
| | | | (SEQ ID NO: | | |
| | | | 168) | | |
| 54 | 7G12 | GAGGTGCAGCTGGT | — | GACATCCAGTTGACC | — |
| | | GGAGTCTGGGGGAG | | CAGTCTCCATCTTCC | |
| | | GCTTGGTACAGCCT | | GTGTCTGCATCTGTA | |
| | | GGGGGGTCCCTGAG | | GGAGAGAGAGTCAC | |
| | | ACTCTCCTGTGCAG | | CATCACTCGTCGGGC | |
| | | CC | | GAGTCAGAATATTG | |
| | | TCTGAATTCACCTTT | | ACAGGTGGTTAGCTT | |
| | | AGCATGAACTGGGT | | GGTATCAGCAGAAA | |
| | | CCGACAGGCTCCAG | | CCAGGGAAAGCCCC | |
| | | GGAAGGGGCTGGAG | | TGACCTCCTGATCTT | |
| | | TGGGTCTCATCAGT | | TGCTGCATCCAGTTT | |
| | | TCGAGGTGGCGGTA | | GCAGAGTGGGGTCC | |
| | | CTGAAACATACTAT | | CATCAAGGTTCAGCG | |
| | | GCAGACTCCGTGAA | | GCAGTGGATCTGGG | |
| | | GGGCCGGTTCACCG | | ACAGATTTCACTCTC | |
| | | TCTCCAGAGACAAT | | ACCATCAGCAGCCTG | |
| | | TCCAAGAACACACT | | CAGCCTGAAGATTTT | |
| | | GTATCTGCAGATGA | | GCAACTTACTATTGT | |
| | | ACAGCCTGAGAGTC | | CAACAGGGTAAAAC | |
| | | GAGGACACGGCCGT | | TTTCCCTCCCACGTT | |
| | | GTATTACTGTACGG | | CGGCCAGGGGACCA | |
| | | TGTGTGTTGTTATGT | | AGCTGGAGATCAAA | |
| | | TTCGGGGATGGTGC | | CGA | |
| | | AACTGGTTCGACCC | | (SEQ ID NO: 172) | |
| | | CTGGGGCCAGGGAA | | | |
| | | CCCTGGTCACCGTC | | | |
| | | TCCTCA | | | |
| | | (SEQ ID NO: 171) | | | |
| 55 | 12F11 | GAGGTGCAGCTGTT | — | GATGTTGTGATGACT | GACATCCAG |
| | | GGAGTCTGGGGGAG | | CAGTCTCCATCGTCC | ATGACCCAG |
| | | GCTTGGTACAGCCT | | CTGTCTGCATCCGTT | TCTCCATCG |
| | | GGGGGGTCCCTGAG | | GGGGACAGAGTCAC | TCCCTGTCT |
| | | ACTCTCCTGTGCAG | | CATCACTTGCCGGGC | GCATCCGTT |
| | | CCTCTGAATTCACCT | | AAGTCAGAGCCTCA | GGGGACAGA |
| | | TTAGCATGAACTGG | | GTAGTTATTTAAATT | GTCACCATC |
| | | GTCCGACAGGCTCC | | GGTATCAGCAGAAA | ACTTGCCGG |
| | | AGGGAAGGGGCTGG | | CCAGGGAAAGCCCC | GCAAGTCAG |
| | | AGTGGGTCTCATCA | | TAAACTGCTCATCTA | AGCCTCAGT |
| | | GTTCGAGGTGGCGG | | TGCTACAACCAACTT | AGTTATTTA |
| | | TACTGAAACATACT | | GCAAAGTGGGGTCC | AATTGGTAT |
| | | ATGCAGACTCCGTG | | CTTCAAGGTTCAGTG | CAGCAGAAA |
| | | AAGGGCCGGTTCAC | | GCAGTGGATCTGGG | CCAGGGAAA |
| | | CGTCTCCAGAGACA | | ACACATTTCACTCTC | GCCCCTAAA |
| | | ATTCCAAGAACACA | | ACCATCGGGAGTCTG | CTGCTCATC |
| | | CTGTATCTGCAGAT | | CAACCTGAAGATTTT | TATGCTACA |
| | | GAACAGCCTGAGAG | | GCAACTTATTACTGT | ACCAACTTG |
| | | TCGAGGACACGGCC | | CAACAGAGTTTCCAG | CAAAGTGGG |
| | | GTGTATTACTGTAC | | ACCCCGCTCACTTTC | GTCCCTTCA |
| | | GGTGTGTGTTGTTAT | | GGCGGAGGGACCAA | AGGTTCAGT |
| | | GTTTCGGGGATGGT | | GGTGGAGATCAAAC | GGCAGTGGA |
| | | GCAACTGGTTCGAC | | GA | TCTGGGACA |
| | | CCCTGGGGCCAGGG | | (SEQ ID NO: 174) | CATTTCACT |
| | | AACCCTGGTCACCG | | | CTCACCATC |
| | | TCTCCTCA(SEQ ID | | | GGGAGTCTG |
| | | NO: 173) | | | CAACCTGAA |
| | | | | | GATTTTGCA |
| | | | | | ACTTATTAC |
| | | | | | TGTCAACAG |
| | | | | | AGTTTCCAG |
| | | | | | ACCCCGCTC |
| | | | | | ACTTTCGGC |
| | | | | | GGAGGG |
| | | | | | ACC AAG |
| | | | | | CTG GAG |
| | | | | | ATC AAA |
| | | | | | CGT |
| | | | | | (SEQ ID NO: |
| | | | | | 175) |
| 56 | 5B12 | CAGGTGCAGCTGGT | — | GACATCCAGATGAC | — |
| | | GGAGTCTGGGGGAG | | CCAGTCTCCAGCCAC | |
| | | GCGTGGTCCAGCCT | | CCTGTCTGCATCTGT | |
| | | GGGAGGTCCCTGAG | | AGGAGACAGAGTCA | |
| | | ACTCTCCTGTGCAG | | CCATCACTTGCCGGG | |
| | | CCTCTGGATTCACCT | | CAAGTCAGAGCATT | |
| | | TCATTAGCTATGTC | | AACAACAATTTAAAT | |
| | | ATGCACTGGGTCCG | | TGGTATCAACAGAA | |
| | | CCAGGCTCCAGGCA | | ACCAGGGCAAGCCC | |
| | | AGGGGCTGGAGTGG | | CTAAGCTCCTGATCT | |
| | | GTGGCAGTTGTTTC | | ATGCTGCATCCACTT | |
| | | ATATGATGGAAGCG | | TACAAGGTGGGGTC | |
| | | AGAAATACTACGCA | | CCATCAAGGTTCAGC | |
| | | GACTCCGTGAAGGG | | GGCAGTGGATCTGG | |
| | | CCGATTCACCATCT | | GACAGAATTCACTCT | |
| | | CCAGAGACAATTCC | | CACAATCAGCAGCCT | |
| | | AAGAACACGCTGTA | | GCAGCCTGAAGATTT | |
| | | TCTGCAAATGAACA | | TGCAACTTATTACTG | |
| | | GCCTGACAGCTGAG | | TCAACAGCTTAATGG | |
| | | GACACGGCTGTCTA | | TTACCCTCTCACTTT | |
| | | TTACTGTGCGAGAG | | CGGCGGAGGGACCA | |
| | | AGCCCTGGGTGGGG | | AGGTGGAGATCAAA | |
| | | ACAATTGACTACTG | | CGA | |
| | | GGGCCAGGGAACCC | | (SEQ ID NO: 177) | |
| | | TGGTCACCGTCTCCT | | | |
| | | CA(SEQ ID NO: 176) | | | |
| 57 | 8C3 | GAGGTGCAGCTGTT | — | GACATCCAGATGAC | — |
| | | GGAGTCTGGGGGAG | | CCAGTCTCCAGACTC | |
| | | GCTTGGTACAGCCT | | CCTGGCTGTGTCTCT | |
| | | GGGGGGTCCCTGAG | | GGGCGAGAGGGCCA | |
| | | ACTCTCCTGTGCAG | | CCATCAACTGCAAGT | |
| | | CCTCTGGATTCACCT | | CCAGCCAGAGTGTTT | |
| | | TTAGCGACTATGCC | | TATACAGCTCCAACA | |
| | | ATGAGTTGGGTCCG | | ATAAGAACTACTTAG | |
| | | CCAGGCTCCAGGGA | | CTTGGTACCAGCAGA | |
| | | AGGGGCTGGAGTGG | | AACCAGGACAGCCT | |
| | | GTCTCAAGTATTAC | | CCTAAGATGCTCATT | |
| | | TGCTAATGGACTTA | | TACTGGGCATCTATC | |
| | | TGACATACTACACA | | CGGGAAACCGGGGT | |
| | | GACTCCGTGAAGGG | | CCCTGACCGATTCAG | |
| | | CCGATTCACCATCT | | TGGCAGCGGGTCTG | |
| | | CCCGAGACAATTCC | | AGACAGATTTCACTC | |
| | | AAGAACACGCTGTA | | TCACCATCAGCAGCC | |
| | | TCTGCAAATGAGCA | | TGCAGGCTGAAGAT | |
| | | GCCTGAGAACTGAC | | GTGGCAGTTTATTAC | |
| | | GACACGGCTGTATA | | TGTCAGCAATATTAT | |
| | | TTACTGTGCGAAAG | | AGTACTCTCACTTTC | |
| | | ATGCCGGATGGGAG | | GGCGGAGGGACCAA | |
| | | GCTTGGTGGTACTT | | GGTGGAGATCAAAC | |
| | | CGACCTCTGGGGCC | | GA | |
| | | GTGGCACCCTGGTC | | (SEQ ID NO: 179) | |
| | | ACCGTCTCCTCA | | | |
| | | (SEQ ID NO: 178) | | | |
| 58 | 2F4 | CAGGTGCAGCTGGT | — | GAGATTGTGCTGACC | — |
| | | GCAGTCTGGGGCTG | | CAGTCTCCAGCCACC | |
| | | AGGTGAAGAAGCCT | | GTGTCTCTGTCTCCC | |
| | | GGGGCCTCAGTGAA | | GGGGAAAGAACCAT | |
| | | GGTTTCCTGCAAGG | | GTTGTCTTGCAGGGC | |
| | | CATCTGGATACACC | | CAGTCAGAATGTTAG | |
| | | TTCACCAGCTACTA | | TAACTACTTAGGGTG | |
| | | TATGCGCTGGCTGC | | GTATCAGCAGAGAC | |
| | | GACAGGCCCCTGGA | | GTGGCCAGCCTCCCA | |
| | | CAAGGGCTTGAGTG | | GACTCCTCATTTCCG | |
| | | GATGGGAATAATCG | | ATGCGTCCAACAGG | |
| | | ACCCTAGTGGTGGT | | GCCTCTGGCGTCCCA | |
| | | AGCACAACCTATGC | | GCCAGGTTCAGTGG | |
| | | ACAGAAGTTCCAGG | | AAGTGGGTCTGGGA | |
| | | GCAGAGTCAGCATA | | CAGACTTCACTCTTA | |
| | | ACCAGGGACACGTC | | CTATCACCAGTCTTC | |
| | | CACGAGCACAGTCT | | AGCCTGAAGATTTTG | |
| | | ACATGGAGCTGAGC | | CAGTTTATTTCTGTC | |
| | | AGCCTGAGATCTGA | | AGCACCGGAGCAGC | |
| | | GGACACGGCCGTGT | | TGGCCCGTCACTTTC | |
| | | ATTACTGTGCGAGA | | GGCGGAGGG | |
| | | GGGAGGGGCGTAGT | | ACCAAGGTGGAGAT | |
| | | ACCAGCTGGCAACC | | CAAACGA | |
| | | CCTCAACGGGGGGG | | (SEQ ID NO: 181) | |
| | | GTCGGCATGGACGT | | | |
| | | CTGGGGCCAAGGGA | | | |
| | | CCACGGTCACCGTC | | | |
| | | TCCTCA(SEQ ID NO: | | | |
| | | 180) | | | |
| 59 | 1C10 | CAGGTGCAGCTGGT | — | GATGTTGTGATGACT | — |
| | | GCAGTCTGGAAGTG | | CAGTCTCCACTCTCC | |
| | | AGGTGAAGAAGCCT | | CTGCCCGTCACCCTT | |
| | | GGGGCCTCAGTGAA | | GGCCAGCCGGCCTCC | |
| | | GGTCTCCTGCAAGG | | ATCTCCTGCAGGTCT | |
| | | CCTCTGGATACACC | | AGTCAAAGCCTCGTC | |
| | | TTTGGTCATTATGGT | | TTCAGTGATGGAAAC | |
| | | ATTAGTTGGGTGCG | | ACCTACTTGAGTTGG | |
| | | CCAGGCCCCTGGAC | | TTTCAACAGAGGCCA | |
| | | AAGGCCTTGAGTGG | | GGCCAATCTCCAAG | |
| | | ATGGCCTGGATCAG | | GCGCCTAATTTATAA | |
| | | CGCTTACAATGGTA | | GGTTTCTAACCGGGA | |
| | | ACACAGACTCCATA | | CTCTGGGGTCCCAGA | |
| | | CAGAAGGTCCAGGG | | CAGATTCAGCGCCA | |
| | | CAGAGTCACCATGA | | GTGGGTCAGGCACT | |
| | | CCACAGACACATCC | | GATTTCACACTGAAA | |
| | | ACGAACACAGCCTA | | ATCAGCAGGGTGGA | |
| | | CTTGGAATTGAGGA | | GGCTGACGATGTTGG | |
| | | GCCTGAGATCTGAC | | GGTTTATTACTGCAT | |
| | | GACACGGCCGTGTA | | GCAAGTTACACACTG | |
| | | TTACTGTGTAAGAG | | GCCTCGGACTTTTGG | |
| | | ATGTCCCGGCCACA | | CCAG | |
| | | GGAGGAGCTGCCAC | | GGGACCAAGCTGGA | |
| | | GGCTGACTACTGGG | | GATCAAACGA | |
| | | GCCAGGGAACCCTG | | (SEQ ID NO: 183) | |
| | | GTCACCGTCTCCTC | | | |
| | | A | | | |
| | | (SEQ ID NO: 182) | | | |
| 60 | 5E9 | CAGGTGCAGCTGGT | — | GATGTTGTGATGACT | — |
| | | GCAGTCTGGGCCTG | | CAGTCTCCACTCTCC | |
| | | AGGTGAAGAAGCCT | | CTGCCCGTCACCCTT | |
| | | GGGTCCTCAGTGAA | | GGCCAGCCGGCCTCC | |
| | | GGTCTCCTGCAAGG | | ATCTCCTGCAGGTCT | |
| | | CCTCTGGATACACC | | AGTCAAAGCCTCGTC | |
| | | TTTGGTCATTATGGT | | TTCAGTGATGGAAAC | |
| | | ATTAGTTGGGTGCG | | ACCTACTTGAGTTGG | |
| | | CCAGGCCCCTGGAC | | TTTCAACAGAGGCCA | |
| | | AAGGCCTTGAGTGG | | GGCCAATCTCCAAG | |
| | | ATGGCCTGGATCAG | | GCGCCTAATTTATAA | |
| | | CGCTTACAATGGTA | | GGTTTCTAACCGGGA | |
| | | ACACAGACTCCATA | | CTCTGGGGTCCCAGA | |
| | | CAGAAGGTCCAGGG | | CAGATTCAGCGCCA | |
| | | CAGAGTCACCATGA | | GTGGGTCAGGCACT | |
| | | CCACAGACACATCC | | GATTTCACACTGAAA | |
| | | ACGAACACAGCCTA | | ATCAGCAGGGTGGA | |
| | | CTTGGAATTGAGGA | | GGCTGACGATGTTGG | |
| | | GCCTGAGATCTGAC | | GGTTTATTACTGCAT | |
| | | GACACGGCCGTGTA | | GCAAGTTACACACTG | |
| | | TTACTGTGTAAGAG | | GCCTCGGACTTTTGG | |
| | | ATGTCCCGGCCACA | | CCAAGGGACACGAC | |
| | | GGAGGAGCTGCCAC | | TGGAGATTAAACGA | |
| | | GGCTGACTACTGGG | | (SEQ ID NO: 185) | |
| | | GCCAGGGAACCACG | | | |
| | | GTCACCGTCTCCTC | | | |
| | | A(SEQ ID NO: 184) | | | |
| 61 | 5C9 | CAGGTGCAGCTGGT | — | GATGTTGTGATGACT | — |
| | | GCAGTCTGGAAGTG | | CAGTCTCCACTCTCC | |
| | | AGGTGAAGAAGCCT | | CTGCCCGTCACCCTT | |
| | | GGGGCCTCAGTGAA | | GGCCAGCCGGCCTCC | |
| | | GGTCTCCTGCAAGG | | ATCTCCTGCAGGTCT | |
| | | CCTCTGGATACACC | | AGTCAAAGCCTCGTC | |
| | | TTTGGTCATTATGGT | | TTCAGTGATGGAAAC | |
| | | ATTAGTTGGGTGCG | | ACCTACTTGAGTTGG | |
| | | CCAGGCCCCTGGAC | | TTTCAACAGAGGCCA | |
| | | AAGGCCTTGAGTGG | | GGCCAATCTCCAAG | |
| | | ATGGCCTGGATCAG | | GCGCCTAATTTATAA | |
| | | CGCTTACAATGGTA | | GGTTTCTAACCGGGA | |
| | | ACACAGACTCCATA | | CTCTGGGGTCCCAGA | |
| | | CAGAAGGTCCAGGG | | CAGATTCAGCGCCA | |
| | | CAGAGTCACCATGA | | GTGGGTCAGGCACT | |
| | | CCACAGACACATCC | | GATTTCACACTGAAA | |
| | | ACGAACACAGCCTA | | ATCAGCAGGGTGGA | |
| | | CTTGGAATTGAGGA | | GGCTGACGATGTTGG | |
| | | GCCTGAGATCTGAC | | GGTTTATTACTGCAT | |
| | | GACACGGCCGTGTA | | GCAAGTTACACACTG | |
| | | TTACTGTGTAAGAG | | GCCTCGGACTTTTGG | |
| | | ATGTCCCGGCCACA | | CCAG | |
| | | GGAGGAGCTGCCAC | | GGGACCAAGCTGGA | |
| | | GGCTGACTACTGGG | | GATCAAACGA | |
| | | GCCAGGGAACCACG | | (SEQ ID NO: 187) | |
| | | GTCACCGTCTCCTC | | | |
| | | A(SEQ ID NO: 186) | | | |
| 62 | 5C7 | CAGGTACAGCTGCA | TGCTGTGGGT | GATGTTGTGATGACA | — |
| | | GCAGTCTGGAAGTG | GAGTGGTAC | CAGTCTCCACTCTCC | |
| | | AGGTGAAGAAGCCT | CTGTGGGCA | TTACCTGTCCCCCTT | |
| | | GGGGCCTCAGTGAA | GGTACAGCT | GGACAGCCGGCCTC | |
| | | GGTCTCCTGCAAGG | GGTGCAGTC | CATCTCCTGCAGGTC | |
| | | CCTCTGGATACACC | TGGAGCTGA | TAGTCAAAGCCTCGT | |
| | | TTTGGTCATTATGGT | GGTGAAGAA | ACACAGTGATGGAA | |
| | | ATTAGTTGGGTGCG | GCCTGGGGC | ACACCTACTTGAGTT | |
| | | CCAGGCCCCTGGAC | CTCAGTGAA | GGTTTCAGCAGAGG | |
| | | AAGGCCTTGAGTGG | GGTCTCCTGC | CCAGGCCAATCTCCA | |
| | | ATGGCCTGGATCAG | AAGGCCTCT | AGGCGCCTAATTTAT | |
| | | CGCTTACAATGGTA | GGATACACC | AAGGTTTCTGACCGG | |
| | | ACACAGACTCCATA | TTTGGTCATT | GACTCTGGGGTCCCA | |
| | | CAGAAGGTCCAGGG | ATGGTATTA | GACAGATTCAGCGG | |
| | | CAGAGTCACCATGA | GTTGGGTGC | CAGTGGGTCAGGCA | |
| | | CCACAGACACATCC | GCCAGGCCC | CTGATTTCACACTGA | |
| | | ACGAACACAGCCTA | CTGGACAAG | AAATCAGCAGGGTG | |
| | | CTTGGAATTGAGGA | GCCTTGAGT | GAGGCTGAGGATGT | |
| | | GCCTGAGATCTGAC | GGATGGCCT | TGGGGTTTATTACTG | |
| | | GACACGGCCGTGTA | GGATCAGCG | CATGCAAGTTACACA | |
| | | TTACTGTGTAAGAG | CTTACAATG | CTGGCCTCGGACTTT | |
| | | ATGTCCCGGCCACA | GTAACACAG | TGGCCAAGGGACAC | |
| | | GGAGGAGCTGCCAC | ACTCCATAC | GACTGGAGATTAAA | |
| | | GGCTGACTACTGGG | AGAAGGTCC | CGA | |
| | | GCCAGGGAACCCTG | AGGGCAGAG | (SEQ ID NO: 190) | |
| | | GTCACCGTCTCCTC | TCACCATGA | | |
| | | A | CCACAGACA | | |
| | | (SEQ ID NO: 188) | CATCCACGA | | |
| | | | ACACAGCCT | | |
| | | | ACTTGGAATT | | |
| | | | GAGGAGCCT | | |
| | | | GAGATCTGA | | |
| | | | CGACACGGC | | |
| | | | CGTGTATTAC | | |
| | | | TGTGTAAGA | | |
| | | | GATGTCCCG | | |
| | | | GCCACAGGA | | |
| | | | GGAGCTGCC | | |
| | | | ACGGCTGAC | | |
| | | | TACTGGGGC | | |
| | | | CAGGGAACC | | |
| | | | CTGGTCACC | | |
| | | | GTCTCCTCAG | | |
| | | | CCTCCACCA | | |
| | | | AGGGCCCAT | | |
| | | | CGGTCTTCCC | | |
| | | | CC | | |
| | | | (SEQ ID NO: | | |
| | | | 189) | | |
| 63 | 8A11 | CAGGTGCAGCTGGT | — | GATGTTGTGATGACT | — |
| | | GCAGTCTGGAAGTG | | CAGTCTCCACTCTCC | |
| | | AGGTGAAGAAGCCT | | CTGCCCGTCACCCTT | |
| | | GGGGCCTCAGTGAA | | GGACAACCGGCCTC | |
| | | GGTCTCCTGCAAGG | | CATCTCCTGCAAGTC | |
| | | CCTCTGGATACACC | | TAGTCGAAGCCTCGT | |
| | | TTTGGTCATTATGGT | | ACACAGTGATGGAA | |
| | | ATTAGTTGGGTGCG | | ACACCTACTTGAGTT | |
| | | CCAGGCCCCTGGAC | | GGTTTCAACAGAGG | |
| | | AAGGCCTTGAGTGG | | CCAGGCCAATCTCCA | |
| | | ATGGCCTGGATCAG | | AGGCGCCTAATTTAT | |
| | | CGCTTACAATGGTA | | AAGGTTTCTAACCGG | |
| | | ACACAGACTCCATA | | GACTCTGGGGTCCCA | |
| | | CAGAAGGTCCAGGG | | GACAGATTCAGCGC | |
| | | CAGAGTCACCATGA | | CAGTGGGTCAGGCA | |
| | | CCACAGACACATCC | | CTGATTTCACACTGA | |
| | | ACGAACACAGCCTA | | AAATCAGCAGGGTG | |
| | | CTTGGAATTGAGGA | | GAGGCTGACGATGTT | |
| | | GCCTGAGATCTGAC | | GGGGTTTATTACTGC | |
| | | GACACGGCCGTGTA | | ATGCAAGTTACACAC | |
| | | TTACTGTGTAAGAG | | TGGCCTCGGACTTTT | |
| | | ATGTCCCGGCCACA | | GGCCAG | |
| | | GGAGGAGCTGCCAC | | GGGACCAAGCTGGA | |
| | | GGCTGACTACTGGG | | GATCAAACGA | |
| | | GCCAGGGAACCCTG | | (SEQ ID NO: 192) | |
| | | GTCACCGTCTCCTC | | | |
| | | A | | | |
| | | (SEQ ID NO: 191) | | | |

TABLE 19
Amino acid sequences of heavy chain and light chain
variable domains of 63 types of clones

| | | Heavy chain | | Light chain | |
|---|---|---|---|---|---|
| | | | Editing with | | Editing with |
| | | | SEQ (amino | | SEQ (amino |
| | | | acid) | | acid) |
| | | | Germline | | Germline |
| | Clone | SEQ | sequence | SEQ | sequence |
| # | name | (amino acid) | (Modified) | (amino acid) | (Modified) |
| 1 | 2G4 | EVQLVESGGGL | — | QSVLTQPPSVSG | — |
| | | VKPGGSLRLSC | | APGQRVTISCTG | |
| | | AASGFTFSSYT | | SSSNIGAGYDVH | |
| | | MHWVRQAPG | | WYQQVPGTAPK | |
| | | KGLEWVSSITG | | LLIFGSTNRPSGV | |
| | | GSSYVDYSASV | | PDRFSGSKSGTS | |
| | | KGRFTISRDNA | | ASLAITGLQADD | |
| | | QSSLYLQMNSL | | EADYYCQSYDR | |
| | | RAEDTAVYYC | | SLSHVFGTGTKV | |
| | | ARDDYGSGSY | | TVLG | |
| | | SNWFDPWGQG | | (SEQ ID NO: 194) | |
| | | TLVTVSS | | | |
| | | (SEQ ID NO: | | | |
| | | 193) | | | |
| 2 | 2H3 | EVQLVESGGG | — | QSVLTQPPSVSG | — |
| | | VVQSGRSLRLS | | APGQRVTISCTG | |
| | | CAGSGFTFSSY | | SSSNIGAGYDVH | |
| | | TMHWVRQAPG | | WYQQVPGTAPK | |
| | | KGLEWVSSITG | | LLIFGSTNRPSGV | |
| | | GSSYVDYSASV | | PDRFSGSKSGAS | |
| | | KGRFTISRDNA | | ASLAITGLQTED | |
| | | QSSLYLQMNSL | | EADYYCQSYDR | |
| | | RAEDTAVYYC | | SLSHVFGTGTKV | |
| | | ARDDYGSGSY | | TVLG | |
| | | SNWFDPWGQG | | (SEQ ID NO: 196) | |
| | | TLVTVSS | | | |
| | | (SEQ ID NO: | | | |
| | | 195) | | | |
| 3 | 4H1 | EVQLVESGGGL | — | QSVLTQPPSVSG | — |
| | | VKPGGSLRLSC | | APGQRVTISCTG | |
| | | AGSGFTFSSYT | | SSSNIGAGYDVH | |
| | | MHWVRQAPG | | WYQQVPGTAPK | |
| | | KGLEWVSSITG | | LLIFGSTNRPSGV | |
| | | GSSYVDYSASV | | PDRFSGSKSGAS | |
| | | KGRFTISRDNA | | ASLAITGLQADD | |
| | | QSSLYLQMNSL | | EADYYCQPYDR | |
| | | RAEDTAVYYC | | SLSHVFGTGTKV | |
| | | ARDDYGSGSY | | TVLR | |
| | | SNWFDPWGQG | | (SEQ ID NO: 198) | |
| | | TLVTVSS | | | |
| | | (SEQ ID NO: | | | |
| | | 197) | | | |
| 4 | 4E3 | EVQLVESGGGL | — | QSVLTQPPSVSG | — |
| | | VKPGGSLRLSC | | APGQRVTISCTG | |
| | | AGSGFTFSSYT | | SSSNIGAGYDVH | |
| | | MHWVRQAPG | | WYQQVPGTAPK | |
| | | KGLEWVSSITG | | LLIFGSTNRPSGV | |
| | | GSSYVDYSASV | | PDRFSGSKSGAS | |
| | | KGRFTISRDNA | | ASLAITGLQADD | |
| | | QSSLYLQMNSL | | EADYYCQSYDR | |
| | | RAEDTAVYYC | | SLSHVFGTGTKV | |
| | | ARDDYGSGSY | | TVLG | |
| | | SNWFDPWGQG | | (SEQ ID NO: 200) | |
| | | TLVTVSS(SEQ | | | |
| | | ID NO: 199) | | | |
| 5 | 4H5 | EVQLVESGGGL | — | QSVLTQPPSVSG | — |
| | | VKPGGSLRLSC | | APGQRVTISCTG | |
| | | AGSGFTFSSYT | | SSSNIGAGYDVH | |
| | | MHWVRQAPG | | WYQQLPGAAPR | |
| | | KGLEWVSSITG | | LLMFGNSNRPSG | |
| | | GSSYVDYSASV | | VPDRFSGSKSGT | |
| | | KGRFTISRDNA | | SASLAITGLQAE | |
| | | QSSLYLQMNSL | | DEADYYCQSYD | |
| | | RAEDTAVYYC | | RSLSHVFGTGTK | |
| | | ARDDYGSGSY | | VTVLG | |
| | | SNWFDPWGQG | | (SEQ ID NO: 202) | |
| | | TLVTVSS(SEQ | | | |
| | | ID NO: 201) | | | |
| 6 | 2E7 | EVQLVESGGGL | — | SYELTQPPSASG | QSVLTQPPSA |
| | | VKPGGSLRLSC | | TPGQRVTISCTG | SGTPGQRVTI |
| | | AGSGFTFSSYT | | SSSNIGAGYDVH | SCTGSSSNIG |
| | | MHWVRQAPG | | WYQQLPGAAPR | AGYDVHWYQ |
| | | KGLEWVSSITG | | LLMFGNSNRPSG | QLPGAAPRLL |
| | | GSSYVDYSASV | | VPDRFSGSKSGT | MFGNSNRPSG |
| | | KGRFTISRDNA | | SASLAITGLQAD | VPDRFSGSKS |
| | | QSSLYLQMNSL | | DEADYYCQSYD | GTSASLAITG |
| | | RAEDTAVYYC | | RSLSHVFGTGTK | LQADDEADY |
| | | ARDDYGSGSY | | VTVLG | YCQSYDRSLS |
| | | SNWFDPWGQG | | (SEQ ID NO: 203) | HVFGTGTKVT |
| | | TLVTVSS(SEQ | | | VLG |
| | | ID NO: 29) | | | (SEQ ID NO: |
| | | | | | 30) |
| 7 | 2H2 | QVQLVQSGGG | — | QSVLTQPPSVSG | — |
| | | LIQPGGSLRLSC | | APGQRVTISCTG | |
| | | AGSGFTFSSYT | | SSSNIGAGYDVH | |
| | | MHWVRQAPG | | WYQQLPGTAPK | |
| | | KGLEWVSSITG | | LLIFANTNRPSG | |
| | | GSSYVDYSASV | | VPDRFSGSKSGA | |
| | | KGRFTISRDNA | | SASLAITGLQAD | |
| | | QSSLYLQMNSL | | DEADYYCQSYD | |
| | | RAEDTAVYYC | | RSLSHVFGTGTK | |
| | | ARDDYGSGSY | | VTVLG | |
| | | SNWFDPWGQG | | (SEQ ID NO: 205) | |
| | | TLVTVSS | | | |
| | | (SEQ ID NO: 204) | | | |
| 8 | 12A1 | EVQLVESGGGL | — | QSVLTQPPSVSG | — |
| | | IQPGGSLRLSC | | APGQRVTISCTG | |
| | | AGSGFTFSSYT | | SSSDIGAGYDVH | |
| | | MHWVRQAPG | | WYQQLPGTAPK | |
| | | KGLEWVSSITG | | LLIFANTNRPSG | |
| | | GSSYVDYSASV | | VPDRFSGSKSGA | |
| | | KGRFTISRDNA | | SASLAITGLQAD | |
| | | QSSLYLQMNSL | | DEADYYCQSYD | |
| | | RAEDTAVYYC | | RSLSHVFGTGTK | |
| | | ARDDYGSGSY | | VTVLG | |
| | | SNWFDPWGQG | | (SEQ ID NO: 20) | |
| | | TLVTVSS | | | |
| | | (SEQ ID NO: | | | |
| | | 19) | | | |
| 9 | 2H1 | QVTLKESGGG | EVQLVESGGG | QSVLTQPPSVSG | — |
| | | VVQSGRSLRLS | VVQSGRSLRL | APGQRVTISCTG | |
| | | CAASGFTFSSY | SCAASGFTFS | SSSNIGAGYDVH | |
| | | TMHWVRQAPG | SYTMHWVRQ | WYQQLPGRAPK | |
| | | KGLEWVSSITG | APGKGLEWV | LLIYANTNRPSG | |
| | | GSSYVDYSASV | SSITGGSSYV | VADRFSGSKSGA | |
| | | KGRFTISRDNA | DYSASVKGRF | SASLAITGLQAD | |
| | | QSSLYLQMNSL | TISRDNAQSS | DEADYYCQSYD | |
| | | RAEDTAVYYC | LYLQMNSLR | RSLSHVFGTGTK | |
| | | ARDDYGSGSY | AEDTAVYYC | VTVLG | |
| | | SNWFDPWGQG | ARDDYGSGS | (SEQ ID NO: 22) | |
| | | TLVTVSS | YSNWFDPWG | | |
| | | (SEQ ID NO: 21) | QGTLVTVSS | | |
| | | | (SEQ ID NO: | | |
| | | | 206) | | |
| 10 | 4G4 | EVQLVESGGGL | — | QSVLTQPPSVSG | — |
| | | VKPGGSLRLSC | | APGQRVTISCTG | |
| | | AGSGFTFSSYT | | NSSNLGAGYDV | |
| | | MHWVRQAPG | | HWYQQLPGTAP | |
| | | KGLEWVSSITG | | KLLIYANTNRPS | |
| | | GSSYVDYSASV | | GVPDRFSGSKSG | |
| | | KGRFTISRDNA | | ASASLAITGLQT | |
| | | QSSLYLQMNSL | | EDEADYYCQSY | |
| | | RAEDTAVYYC | | DRSLSHVFGTGT | |
| | | ARDDYGSGSY | | KVTVLG | |
| | | SNWFDPWGQG | | (SEQ ID NO: 208) | |
| | | TLVTVSS | | | |
| | | (SEQ ID NO: | | | |
| | | 207) | | | |
| 11 | 12E9 | EVQLVESGGGL | — | QTVVTQEPSVSG | QSVLTQPPSV |
| | | VKPGGSLRLSC | | APGQRVTISCTG | SGAPGQRVTI |
| | | AASGFTFTSYR | | SSSNIGAGYDVH | SCTGSSSNIG |
| | | MHWVRQAPG | | WYQQLPGRAPK | AGYDVHWYQ |
| | | KGLEWVSSITG | | LLIFANTNRPSG | QLPGRAPKLL |
| | | GGNYIEYADSV | | VPDRFSGSKSGA | IFANTNRPSG |
| | | KGRFTISRDNA | | SASLAITGLQAD | VPDRESGSKS |
| | | QSSLYLQMNSL | | DEADYYCQSYD | GASASLAITG |
| | | RAEDTAVYYC | | RSLSHVFGTGTK | LQADDEADY |
| | | ARDDYGSGSY | | VTVLG | YCQSYDRSLS |
| | | SNWFDPWGQG | | (SEQ ID NO: 209) | HVFGTGTKVT |
| | | TLVTVSS | | | VLG |
| | | (SEQ ID NO: 31) | | | (SEQ ID NO: |
| | | | | | 24) |
| 12 | 4F11 | EVQLVESGGGL | — | QPVLTQPPSVSG | QSVLTQPPSV |
| | | VKPGGSLRLSC | | APGQRVTISCTG | SGAPGQRVTI |
| | | AGSGFAFSSYT | | SSSNIGAGYDVH | SCTGSSSNIG |
| | | MHWVRQAPG | | WYQQVPGTAPK | AGYDVHWYQ |
| | | KGLEWVSSITG | | LLIFGSTNRPSGV | QVPGTAPKLL |
| | | GSSYLDYAHSV | | PDRFSGSKSGAS | IFGSTNRPSG |
| | | KGRFTISRDNG | | ASLAITGLQTED | VPDRFSGSKS |
| | | QNSLFLQMNSL | | EADYYCQSYDR | GASASLAITG |
| | | RTEDTAVYYC | | SLSHVFGTGTKV | LQTEDEADY |
| | | ARDDYGSGSY | | TVLG | YCQSYDRSLS |
| | | SNYFDPWGQG | | (SEQ ID NO: 210) | HVFGTGTKVT |
| | | TLVTVSS | | | VAR |
| | | (SEQ ID NO: 32) | | | (SEQ ID NO: |
| | | | | | 33) |
| 13 | 10E7 | EVQLVESGGGL | — | QSVLTQPPSVSG | — |
| | | VKPGGSLRLSC | | APGQRVTISCTG | |
| | | AASGFTFTSYR | | SSSNIGAGYDVH | |
| | | MHWVRQAPG | | WYQQLPGRAPK | |
| | | KGLEWVSSITG | | LLIFANTNRPSG | |
| | | GGNYIEYADSV | | VPDRESGSKSGA | |
| | | KGRFTISRDNA | | SASLAITGLQAD | |
| | | KNSLDLQMNS | | DEADYYCQSYD | |
| | | LRAEDTAIYYC | | RSLSHVFGTGTK | |
| | | ARDMYGLGSY | | VTVLG | |
| | | YSPNYFDSWG | | (SEQ ID NO: 24) | |
| | | QGTLVTVSS | | | |
| | | (SEQ ID NO: 23) | | | |
| 14 | 2E9 | QVTLKESGGGL | — | QSVLTQPPSVSG | — |
| | | VKPGGSLRLSC | | APGQRVTISCTG | |
| | | AGSGFTFSSYT | | SSSNIGAGYDVH | |
| | | MHWVRQAPG | | WYQQLPGRAPK | |
| | | KGLEWVSSITG | | LLIFANTNRPSG | |
| | | GSSYVDYSASV | | VPDRFSGSKSGA | |
| | | KGRFTISRDNA | | SASLAITGLQAD | |
| | | KNSLFLQMNSL | | DEADYYCQSYD | |
| | | RAEDTAVYYC | | RSLSHVFGTGTK | |
| | | ARDDYGSGSY | | VTVLG | |
| | | SNWFDPWGQG | | (SEQ ID NO: 212) | |
| | | TLVTVSS | | | |
| | | (SEQ ID NO: 211) | | | |
| 15 | 12B11 | EVQLVESGGG | — | SYELTQPPSVSG | QSVLTQPPSV |
| | | VVQPGRSLRLA | | APGQRVTISCTG | SGAPGQRVTI |
| | | CAASGFTFNSY | | SSSNIGAGYDVH | SCTGSSSNIG |
| | | AMHWVRQAP | | WYQQLPGRAPK | AGYDVHWYQ |
| | | GKGLEWVSSIT | | LLIFANTNRPSG | QLPGRAPKLL |
| | | GGSSYVDYSAS | | VPDRFSGSKSGT | IFANTNRPSG |
| | | VKGRFTISRDN | | SASLAITGLQAE | VPDRFSGSKS |
| | | AQSSLYLQMN | | DEADYYCQSYD | GTSASLAITG |
| | | SLRAEDTAVY | | SSLSYVFGTGTK | LQAEDEADY |
| | | YCARDMYGLG | | VTVLG | YCQSYDSSLS |
| | | SYYSPNYFDPW | | (SEQ ID NO: 214) | YVFGTGTKVT |
| | | GQGTLVTVSS | | | VLG |
| | | (SEQ ID NO: 213) | | | (SEQ ID NO: |
| | | | | | 215) |
| 16 | 3H8 | EVQLVESGGGL | — | QPVLTQPLSASG | QSVLTQPLSA |
| | | VKPGGSLRLSC | | TPGQRVTISCTG | SGTPGQRVTI |
| | | AGSGFTFSSYT | | SSSNIGAGYDVH | SCTGSSSNIG |
| | | MHWVRQAPG | | WYQQLPGRAPK | AGYDVHWYQ |
| | | KGLEWVSSITG | | LLIFANTNRPSG | QLPGRAPKLL |
| | | GSSYVDYSASV | | VPDRFSGSKSGT | IFANTNRPSG |
| | | KGRFTISRDNA | | SASLAITGLQAE | VPDRFSGSKS |
| | | QSSLYLQMNSL | | DEADYYCQSYD | GTSASLAITG |
| | | RAEDTAVYYC | | RSLSHVFGTGTK | LQAEDEADY |
| | | ARDDYGSGSY | | VTVLG | YCQSYDRSLS |
| | | SNWFDPWGRG | | (SEQ ID NO: 26) | HVFGTGTKVT |
| | | TTVTVSS | | | VLG |
| | | (SEQ ID NO: 25) | | | (SEQ ID NO: |
| | | | | | 216) |
| 17 | 4E1 | EVQLVESGGGL | — | SYELTQPPSVSG | QSVLTQPPSV |
| | | VQPGGSLRLAC | | APGQRVTISCTG | SGAPGQRVTI |
| | | AASGFTFSSYT | | SSSNIGAGYDVH | SCTGSSSNIG |
| | | MHWVRQAPG | | WYQQLPGRAPK | AGYDVHWYQ |
| | | KGLEWVSSITG | | LLIFANTNRPSG | QLPGRAPKLL |
| | | GSSYVDYSASV | | VPDRESGSKSGA | IFANTNRPSG |
| | | KGRFTISRDNA | | SASLAITGLQAE | VPDRFSGSKS |
| | | QSSLYLQMNSL | | DEADYYCQSYD | GASASLAITG |
| | | RAEDTAVYYC | | RSLSHVFGTGTK | LQAEDEADY |
| | | ARDDYGSGSY | | VTVLG | YCQSYDRSLS |
| | | SNWFDPWGQG | | (SEQ ID NO: 218) | HVFGTGTKVT |
| | | TLVTVSS | | | VLG |
| | | (SEQ ID NO: 217) | | | (SEQ ID NO: |
| | | | | | 219) |
| 18 | 2H9 | QVTLKESGGGL | — | SYELTQPLSVSG | QSVLTQPLSV |
| | | VKPGGSLRLSC | | APGQRVTISCTG | SGAPGQRVTI |
| | | AASGFTFTSYR | | SSSNIGAGYDVH | SCTGSSSNIG |
| | | MHWVRQAPG | | WYQQVPGTAPK | AGYDVHWYQ |
| | | KGLEWVSSITG | | LLIFGSTNRPSGV | QVPGTAPKLL |
| | | GGNYIEYADSV | | PDRFSGSKSGAS | IFGSTNRPSG |
| | | KGRFTISRDNA | | ASLAITGLQADD | VPDRFSGSKS |
| | | KNSLDLQMNS | | EADYYCQSYDR | GASASLAITG |
| | | LRAEDTAIYYC | | SLSHVFGTGTKV | LQADDEADY |
| | | ARDMYGLGSY | | TVLG | YCQSYDRSLS |
| | | YSPNYFDPWG | | (SEQ ID NO: 221) | HVFGTGTKVT |
| | | QGTLVTVSS | | | VLG |
| | | (SEQ ID NO: | | | (SEQ ID NO: |
| | | 220) | | | 222) |
| 19 | 12C11 | EVQLVESGGGL | — | QSVLTQPPSVSG | — |
| | | VKPGGSLRLSC | | APGQRVTISCTG | |
| | | AASGFTFTSYR | | SSSNIGAGYDVH | |
| | | MHWVRQAPG | | WYQQVPGTAPK | |
| | | KRLEWVSSITG | | LLIFGSTNRPSGV | |
| | | GGNYIEYADSV | | PDRFSGSKSGAS | |
| | | KGRFTISRDNA | | ASLAITGLQTED | |
| | | KNSLDLQMNS | | EADYYCQSYDR | |
| | | LRAEDTAIYYC | | SLSHVFGTGTKV | |
| | | ARDMYGLGSY | | TVLG | |
| | | YSPNYFDPWG | | (SEQ ID NO: 224) | |
| | | QGTLVTVSS | | | |
| | | (SEQ ID NO: 223) | | | |
| 20 | 3E10 | EVQLVESGGGL | — | QPVLTQPPSVSG | QSVLTQPPSV |
| | | VKPGGSLRLSC | | APGQRVTISCTG | SGAPGQRVTI |
| | | AASGFTFTSYR | | SSSNIGAGYDVH | SCTGSSSNIG |
| | | MHWVRQAPG | | WYQQVPGTAPK | AGYDVHWYQ |
| | | KGLEWVSSITG | | LLIFGSTNRPSGV | QVPGTAPKLL |
| | | GGNYIEYADSV | | PDRFSGSKSGAS | IFGSTNRPSG |
| | | KGRFTISRDNA | | ASLAITGLQTED | VPDRFSGSKS |
| | | KNSLDLQMNS | | EADYYCQSYDR | GASASLAITG |
| | | LRAEDTAIYYC | | SLSHVFGTGTKV | LQTEDEADY |
| | | ARDMYGLGSY | | TVLG | YCQSYDRSLS |
| | | YSPNYFDPWG | | (SEQ ID NO: 226) | HVFGTGTKVT |
| | | QGTLVTVSS | | | VLG |
| | | (SEQ ID NO: | | | (SEQ ID NO: |
| | | 225) | | | 227) |
| 21 | 2G1 | QVTLKESGGGL | — | QSVLTQPPSVSG | — |
| | | VKPGGSLRLSC | | APGQRVTISCTG | |
| | | AASGFTFTSYR | | SSSNIGAGYDVH | |
| | | MHWVRQAPG | | WYQQLPGTAPK | |
| | | KGLEWVSSITG | | LLIFGSTNRPSGV | |
| | | GGNYIEYADSV | | PDRFSGSKSGAS | |
| | | KGRFTISRDNA | | ASLAITGLQTED | |
| | | KNSLDLQMNS | | EADYYCQSYDR | |
| | | LRAEDTAIYYC | | SLSHVFGTGTKV | |
| | | ARDMYGLGSY | | TVLG | |
| | | YSPNYFDPWG | | (SEQ ID NO: 229) | |
| | | QGTLVTVSS | | | |
| | | (SEQ ID NO: | | | |
| | | 228) | | | |
| 22 | 1E6 | EVQLVESGGGL | — | QSVLTQPPSVSG | — |
| | | VKPGGSLRLSC | | APGQRVTISCTG | |
| | | AASGFTFTSYR | | SSSNIGAGYDVH | |
| | | MHWVRQAPG | | WYQQLPGRAPK | |
| | | KGLEWVSSITG | | LLIFANTNRPSG | |
| | | GGNYIEYADSV | | VPDRFSGSKSGA | |
| | | KGRFTISRDNA | | SASLAITGLQAD | |
| | | KNSLDLQMNS | | DEADYYCQSYD | |
| | | LRAEDTAIYYC | | RSLSHVFGTGTK | |
| | | ARDMYGLGSY | | VTVLG | |
| | | YSPNYFDPWG | | (SEQ ID NO: 231) | |
| | | QGTLVTVSS | | | |
| | | (SEQ ID NO: | | | |
| | | 230) | | | |
| 23 | 2H11 | QVQLVQSGGG | — | QSVLTQPPSVSG | — |
| | | LVKPGGSLRLS | | APGQRVTISCTG | |
| | | CAASGFTFTSY | | SSSNIGAGYDVH | |
| | | RMHWVRQAPG | | WYQQLPGRAPK | |
| | | KGLEWVSSITG | | LLIFANTNRPSG | |
| | | GGNYIEYADSV | | VPDRFSGSKSGA | |
| | | KGRFTISRDNA | | SASLAISGLQAE | |
| | | KNSLDLQMNS | | DEAHYYCQSYD | |
| | | LRAEDTAIYYC | | RSLNVVFGGGTE | |
| | | ARDMYGLGSY | | LTVLG | |
| | | YSPNYFDPWG | | (SEQ ID NO: 233) | |
| | | QGTTVTVSS | | | |
| | | (SEQ ID NO: 232) | | | |
| 24 | 2F1 | EVQLVESGGGL | — | QSVLTQPPSVSG | — |
| | | VKPGGSLRLSC | | APGQRVTISCTG | |
| | | AASGFTFTSYR | | SSSNIGAGYDVH | |
| | | MHWVRQAPG | | WYQQVPGTAPK | |
| | | KGLEWVSSITG | | LLIFGSTNRPSGV | |
| | | GGNYIEYADSV | | PDRFSGSKSGAS | |
| | | KGRFTISRDNA | | ASLAITGLQTED | |
| | | KNSLDLQMNS | | EADYYCQSYDR | |
| | | LRAEDTAIYYC | | SLSAWVFGGGT | |
| | | ARDMYGLGSY | | KLTVLG | |
| | | YSPNYFDPWG | | (SEQ ID NO: 235) | |
| | | QGTLVTVSS | | | |
| | | (SEQ ID NO: | | | |
| | | 234) | | | |
| 25 | 13C2 | EVQLVESGGGL | — | QSVLTQPPSVSG | — |
| | | VKPGGSLRLSC | | APGQRVTISCTG | |
| | | AASGFTFTSYR | | SSSNIGAGYDVH | |
| | | MHWVRQAPG | | WYQQVPGTAPK | |
| | | KGLEWVSSITG | | LLIFGSTDRPSGV | |
| | | GGNYIEYADSV | | PDRFSGSKSGNT | |
| | | KGRFTISRDNA | | ASLTISGLQAED | |
| | | KNSLDLQMNS | | EADYYCQSYDR | |
| | | LRAEDTAIYYC | | SLSHVFGTGTKV | |
| | | ARDMYGLGSY | | TVLG | |
| | | YSPNYFDPWG | | (SEQ ID NO: 237) | |
| | | QGTLVTVSS | | | |
| | | (SEQ ID NO: | | | |
| | | 236) | | | |
| 26 | 2H8 | EVQLVESGGGL | — | QSVLTQPPSVSG | — |
| | | AKPGGSLRLSC | | APGQRVTISCTG | |
| | | AASGFTFTSYR | | SSSNIGAGYDVH | |
| | | MHWVRQAPG | | WYQQLPGAAPR | |
| | | KGLEWVSSITG | | LLMFGNSNRPSG | |
| | | GGNYIEYADSV | | VPDRFSGSKSGT | |
| | | KGRFTISRDNA | | SASLAITGLQAE | |
| | | KNSLDLQMNS | | DEADYYCQSYD | |
| | | LRAEDTAIYYC | | RSLSHVFGTGTK | |
| | | ARDMYGLGSY | | VTVLG | |
| | | YSPNYFDPWG | | (SEQ ID NO: 239) | |
| | | QGTLVTVSS | | | |
| | | (SEQ ID NO: 238) | | | |
| 27 | 10A6 | EVQLVESGGGL | — | SYELTQPPSVSG | QSVLTQPPSV |
| | | VKPGGSLRLSC | | APGQRVTISCTG | SGAPGQRVTI |
| | | AASGFTFTSYR | | SSSNIGAGYDVH | SCTGSSSNIG |
| | | MHWVRQAPG | | WYQQLPGAAPR | AGYDVHWYQ |
| | | KGLEWVSSITG | | LLMFGNSNRPSG | QLPGAAPRLL |
| | | GGNYIEYADSV | | VPDRFSGSKSGT | MFGNSNRPSG |
| | | KGRFTISRDNA | | SASLAITGLQAE | VPDRFSGSKS |
| | | KNSLDLQMNS | | DEADYYCQSYD | GTSASLAITG |
| | | LRAEDTAIYYC | | SSLSYVFGTGTK | LQAEDEADY |
| | | ARDMYGLGSY | | VTVLG | YCQSYDSSLS |
| | | YSPNYFDSWG | | (SEQ ID NO: 241) | YVFGTGTKVT |
| | | QGTLVTVSS | | | VLG |
| | | (SEQ ID NO: 240) | | | (SEQ ID NO: |
| | | | | | 242) |
| 28 | 4G2 | EVQLVESGGGL | — | QSVLTQPPSVSG | — |
| | | VKPGGSLRLSC | | APGQRVTISCTG | |
| | | AASGFTFTSYR | | SSSNIGAGYDVH | |
| | | MHWVRQAPG | | WYQQLPGAAPR | |
| | | KGLEWVSSITG | | LLMFGNSNRPSG | |
| | | GGNYIEYADSV | | VPDRFSGSKSGT | |
| | | KGRFTISRDNA | | SASLAITGLQAE | |
| | | KNSLDLQMNS | | DEADYYCQSYD | |
| | | LRAEDTAIYYC | | SSLSHVFGTGTK | |
| | | ARDMYGLGSY | | VTVLG | |
| | | YSPNYFDPWG | | (SEQ ID NO: 244) | |
| | | QGTLVTVSS | | | |
| | | (SEQ ID NO: 243) | | | |
| 29 | 2G11 | EVQLVESGGGL | — | QSVLTQPPSVSG | — |
| | | VQPGGSLRLSC | | APGQRVTISCTG | |
| | | AASGFTFTSYR | | SSSNIGAGYDVH | |
| | | MHWVRQAPG | | WYQQLPGAAPR | |
| | | KGLEWVSSITG | | LLMFGNSNRPSG | |
| | | GGNYIEYADSV | | VPDRFSGSKSGT | |
| | | KGRFTISRDNA | | SASLAITGLQAE | |
| | | KNSLDLQMNS | | DEADYYCQSYD | |
| | | LRAEDTAIYYC | | SSLSYVFGTGTK | |
| | | ARDMYGLGSY | | VTVLG | |
| | | YSPNYFDPWG | | (SEQ ID NO: 246) | |
| | | QGTLVTVSS | | | |
| | | (SEQ ID NO: 245) | | | |
| 30 | 11H9 | EVQLVESGGGL | — | QSVLTQPPSVSG | QSVLTQPPSV |
| | | VQPGGSLRLSC | | APGQRVTISCTG | SGAPGQRVTI |
| | | AASEFTFSMN | | SSSNIGAGYDVH | SCTGSSSNIG |
| | | WVRQAPGKGL | | WYQQLPGRAPK | AGYDVHWYQ |
| | | EWVSSVRGGG | | LLIFANTNRPSG | QLPGRAPKLL |
| | | TETYYADSVK | | VPDRESGSKSGA | IFANTNRPSG |
| | | GRFTVSRDNSK | | SASLAITGLQAD | VPDRFSGSKS |
| | | NTLHLQMNSL | | DEADYYCQSYD | GASASLAITG |
| | | RAEDTALYYC | | SKHHVVFGTGT | LQADDEADY |
| | | AGGPIVEPNID | | KLTVLG | YCQSYDSKH |
| | | YFNSWGQGTL | | (SEQ ID NO: 248) | HVVFGTGTKL |
| | | VTVSS | | | TVLG |
| | | (SEQ ID NO: | | | (SEQ ID NO: |
| | | 247) | | | 249) |
| 31 | 12C9 | EVQLVESGGGL | — | QSVLTQPPSVSG | |
| | | VQPGGSLRLSC | | APGQRVTISCTG | |
| | | AASGFSFRSYW | | SSSNIGAGYDVH | |
| | | MSWLRQAPGK | | WYQQVPGTAPK | |
| | | GLQWVANIKP | | LLIFGSTNRPSGV | |
| | | DGSVESYVDSV | | PDRFSGSKSGAS | |
| | | EGRFTISRDNA | | ASLAIIGLQTEDE | |
| | | KNSLFLQMNSL | | ADYYCQSYDRS | |
| | | SAEDMAVYYC | | LSHVFGTGTQLT | |
| | | ARTDDGSSWF | | DLG | |
| | | VSTSSFYGMDV | | (SEQ ID NO: 251) | |
| | | WGQGTTVTVS | | | |
| | | S | | | |
| | | (SEQ ID NO: | | | |
| | | 250) | | | |
| 32 | 7A10 | EVQLVESGGGL | — | QSVLTQPPSVSG | — |
| | | VKPGGSLRLSC | | APGQRVTISCTG | |
| | | AASGFTFSSYT | | NSSNLGAGYDV | |
| | | MHWVRQAPG | | HWYQQVPGTAP | |
| | | KGLEWVSSITG | | KLLIFGSTNRPSG | |
| | | GSSYVDYSASV | | VPDRFSGSKSGA | |
| | | KGRFTISRDNS | | SASLTISGLQAE | |
| | | KNTLYLQMSSL | | DEADYYCCSYA | |
| | | RTDDTAVYFC | | ASSSRVFGTGTK | |
| | | AKDQRGDSYD | | VTVLG | |
| | | SVMNWYFDL | | (SEQ ID NO: 253) | |
| | | WGRGTTVTVSS | | | |
| | | (SEQ ID NO: | | | |
| | | 252) | | | |
| 33 | 4G1 | QVTLKESGGGL | EVQLVESGGG | QSVLTQPPSVSG | — |
| | | VKPGGSLRLSC | LVKPGGSLRL | APGQRVTISCTG | |
| | | AASGFRLSSYG | SCAASGFRLS | NSSNLGAGYDV | |
| | | MNWVRQAPG | SYGMNWVRQ | HWYQQLPGRAP | |
| | | KGLEWVSSISA | APGKGLEWVS | KLLIFANTNRPS | |
| | | SSSFINYADSV | SISASSSFIN | GVPDRFSGSKSG | |
| | | RDRFTISRDNA | YADSVRDRFT | ASASLAITSLQA | |
| | | KNSLYLQMNS | ISRDNAKNSL | DDEADYYCQSY | |
| | | LRAEDTAVYY | YLQMNSLRA | DRSLSHVFGTGT | |
| | | CARDDYGSGS | EDTAVYYCA | KVTVLG | |
| | | YSNWFDPWGQ | RDDYGSGSYS | (SEQ ID NO: 28) | |
| | | GTLVTVSS | NWFDPWGQG | | |
| | | (SEQ ID NO: 27) | TLVTVSS | | |
| | | | (SEQ ID NO: | | |
| | | | 254) | | |
| 34 | 7C6 | EVQLVESGGGL | QVQLVQSGA | QSVLTQPPSVSG | — |
| | | VKPGGSLRLSC | EVKKPGASV | APGQRVTISCTG | |
| | | AASGFTFSHYG | KVSCKASGFT | SSSNIGAGYDVH | |
| | | ISWVRQAPGQ | FSHYGISWVR | WYQQVPGTAPK | |
| | | GLEWMAWISA | QAPGQGLEW | LLIFGSTNRPSGV | |
| | | YNGNTDSIQKV | MAWISAYNG | PDRFSGSKSGAS | |
| | | QGRVTMTTDT | NTDSIQKVQG | ASLAITGLQAED | |
| | | STNTAYLELRS | RVTMTTDTST | EADYYCSSYTSS | |
| | | LRSDDTAVYY | NTAYLELRSL | STFVVFGGGTQL | |
| | | CVRDVPATGG | RSDDTAVYY | IILG | |
| | | AATADYWGQG | CVRDVPATG | (SEQ ID NO: 257) | |
| | | TLVTVSS | GAATADYWG | | |
| | | (SEQ ID NO: | QGTLVTVSS | | |
| | | 255) | (SEQ ID NO: | | |
| | | | 256) | | |
| 35 | 7A7 | QVQLVESGGG | — | QSALTQPASVSG | — |
| | | VVQPGRSLRLS | | SPGQSITISCTGT | |
| | | CAASGFTFNSY | | SSDVGGYNYVS | |
| | | AMHWVRQAP | | WFQQYPGKAPK | |
| | | GNGLDWVAGI | | LMIYDVSKRPSG | |
| | | SYDGGNKYYA | | VSNRFSGSKSGN | |
| | | DSVKDRFTISR | | TASLTISELQAED | |
| | | DNSKNTLYLQ | | EADYYCSSYTSS | |
| | | MSSLRTDDTA | | STGVFGGGTKLT | |
| | | VYYCAKDAG | | VLG | |
| | | WEAWWYFDL | | (SEQ ID NO: 259) | |
| | | WGRGTLVTVS | | | |
| | | S | | | |
| | | (SEQ ID NO: | | | |
| | | 258) | | | |
| 36 | 6A9 | EVQLVESGGGL | — | QSALTQPRSVSG | — |
| | | VQPGGSLRLSC | | SPGQSVTISCTGT | |
| | | AASGTFTKYA | | SSDVGSYNLVS | |
| | | MTWVRQALGK | | WYQQHPGKAPK | |
| | | GLEWVSTIGSG | | LMIYDVSKRPSG | |
| | | SDTHYADSVK | | VSNRFSGSKSGN | |
| | | GRFTISRDNSR | | TASLTISGLQAE | |
| | | NTLSLQMNSLR | | DEADYYCSSYA | |
| | | AEDTAVYYCA | | GSGNVVFGGGT | |
| | | KYLGITVGDTG | | QLIILG | |
| | | FRTFDYWGQG | | (SEQ ID NO: 261) | |
| | | TTVTVSS | | | |
| | | (SEQ ID NO: | | | |
| | | 260) | | | |
| 37 | 12B8 | QVQLVESGGG | — | QSALTQPPSASG | — |
| | | VVQPGRSLRLS | | SPGQSVTISCTGT | |
| | | CAASGFTFSIY | | SSDVGGYNYVS | |
| | | AMHWVRQAP | | WYRQHPGKAPK | |
| | | GKGLEWVAVV | | LLIYEVNNRPSG | |
| | | SYDGSEKYYA | | VPSRFSGSKSGN | |
| | | DSVQGRFTISR | | TASLTISGLQAE | |
| | | DKSKNTLYLQ | | DEADYYCSSYTS | |
| | | MNSLTAEDTA | | SSTFVVLGGGTQ | |
| | | VYYCAREPWV | | LIILG | |
| | | GTIGYWGQGT | | (SEQ ID NO: 263) | |
| | | LVTVSS | | | |
| | | (SEQ ID NO: | | | |
| | | 262) | | | |
| 38 | 12F7 | QMQLVQSGPE | QVQLVQSGA | QSVLTQPPSASG | QSVLTQPPSA |
| | | VKKPGSSVKVS | EVKKPGASV | TPGQRVTISCSGS | SGAPGQRVIIS |
| | | CKASGYTFTDY | KVSCKASGY | SSNVGSNTVNW | CSGSSSNVGS |
| | | GLSWVRQAPG | TFTDYGLSW | YQQLPGTAPKLL | NTVNWYQQL |
| | | HGLEWMGWIT | VRQAPGHGL | IYDKNERPSGVP | PGTAPKLLIY |
| | | AYNGDTNYAQ | EWMGWITAY | DRFSASKSGTSA | DKNERPSGVP |
| | | KFQDRLSVTTD | NGDTNYAQK | SLAISGLQSEDE | DRFSASKSGT |
| | | ISTSTAYMELR | FQDRLSVTTD | ADYYCAAWDDS | SASLAISGLQS |
| | | SLKSDDTAVY | ISTSTAYMEL | LNGWVFGGGTE | EDEADYYCA |
| | | YCARHVFCSG | RSLKSDDTAV | LTVLG | AWDDSLNGW |
| | | DGCYSGLGSW | YYCARHVFC | (SEQ ID NO: 266) | VFGGGTKLTV |
| | | GQGTLVTVSS | SGDGCYSGL | | LG |
| | | (SEQ ID NO: | GSWGQGTLV | | (SEQ ID NO: |
| | | 264) | TVSS | | 267) |
| | | | (SEQ ID NO: | | |
| | | | 265) | | |
| 39 | 7A11 | EVQLVESGGGL | — | QSALTQPASVSG | — |
| | | VKPGGSLRLSC | | SPGQSITISCTGT | |
| | | AASGFRLSSYG | | SSDVGSYNLVS | |
| | | MNWVRQAPG | | WYQQHPGKAPK | |
| | | KGLEWVSSISA | | LLIYEDTKRPSGI | |
| | | SSSFINYADSV | | PDRFSGSSSGNT | |
| | | RGRFTISRDKS | | ASLTITGAQAED | |
| | | KNTLYLQMNS | | EAEYYCSSRDSS | |
| | | LTAEDTAVYY | | GNHLVFGGGTK | |
| | | CAREPWVGTID | | LTVLG | |
| | | YWGQGTLVTV | | (SEQ ID NO: 269) | |
| | | SS | | | |
| | | (SEQ ID NO: | | | |
| | | 268) | | | |
| 40 | 4H11 | QVQLVQSGAE | — | SYELTQPPSASG | QSVLTQPPSA |
| | | VKKPGASVKV | | APGQRVIISCSGS | SGAPGQRVIIS |
| | | SCKASGYTFTD | | SSNVGSNTVNW | CSGSSSNVGS |
| | | YGLSWVRQAP | | YQQLPGRAPKLL | NTVNWYQQL |
| | | GHGLEWMGWI | | IFANTNRPSGVP | PGRAPKLLIF |
| | | TAYNGDTNYA | | DRFSGSKSGTSA | ANTNRPSGVP |
| | | QKFQDRLSVTT | | SLAISGLQSEDE | DRFSGSKSGT |
| | | DISTSTAYMEL | | ADYYCAAWDDS | SASLAISGLQS |
| | | RSLKSDDTAVY | | LNGWVFGGGTK | EDEADYYCA |
| | | YCARHVFCSG | | LTVLG | AWDDSLNGW |
| | | DGCYSGLGSW | | (SEQ ID NO: 271) | VFGGGTKLTV |
| | | GQGTLVTVSS | | | LG |
| | | (SEQ ID NO: | | | (SEQ ID NO: |
| | | 270) | | | 272) |
| 41 | 2G3 | QVQLVQSGAE | — | SYELTQPLSASG | QSVLTQPLSA |
| | | VKKPGASVKV | | TPGQRVTISCSGS | SGTPGQRVTI |
| | | SCKASGYTFTD | | SSNIGSGTVNWY | SCSGSSSNIGS |
| | | YGLSWVRQAP | | QQLSGTAPKLL | GTVNWYQQL |
| | | GHGLEWMGWI | | MHSDNQRPSGV | SGTAPKLLMH |
| | | TAYNGDTNYA | | PDRFSGSKSGTS | SDNQRPSGVP |
| | | QKFQDRLSVTT | | ASLAISGLQSED | DRFSGSKSGT |
| | | DISTSTAYMEL | | EADYYCAAWDD | SASLAISGLQS |
| | | RSLKSDDTAVY | | SLNGWVFGGGT | EDEADYYCA |
| | | YCARHVFCSG | | KLTVLG | AWDDSLNGW |
| | | DGCYSGLGSW | | (SEQ ID NO: 274) | VFGGGTKLTV |
| | | GQGTLVTVSS | | | LG |
| | | (SEQ ID NO: | | | (SEQ ID NO: |
| | | 273) | | | 275) |
| 42 | 12C10 | QVQLVQSGSE | — | QPVLTQPPSVSG | QSVLTQPPSV |
| | | VKKPGASVKV | | TPGQRVTISCSGS | SGTPGQRVTI |
| | | SCKASGYTFTD | | SSNIGSDTVNWY | SCSGSSSNIGS |
| | | YGLSWVRQAP | | QQLSGTAPKLL | DTVNWYQQL |
| | | GHGLEWMGWI | | MHSDNQRPSGV | SGTAPKLLMH |
| | | TAYNGDTNYA | | PDRFSGSKSGTS | SDNQRPSGVP |
| | | QKFQDRLSVTT | | ASLAISGLQSED | DRFSGSKSGT |
| | | DISTSTAYMEL | | EADYYCAAWDD | SASLAISGLQS |
| | | RSLKSDDTAVY | | SLNGWVFGGGT | EDEADYYCA |
| | | YCARHVFCSG | | KLTVLG | AWDDSLNGW |
| | | DGCYSGLGSW | | (SEQ ID NO: 277) | VFGGGTKLTV |
| | | GQGTLVTVSS | | | LG |
| | | (SEQ ID NO: | | | (SEQ ID NO: |
| | | 276) | | | 278) |
| 43 | 3E8 | QVQLVQSGSE | QVQLVQSGSE | QPVLTQPPSASG | QSVLTQPPSA |
| | | VKKPGASVKV | VKKPGASVK | TPGQRVTISCSGS | SGTPGQRVTI |
| | | SCKASGYTFTD | VSCKASGYTF | SSNIGSDTVNWY | SCSGSSSNIGS |
| | | YGLSWVRQAP | TDYGLSWVR | QQLSGTAPKLL | DTVNWYQQL |
| | | GHGLEWMGWI | QAPGHGLEW | MHSDNQRPSGV | SGTAPKLLMH |
| | | TAYNGDTNYA | MGWITAYNG | PDRFSGSKSGTS | SDNQRPSGVP |
| | | QKFQDRLSVTT | DTNYAQKFQD | ASLAISGLQSED | DRFSGSKSGT |
| | | DISTSTAYMEL | RLSVTTDIST | EADYYCAAWDD | SASLAISGLQS |
| | | RSLKSDDTAVY | STAYMELRSL | SLNGWVFGGGT | EDEADYYCA |
| | | YCARHVFCSG | KSDDTAVYY | KLTVLG | AWDDSLNGW |
| | | DGCYSGLGSW | CARHVFCSG | (SEQ ID NO: 281) | VFGGGTKLTV |
| | | GQGTLVTVSS | DGCYSGLGS | | LG |
| | | (SEQ ID NO: | WGQGTLVTV | | (SEQ ID NO: |
| | | 279) | SS | | 282) |
| | | | (SEQ ID NO: | | |
| | | | 280) | | |
| 44 | 7D1 | QVQLVQSGAE | — | QSVLTQPPSASG | — |
| | | VKKPGASVKV | | TPGQRVTISCSGS | |
| | | SCKASGYTFTD | | SSNIGSNTVNWY | |
| | | YGLSWVRQAP | | QQLPGTAPKLLI | |
| | | GHGLEWMGWI | | YSNNQRPSGVPD | |
| | | TAYNGDTNYA | | RFSGSTSGTSASL | |
| | | QKFQDRLSVTT | | AISGLQSEDEAD | |
| | | DISTSTAYMEL | | YYCAAWDDSLN | |
| | | RSLKSDDTAVY | | GWVFGGGTKLT | |
| | | YCARHVFCSG | | VLG | |
| | | DGCYSGLGSW | | (SEQ ID NO: 284) | |
| | | GQGTLVTVSS | | | |
| | | (SEQ ID NO: | | | |
| | | 283) | | | |
| 45 | 4G5 | QVQLVQSGSE | — | SYELTQPLSASG | QSVLTQPLSA |
| | | VKKPGASVKV | | TPGQRVTISCSGS | SGTPGQRVTI |
| | | SCKASGYTFTD | | SSNIGSNTVNWY | SCSGSSSNIGS |
| | | YGLSWVRQAP | | QQLPGTAPKLLI | NTVNWYQQL |
| | | GHGLEWMGWI | | YSNNQRPSGVPD | PGTAPKLLIYS |
| | | TAYNGDTNYA | | RFSGSKSGTSAS | NNQRPSGVPD |
| | | QKFQDRLSVTT | | LAISGLQSEDEA | RFSGSKSGTS |
| | | DISTSTAYMEL | | DYYCAAWDDSL | ASLAISGLQSE |
| | | RSLKSDDTAVY | | NGWVFGGGTKL | DEADYYCAA |
| | | YCARHVFCSG | | TVLG | WDDSLNGWV |
| | | DGCYSGLGSW | | (SEQ ID NO: 286) | FGGGTKLTVL |
| | | GQGTLVTVSS | | | G |
| | | (SEQ ID NO: 285) | | | (SEQ ID NO: |
| | | | | | 287) |
| 46 | 2E8 | QVQLVQSGPE | — | SYELTQPPSASG | QSVLTQPPSA |
| | | VKKPGSSVKVS | | APGQRVIISCSGS | SGAPGQRVIIS |
| | | CKASGYTFTDY | | SSNVGSNTVNW | CSGSSSNVGS |
| | | GLSWVRQAPG | | YQQLPGTAPKLL | NTVNWYQQL |
| | | HGLEWMGWIT | | IYDKNERPSGVP | PGTAPKLLIY |
| | | AYNGDTNYAQ | | DRESASKSGTSA | DKNERPSGVP |
| | | KFQDRLSVTTD | | SLAISGLQSEDE | DRFSASKSGT |
| | | ISTSTAYMELR | | ADYYCAAWDDS | SASLAISGLQS |
| | | SLKSDDTAVY | | LNGWVFGGGTK | EDEADYYCA |
| | | YCARHVFCSG | | LTVLG | AWDDSLNGW |
| | | DGCYSGLGSW | | (SEQ ID NO: 289) | VFGGGTKLTV |
| | | GQGTLVTVSS | | | LG |
| | | (SEQ ID NO: 288) | | | (SEQ ID NO: |
| | | | | | 290) |
| 47 | 13H9 | QVQLVQSGAE | — | SYELIQPPSASGA | QSVLTQPPSA |
| | | VKKPGASVKV | | PGQRVIISCSGSS | SGAPGQR VIIS |
| | | SCKASGYTGYT | | SNVGSNTVNWY | CSGSSSNVGS |
| | | FTDYGLSWVR | | QQLPGTAPKLLI | NTVNWYQQL |
| | | QAPGHGLEWM | | YDKNERPSGVPD | PGTAPKLLIY |
| | | GWITAYNGDT | | RFSASKSGTSAS | DKNERPSGVP |
| | | NYAQKFQDRL | | LAISGLQSEDEA | DRFSASKSGT |
| | | SVTTDISTSTAY | | DYYCAAWDDSL | SASLAISGLQS |
| | | MELRSLKSDDT | | NGWVFGGGTEL | EDEADYYCA |
| | | AVYYCARHVF | | TVLG | AWDDSLNGW |
| | | CSGDGCYSGL | | (SEQ ID NO: 292) | VFGGGTKLTV |
| | | GSWGQGTLVT | | | LG |
| | | VSS | | | (SEQ ID NO: |
| | | (SEQ ID NO: 291) | | | 293) |
| 48 | 4E9 | QVQLVQSGAE | — | QSVLTQPPSVSG | — |
| | | VKKPGSSVKVS | | APGQRVTISCSG | |
| | | CKASGYTFTDY | | SSSNVGSNTVN | |
| | | GLSWVRQAPG | | WYQQLPGTAPK | |
| | | HGLEWMGWIT | | LLIYDKNERPSG | |
| | | AYNGDTNYAQ | | VPDRFSASKSGT | |
| | | KFQDRLSVTTD | | SSSLAIGGLRSED | |
| | | ISTSTAYMELR | | EADYYCGTWDD | |
| | | SLKSDDTAVY | | NLNGWVFGGGT | |
| | | YCARHVFCSG | | KLTVLG | |
| | | DGCYSGLGSW | | (SEQ ID NO: 295) | |
| | | GQGTLVTVSS | | | |
| | | (SEQ ID NO: 294) | | | |
| 49 | 4H7 | QVQLVQSGAE | — | SYELTQPPSASG | QSVLTQPPSA |
| | | VKKPGASVKV | | APGQRVIISCSGS | SGAPGQRVIIS |
| | | SCKASGYTFTD | | SSNVGSNTVNW | CSGSSSNVGS |
| | | YGLSWVRQAP | | YQQLPGTAPKLL | NTVNWYQQL |
| | | GHGLEWMGWI | | IYDKNERPSGVP | PGTAPKLLIY |
| | | TAYNGDTNYA | | DRFSASKSGTSS | DKNERPSGVP |
| | | QKFQDRLSVTT | | SLAIGGLRSEDE | DRFSASKSGT |
| | | DISTSTAYMEL | | ADYYCATWDDN | SSSLAIGGLRS |
| | | RSLKSDDTAVY | | LNGWVFGGGTK | EDEADYYCA |
| | | YCARHVFCSG | | LTVLG | TWDDNLNGW |
| | | DGCYSGLGSW | | (SEQ ID NO: 297) | VFGGGTKLTV |
| | | GQGTLVTVSS | | | LG |
| | | (SEQ ID NO: 296) | | | (SEQ ID NO: |
| | | | | | 298) |
| 50 | 2F8 | QVQLVQSGAE | — | SYELTQPPSASG | QSVLTQPPSA |
| | | VKKPGASVKV | | APGQRVIISCSGS | SGAPGQRVIIS |
| | | SCKASGYTFTD | | SSNVGSNTVNW | CSGSSSNVGS |
| | | YGLSWVRQAP | | YQQLPGTAPKLL | NTVNWYQQL |
| | | GHGLEWMGWI | | IYDKNERPSGVP | PGTAPKLLIY |
| | | TAYNGDTNYA | | DRFSASKSGTSS | DKNERPSGVP |
| | | QKFQDRLSVTT | | SLAIGGLRSEDE | DRFSASKSGT |
| | | DISTSTAYMEL | | ADYYCATWDDN | SSSLAIGGLRS |
| | | RSLKSDDTAVY | | LNGWVFGGGTK | EDEADYYCG |
| | | YCARHVFCSG | | LTVLG | TWDDNLNGW |
| | | DGCYSGLGSW | | (SEQ ID NO: 300) | VFGGGTKLTV |
| | | GQGTLVTVSS | | | LG |
| | | (SEQ ID NO: 299) | | | (SEQ ID NO: |
| | | | | | 301) |
| 51 | 3E7 | EVQLVESGTEV | EVQLVQSGAE | QSVLTQPPSASG | — |
| | | KKPGASVKVS | VKKPGASVK | APGQRVIISCSGS | |
| | | CKASGYTFTDY | VSCKASGYTF | SSNVGSNTVNW | |
| | | GLSWVRQAPG | TDYGLSWVR | YQQLPGTAPKLL | |
| | | HGLEWMGWIT | QAPGHGLEW | IYDKNERPSGVP | |
| | | AYNGDTNYAQ | MGWITAYNG | DRFSASKSGTSS | |
| | | KFQDRLSVTTD | DTNYAQKFQ | SLAIGGLRSEDE | |
| | | ISTSTAYMELR | DRLSVTTDIST | ADYYCGTWDDN | |
| | | SLKSDDTAVY | STAYMELRSL | LNGWVFGGGTK | |
| | | YCARHVFCSG | KSDDTAVYY | LTVLG | |
| | | DGCYSGLGSW | CARHVFCSG | (SEQ ID NO: 304) | |
| | | GQGTLVTVSS | DGCYSGLGS | | |
| | | (SEQ ID NO: | WGQGTLVTV | | |
| | | 302) | SS | | |
| | | | (SEQ ID NO: | | |
| | | | 303) | | |
| 52 | 9H4 | QVQLVQSGGG | EQVQLVESGG | EIVLTQSPGIQSL | — |
| | | LVQPGGSLRIS | GLVQPGGSLR | SPGETATLSCRA | |
| | | CTASGFTFRNY | ISCTASGFTFR | SESISSSYFAWY | |
| | | EMNWVRQAPG | NYEMNWVRQ | QQKPGQAPRLLI | |
| | | KGLEWVAYISS | APGKGLEWV | YGASSRASGIPD | |
| | | SGSSRYYADSV | AYISSSGSSRY | RFSGSGSGTDFT | |
| | | KGRFTISRDNA | YADSVKGRF | LTISRLEPEDFAV | |
| | | KNSLFLQMNSL | TISRDNAKNS | YYCQQYGSSPPI | |
| | | RAEDMAVYYC | LFLQMNSLRA | TFGQGTRLEIKR | |
| | | ARTDDGSSWF | EDMAVYYCA | (SEQ ID NO: 307) | |
| | | VSTSSFYGMDV | RTDDGSSWF | | |
| | | WGQGTTVTVS | VSTSSFYGMD | | |
| | | S | VWGQGTTVT | | |
| | | (SEQ ID NO: | VSS | | |
| | | 305) | (SEQ ID NO: | | |
| | | | 306) | | |
| 53 | 8B1 | QVQLVQSGGG | EQVQLVESGG | DVVMTQSPGTLS | EIVLTQSPGTL |
| | | LVQPGGSLRIS | GLVQPGGSLR | LSPGERATLSCR | SLSPGERATL |
| | | CTASGFTFRNY | ISCTASGFTFR | ASQSVSSSYLAW | SCRASQSVSS |
| | | EMNWVRQAPG | NYEMNWVRQ | YQQKPGQAPRL | SYLAWYQQK |
| | | KGLEWVAYISS | APGKGLEWV | LIYGVSSRATGIP | PGQAPRLLIY |
| | | SGSSRYYADSV | AYISSSGSSRY | DRFSGSGSGTDF | GVSSRATGIP |
| | | KGRFTISRDNA | YADSVKGRF | TLTISRLEPEDFA | DRFSGSGSGT |
| | | KNTLYLQMSSL | TISRDNAKNT | VYYCQQYGSSPP | DFTLTISRLEP |
| | | RSDDTAVYYC | LYLQMSSLRS | ITFGQGTKLEIKR | EDFAVYYCQ |
| | | ARTDDGSSWF | DDTAVYYCA | (SEQ ID NO: 310) | QYGSSPPITFG |
| | | VSTSSFYGMDV | RTDDGSSWF | | QGTKLEIKR |
| | | WGQGTTVTVSS | VSTSSFYGMD | | (SEQ ID NO: |
| | | (SEQ ID NO: | VWGQGTTVT | | 311) |
| | | 308) | VSS | | |
| | | | (SEQ ID NO: | | |
| | | | 309) | | |
| 54 | 7G12 | EVQLVESGGGL | — | DIQLTQSPSSVSA | — |
| | | VQPGGSLRLSC | | SVGERVTITRRA | |
| | | AASEFTFSMN | | SQNIDRWLAWY | |
| | | WVRQAPGKGL | | QQKPGKAPDLLI | |
| | | EWVSSVRGGG | | FAASSLQSGVPS | |
| | | TETYYADSVK | | RFSGSGSGTDFT | |
| | | GRFTVSRDNSK | | LTISSLQPEDFAT | |
| | | NTLYLQMNSL | | YYCQQGKTFPPT | |
| | | RVEDTAVYYC | | FGQGTKLEIKR | |
| | | TVCVVMFRGW | | (SEQ ID NO: 313) | |
| | | CNWFDPWGQG | | | |
| | | TLVTVSS | | | |
| | | (SEQ ID NO: | | | |
| | | 312) | | | |
| 55 | 12F11 | EVQLLESGGGL | — | DVVMTQSPSSLS | DIQMTQSPSS |
| | | VQPGGSLRLSC | | ASVGDRVTITCR | LSASVGDRVT |
| | | AASEFTFSMN | | ASQSLSSYLNWY | ITCRASQSLSS |
| | | WVRQAPGKGL | | QQKPGKAPKLLI | YLNWYQQKP |
| | | EWVSSVRGGG | | YATTNLQSGVPS | GKAPKLLIYA |
| | | TETYYADSVK | | RFSGSGSGTHFT | TTNLQSGVPS |
| | | GRFTVSRDNSK | | LTIGSLQPEDFAT | RFSGSGSGTH |
| | | NTLYLQMNSL | | YYCQQSFQTPLT | FTLTIGSLQPE |
| | | RVEDTAVYYC | | FGGGTKVEIKR | DFATYYCQQ |
| | | TVCVVMFRGW | | (SEQ ID NO: 315) | SFQTPLTFGG |
| | | CNWFDPWGQG | | | GTKLEIKR |
| | | TLVTVSS | | | (SEQ ID NO: |
| | | (SEQ ID NO: 314) | | | 316) |
| 56 | 5B12 | QVQLVESGGG | — | DIQMTQSPATLS | — |
| | | VVQPGRSLRLS | | ASVGDRVTITCR | |
| | | CAASGFTFISY | | ASQSINNNLNW | |
| | | VMHWVRQAP | | YQQKPGQAPKL | |
| | | GKGLEWVAVV | | LIYAASTLQGGV | |
| | | SYDGSEKYYA | | PSRFSGSGSGTEF | |
| | | DSVKGRFTISR | | TLTISSLQPEDFA | |
| | | DNSKNTLYLQ | | TYYCQQLNGYP | |
| | | MNSLTAEDTA | | LTFGGGTKVEIK | |
| | | VYYCAREPWV | | R | |
| | | GTIDYWGQGT | | (SEQ ID NO: 318) | |
| | | LVTVSS | | | |
| | | (SEQ ID NO: 317) | | | |
| 57 | 8C3 | EVQLLESGGGL | — | DIQMTQSPDSLA | |
| | | VQPGGSLRLSC | | VSLGERATINCK | |
| | | AASGFTFSDYA | | SSQSVLYSSNNK | |
| | | MSWVRQAPGK | | NYLAWYQQKPG | |
| | | GLEWVSSITAN | | QPPKMLIYWASI | |
| | | GLMTYYTDSV | | RETGVPDRFSGS | |
| | | KGRFTISRDNS | | GSETDFTLTISSL | |
| | | KNTLYLQMSSL | | QAEDVAVYYCQ | |
| | | RTDDTAVYYC | | QYYSTLTFGGGT | |
| | | AKDAGWEAW | | KVEIKR | |
| | | WYFDLWGRGT | | (SEQ ID NO: 320) | |
| | | LVTVSS(SEQ ID | | | |
| | | NO: 319) | | | |
| 58 | 2F4 | QVQLVQSGAE | — | EIVLTQSPATVSL | |
| | | VKKPGASVKV | | SPGERTMLSCRA | |
| | | SCKASGYTFTS | | SQNVSNYLGWY | |
| | | YYMRWLRQAP | | QQRRGQPPRLLI | |
| | | GQGLEWMGIID | | SDASNRASGVPA | |
| | | PSGGSTTYAQK | | RFSGSGSGTDFT | |
| | | FQGRVSITRDT | | LTITSLQPEDFAV | |
| | | STSTVYMELSS | | YFCQHRSSWPVT | |
| | | LRSEDTAVYYC | | FGGGTKVEIKR | |
| | | ARGRGVVPAG | | (SEQ ID NO: 322) | |
| | | NPSTGGVGMD | | | |
| | | VWGQGTTVTV | | | |
| | | SS | | | |
| | | (SEQ ID NO: 321) | | | |
| 59 | 1C10 | QVQLVQSGSE | — | DVVMTQSPLSLP | |
| | | VKKPGASVKV | | VTLGQPASISCRS | |
| | | SCKASGYTFGH | | SQSLVFSDGNTY | |
| | | YGISWVRQAP | | LSWFQQRPGQSP | |
| | | GQGLEWMAWI | | RRLIYKVSNRDS | |
| | | SAYNGNTDSIQ | | GVPDRFSASGSG | |
| | | KVQGRVTMTT | | TDFTLKISRVEA | |
| | | DTSTNTAYLEL | | DDVGVYYCMQ | |
| | | RSLRSDDTAVY | | VTHWPRTFGQG | |
| | | YCVRDVPATG | | TKLEIKR | |
| | | GAATADYWGQ | | (SEQ ID NO: 324) | |
| | | GTLVTVSS | | | |
| | | (SEQ ID NO: | | | |
| | | 323) | | | |
| 60 | 5E9 | QVQLVQSGPE | — | DVVMTQSPLSLP | — |
| | | VKKPGSSVKVS | | VTLGQPASISCRS | |
| | | CKASGYTFGH | | SQSLVFSDGNTY | |
| | | YGISWVRQAP | | LSWFQQRPGQSP | |
| | | GQGLEWMAWI | | RRLIYKVSNRDS | |
| | | SAYNGNTDSIQ | | GVPDRFSASGSG | |
| | | KVQGRVTMTT | | TDFTLKISRVEA | |
| | | DTSTNTAYLEL | | DDVGVYYCMQ | |
| | | RSLRSDDTAVY | | VTHWPRTFGQG | |
| | | YCVRDVPATG | | TRLEIKR | |
| | | GAATADYWGQ | | (SEQ ID NO: 326) | |
| | | GTTVTVSS | | | |
| | | (SEQ ID NO: 325) | | | |
| 61 | 5C9 | QVQLVQSGSE | — | DVVMTQSPLSLP | — |
| | | VKKPGASVKV | | VTLGQPASISCRS | |
| | | SCKASGYTFGH | | SQSLVFSDGNTY | |
| | | YGISWVRQAP | | LSWFQQRPGQSP | |
| | | GQGLEWMAWI | | RRLIYKVSNRDS | |
| | | SAYNGNTDSIQ | | GVPDRFSASGSG | |
| | | KVQGRVTMTT | | TDFTLKISRVEA | |
| | | DTSTNTAYLEL | | DDVGVYYCMQ | |
| | | RSLRSDDTAVY | | VTHWPRTFGQG | |
| | | YCVRDVPATG | | TKLEIKR | |
| | | GAATADYWGQ | | (SEQ ID NO: 328) | |
| | | GTTVTVSS | | | |
| | | (SEQ ID NO: 327) | | | |
| 62 | 5C7 | QVQLQQSGSE | QVQLVQSGA | DVVMTQSPLSLP | — |
| | | VKKPGASVKV | EVKKPGASV | VPLGQPASISCRS | |
| | | SCKASGYTFGH | KVSCKASGY | SQSLVHSDGNTY | |
| | | YGISWVRQAP | TFGHYGISWV | LSWFQQRPGQSP | |
| | | GQGLEWMAWI | RQAPGQGLE | RRLIYKVSDRDS | |
| | | SAYNGNTDSIQ | WMAWISAYN | GVPDRFSGSGSG | |
| | | KVQGRVTMTT | GNTDSIQKVQ | TDFTLKISRVEA | |
| | | DTSTNTAYLEL | GRVTMTTDTS | EDVGVYYCMQV | |
| | | RSLRSDDTAVY | TNTAYLELRS | THWPRTFGQGT | |
| | | YCVRDVPATG | LRSDDTAVY | RLEIKR | |
| | | GAATADYWGQ | YCVRDVPAT | (SEQ ID NO: 331) | |
| | | GTLVTVSS | GGAATADYW | | |
| | | (SEQ ID NO: | GQGTLVTVSS | | |
| | | 329) | (SEQ ID NO: | | |
| | | | 330) | | |
| 63 | 8A11 | QVQLVQSGSE | — | DVVMTQSPLSLP | — |
| | | VKKPGASVKV | | VTLGQPASISCK | |
| | | SCKASGYTFGH | | SSRSLVHSDGNT | |
| | | YGISWVRQAP | | YLSWFQQRPGQ | |
| | | GQGLEWMAWI | | SPRRLIYKVSNR | |
| | | SAYNGNTDSIQ | | DSGVPDRESASG | |
| | | KVQGRVTMTT | | SGTDFTLKISRVE | |
| | | DTSTNTAYLEL | | ADDVGVYYCM | |
| | | RSLRSDDTAVY | | QVTHWPRTFGQ | |
| | | YCVRDVPATG | | GTKLEIKR | |
| | | GAATADYWGQ | | (SEQ ID NO: 333) | |
| | | GTLVTVSS | | | |
| | | (SEQ ID NO: | | | |
| | | 332) | | | |

### 4. Result of Screening Anti-RSV Neutralizing Antibodies

The neutralizing activity against RSV virus for 127 IgG supernatant samples (1^{st}: 88, 2^{nd}: 39) was confirmed by a FRNT test method. As a result, a total of 75 samples (1^{st}: 43, 2^{nd}: 32) exhibiting 50% or more neutralizing activity at a 1:10 dilution were confirmed. Although there was a difference in neutralizing activity according to 1:100 and 1:1000 dilutions, it is considered to be a result that can appear due to the difference in the expression level and antibody concentration of each sample.

TABLE 20
Samples with 50% or more neutralizing
activity according to dilution

Neutralizing activity >50%

| Dilution | 1^{st} (88 samples) | 2^{nd} (39 samples) |
|---|---|---|
| 1:10 | 43 | 32 |
| 1:100 | 36 | 31 |
| 1:1000 | 6 | 26 |

### Example 5. Analysis Result for Selected RSV Neutralizing Antibodies

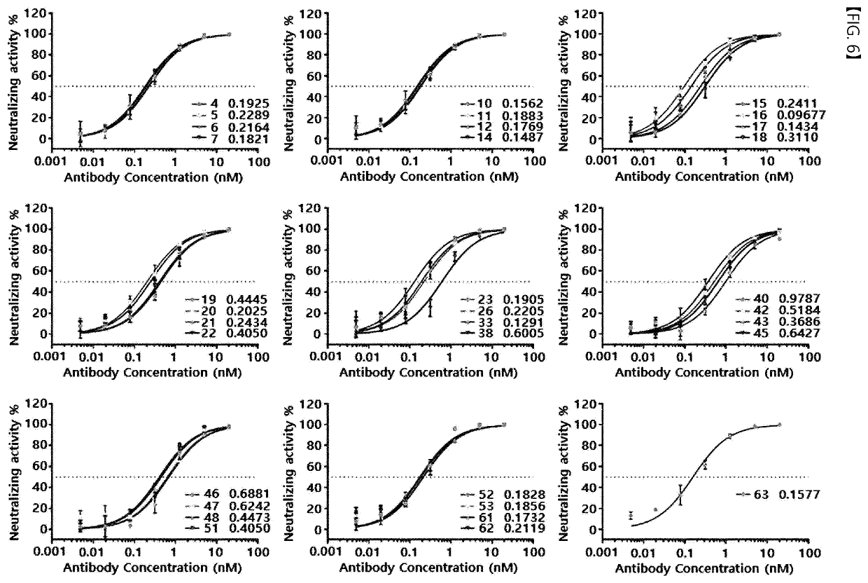

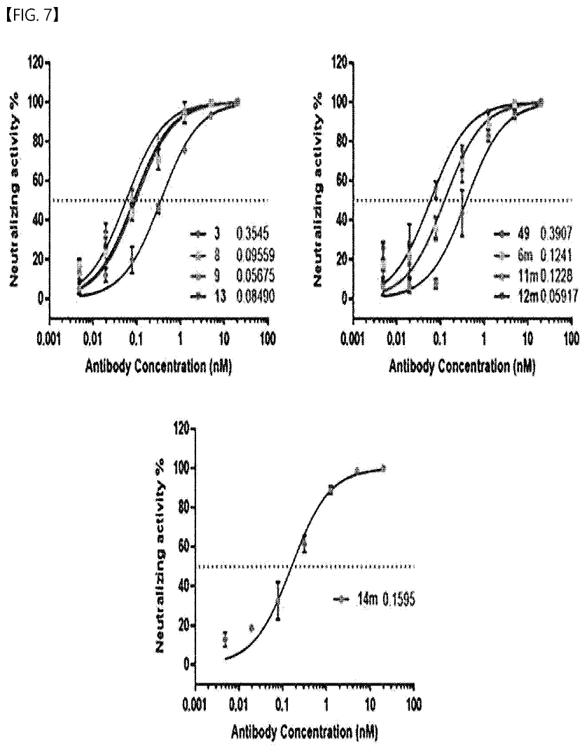

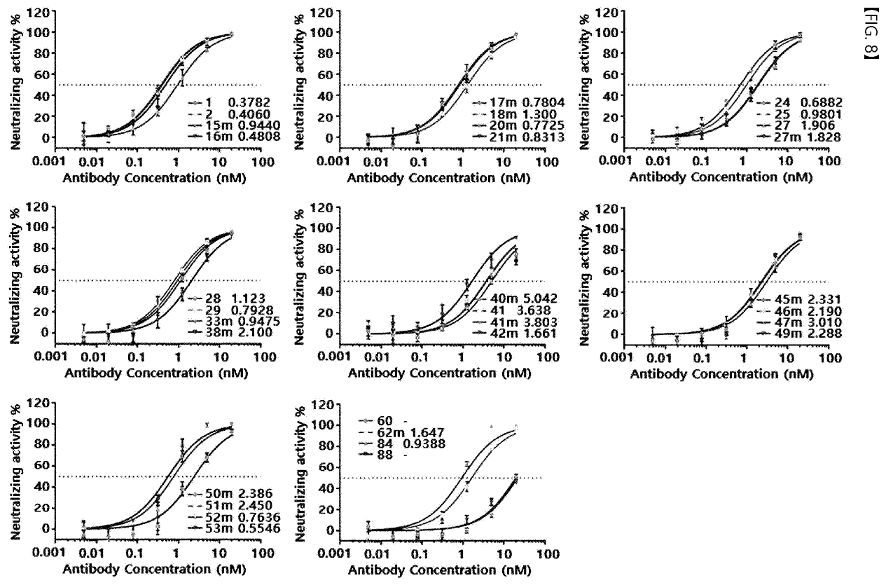

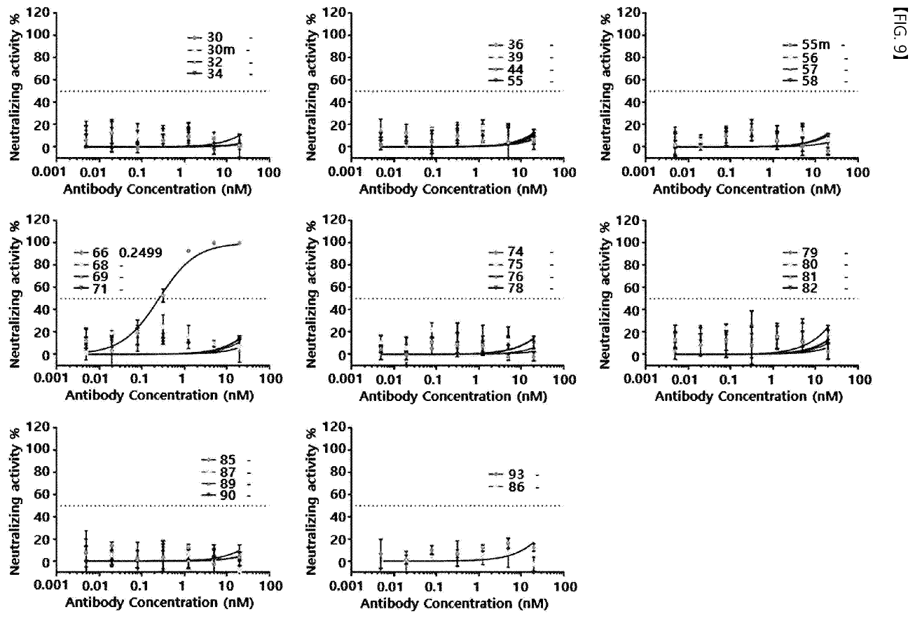

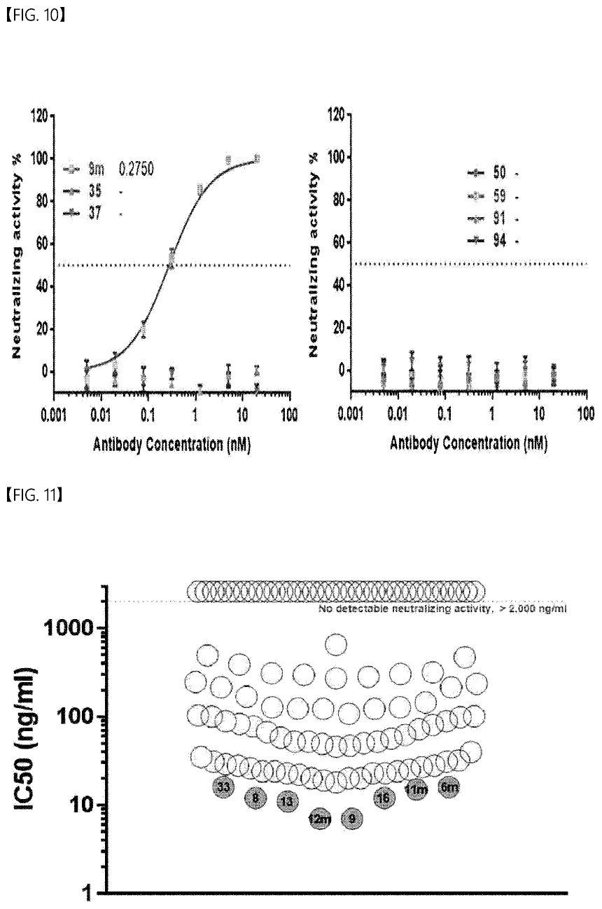

The result of the neutralizing activity test for the selected RSV antibodies are shown in FIGS. 6 to 10, and the IC₅₀ and range (confidence interval: 95%) of each antibody are shown in Table 21 below.

TABLE 21

| Sample | | | |
|---|---|---|---|
| Name | IC₅₀ (pM) | Range (95% CI) | IC50 (ng/mL) |
| 1 | 378.20 | 334.4-427.8 | 49.17 |
| 2 | 406.00 | 331.1-497.8 | 52.78 |
| 3 | 354.50 | 304.8-412.3 | 46.09 |
| 4 | 192.50 | 158.6-233.8 | 25.03 |
| 5 | 228.90 | 199.8-262.1 | 29.76 |
| 6 | 216.40 | 193.4-242.1 | 28.13 |
| 7 | 182.10 | 167.5-198.0 | 23.67 |
| 8 | 95.59 | 76.34-119.7 | 12.43 |
| 9 | 56.75 | 47.29-68.11 | 7.38 |
| 10 | 156.20 | 125.7-194.1 | 20.31 |
| 11 | 188.30 | 162.1-218.9 | 24.48 |
| 12 | 176.90 | 151.8-206.1 | 23.00 |
| 13 | 84.90 | 66.47-108.5 | 11.04 |
| 14 | 148.70 | 130.7-169.3 | 19.33 |
| 15 | 241.10 | 210.4-276.2 | 31.34 |
| 16 | 96.77 | 80.97-115.7 | 12.58 |
| 17 | 143.40 | 110.0-187.1 | 18.64 |
| 18 | 311.00 | 251.4-384.7 | 40.43 |
| 19 | 444.50 | 371.7-531.5 | 57.79 |
| 20 | 202.50 | 168.8-242.8 | 26.33 |
| 21 | 243.40 | 202.5-292.4 | 31.64 |
| 22 | 405.00 | 323.0-507.8 | 52.65 |
| 23 | 190.50 | 153.9-235.9 | 24.77 |
| 24 | 688.20 | 565.6-837.3 | 89.47 |
| 24 | 980.10 | 828.3-1160 | 127.41 |
| 26 | 220.50 | 185.4-262.2 | 28.67 |
| 27 | 1906.00 | 1606-2263 | 247.78 |
| 28 | 1123.00 | 901.8-1398 | 145.99 |
| 29 | 792.80 | 693.0-906.9 | 103.06 |
| 30 | ND | ND | ND |
| 32 | ND | ND | ND |
| 33 | 129.10 | 104.0-160.4 | 16.78 |
| 34 | ND | ND | ND |
| 35 | ND | ND | ND |
| 36 | ND | ND | ND |
| 37 | ND | ND | ND |
| 38 | 600.50 | 471.9-764.2 | 78.07 |
| 39 | ND | ND | ND |
| 40 | 978.70 | 791.1-1211 | 127.23 |
| 41 | 3638.00 | 2935-4510 | 472.94 |
| 42 | 518.40 | 416.9-644.6 | 67.39 |
| 43 | 368.60 | 310.9-437.1 | 47.92 |
| 44 | ND | ND | ND |
| 45 | 642.70 | 573.6-720.2 | 83.55 |
| 46 | 688.10 | 537.0-881.7 | 89.45 |
| 47 | 624.20 | 478.8-813.8 | 81.15 |
| 48 | 447.30 | 363.3-550.8 | 58.15 |
| 49 | 390.70 | 311.2-490.5 | 50.79 |
| 50 | ND | ND | ND |
| 51 | 405.00 | 340.2-482.0 | 52.65 |
| 52 | 182.80 | 150.6-221.8 | 23.76 |
| 53 | 185.60 | 146.2-235.6 | 24.13 |
| 55 | ND | ND | ND |
| 56 | ND | ND | ND |
| 57 | ND | ND | ND |
| 58 | ND | ND | ND |
| 59 | ND | ND | ND |
| 60 | ND | ND | ND |
| 61 | 173.20 | 145.3-206.5 | 22.52 |
| 62 | 211.90 | 166.6-269.6 | 27.55 |
| 63 | 157.70 | 126.2-197.1 | 20.50 |
| 66 | 249.9 | 184.8-338.1 | 32.49 |
| 68 | ND | ND | ND |
| 69 | ND | ND | ND |
| 71 | ND | ND | ND |
| 74 | ND | ND | ND |
| 75 | ND | ND | ND |
| 76 | ND | ND | ND |
| 78 | ND | ND | ND |
| 79 | ND | ND | ND |
| 80 | ND | ND | ND |
| 81 | ND | ND | ND |
| 82 | ND | ND | ND |
| 84 | 938.80 | 602.6-1463 | 122.04 |
| 85 | ND | ND | ND |
| 86 | ND | ND | ND |
| 87 | ND | ND | ND |
| 88 | ND | ND | ND |
| 89 | ND | ND | ND |
| 90 | ND | ND | ND |
| 91 | ND | ND | ND |
| 93 | ND | ND | ND |
| 94 | ND | ND | ND |
| 11m | 122.80 | 92.80-162.5 | 15.96 |
| 12m | 59.17 | 44.05-79.49 | 7.69 |
| 14m | 159.50 | 128.1-198.7 | 20.74 |
| 15m | 944.00 | 816.0-1092 | 122.72 |
| 16m | 480.80 | 405.9-569.6 | 62.50 |
| 17m | 780.40 | 613.2-993.2 | 101.45 |
| 18m | 1300.00 | 1094-1543 | 169.00 |
| 20m | 772.50 | 654.7-911.4 | 100.43 |
| 21m | 831.30 | 707.5-976.9 | 108.07 |
| 27m | 1828.00 | 1550-2156 | 237.64 |
| 30m | ND | ND | ND |
| 33m | 947.50 | 794.5-1130 | 123.18 |
| 38m | 2100.00 | 1661-2655 | 273.00 |
| 40m | 5042.00 | 4253-5978 | 655.46 |
| 41m | 3803.00 | 2766-5228 | 494.39 |
| 42m | 1661.00 | 1341-2059 | 215.93 |
| 45m | 2331.00 | 1878-2892 | 303.03 |
| 46m | 2190.00 | 1834-2615 | 284.70 |
| 47m | 3010.00 | 2348-3857 | 391.30 |
| 49m | 2288.00 | 1748-2996 | 297.44 |
| 50m | 2386.00 | 1813-3140 | 310.18 |
| 51m | 2450.00 | 1965-3054 | 318.50 |
| 52m | 763.60 | 533.2-1094 | 99.27 |
| 53m | 554.60 | 424.1-725.3 | 72.10 |
| 55m | ND | ND | ND |
| 5C4 | 91.42 | 76.90-108.7 | 11.88 |
| 62m | 1647.00 | 1359-1995 | 214.11 |
| 6m | 124.10 | 95.44-161.4 | 16.13 |
| 9m | 275 | 235.2-321.6 | 35.75 |

The IC₅₀ values of an RSV A2 neutralizing antibody for 112 RSV screening antibody samples were confirmed by an FRNT test. As a result, 8 clones (8, 9, 13, 16, 33, 6m, 11m, and 12m) were confirmed as antibodies exhibiting excellent RSV neutralizing activity (FIG. 11).

TABLE 22
IC₅₀ of RSV neutralizing antibody against selected antibodies

| Clone | IC₅₀ (ng/mL) | Range | Name | Library |
|---|---|---|---|---|
| 8 | 12.43 | 9.92-15.56 | 12A1 | Chromium |
| 9 | 7.38 | 6.15-8.85 | 2H1 | Chromium |
| 13 | 11.04 | 8.64-14.11 | 10E7 | Manual |
| 16 | 12.58 | 10.53-15.04 | 3H8 | Manual |
| 33 | 16.78 | 13.52-20.85 | 4G1 | Chromium |
| 6m | 16.13 | 12.41-20.98 | 2E7 | Chromium |
| 11m | 15.96 | 12.06-23.13 | 12E9 | Chromium |
| 12m | 7.69 | 5.73-10.33 | 4F11 | Chromium |

### Example 6. Quantitative Measurement Result for Binding Ability of F-Protein

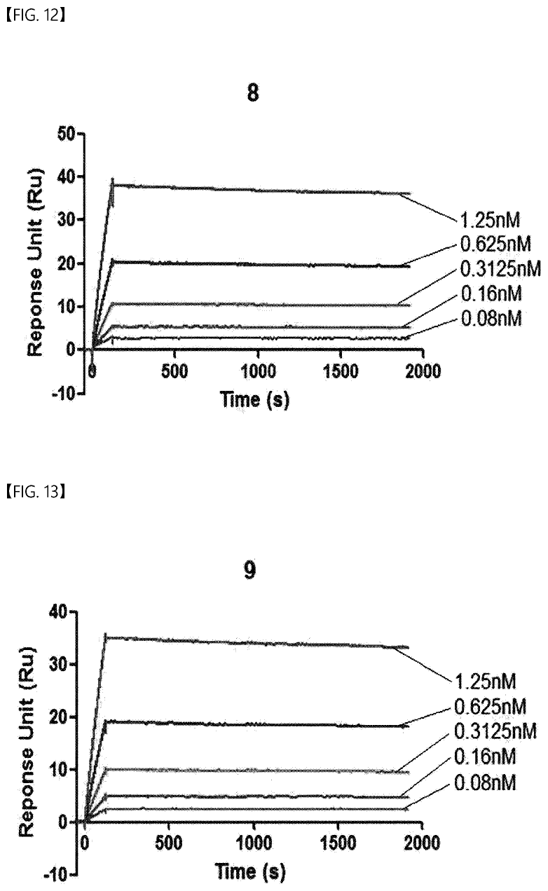

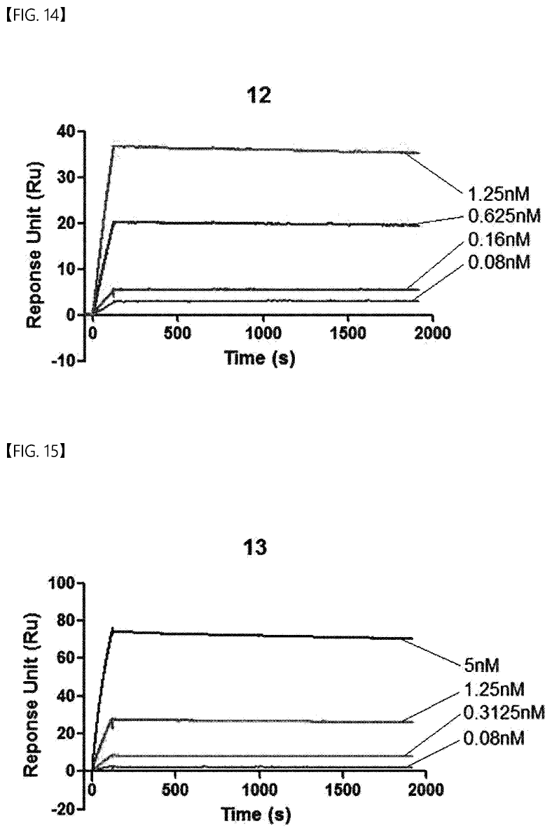

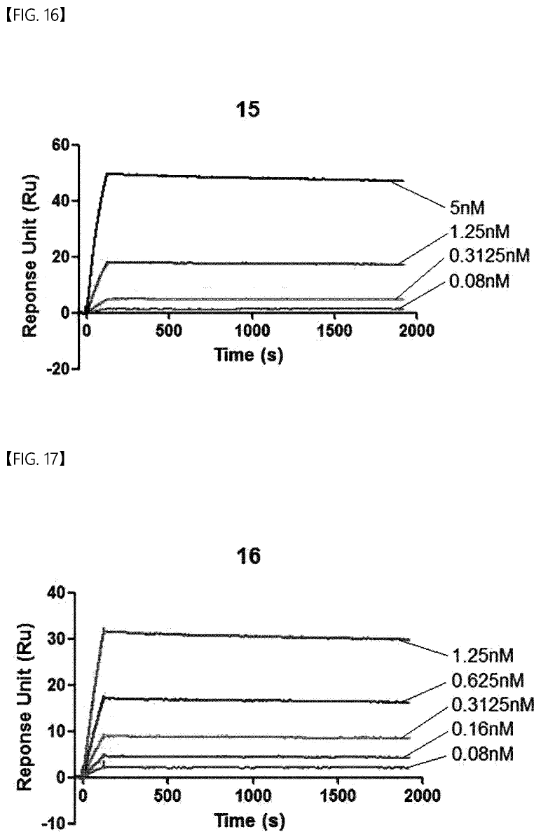

Table 23 below shows the affinity measurement results for the anti-F antibodies selected from the results of the neutralizing antibody analysis as the kinetic rate constants (Kₒₙ and K_{off}) and the equilibrium dissociation constant (K_{D}) (FIGS. 12 to 17).

TABLE 23
Equilibrium dissociation constant of anti-F antibody

| Clone | Kₒₙ | K_{off} | K_{D} |
|---|---|---|---|
| 8 | 2.80 × 10⁶ | 3.18 × 10⁻⁵ | 1.14 × 10⁻¹¹ |
| 9 | 1.50 × 10⁷ | 5.74 × 10⁻⁵ | 3.84 × 10⁻¹² |
| 12m | 3.06 × 10⁶ | 2.07 × 10⁻⁵ | 6.77 × 10⁻¹² |
| 13 | 2.03 × 10⁶ | 2.81 × 10⁻⁵ | 1.38 × 10⁻¹¹ |
| 15 | 2.52 × 10⁶ | 2.89 × 10⁻⁵ | 1.15 × 10⁻¹¹ |
| 16 | 1.53 × 10⁷ | 6.06 × 10⁻⁵ | 3.97 × 10⁻¹² |
| 33 | 1.77 × 10⁶ | 6.02 × 10⁻⁵ | 3.41 × 10⁻¹¹ |

### Example 7. Confirmation of Sequences of Finally-Selected 8 Types of Anti-RSV Antibodies

As described above, 8 types of antibodies having an excellent effect were finally selected by measuring IC₅₀ values, and the sequence information on heavy chains CDR1 to CDR3 and light chains CDR1 to CDR3, and heavy chain variable domains and light chain variable domains of the antibodies are shown in Tables 24 to 26.

TABLE 24
Amino acid sequences of heavy chain CDR1 to CDR3
and light chain CDR1 to CDR3 for 8 types of antibodies

| | | | SEQ | | SEQ | | SEQ |
|---|---|---|---|---|---|---|---|
| | Variable | | ID | | ID | | ID |
| Clone | domain | CDR1 | NO: | CDR2 | NO: | CDR3 | NO: |
| 8 | Heavy | SSYTMH | 1 | SITGGSSYVDYSASVK | 2 | DDYGSGSYSNWF | 3 |
| | chain | | | G | | | |
| | Light | SSSDIGAGYDV | 4 | ANTNRPS | 5 | QSYDRSLS | 6 |
| | chain | H | | | | | |
| 9 | Heavy | SSYTMH | 1 | SITGGSSYVDYSASVK | 2 | DDYGSGSYSNWF | 3 |
| | chain | | | G | | | |
| | Light | SSSNIGAGYDV | 7 | ANTNRPS | 5 | QSYDRSLS | 6 |
| | chain | H | | | | | |
| 13 | Heavy | TSYRMH | 8 | SITGGGNYIEYADSVK | 9 | DMYGLGSYYSPNY | 10 |
| | chain | | | G | | F | |
| | Light | SSSNIGAGYDV | 7 | ANTNRPS | 5 | QSYDRSLS | 6 |
| | chain | H | | | | | |
| 16 | Heavy | SSYTMH | 1 | SITGGSSYVDYSASVK | 2 | DDYGSGSYSNWF | 3 |
| | chain | | | G | | | |
| | Light | SSSNIGAGYDV | 7 | ANTNRPS | 5 | QSYDRSLS | 6 |
| | chain | H | | | | | |
| 33 | Heavy | SSYGMN | 11 | SISASSSFINYADSVRD | 12 | DDYGSGSYSNWF | 3 |
| | chain | | | | | | |
| | Light | SSNLGAGYDVH | 13 | ANTNRPS | 5 | QSYDRSLS | 6 |
| | chain | | | | | | |
| 6m | Heavy | SSYTMH | 1 | SITGGSSYVDYSASVK | 2 | DDYGSGSYSNWF | 3 |
| | chain | | | G | | | |
| | Light | SSNIGAGYDVH | 14 | GNSNRPS | 15 | QSYDRSLS | 6 |
| | chain | | | | | | |
| 11m | Heavy | TSYRMH | 8 | SITGGGNYIEYADSVK | 9 | DDYGSGSYSNWF | 3 |
| | chain | | | G | | | |
| | Light | SSNIGAGYDVH | 14 | ANTNRPS | 5 | QSYDRSLS | 6 |
| | chain | | | | | | |
| 12m | Heavy | SSYTMH | 1 | SITGGSSYLDYAHSVK | 16 | DDYGSGSYSNYF | 17 |
| | chain | | | G | | | |
| | Light | SSNIGAGYDVH | 14 | GSTNRPS | 18 | QSYDRSLS | 6 |
| | chain | | | | | | |

TABLE 25
Amino acid sequences of heavy chain variable domain and light chain variable
domain for 8 types of antibodies (each CDR region is underlined)

| | Variable | | SEQ ID |
|---|---|---|---|
| Clone | domain | Amino acid sequence | NO: |
| 8 | Heavy chain | EVQLVESGGGLIQPGGSLRLSCAGSGFTFSSYTMHWVRQ | 19 |
| | | APGKGLEWVSSITGGSSYVDYSASVKGRFTISRDNAQSSL | |
| | | YLQMNSLRAEDTAVYYCARDDYGSGSYSNWFDPWGQG | |
| | | TLVTVSS | |
| | Light chain | QSVLTQPPSVSGAPGQRVTISCTGSSSDIGAGYDVHWYQ | 20 |
| | | QLPGTAPKLLIFANTNRPSGVPDRESGSKSGASASLAITGL | |
| | | QADDEADYYCQSYDRSLSHVFGTGTKVTVLG | |
| 9 | Heavy chain | QVTLKESGGGVVQSGRSLRLSCAASGFTFSSYTMHWVR | 21 |
| | | QAPGKGLEWVSSITGGSSYVDYSASVKGRFTISRDNAQSS | |
| | | LYLQMNSLRAEDTAVYYCARDDYGSGSYSNWFDPWGQ | |
| | | GTLVTVSS | |
| | Light chain | QSVLTQPPSVSGAPGQRVTISCTGSSSNIGAGYDVHWYQ | 22 |
| | | QLPGRAPKLLIYANTNRPSGVADRFSGSKSGASASLAITG | |
| | | LQADDEADYYCQSYDRSLSHVFGTGTKVTVLG | |
| 13 | Heavy chain | EVQLVESGGGLVKPGGSLRLSCAASGFTFTSYRMHWVR | 23 |
| | | QAPGKGLEWVSSITGGGNYIEYADSVKGRFTISRDNAKN | |
| | | SLDLQMNSLRAEDTAIYYCARDMYGLGSYYSPNYFDSW | |
| | | GQGTLVTVSS | |
| | Light chain | QSVLTQPPSVSGAPGQRVTISCTGSSSNIGAGYDVHWYQ | 24 |
| | | QLPGRAPKLLIFANTNRPSGVPDRFSGSKSGASASLAITGL | |
| | | QADDEADYYCQSYDRSLSHVFGTGTKVTVLG | |
| 16 | Heavy chain | EVQLVESGGGLVKPGGSLRLSCAGSGFTFSSYTMHWVRQ | 25 |
| | | APGKGLEWVSSITGGSSYVDYSASVKGRFTISRDNAQSSL | |
| | | YLQMNSLRAEDTAVYYCARDDYGSGSYSNWEDPWGRG | |
| | | TTVTVSS | |
| | Light chain | QPVLTQPLSASGTPGQRVTISCTGSSSNIGAGYDVHWYQ | 26 |
| | | QLPGRAPKLLIFANTNRPSGVPDRFSGSKSGTSASLAITGL | |
| | | QAEDEADYYCQSYDRSLSHVFGTGTKVTVLG | |
| 33 | Heavy chain | QVTLKESGGGLVKPGGSLRLSCAASGFRLSSYGMNWVR | 27 |
| | | QAPGKGLEWVSSISASSSFINYADSVRDRFTISRDNAKNS | |
| | | LYLQMNSLRAEDTAVYYCARDDYGSGSYSNWFDPWGQ | |
| | | GTLVTVSS | |
| | Light chain | QSVLTQPPSVSGAPGQRVTISCTGNSSNLGAGYDVHWYQ | 28 |
| | | QLPGRAPKLLIFANTNRPSGVPDRFSGSKSGASASLAITSL | |
| | | QADDEADYYCQSYDRSLSHVFGTGTKVTVLG | |
| 6m | Heavy chain | EVQLVESGGGLVKPGGSLRLSCAGSGFTFSSYTMHWVRQ | 29 |
| | | APGKGLEWVSSITGGSSYVDYSASVKGRFTISRDNAQSSL | |
| | | YLQMNSLRAEDTAVYYCARDDYGSGSYSNWFDPWGQG | |
| | | TLVTVSS | |
| | Light chain | QSVLTQPPSASGTPGQRVTISCTGSSSNIGAGYDVHWYQQ | 30 |
| | | LPGAAPRLLMFGNSNRPSGVPDRESGSKSGTSASLAITGL | |
| | | QADDEADYYCQSYDRSLSHVFGTGTKVTVLG | |
| 11m | Heavy chain | EVQLVESGGGLVKPGGSLRLSCAASGFTFTSYRMHWVR | 31 |
| | | QAPGKGLEWVSSITGGGNYIEYADSVKGRFTISRDNAQSS | |
| | | LYLQMNSLRAEDTAVYYCARDDYGSGSYSNWFDPWGQ | |
| | | GTLVTVSS | |
| | Light chain | QSVLTQPPSVSGAPGQRVTISCTGSSSNIGAGYDVHWYQ | 24 |
| | | QLPGRAPKLLIFANTNRPSGVPDRESGSKSGASASLAITGL | |
| | | QADDEADYYCQSYDRSLSHVFGTGTKVTVLG | |
| 12m | Heavy chain | EVQLVESGGGLVKPGGSLRLSCAGSGFAFSSYTMHWVR | 32 |
| | | QAPGKGLEWVSSITGGSSYLDYAHSVKGRFTISRDNGQN | |
| | | SLFLQMNSLRTEDTAVYYCARDDYGSGSYSNYFDPWGQ | |
| | | GTLVTVSS | |
| | Light chain | QSVLTQPPSVSGAPGQRVTISCTGSSSNIGAGYDVHWYQ | 33 |
| | | QVPGTAPKLLIFGSTNRPSGVPDRFSGSKSGASASLAITGL | |
| | | QTEDEADYYCQSYDRSLSHVFGTGTKVTVLG | |

TABLE 26
Polynucleotide sequences of heavy chain
variable domain and light chain variable
domain for 8 types of antibodies

| | Variable | | SEQ ID |
|---|---|---|---|
| Clone | domain | DNA sequence | NO: |
| 8 | Heavy chain | GAGGTGCAGCTGGTGGAGTCTGGGGGAGGCCTGATAC | 34 |
| | | AGCCTGGGGGGTCCCTGAGACTCTCCTGTGCAGGCTCT | |
| | | GGATTCACCTTCAGTAGCTATACCATGCACTGGGTCCG | |
| | | CCAGGCTCCAGGGAAGGGGCTGGAGTGGGTCTCGTCC | |
| | | ATAACTGGTGGCAGTAGTTATGTCGACTACTCAGCCTC | |
| | | AGTGAAGGGCCGATTCACCATCTCCAGAGACAACGCC | |
| | | CAGAGCTCACTTTATCTGCAAATGAACAGCCTGAGAGC | |
| | | CGAGGACACGGCCGTGTATTACTGTGCGAGAGATGAT | |
| | | TATGGTTCGGGGAGTTATTCCAACTGGTTCGACCCCTG | |
| | | GGGCCAGGGAACCCTGGTCACCGTCTCCTCA | |
| | Light chain | CAGTCTGTCCTGACGCAGCCGCCCTCAGTGTCTGGGGC | 35 |
| | | CCCAGGGCAGAGGGTCACCATCTCCTGCACTGGGAGC | |
| | | AGCTCCGACATCGGGGCAGGTTATGATGTACACTGGTA | |
| | | CCAGCAACTTCCAGGAACAGCCCCCAAACTCCTCATCT | |
| | | TTGCTAACACCAATCGGCCCTCAGGGGTCCCTGATCGA | |
| | | TTCTCTGGCTCCAAGTCTGGCGCCTCTGCCTCCCTGGCC | |
| | | ATCACTGGCCTCCAGGCTGACGATGAGGCTGATTATTA | |
| | | CTGCCAGTCCTATGACCGCAGCCTGAGTCATGTCTTCG | |
| | | GAACTGGGACCAAGGTCACCGTCCTAGGC | |
| 9 | Heavy chain | CAGGTCACCTTGAAGGAGTCTGGGGGAGGCGTGGTCC | 36 |
| | | AGTCTGGGAGGTCCCTGAGACTCTCCTGTGCAGCCTCT | |
| | | GGATTCACCTTCAGTAGCTATACCATGCACTGGGTCCG | |
| | | CCAGGCTCCAGGGAAGGGGCTGGAGTGGGTCTCGTCC | |
| | | ATAACTGGTGGCAGTAGTTATGTCGACTACTCAGCCTC | |
| | | AGTGAAGGGCCGATTCACCATCTCCAGAGACAACGCC | |
| | | CAGAGCTCACTTTATCTGCAAATGAACAGCCTGAGAGC | |
| | | CGAGGACACGGCTGTGTATTACTGTGCGAGAGATGATT | |
| | | ATGGTTCGGGGAGTTATTCCAACTGGTTCGACCCCTGG | |
| | | GGCCAGGGAACCCTGGTCACCGTCTCCTCA | |
| | Light chain | CAGTCTGTGCTGACGCAGCCGCCCTCAGTGTCTGGGGC | 37 |
| | | CCCAGGGCAGAGGGTCACCATCTCCTGCACTGGGAGC | |
| | | AGCTCCAACATCGGGGCAGGTTATGATGTACACTGGTA | |
| | | CCAGCAGCTTCCAGGAAGAGCCCCCAAACTCCTCATCT | |
| | | ATGCTAACACCAATCGGCCCTCAGGGGTCGCTGACCG | |
| | | ATTCTCTGGCTCCAAGTCTGGCGCCTCTGCCTCCCTGG | |
| | | CCATCACTGGCCTCCAGGCTGACGATGAGGCTGATTAT | |
| | | TACTGCCAGTCCTATGACCGCAGCCTGAGTCATGTCTT | |
| | | CGGAACTGGGACCAAGGTCACCGTCCTAGGC | |
| 13 | Heavy chain | GAGGTGCAGCTGGTGGAGTCTGGGGGAGGCCTGGTCA | 38 |
| | | AGCCTGGGGGGTCCCTGAGACTCTCCTGTGCAGCCTCT | |
| | | GGATTCACCTTCACTAGTTATAGGATGCATTGGGTCCG | |
| | | CCAGGCTCCAGGGAAGGGGCTGGAGTGGGTCTCATCA | |
| | | ATTACTGGTGGTGGTAATTATATAGAGTACGCAGACTC | |
| | | AGTGAAGGGCCGGTTCACCATCTCCAGAGACAACGCC | |
| | | AAGAACTCACTGGATCTGCAAATGAACAGCCTGAGAG | |
| | | CCGAGGACACGGCTATTTATTACTGTGCGAGAGATATG | |
| | | TATGGTTTGGGGAGTTATTATTCGCCTAACTACTTCGA | |
| | | CTCCTGGGGCCAGGGAACCCTGGTCACCGTCTCCTCA | |
| | Light chain | CAGTCTGTGCTGACGCAGCCGCCCTCAGTGTCTGGGGC | 39 |
| | | CCCAGGGCAGAGGGTCACCATCTCCTGCACTGGGAGC | |
| | | AGCTCCAACATCGGGGCAGGTTATGATGTACACTGGTA | |
| | | CCAGCAGCTTCCAGGAAGAGCCCCCAAACTCCTCATCT | |
| | | TTGCTAACACCAATCGGCCCTCAGGGGTCCCTGACCGA | |
| | | TTCTCTGGCTCCAAGTCTGGCGCCTCTGCCTCCCTGGCC | |
| | | ATCACTGGCCTCCAGGCTGACGATGAGGCTGATTATTA | |
| | | CTGCCAGTCCTATGACCGCAGCCTGAGTCATGTCTTCG | |
| | | GAACTGGGACCAAGGTCACCGTCCTAGGC | |
| 16 | Heavy chain | GAGGTGCAGCTGGTGGAGTCTGGGGGAGGCCTGGTCA | 40 |
| | | AGCCTGGGGGGTCCCTGAGACTCTCCTGTGCAGGCTCT | |
| | | GGATTCACCTTCAGTAGCTATACCATGCACTGGGTCCG | |
| | | CCAGGCTCCAGGGAAGGGGCTGGAGTGGGTCTCGTCC | |
| | | ATAACTGGTGGCAGTAGTTATGTCGACTACTCAGCCTC | |
| | | AGTGAAGGGCCGATTCACCATCTCCAGAGATAACGCC | |
| | | CAGAGCTCACTTTATCTGCAAATGAACAGCCTGAGAGC | |
| | | CGAGGACACGGCTGTGTATTACTGTGCGAGAGATGATT | |
| | | ATGGTTCGGGGAGTTATTCCAACTGGTTCGACCCCTGG | |
| | | GGCCGGGGAACCACGGTCACCGTCTCCTCA | |
| | Light chain | CAGCCTGTGCTGACTCAGCCACTCTCAGCGTCTGGGAC | 41 |
| | | CCCCGGGCAGAGGGTCACCATCTCCTGCACTGGGAGC | |
| | | AGCTCCAACATCGGGGCAGGTTATGATGTACACTGGTA | |
| | | CCAGCAGCTTCCAGGAAGAGCCCCCAAACTCCTCATCT | |
| | | TTGCTAACACCAATCGGCCCTCAGGGGTCCCTGACCGA | |
| | | TTCTCTGGCTCCAAGTCGGGCACCTCAGCCTCCCTGGC | |
| | | CATCACTGGGCTCCAGGCTGAGGATGAGGCTGATTATT | |
| | | ACTGCCAGTCCTATGACCGCAGCCTGAGTCATGTCTTC | |
| | | GGAACTGGGACCAAGGTCACCGTCCTAGGC | |
| 33 | Heavy chain | CAGGTCACCTTGAAGGAGTCTGGGGGCGGCCTGGTTA | 42 |
| | | AGCCTGGGGGGTCCCTGAGACTCTCGTGTGCAGCCTCT | |
| | | GGGTTCAGGCTCAGTAGCTATGGCATGAACTGGGTCCG | |
| | | CCAGGCTCCAGGGAAGGGGCTGGAGTGGGTCTCATCC | |
| | | ATTTCTGCTAGTAGTAGTTTTATAAACTATGCAGACTC | |
| | | AGTGAGGGACCGATTCACCATCTCCAGAGACAACGCC | |
| | | AAGAACTCACTGTATCTGCAAATGAACAGCCTGAGAG | |
| | | CCGAGGACACGGCTGTGTATTACTGTGCGAGAGATGA | |
| | | TTATGGTTCGGGGAGTTATTCCAACTGGTTCGACCCCT | |
| | | GGGGCCAGGGAACCCTGGTCACCGTCTCCTCA | |
| | Light chain | CAGTCTGTGCTGACGCAGCCGCCCTCAGTGTCTGGGGC | 43 |
| | | CCCAGGGCAGAGGGTCACCATTTCCTGCACTGGGAAC | |
| | | AGCTCCAACCTCGGGGCAGGTTATGATGTACACTGGTA | |
| | | CCAGCAGCTTCCAGGAAGAGCCCCCAAACTCCTCATCT | |
| | | TTGCTAACACCAATCGGCCCTCAGGGGTCCCTGACCGA | |
| | | TTCTCTGGCTCCAAGTCTGGCGCCTCTGCCTCCCTGGCC | |
| | | ATCACTAGCCTCCAGGCTGACGATGAGGCTGATTATTA | |
| | | CTGCCAGTCCTATGACCGCAGCCTGAGTCATGTCTTCG | |
| | | GAACTGGGACCAAGGTCACCGTCCTAGGC | |
| 6m | Heavy chain | GAGGTGCAGCTGGTGGAGTCTGGGGGAGGCCTGGTCA | 44 |
| | | AGCCTGGGGGGTCCCTGAGACTCTCCTGTGCAGGCTCT | |
| | | GGATTCACCTTCAGTAGCTATACCATGCACTGGGTCCG | |
| | | CCAGGCTCCAGGGAAGGGGCTGGAGTGGGTCTCGTCC | |
| | | ATAACTGGTGGCAGTAGTTATGTCGACTACTCAGCCTC | |
| | | AGTGAAGGGCCGATTCACCATCTCCAGAGACAACGCC | |
| | | CAGAGCTCACTTTATCTGCAAATGAACAGCCTGAGAGC | |
| | | CGAGGACACGGCTGTATATTACTGTGCGAGAGATGATT | |
| | | ATGGTTCGGGGAGTTATTCCAACTGGTTCGACCCCTGG | |
| | | GGCCAGGGAACCCTGGTCACCGTCTCCTCA | |
| | Light chain | CAGTCTGTGCTGACGCAGCCGCCCTCAGCGTCTGGGAC | 45 |
| | | CCCCGGGCAGAGGGTCACCATCTCCTGCACTGGGAGC | |
| | | AGCTCCAACATCGGGGCAGGTTATGATGTACACTGGTA | |
| | | TCAGCAGCTTCCGGGAGCAGCCCCCAGACTCCTCATGT | |
| | | TTGGTAACAGCAATCGGCCCTCGGGGGTACCTGACCGC | |
| | | TTCTCTGGCTCCAAGTCTGGCACCTCCGCCTCCCTGGC | |
| | | CATCACTGGCCTCCAGGCTGACGATGAGGCTGATTATT | |
| | | ACTGCCAGTCCTATGACCGCAGCCTGAGTCATGTCTTC | |
| | | GGAACTGGGACCAAGGTCACCGTCCTAGGC | |
| 11m | Heavy chain | GAGGTGCAGCTGGTGGAGTCTGGGGGAGGCCTGGTCA | 46 |
| | | AGCCTGGGGGGTCCCTGAGACTCTCCTGTGCAGCCTCT | |
| | | GGATTCACCTTCACTAGTTATAGGATGCATTGGGTCCG | |
| | | CCAGGCTCCAGGGAAGGGGCTGGAGTGGGTCTCATCA | |
| | | ATTACTGGTGGTGGTAATTATATAGAGTACGCAGACTC | |
| | | AGTGAAGGGCCGATTCACCATCTCCAGAGACAACGCC | |
| | | CAGAGCTCACTTTATCTGCAAATGAACAGCCTGAGAGC | |
| | | CGAGGACACGGCTGTGTATTACTGTGCGAGAGATGATT | |
| | | ATGGTTCGGGGAGTTATTCCAACTGGTTCGACCCCTGG | |
| | | GGCCAGGGAACCCTGGTCACCGTCTCCTCA | |
| | Light chain | CAGTCTGTGCTGACGCAGCCGCCCTCAGTGTCTGGGGC | 39 |
| | | CCCAGGGCAGAGGGTCACCATCTCCTGCACTGGGAGC | |
| | | AGCTCCAACATCGGGGCAGGTTATGATGTACACTGGTA | |
| | | CCAGCAGCTTCCAGGAAGAGCCCCCAAACTCCTCATCT | |
| | | TTGCTAACACCAATCGGCCCTCAGGGGTCCCTGACCGA | |
| | | TTCTCTGGCTCCAAGTCTGGCGCCTCTGCCTCCCTGGCC | |
| | | ATCACTGGCCTCCAGGCTGACGATGAGGCTGATTATTA | |
| | | CTGCCAGTCCTATGACCGCAGCCTGAGTCATGTCTTCG | |
| | | GAACTGGGACCAAGGTCACCGTCCTAGGC | |
| 12m | Heavy chain | GAGGTGCAGCTGGTGGAGTCTGGGGGAGGCCTGGTCA | 47 |
| | | AGCCTGGGGGGTCCCTGAGACTTTCCTGTGCTGGCTCT | |
| | | GGATTCGCCTTCAGTAGTTACACTATGCACTGGGTGCG | |
| | | CCAGGCTCCAGGGAAGGGGCTGGAGTGGGTCTCATCC | |
| | | ATCACTGGCGGCAGTAGTTACCTAGACTACGCACACTC | |
| | | AGTGAAGGGCCGATTCACCATCTCCAGAGACAATGGC | |
| | | CAGAACTCACTGTTTCTGCAAATGAACAGCCTGAGGAC | |
| | | CGAGGACACGGCTGTATATTACTGTGCGAGAGATGAC | |
| | | TATGGTTCGGGGAGTTATTCCAACTACTTCGACCCCTG | |
| | | GGGCCAGGGAACCCTGGTCACCGTCTCCTCA | |
| | Light chain | CAGTCTGTGCTGACGCAGCCGCCCTCAGTGTCTGGGGC | 48 |
| | | CCCAGGGCAGAGGGTCACCATCTCCTGCACTGGGAGC | |
| | | AGCTCCAACATCGGGGCAGGTTATGATGTACACTGGTA | |
| | | CCAGCAGGTTCCAGGAACGGCCCCCAAACTCCTCATCT | |
| | | TTGGTAGCACCAATCGGCCCTCAGGGGTCCCTGACCGA | |
| | | TTCTCTGGCTCCAAGTCTGGCGCCTCAGCCTCCCTGGC | |
| | | CATCACTGGGCTCCAGACTGAGGATGAGGCTGATTATT | |
| | | ACTGCCAGTCCTATGACCGCAGCCTGAGTCATGTCTTC | |
| | | GGAACTGGGACCAAGGTCACCGTCCTAGGC | |

1. An antibody or an antigen-binding fragment thereof specifically binding to respiratory syncytial virus (RSV) selected from the group consisting of the following (i) to (vii):
(i) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 2, and heavy chain CDR3 comprising the amino acid sequence of SEQ ID NO: 3, and a light chain variable domain which comprises light chain CDR1 comprising the amino acid sequence of SEQ ID NO: 4, light chain CDR2 comprising the amino acid sequence of SEQ ID NO: 5, and light chain CDR3 comprising the amino acid sequence of SEQ ID NO: 6;
(ii) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 2, and heavy chain CDR3 comprising the amino acid sequence of SEQ ID NO: 3, and a light chain variable domain which comprises light chain CDR1 comprising the amino acid sequence of SEQ ID NO: 7, light chain CDR2 comprising the amino acid sequence of SEQ ID NO: 5, and light chain CDR3 comprising the amino acid sequence of SEQ ID NO: 6;
(iii) an antibody specifically binding to RSV, comprising a heavy chain variable domain which comprises heavy chain CDR1 comprising the amino acid sequence of SEQ ID NO: 8, heavy chain CDR2 comprising the amino acid sequence of SEQ ID NO: 9, and heavy chain CDR3 comprising the amino acid sequence of SEQ ID NO: 10, and a light chain variable domain which comprises light chain CDR1 comprising the amino acid sequence of SEQ ID NO: 7, light chain CDR2 comprising the amino acid sequence of SEQ ID NO: 5, and light chain CDR3 comprising the amino acid sequence of SEQ ID NO: 6;
(iv) an antibody specifically binding to RSV, comprising a heavy chain variable domain which comprises heavy chain CDR1 comprising the amino acid sequence of SEQ ID NO: 11, heavy chain CDR2 comprising the amino acid sequence of SEQ ID NO: 12, and heavy chain CDR3 comprising the amino acid sequence of SEQ ID NO: 3, and a light chain variable domain which comprises light chain CDR1 comprising the amino acid sequence of SEQ ID NO: 13, light chain CDR2 comprising the amino acid sequence of SEQ ID NO: 5, and light chain CDR3 comprising the amino acid sequence of SEQ ID NO: 6;
(v) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 2, and heavy chain CDR3 comprising the amino acid sequence of SEQ ID NO: 3, and a light chain variable domain which comprises light chain CDR1 comprising the amino acid sequence of SEQ ID NO: 14, light chain CDR2 comprising the amino acid sequence of SEQ ID NO: 15, and light chain CDR3 comprising the amino acid sequence of SEQ ID NO: 6;
(vi) an antibody specifically binding to RSV, comprising a heavy chain variable domain which comprises heavy chain CDR1 comprising the amino acid sequence of SEQ ID NO: 8, heavy chain CDR2 comprising the amino acid sequence of SEQ ID NO: 9, and heavy chain CDR3 comprising the amino acid sequence of SEQ ID NO: 3, and a light chain variable domain which comprises light chain CDR1 comprising the amino acid sequence of SEQ ID NO: 14, light chain CDR2 comprising the amino acid sequence of SEQ ID NO: 5, and light chain CDR3 comprising the amino acid sequence of SEQ ID NO: 6; and
(vii) an antibody specifically binding to RSV, comprising a heavy chain variable domain which comprises heavy chain CDR1 comprising the amino acid sequence of SEQ ID NO: 1, heavy chain CDR2 comprising the amino acid sequence of SEQ ID NO: 16, and heavy chain CDR3 comprising the amino acid sequence of SEQ ID NO: 17, and a light chain variable domain which comprises light chain CDR1 comprising the amino acid sequence of SEQ ID NO: 14, light chain CDR2 comprising the amino acid sequence of SEQ ID NO: 18, and light chain CDR3 comprising the amino acid sequence of SEQ ID NO: 6.

2. The antibody or an antigen-binding fragment thereof of claim 1, wherein the antibody is an antibody specifically binding to respiratory syncytial virus (RSV) selected from the group consisting of the following (viii) to (xv):
(viii) an antibody specifically binding to RSV, comprising a heavy chain variable domain comprising the amino acid sequence of SEQ ID NO: 19 and a light chain variable domain comprising the amino acid sequence of SEQ ID NO: 20;
(ix) an antibody specifically binding to RSV, comprising a heavy chain variable domain comprising the amino acid sequence of SEQ ID NO: 21 and a light chain variable domain comprising the amino acid sequence of SEQ ID NO: 22;
(x) an antibody specifically binding to RSV, comprising a heavy chain variable domain comprising the amino acid sequence of SEQ ID NO: 23 and a light chain variable domain comprising the amino acid sequence of SEQ ID NO: 24;
(xi) an antibody specifically binding to RSV, comprising a heavy chain variable domain comprising the amino acid sequence of SEQ ID NO: 25 and a light chain variable domain comprising the amino acid sequence of SEQ ID NO: 26;
(xii) an antibody specifically binding to RSV, comprising a heavy chain variable domain comprising the amino acid sequence of SEQ ID NO: 27 and a light chain variable domain comprising the amino acid sequence of SEQ ID NO: 28;
(xiii) an antibody specifically binding to RSV, comprising a heavy chain variable domain comprising the amino acid sequence of SEQ ID NO: 29 and a light chain variable domain comprising the amino acid sequence of SEQ ID NO: 30;
(xiv) an antibody specifically binding to RSV, comprising a heavy chain variable domain comprising the amino acid sequence of SEQ ID NO: 31 and a light chain variable domain comprising the amino acid sequence of SEQ ID NO: 24; and
(xv) an antibody specifically binding to RSV, comprising a heavy chain variable domain comprising the amino acid sequence of SEQ ID NO: 32 and a light chain variable domain comprising the amino acid sequence of SEQ ID NO: 33.

3. The antibody or an antigen-binding fragment thereof of claim 1, wherein the antibody is an antibody specifically binding to respiratory syncytial virus (RSV), selected from the group consisting of the following (xvi) to (xxiii):
(xvi) an antibody specifically binding to RSV, comprising a heavy chain variable domain encoded by the nucleotide sequence of SEQ ID NO: 34 and a light chain variable domain encoded by the nucleotide sequence of SEQ ID NO: 35;
(xvii) an antibody specifically binding to RSV, comprising a heavy chain variable domain encoded by the nucleotide sequence of SEQ ID NO: 36 and a light chain variable domain encoded by the nucleotide sequence of SEQ ID NO: 37;
(xviii) an antibody specifically binding to RSV, comprising a heavy chain variable domain encoded by the nucleotide sequence of SEQ ID NO: 38 and a light chain variable domain encoded by the nucleotide sequence of SEQ ID NO: 39;
(xix) an antibody specifically binding to RSV, comprising a heavy chain variable domain encoded by the nucleotide sequence of SEQ ID NO: 40 and a light chain variable domain encoded by the nucleotide sequence of SEQ ID NO: 41;
(xx) an antibody specifically binding to RSV, comprising a heavy chain variable domain encoded by the nucleotide sequence of SEQ ID NO: 42 and a light chain variable domain encoded by the nucleotide sequence of SEQ ID NO: 43;
(xxi) an antibody specifically binding to RSV, comprising a heavy chain variable domain encoded by the nucleotide sequence of SEQ ID NO: 44 and a light chain variable domain encoded by the nucleotide sequence of SEQ ID NO: 45;
(xxii) an antibody specifically binding to RSV, comprising a heavy chain variable domain encoded by the nucleotide sequence of SEQ ID NO: 46 and a light chain variable domain encoded by the nucleotide sequence of SEQ ID NO: 39; and
(xxiii) an antibody specifically binding to RSV, comprising a heavy chain variable domain encoded by the nucleotide sequence of SEQ ID NO: 47 and a light chain variable domain encoded by the nucleotide sequence of SEQ ID NO: 48.

4. The antibody or an antigen-binding fragment thereof of claim 1, wherein the antibody specifically binds to the F-protein of RSV.

5. The antibody or an antigen-binding fragment thereof of claim 1, wherein the antibody is a humanized antibody.

6. The antibody or an antigen-binding fragment thereof of claim 1, wherein the antibody is a neutralizing antibody.

7. The antibody or an antigen-binding fragment thereof of claim 1, wherein the antigen-binding fragment of the antibody is a Fab fragment, a Fab′ fragment, a F(ab′)₂ fragment, or a scFv fragment.

8. A polynucleotide encoding a light chain variable domain and a light chain variable domain of the antibody specifically bind to respiratory syncytial virus (RSV) according to claim 1.

9. An expression vector comprising the polynucleotide of claim 8.

10. A host cell transformed with the expression vector of claim 9.

11. A method of preparing an antibody or an antigen-binding fragment thereof specifically binding to respiratory syncytial virus (RSV), comprising the step of culturing the host cell of claim 10.

12. A pharmaceutical composition comprising the antibody or antigen-binding fragment thereof of claim 1 and a pharmaceutically acceptable carrier.

13. A method of treating a subject having a respiratory syncytial virus (RSV) infection comprising administering the antibody or an antigen-binding fragment thereof of claim 1 to the subject.

14. A method of treating a subject having a respiratory syncytial virus (RSV) infection comprising administering the antibody or an antigen-binding fragment thereof of claim 2 to the subject.

15. A method of treating a subject having a respiratory syncytial virus (RSV) infection comprising administering the antibody or an antigen-binding fragment thereof of claim 3 to the subject.

16. A method of treating a subject having a respiratory syncytial virus (RSV) infection comprising administering the antibody or an antigen-binding fragment thereof of claim 4 to the subject.

17. A method of treating a subject having a respiratory syncytial virus (RSV) infection comprising administering the antibody or an antigen-binding fragment thereof of claim 5 to the subject.

18. A method of treating a subject having a respiratory syncytial virus (RSV) infection comprising administering the antibody or an antigen-binding fragment thereof of claim 6 to the subject.

19. A method of treating a subject having a respiratory syncytial virus (RSV) infection comprising administering the antibody or an antigen-binding fragment thereof of claim 7 to the subject.

20. A method of treating a subject having a respiratory syncytial virus (RSV) infection comprising administering the pharmaceutical composition of claim 12 to the subject.


SEQUENCE LISTING (530 sequences)

SEQ ID NO: 1 - protein, 6 aa, Artificial Sequence
  clone 8 heavy chain CDR1
SSYTMH

SEQ ID NO: 2 - protein, 17 aa, Artificial Sequence
  clone 8 heavy chain CDR2
SITGGSSYVDYSASVKG

SEQ ID NO: 3 - protein, 12 aa, Artificial Sequence
  clone 8 heavy chain CDR3
DDYGSGSYSNWF

SEQ ID NO: 4 - protein, 12 aa, Artificial Sequence
  clone 8 light chain CDR1
SSSDIGAGYDVH

SEQ ID NO: 5 - protein, 7 aa, Artificial Sequence
  clone 8 light chain CDR2
ANTNRPS

SEQ ID NO: 6 - protein, 8 aa, Artificial Sequence
  clone 8 light chain CDR3
QSYDRSLS

SEQ ID NO: 7 - protein, 12 aa, Artificial Sequence
  clone 9 light chain CDR1
SSSNIGAGYDVH

SEQ ID NO: 8 - protein, 6 aa, Artificial Sequence
  clone 13 heavy chain CDR1
TSYRMH

SEQ ID NO: 9 - protein, 17 aa, Artificial Sequence
  clone 13 heavy chain CDR2
SITGGGNYIEYADSVKG

SEQ ID NO: 10 - protein, 14 aa, Artificial Sequence
  clone 13 heavy chain CDR3
DMYGLGSYYSPNYF

SEQ ID NO: 11 - protein, 6 aa, Artificial Sequence
  clone 33 heavy chain CDR1
SSYGMN

SEQ ID NO: 12 - protein, 17 aa, Artificial Sequence
  clone 33 heavy chain CDR2
SISASSSFINYADSVRD

SEQ ID NO: 13 - protein, 11 aa, Artificial Sequence
  clone 33 light chain CDR1
SSNLGAGYDVH

SEQ ID NO: 14 - protein, 11 aa, Artificial Sequence
  clone 6m light chain CDR1
SSNIGAGYDVH

SEQ ID NO: 15 - protein, 7 aa, Artificial Sequence
  clone 6m light chain CDR2
GNSNRPS

SEQ ID NO: 16 - protein, 17 aa, Artificial Sequence
  clone 12m heavy chain CDR2
SITGGSSYLDYAHSVKG

SEQ ID NO: 17 - protein, 12 aa, Artificial Sequence
  clone 12m heavy chain CDR3
DDYGSGSYSNYF

SEQ ID NO: 18 - protein, 7 aa, Artificial Sequence
  clone 12m light chain CDR2
GSTNRPS

SEQ ID NO: 19 - protein, 123 aa, Artificial Sequence
  clone 8 heavy chain
EVQLVESGGGLIQPGGSLRLSCAGSGFTFSSYTMHWVRQAPGKGLEWVSSITGGSSYVDYSASVKGRFTISRDNAQSSLYLQMNSLRAEDTAVYYCARDDYGSGSYSNWFDPWGQGTLVTVSS

SEQ ID NO: 20 - protein, 111 aa, Artificial Sequence
  clone 8 light chain
QSVLTQPPSVSGAPGQRVTISCTGSSSDIGAGYDVHWYQQLPGTAPKLLIFANTNRPSGVPDRFSGSKSGASASLAITGLQADDEADYYCQSYDRSLSHVFGTGTKVTVLG

SEQ ID NO: 21 - protein, 123 aa, Artificial Sequence
  clone 9 heavy chain
QVTLKESGGGVVQSGRSLRLSCAASGFTFSSYTMHWVRQAPGKGLEWVSSITGGSSYVDYSASVKGRFTISRDNAQSSLYLQMNSLRAEDTAVYYCARDDYGSGSYSNWFDPWGQGTLVTVSS

SEQ ID NO: 22 - protein, 111 aa, Artificial Sequence
  clone 9 light chain
QSVLTQPPSVSGAPGQRVTISCTGSSSNIGAGYDVHWYQQLPGRAPKLLIYANTNRPSGVADRFSGSKSGASASLAITGLQADDEADYYCQSYDRSLSHVFGTGTKVTVLG

SEQ ID NO: 23 - protein, 125 aa, Artificial Sequence
  clone 13 heavy chain
EVQLVESGGGLVKPGGSLRLSCAASGFTFTSYRMHWVRQAPGKGLEWVSSITGGGNYIEYADSVKGRFTISRDNAKNSLDLQMNSLRAEDTAIYYCARDMYGLGSYYSPNYFDSWGQGTLVTVSS

SEQ ID NO: 24 - protein, 111 aa, Artificial Sequence
  clone 13 light chain
QSVLTQPPSVSGAPGQRVTISCTGSSSNIGAGYDVHWYQQLPGRAPKLLIFANTNRPSGVPDRFSGSKSGASASLAITGLQADDEADYYCQSYDRSLSHVFGTGTKVTVLG

SEQ ID NO: 25 - protein, 123 aa, Artificial Sequence
  clone 16 heavy chain
EVQLVESGGGLVKPGGSLRLSCAGSGFTFSSYTMHWVRQAPGKGLEWVSSITGGSSYVDYSASVKGRFTISRDNAQSSLYLQMNSLRAEDTAVYYCARDDYGSGSYSNWFDPWGRGTTVTVSS

SEQ ID NO: 26 - protein, 111 aa, Artificial Sequence
  clone 16 light chain
QPVLTQPLSASGTPGQRVTISCTGSSSNIGAGYDVHWYQQLPGRAPKLLIFANTNRPSGVPDRFSGSKSGTSASLAITGLQAEDEADYYCQSYDRSLSHVFGTGTKVTVLG

SEQ ID NO: 27 - protein, 123 aa, Artificial Sequence
  clone 33 heavy chain
QVTLKESGGGLVKPGGSLRLSCAASGFRLSSYGMNWVRQAPGKGLEWVSSISASSSFINYADSVRDRFTISRDNAKNSLYLQMNSLRAEDTAVYYCARDDYGSGSYSNWFDPWGQGTLVTVSS

SEQ ID NO: 28 - protein, 111 aa, Artificial Sequence
  clone 33 light chain
QSVLTQPPSVSGAPGQRVTISCTGNSSNLGAGYDVHWYQQLPGRAPKLLIFANTNRPSGVPDRFSGSKSGASASLAITSLQADDEADYYCQSYDRSLSHVFGTGTKVTVLG

SEQ ID NO: 29 - protein, 123 aa, Artificial Sequence
  clone 6m heavy chain
EVQLVESGGGLVKPGGSLRLSCAGSGFTFSSYTMHWVRQAPGKGLEWVSSITGGSSYVDYSASVKGRFTISRDNAQSSLYLQMNSLRAEDTAVYYCARDDYGSGSYSNWFDPWGQGTLVTVSS

SEQ ID NO: 30 - protein, 111 aa, Artificial Sequence
  clone 6m light chain
QSVLTQPPSASGTPGQRVTISCTGSSSNIGAGYDVHWYQQLPGAAPRLLMFGNSNRPSGVPDRFSGSKSGTSASLAITGLQADDEADYYCQSYDRSLSHVFGTGTKVTVLG

SEQ ID NO: 31 - protein, 123 aa, Artificial Sequence
  clone 11m heavy chain
EVQLVESGGGLVKPGGSLRLSCAASGFTFTSYRMHWVRQAPGKGLEWVSSITGGGNYIEYADSVKGRFTISRDNAQSSLYLQMNSLRAEDTAVYYCARDDYGSGSYSNWFDPWGQGTLVTVSS

SEQ ID NO: 32 - protein, 123 aa, Artificial Sequence
  clone 12m heavy chain
EVQLVESGGGLVKPGGSLRLSCAGSGFAFSSYTMHWVRQAPGKGLEWVSSITGGSSYLDYAHSVKGRFTISRDNGQNSLFLQMNSLRTEDTAVYYCARDDYGSGSYSNYFDPWGQGTLVTVSS

SEQ ID NO: 33 - protein, 111 aa, Artificial Sequence
  clone 12m light chain
QSVLTQPPSVSGAPGQRVTISCTGSSSNIGAGYDVHWYQQVPGTAPKLLIFGSTNRPSGVPDRFSGSKSGASASLAITGLQTEDEADYYCQSYDRSLSHVFGTGTKVTVLG

SEQ ID NO: 34 - DNA, 369 bp, Artificial Sequence
  clone 8 heavy chain
gaggtgcagctggtggagtctgggggaggcctgatacagcctggggggtccctgagactctcctgtgcaggctctggattcaccttcagtagctataccatgcactgggtccgccaggctccagggaaggggctggagtgggtctcgtccataactggtggcagtagttatgtcgactactcagcctcagtgaagggccgattcaccatctccagagacaacgcccagagctcactttatctgcaaatgaacagcctgagagccgaggacacggccgtgtattactgtgcgagagatgattatggttcggggagttattccaactggttcgacccctggggccagggaaccctggtcaccgtctcctca

SEQ ID NO: 35 - DNA, 333 bp, Artificial Sequence
  clone 8 light chain
cagtctgtcctgacgcagccgccctcagtgtctggggccccagggcagagggtcaccatctcctgcactgggagcagctccgacatcggggcaggttatgatgtacactggtaccagcaacttccaggaacagcccccaaactcctcatctttgctaacaccaatcggccctcaggggtccctgatcgattctctggctccaagtctggcgcctctgcctccctggccatcactggcctccaggctgacgatgaggctgattattactgccagtcctatgaccgcagcctgagtcatgtcttcggaactgggaccaaggtcaccgtcctaggc

SEQ ID NO: 36 - DNA, 369 bp, Artificial Sequence
  clone 9 heavy chain
caggtcaccttgaaggagtctgggggaggcgtggtccagtctgggaggtccctgagactctcctgtgcagcctctggattcaccttcagtagctataccatgcactgggtccgccaggctccagggaaggggctggagtgggtctcgtccataactggtggcagtagttatgtcgactactcagcctcagtgaagggccgattcaccatctccagagacaacgcccagagctcactttatctgcaaatgaacagcctgagagccgaggacacggctgtgtattactgtgcgagagatgattatggttcggggagttattccaactggttcgacccctggggccagggaaccctggtcaccgtctcctca

SEQ ID NO: 37 - DNA, 333 bp, Artificial Sequence
  clone 9 light chain
cagtctgtgctgacgcagccgccctcagtgtctggggccccagggcagagggtcaccatctcctgcactgggagcagctccaacatcggggcaggttatgatgtacactggtaccagcagcttccaggaagagcccccaaactcctcatctatgctaacaccaatcggccctcaggggtcgctgaccgattctctggctccaagtctggcgcctctgcctccctggccatcactggcctccaggctgacgatgaggctgattattactgccagtcctatgaccgcagcctgagtcatgtcttcggaactgggaccaaggtcaccgtcctaggc

SEQ ID NO: 38 - DNA, 375 bp, Artificial Sequence
  clone 13 heavy chain
gaggtgcagctggtggagtctgggggaggcctggtcaagcctggggggtccctgagactctcctgtgcagcctctggattcaccttcactagttataggatgcattgggtccgccaggctccagggaaggggctggagtgggtctcatcaattactggtggtggtaattatatagagtacgcagactcagtgaagggccggttcaccatctccagagacaacgccaagaactcactggatctgcaaatgaacagcctgagagccgaggacacggctatttattactgtgcgagagatatgtatggtttggggagttattattcgcctaactacttcgactcctggggccagggaaccctggtcaccgtctcctca

SEQ ID NO: 39 - DNA, 333 bp, Artificial Sequence
  clone 13 light chain
cagtctgtgctgacgcagccgccctcagtgtctggggccccagggcagagggtcaccatctcctgcactgggagcagctccaacatcggggcaggttatgatgtacactggtaccagcagcttccaggaagagcccccaaactcctcatctttgctaacaccaatcggccctcaggggtccctgaccgattctctggctccaagtctggcgcctctgcctccctggccatcactggcctccaggctgacgatgaggctgattattactgccagtcctatgaccgcagcctgagtcatgtcttcggaactgggaccaaggtcaccgtcctaggc

SEQ ID NO: 40 - DNA, 369 bp, Artificial Sequence
  clone 16 heavy chain
gaggtgcagctggtggagtctgggggaggcctggtcaagcctggggggtccctgagactctcctgtgcaggctctggattcaccttcagtagctataccatgcactgggtccgccaggctccagggaaggggctggagtgggtctcgtccataactggtggcagtagttatgtcgactactcagcctcagtgaagggccgattcaccatctccagagataacgcccagagctcactttatctgcaaatgaacagcctgagagccgaggacacggctgtgtattactgtgcgagagatgattatggttcggggagttattccaactggttcgacccctggggccggggaaccacggtcaccgtctcctca

SEQ ID NO: 41 - DNA, 333 bp, Artificial Sequence
  clone 16 light chain
cagcctgtgctgactcagccactctcagcgtctgggacccccgggcagagggtcaccatctcctgcactgggagcagctccaacatcggggcaggttatgatgtacactggtaccagcagcttccaggaagagcccccaaactcctcatctttgctaacaccaatcggccctcaggggtccctgaccgattctctggctccaagtcgggcacctcagcctccctggccatcactgggctccaggctgaggatgaggctgattattactgccagtcctatgaccgcagcctgagtcatgtcttcggaactgggaccaaggtcaccgtcctaggc

SEQ ID NO: 42 - DNA, 369 bp, Artificial Sequence
  clone 33 heavy chain
caggtcaccttgaaggagtctgggggcggcctggttaagcctggggggtccctgagactctcgtgtgcagcctctgggttcaggctcagtagctatggcatgaactgggtccgccaggctccagggaaggggctggagtgggtctcatccatttctgctagtagtagttttataaactatgcagactcagtgagggaccgattcaccatctccagagacaacgccaagaactcactgtatctgcaaatgaacagcctgagagccgaggacacggctgtgtattactgtgcgagagatgattatggttcggggagttattccaactggttcgacccctggggccagggaaccctggtcaccgtctcctca

SEQ ID NO: 43 - DNA, 333 bp, Artificial Sequence
  clone 33 light chain
cagtctgtgctgacgcagccgccctcagtgtctggggccccagggcagagggtcaccatttcctgcactgggaacagctccaacctcggggcaggttatgatgtacactggtaccagcagcttccaggaagagcccccaaactcctcatctttgctaacaccaatcggccctcaggggtccctgaccgattctctggctccaagtctggcgcctctgcctccctggccatcactagcctccaggctgacgatgaggctgattattactgccagtcctatgaccgcagcctgagtcatgtcttcggaactgggaccaaggtcaccgtcctaggc

SEQ ID NO: 44 - DNA, 369 bp, Artificial Sequence
  clone 6m heavy chain
gaggtgcagctggtggagtctgggggaggcctggtcaagcctggggggtccctgagactctcctgtgcaggctctggattcaccttcagtagctataccatgcactgggtccgccaggctccagggaaggggctggagtgggtctcgtccataactggtggcagtagttatgtcgactactcagcctcagtgaagggccgattcaccatctccagagacaacgcccagagctcactttatctgcaaatgaacagcctgagagccgaggacacggctgtatattactgtgcgagagatgattatggttcggggagttattccaactggttcgacccctggggccagggaaccctggtcaccgtctcctca

SEQ ID NO: 45 - DNA, 333 bp, Artificial Sequence
  clone 6m light chain
cagtctgtgctgacgcagccgccctcagcgtctgggacccccgggcagagggtcaccatctcctgcactgggagcagctccaacatcggggcaggttatgatgtacactggtatcagcagcttccgggagcagcccccagactcctcatgtttggtaacagcaatcggccctcgggggtacctgaccgcttctctggctccaagtctggcacctccgcctccctggccatcactggcctccaggctgacgatgaggctgattattactgccagtcctatgaccgcagcctgagtcatgtcttcggaactgggaccaaggtcaccgtcctaggc

SEQ ID NO: 46 - DNA, 369 bp, Artificial Sequence
  clone 11m heavy chain
gaggtgcagctggtggagtctgggggaggcctggtcaagcctggggggtccctgagactctcctgtgcagcctctggattcaccttcactagttataggatgcattgggtccgccaggctccagggaaggggctggagtgggtctcatcaattactggtggtggtaattatatagagtacgcagactcagtgaagggccgattcaccatctccagagacaacgcccagagctcactttatctgcaaatgaacagcctgagagccgaggacacggctgtgtattactgtgcgagagatgattatggttcggggagttattccaactggttcgacccctggggccagggaaccctggtcaccgtctcctca

SEQ ID NO: 47 - DNA, 369 bp, Artificial Sequence
  clone 12m heavy chain
gaggtgcagctggtggagtctgggggaggcctggtcaagcctggggggtccctgagactttcctgtgctggctctggattcgccttcagtagttacactatgcactgggtgcgccaggctccagggaaggggctggagtgggtctcatccatcactggcggcagtagttacctagactacgcacactcagtgaagggccgattcaccatctccagagacaatggccagaactcactgtttctgcaaatgaacagcctgaggaccgaggacacggctgtatattactgtgcgagagatgactatggttcggggagttattccaactacttcgacccctggggccagggaaccctggtcaccgtctcctca

SEQ ID NO: 48 - DNA, 333 bp, Artificial Sequence
  clone 12m light chain
cagtctgtgctgacgcagccgccctcagtgtctggggccccagggcagagggtcaccatctcctgcactgggagcagctccaacatcggggcaggttatgatgtacactggtaccagcaggttccaggaacggcccccaaactcctcatctttggtagcaccaatcggccctcaggggtccctgaccgattctctggctccaagtctggcgcctcagcctccctggccatcactgggctccagactgaggatgaggctgattattactgccagtcctatgaccgcagcctgagtcatgtcttcggaactgggaccaaggtcaccgtcctaggc

SEQ ID NO: 49 - DNA, 369 bp, Artificial Sequence
  clone 1 heavy chain
gaggtgcagctggtggagtctgggggaggcctggtcaagcctggggggtccctgagactctcctgtgcagcctctggattcacgttcagtagctataccatgcactgggtccgccaggctccagggaaggggctggagtgggtctcgtccataactggtggcagtagttatgtcgactactcagcctcagtgaagggccgattcaccatctccagagacaacgctcagagctcactttatctgcaaatgaacagcctgagagccgaggacacggctgtgtattactgtgcgagagatgattatggttcggggagttattccaactggttcgacccctggggccagggaaccctggtcaccgtctcctca

SEQ ID NO: 50 - DNA, 333 bp, Artificial Sequence
  clone 1 light chain
cagtctgtgctgacgcagccgccctcagtgtctggggccccagggcagagggtcaccatctcctgcactgggagcagctccaacatcggggcaggttatgatgtacactggtaccagcaggttccaggaacagcccccaaactcctcatctttggtagcaccaatcggccctcaggggtccctgaccgattctctggctccaagtctggcacctcagcctccctggccatcactggcctccaggctgacgatgaggctgattattactgccagtcctatgaccgcagcctgagtcatgtcttcggaactgggaccaaggtcaccgtcctaggc

SEQ ID NO: 51 - DNA, 369 bp, Artificial Sequence
  clone 2 heavy chain
gaggtgcagctggtggagtctgggggaggcgtggtccagtctgggaggtccctgagactctcctgtgcaggctctggattcaccttcagtagctataccatgcactgggtccgccaggctccagggaaggggctggagtgggtctcgtccataactggtggcagtagttatgtcgactactcagcctcagtgaagggccgattcaccatctccagagacaacgcccagagctcactttatctgcaaatgaacagcctgagagccgaggacacggctgtgtattactgtgcgagagatgattatggttcggggagttattccaactggttcgacccctggggccagggaaccctggtcaccgtctcctca

SEQ ID NO: 52 - DNA, 333 bp, Artificial Sequence
  clone 2 light chain
cagtctgtcctgacgcagccgccctcagtgtctggggccccagggcagagggtcaccatctcctgcactgggagcagctccaacatcggggcaggttatgatgtacactggtaccagcaggttccaggaacagcccccaaactcctcatctttggtagcaccaatcggccctcaggggtccctgaccgattctctggctccaagtctggcgcctcagcctccctggccatcactgggctccagactgaggatgaggctgattattactgccagtcctatgaccgcagcctgagtcatgtcttcggaactgggaccaaggtcaccgtcctaggc

SEQ ID NO: 53 - DNA, 369 bp, Artificial Sequence
  clone 3 heavy chain
gaggtgcagctggtggagtctgggggaggcctggtcaagcctggggggtccctgagactctcctgtgcaggctctggattcaccttcagtagctataccatgcactgggtccgccaggctccagggaaggggctggagtgggtctcgtccataactggtggcagtagttatgtcgactactcagcctcagtgaagggccgattcaccatctccagagacaacgcccagagctcactttatctgcaaatgaacagcctgagagccgaggacacggctgtgtattactgtgcgagagatgattatggttcggggagttattccaactggttcgacccctggggccagggaaccctggtcaccgtctcctca

SEQ ID NO: 54 - DNA, 333 bp, Artificial Sequence
  clone 3 light chain
cagtctgtgctgacgcagccgccctcagtgtctggggccccagggcagagggtcaccatctcctgcactgggagcagctccaacatcggggcaggttacgatgtacactggtaccagcaggttccaggaacagcccccaaactcctcatctttggtagcaccaatcggccctcaggggtccctgaccgattctctggttccaagtctggcgcctctgcctccctggccatcactggcctccaggctgacgatgaggctgattattactgccagccctatgaccgcagcctgagtcatgtcttcggaactgggaccaaggtcaccgtcctacgc

SEQ ID NO: 55 - DNA, 369 bp, Artificial Sequence; identical to SEQ ID NO: 53
  clone 4 heavy chain

SEQ ID NO: 56 - DNA, 333 bp, Artificial Sequence
  clone 4 light chain
cagtctgtgctgacgcagccgccctcagtgtctggggccccagggcagagggtcaccatctcctgcactgggagcagctccaacatcggggcaggttacgatgtacactggtaccagcaggttccaggaacagcccccaaactcctcatctttggtagcaccaatcggccctcaggggtccctgaccgattctctggctccaagtctggcgcctctgcctccctggccatcactggcctccaggctgacgatgaggctgattattactgccagtcctatgaccgcagcctgagtcatgtcttcggaactgggaccaaggtcaccgtcctaggc

SEQ ID NO: 57 - DNA, 369 bp, Artificial Sequence; identical to SEQ ID NO: 53
  clone 5 heavy chain

SEQ ID NO: 58 - DNA, 333 bp, Artificial Sequence
  clone 5 light chain
cagtctgtgctgacgcagccgccctcagtgtctggggccccagggcagagggtcaccatctcctgcactgggagcagctccaacatcggggcaggttatgatgtacactggtatcagcagcttccgggagcagcccccagactcctcatgtttggtaacagcaatcggccctcgggggtacctgaccgcttctctggctccaagtctggcacctccgcctccctggccatcactggtctccaggctgaggatgaggctgattattactgccagtcctatgaccgcagcctgagtcatgtcttcggaactggcaccaaggtgaccgtcctcggc

SEQ ID NO: 59 - DNA, 333 bp, Artificial Sequence
  clone 6 light chain
tcctatgagctgactcagccaccctcagcgtctgggacccccgggcagagggtcaccatctcctgcactgggagcagctccaacatcggggcaggttatgatgtacactggtatcagcagcttccgggagcagcccccagactcctcatgtttggtaacagcaatcggccctcgggggtacctgaccgcttctctggctccaagtctggcacctccgcctccctggccatcactggcctccaggctgacgatgaggctgattattactgccagtcctatgaccgcagcctgagtcatgtcttcggaactgggaccaaggtcaccgtcctaggc

SEQ ID NO: 60 - DNA, 369 bp, Artificial Sequence
  clone 7 heavy chain
caggtgcagctggtgcagtctgggggaggcttgatacagcctggggggtccctgagactctcctgtgcaggctctggattcaccttcagtagctataccatgcactgggtccgccaggctccagggaaggggctggagtgggtctcgtccataactggtggcagtagttatgtcgactactcagcctcagtgaagggccgattcaccatctccagagacaacgcccagagctcactttatctgcaaatgaacagcctgagagccgaggacacggccgtgtattactgtgcgagagatgattatggttcggggagttattccaactggttcgacccctggggccagggaaccctggtcaccgtctcctca

SEQ ID NO: 61 - DNA, 333 bp, Artificial Sequence
  clone 7 light chain
cagtctgtgctgacgcagccgccctcagtgtctggggccccagggcagagggtcaccatctcctgcactgggagcagctccaacatcggggcaggttatgatgtacactggtaccagcaacttccaggaacagcccccaaactcctcatctttgctaacaccaatcggccctcaggggtccctgatcgattctctggctccaagtctggcgcctctgcctccctggccatcactggcctccaggctgacgatgaggctgattattactgccagtcctatgaccgcagcctgagtcatgtcttcggaactgggaccaaggtcaccgtcctaggc

SEQ ID NO: 62 - DNA, 426 bp, Artificial Sequence
  clone 9m heavy chain
tgctgtgggtgagtggtacctgtggggaggtgcagctggtggagtctgggggaggcgtggtccagtctgggaggtccctgagactctcctgtgcagcctctggattcaccttcagtagctataccatgcactgggtccgccaggctccagggaaggggctggagtgggtctcgtccataactggtggcagtagttatgtcgactactcagcctcagtgaagggccgattcaccatctccagagacaacgcccagagctcactttatctgcaaatgaacagcctgagagccgaggacacggctgtgtattactgtgcgagagatgattatggttcggggagttattccaactggttcgacccctggggccagggaaccctggtcaccgtctcctcagcctccaccaagggcccatcggtcttccccc

SEQ ID NO: 63 - DNA, 369 bp, Artificial Sequence
  clone 10 heavy chain
gaggtgcagctggtggagtctgggggaggcctggtcaagcctggggggtccctgagactctcctgtgcaggctctggattcaccttcagtagctataccatgcactgggtccgccaggctccagggaaggggctggagtgggtctcgtccataactggtggcagtagttatgtcgactactcagcctcagtgaagggccgattcaccatctccagagacaacgcccagagctcactttatctgcaaatgaacagcctgagagccgaggacacggctgtgtattactgtgcgagagatgattatggttcggggagttattctaactggttcgacccctggggccagggaaccctggtcaccgtctcctca

SEQ ID NO: 64 - DNA, 333 bp, Artificial Sequence
  clone 10 light chain
cagtctgtgctgacgcagccgccctcagtgtctggggccccagggcagagggtcaccatttcctgcactgggaacagctccaacctcggggcaggttatgatgtacactggtaccagcaacttccaggaacagcccccaaactcctcatctatgctaacaccaatcggccctcaggggtccctgaccgattctctggctccaagtctggcgcctcagcctccctggccatcactgggctccagactgaggatgaggctgattattactgccagtcctatgaccgcagcctgagtcatgtcttcggaactgggaccaaggtcaccgtcctaggc

SEQ ID NO: 65 - DNA, 333 bp, Artificial Sequence
  clone 11 light chain
cagactgtggtgactcaggagccctcagtgtctggggccccagggcagagggtcaccatctcctgcactgggagcagctccaacatcggggcaggttatgatgtacactggtaccagcagcttccaggaagagcccccaaactcctcatctttgctaacaccaatcggccctcaggggtccctgaccgattctctggctccaagtctggcgcctctgcctccctggccatcactggcctccaggctgacgatgaggctgattattactgccagtcctatgaccgcagcctgagtcatgtcttcggaactgggaccaaggtcaccgtcctaggc

SEQ ID NO: 66 - DNA, 333 bp, Artificial Sequence
  clone 12 light chain
cagcctgtgctgactcagccaccctcagtgtctggggccccagggcagagggtcaccatctcctgcactgggagcagctccaacatcggggcaggttatgatgtacactggtaccagcaggttccaggaacggcccccaaactcctcatctttggtagcaccaatcggccctcaggggtccctgaccgattctctggctccaagtctggcgcctcagcctccctggccatcactgggctccagactgaggatgaggctgattattactgccagtcctatgaccgcagcctgagtcatgtcttcggaactgggaccaaggtcaccgtcctaggc

SEQ ID NO: 67 - DNA, 369 bp, Artificial Sequence
  clone 14 heavy chain
caggtcaccttgaaggagtctgggggaggcctggtcaagcctggggggtccctgagactctcctgtgcaggctctggattcaccttcagtagctataccatgcactgggtccgccaggctccagggaaggggctggagtgggtctcgtccataactggtggcagtagttatgtcgactactcagcctcagtgaagggccgattcaccatctccagagacaacgccaagaactcactgtttctgcaaatgaacagcctgagagccgaggacacggctgtgtattactgtgcgagagatgattatggttcggggagttattccaactggttcgacccctggggccagggaaccctggtcaccgtctcctca

SEQ ID NO: 68 - DNA, 368 bp, Artificial Sequence
  clone 14m heavy chain
aggtgcagctggtggagtctgggggaggcctggtcaagcctggggggtccctgagactctcctgtgcaggctctggattcaccttcagtagctataccatgcactgggtccgccaggctccagggaaggggctggagtgggtctcgtccataactggtggcagtagttatgtcgactactcagcctcagtgaagggccgattcaccatctccagagacaacgccaagaactcactgtttctgcaaatgaacagcctgagagccgaggacacggctgtgtattactgtgcgagagatgattatggttcggggagttattccaactggttcgacccctggggccagggaaccctggtcaccgtctcctca

SEQ ID NO: 69 - DNA, 333 bp, Artificial Sequence
  clone 14 light chain
cagtctgtgctgacgcagccgccctcagtttctggggccccagggcagagggtcaccatctcctgcactgggagcagctccaacatcggggcaggttatgatgtacactggtaccagcagcttccaggaagagcccccaaactcctcatctttgctaacaccaatcggccctcaggggtccctgaccgattctctggctccaagtctggcgcctctgcctccctggccatcactggcctccaggctgacgatgaggctgattattactgccagtcctatgaccgcagcctgagtcatgtcttcggaactgggaccaaggtcaccgtcctaggc

SEQ ID NO: 70 - DNA, 375 bp, Artificial Sequence
  clone 15 heavy chain
gaggtgcagctggtggagtctgggggaggcgtggtccagcctgggaggtccctgagactcgcctgtgcagcctctggattcaccttcaatagctatgctatgcactgggtccgccaggctccagggaaggggctggagtgggtctcgtccataactggtggcagtagttatgtcgactactcagcctcagtgaagggccgattcaccatctccagagacaacgcccagagctcactttatctgcaaatgaacagcctgagagccgaggacacggctgtgtattactgtgcgagagatatgtatggtttggggagttattattcgcctaactacttcgacccctggggccagggaaccctggtcactgtctcctca

SEQ ID NO: 71 - DNA, 333 bp, Artificial Sequence
  clone 15 light chain
tcctatgagctgactcagccaccctcagtgtctggggccccagggcagagggtcaccatctcctgcactgggagcagctccaacatcggggcaggttatgatgtacactggtaccagcagcttccaggaagagcccccaaactcctcatctttgctaacaccaatcggccctcaggggtccctgaccgattctctggctccaagtctggcacctccgcctccctggccatcactggtctccaggctgaggatgaggctgattattactgccagtcctatgacagtagcctgagttatgtcttcggaactgggaccaaggtcaccgtcctaggc

SEQ ID NO: 72 - DNA, 333 bp, Artificial Sequence
  clone 15m light chain
cagtctgtgctgacgcagccgccctcagtgtctggggccccagggcagagggtcaccatctcctgcactgggagcagctccaacatcggggcaggttatgatgtacactggtaccagcagcttccaggaagagcccccaaactcctcatctttgctaacaccaatcggccctcaggggtccctgaccgattctctggctccaagtctggcacctccgcctccctggccatcactggtctccaggctgaggatgaggctgattattactgccagtcctatgacagtagcctgagttatgtcttcggaactgggaccaaggtcaccgtcctaggc

SEQ ID NO: 73 - DNA, 333 bp, Artificial Sequence
  clone 16m light chain
cagtctgtgctgacgcagccgctctcagcgtctgggacccccgggcagagggtcaccatctcctgcactgggagcagctccaacatcggggcaggttatgatgtacactggtaccagcagcttccaggaagagcccccaaactcctcatctttgctaacaccaatcggccctcaggggtccctgaccgattctctggctccaagtcgggcacctcagcctccctggccatcactgggctccaggctgaggatgaggctgattattactgccagtcctatgaccgcagcctgagtcatgtcttcggaactgggaccaaggtcaccgtcctaggc

SEQ ID NO: 74 - DNA, 369 bp, Artificial Sequence
  clone 17 heavy chain
gaggtgcagctggtggagtctgggggaggcttggtacagcctggagggtccctgagactcgcctgtgcagcctctggattcaccttcagtagctataccatgcactgggtccgccaggctccagggaaggggctggagtgggtctcgtccataactggtggcagtagttatgtcgactactcagcctcagtgaagggccgattcaccatctccagagacaacgcccagagctcactttatctgcaaatgaacagcctaagagccgaggacacggctgtgtattactgtgcgagagatgattatggttcggggagttattccaactggttcgacccctggggccagggaaccctggtcaccgtctcctca

SEQ ID NO: 75 - DNA, 333 bp, Artificial Sequence
  clone 17 light chain
tcctatgagctgactcagccaccctcagtgtctggggccccagggcagagggtcaccatctcctgcactgggagcagctccaacatcggggcaggttatgatgtacactggtaccagcagcttccaggaagagcccccaaactcctcatctttgctaacaccaatcggccctcaggggtccctgaccgattctctggctccaagtctggcgcctctgcctccctggccatcactggcctccaggctgaggatgaggctgattattactgccagtcctatgaccgcagcctgagtcatgtcttcggaactgggaccaaggtcaccgtcctaggc

SEQ ID NO: 76 - DNA, 333 bp, Artificial Sequence
  clone 17m light chain
cagtctgtgctgacgcagccgccctcagtgtctggggccccagggcagagggtcaccatctcctgcactgggagcagctccaacatcggggcaggttatgatgtacactggtaccagcagcttccaggaagagcccccaaactcctcatctttgctaacaccaatcggccctcaggggtccctgaccgattctctggctccaagtctggcgcctctgcctccctggccatcactggcctccaggctgaggatgaggctgattattactgccagtcctatgaccgcagcctgagtcatgtcttcggaactgggaccaaggtcaccgtcctaggc

SEQ ID NO: 77 - DNA, 375 bp, Artificial Sequence
  clone 18 heavy chain
caggtcaccttgaaggagtctgggggaggcctggtcaagcctggggggtccctgagactctcctgtgcagcctctggattcaccttcactagttataggatgcattgggtccgccaggctccagggaaggggctggagtgggtctcatcaattactggtggtggtaattatatagagtacgcagactcagtgaagggccggttcaccatctccagagacaacgccaagaactcactggatctgcaaatgaacagcctgagagccgaggacacggctatttattactgtgcgagagatatgtatggtttggggagttattattcgcctaactacttcgacccctggggccagggaaccctggtcaccgtctcctca

SEQ ID NO: 78 - DNA, 375 bp, Artificial Sequence
  clone 18m heavy chain
gaggtgcagctggtggagtctgggggaggcctggtcaagcctggggggtccctgagactctcctgtgcagcctctggattcaccttcactagttataggatgcattgggtccgccaggctccagggaaggggctggagtgggtctcatcaattactggtggtggtaattatatagagtacgcagactcagtgaagggccggttcaccatctccagagacaacgccaagaactcactggatctgcaaatgaacagcctgagagccgaggacacggctatttattactgtgcgagagatatgtatggtttggggagttattattcgcctaactacttcgacccctggggccagggaaccctggtcaccgtctcctca

SEQ ID NO: 79 - DNA, 333 bp, Artificial Sequence
  clone 18 light chain
tcctatgagctgactcagccactctcagtgtctggggccccagggcagagggtcaccatttcctgcactgggagcagctccaacatcggggcaggttatgatgtacactggtaccagcaggttccaggaacagcccccaaactcctcatctttggtagcaccaatcggccctcaggggtccctgaccgattctctggctccaagtctggcgcctctgcctccctggccatcactggcctccaggctgacgatgaggctgattattactgccagtcctatgaccgcagcctgagtcatgtcttcggaactgggaccaaggtcaccgtcctaggc

SEQ ID NO: 80 - DNA, 333 bp, Artificial Sequence
  clone 18m light chain
cagtctgtgctgacgcagccgctctcagtgtctggggccccagggcagagggtcaccatttcctgcactgggagcagctccaacatcggggcaggttatgatgtacactggtaccagcaggttccaggaacagcccccaaactcctcatctttggtagcaccaatcggccctcaggggtccctgaccgattctctggctccaagtctggcgcctctgcctccctggccatcactggcctccaggctgacgatgaggctgattattactgccagtcctatgaccgcagcctgagtcatgtcttcggaactgggaccaaggtcaccgtcctaggc

SEQ ID NO: 81 - DNA, 375 bp, Artificial Sequence
  clone 19 heavy chain
gaggtgcagctggtggagtctgggggaggcctggtcaagcctggggggtccctgagactctcctgtgcagcctctggattcaccttcactagttataggatgcattgggtccgccaggctccagggaagaggctggagtgggtctcatcaattactggtggtggtaattatatagagtacgcagactcagtgaagggccggttcaccatctccagagacaacgccaagaactcactggatctgcaaatgaacagcctgagagccgaggacacggctatttattactgtgcgagagatatgtatggtttggggagttattattcgcctaactacttcgacccctggggccagggaaccctggtcaccgtctcctca

SEQ ID NO: 82 - DNA, 333 bp, Artificial Sequence
  clone 19 light chain
cagtctgtgctgacgcagccgccctcagtgtctggggctccagggcagagggtcaccatctcctgcactgggagcagctccaacatcggggcaggttatgatgtacactggtaccagcaggttccaggaacagcccccaaactcctcatctttggtagcaccaatcggccctcaggggtccctgaccgattctctggctccaagtctggcgcctcagcctccctggccatcactgggctccagactgaggatgaggctgattattactgccagtcctatgaccgcagcctgagtcatgtcttcggaactgggaccaaggtcaccgtcctaggc

SEQ ID NO: 83 - DNA, 375 bp, Artificial Sequence; identical to SEQ ID NO: 78
  clone 20 heavy chain

SEQ ID NO: 84 - DNA, 333 bp, Artificial Sequence
  clone 20 light chain
cagcctgtgctgactcagccaccctcagtgtctggggccccagggcagagggtcaccatctcctgcactgggagcagctccaacatcggggcaggttatgatgtacactggtaccagcaggttccaggaacagcccccaaactcctcatctttggtagcaccaatcggccctcaggggtccctgaccgattctctggctccaagtctggcgcctcagcctccctggccatcactgggctccagactgaggatgaggctgattattactgccagtcctatgaccgcagcctgagtcatgtcttcggaactgggaccaaggtcaccgtcctaggc

SEQ ID NO: 85 - DNA, 333 bp, Artificial Sequence
  clone 20m light chain
cagtctgtgctgacgcagccgccctcagtgtctggggccccagggcagagggtcaccatctcctgcactgggagcagctccaacatcggggcaggttatgatgtacactggtaccagcaggttccaggaacagcccccaaactcctcatctttggtagcaccaatcggccctcaggggtccctgaccgattctctggctccaagtctggcgcctcagcctccctggccatcactgggctccagactgaggatgaggctgattattactgccagtcctatgaccgcagcctgagtcatgtcttcggaactgggaccaaggtcaccgtcctaggc

SEQ ID NO: 86 - DNA, 375 bp, Artificial Sequence; identical to SEQ ID NO: 77
  clone 21 heavy chain

SEQ ID NO: 87 - DNA, 375 bp, Artificial Sequence; identical to SEQ ID NO: 78
  clone 21m heavy chain

SEQ ID NO: 88 - DNA, 375 bp, Artificial Sequence; identical to SEQ ID NO: 78
  clone 21 light chain

SEQ ID NO: 89 - DNA, 375 bp, Artificial Sequence
  clone 22 heavy chain
gaggtgcagctggtggagtctgggggaggcctggtcaagcctggggggtccctgagactctcctgtgcagcctctggattcaccttcactagttataggatgcattgggtccgccaggctccagggaaggggctggagtgggtctcatcaattactggtggtggtaattatatagagtacgcagactcagtgaagggccggttcaccatctccagagacaacgccaagaactcactggacctgcaaatgaacagcctgagagccgaggacacggctatttattactgtgcgagagatatgtatggtttggggagttattattcgcctaactacttcgacccctggggccagggaaccctggtcactgtctcctca

SEQ ID NO: 90 - DNA, 333 bp, Artificial Sequence; identical to SEQ ID NO: 69
  clone 22 light chain

SEQ ID NO: 91 - DNA, 375 bp, Artificial Sequence
  clone 23 heavy chain
caggtccagctggtacagtctgggggaggcctggtcaagcctggggggtccctgagactctcctgtgcagcctctggattcaccttcactagttataggatgcattgggtccgccaggctccagggaaggggctggagtgggtctcatcaattactggtggtggtaattatatagagtacgcagactcagtgaagggccggttcaccatctccagagacaacgccaagaactcactggatctgcaaatgaacagcctgagagccgaggacacggctatttattactgtgcgagagatatgtatggtttggggagttattattcgcctaactacttcgacccctggggccagggaaccacggtcaccgtctcctca

SEQ ID NO: 92 - DNA, 333 bp, Artificial Sequence
  clone 23 light chain
cagtctgtgttgacgcagccgccctcagtgtctggggccccagggcagagggtcaccatctcctgcactgggagcagctccaacatcggggcaggttatgatgtacactggtaccagcagcttccaggaagagcccccaaactcctcatctttgctaacaccaatcggccctcaggggtccctgaccgattctctggctccaagtctggcgcctctgcctccctggccatctctgggctccaggctgaggatgaggctcattattactgccagtcctatgacaggagcctgaatgtggttttcggcggagggaccgagctgaccgtcctaggc

SEQ ID NO: 93 - DNA, 375 bp, Artificial Sequence; identical to SEQ ID NO: 78
  clone 24 heavy chain

SEQ ID NO: 94 - DNA, 336 bp, Artificial Sequence
  clone 24 light chain
cagtctgtgctgacgcagccgccctcagtgtctggggccccagggcagagggtcaccatctcctgcactgggagcagctccaacatcggggcaggttatgatgtacactggtaccagcaggttccaggaacagcccccaaactcctcatctttggtagcaccaatcggccctcaggggtccctgaccgattctctggctccaagtctggcgcctcagcctccctggccatcactgggctccagactgaggatgaggctgattattattgccagtcctatgacagaagcctgagtgcttgggtgttcggcggagggaccaagctgaccgtcctaggc

SEQ ID NO: 95 - DNA, 375 bp, Artificial Sequence; identical to SEQ ID NO: 78
  clone 25 heavy chain

SEQ ID NO: 96 - DNA, 333 bp, Artificial Sequence
  clone 25 light chain
cagtctgtcctgacgcagccgccctcagtgtctggggccccagggcagagggtcaccatctcctgcactgggagcagctccaacatcggggcaggttatgatgtacactggtaccagcaggttccaggaacagcccccaaactcctcatctttggtagcaccgatcggccctcaggggtccctgatcgcttctctggctccaagtctggcaacacggcctccctgaccatctctgggctccaggctgaggatgaggctgattattattgccagtcctatgaccgcagcctgagtcatgtcttcggaactgggaccaaggtcaccgtcctaggc

SEQ ID NO: 97 - DNA, 375 bp, Artificial Sequence
  clone 26 heavy chain
gaggtgcagctggtggagtctgggggaggcctggccaagcctggggggtccctgagactctcctgtgcagcctctggattcaccttcactagttataggatgcattgggtccgccaggctccagggaaggggctggagtgggtctcatcaattactggtggtggtaattatatagagtacgcagactcagtgaagggccggttcaccatctccagagacaacgccaagaactcactggatctgcaaatgaacagcctgagagccgaggacacggctatttattactgtgcgagagatatgtatggtttggggagttattattcgcctaactacttcgacccctggggccagggaaccctggtcaccgtctcctca

SEQ ID NO: 98 - DNA, 333 bp, Artificial Sequence
  clone 26 light chain
cagtctgtgctgacgcagccgccctcagtgtctggggccccagggcagagggtcaccatctcctgcactgggagcagctccaacatcggggcaggttatgatgtacactggtatcagcagcttccgggagcagcccccagactcctcatgtttggtaacagcaatcggccctcgggggtacctgaccgcttctctggctccaagtctggcacctccgcctccctggccatcactggtctccaggctgaggatgaggctgattattactgccagtcctatgaccgcagcctgagtcatgtcttcggaactgggaccaaggtcaccgtcctaggc

SEQ ID NO: 99 - DNA, 375 bp, Artificial Sequence; identical to SEQ ID NO: 38
  clone 27 heavy chain

SEQ ID NO: 100 - DNA, 333 bp, Artificial Sequence
  clone 27 light chain
tcctatgagctgactcagccaccctcagtgtctggggccccagggcagagggtcaccatctcctgcactgggagcagctccaacatcggggcaggttatgatgtacactggtatcagcagcttccgggagcagcccccagactcctcatgtttggtaacagcaatcggccctcgggggtacctgaccgcttctctggctccaagtctggcacctccgcctccctggccatcactggtctccaggctgaggatgaggctgattattactgccagtcctatgacagtagcctgagttatgtcttcggaactgggaccaaggtcaccgtcctaggc

SEQ ID NO: 101 - DNA, 333 bp, Artificial Sequence
  clone 27m light chain
cagtctgtgctgacgcagccgccctcagtgtctggggccccagggcagagggtcaccatctcctgcactgggagcagctccaacatcggggcaggttatgatgtacactggtatcagcagcttccgggagcagcccccagactcctcatgtttggtaacagcaatcggccctcgggggtacctgaccgcttctctggctccaagtctggcacctccgcctccctggccatcactggtctccaggctgaggatgaggctgattattactgccagtcctatgacagtagcctgagttatgtcttcggaactgggaccaaggtcaccgtcctaggc

SEQ ID NO: 102 - DNA, 375 bp, Artificial Sequence; identical to SEQ ID NO: 78
  clone 28 heavy chain

SEQ ID NO: 103 - DNA, 333 bp, Artificial Sequence
  clone 28 light chain
cagtctgtgctgacgcagccgccctcagtgtctggggccccagggcagagggtcaccatctcctgcactgggagcagctccaacatcggggcaggttatgatgtacactggtatcagcagcttccgggagcagcccccagactcctcatgtttggtaacagcaatcggccctcgggggtacctgaccgcttctctggctccaagtctggcacctccgcctccctggccatcactggtctccaggctgaggatgaggctgattattactgccagtcctatgacagtagcctgagtcatgtcttcggaactgggaccaaggtcaccgtcctaggc

SEQ ID NO: 104 - DNA, 375 bp, Artificial Sequence
  clone 29 heavy chain
gaggtgcagctggtggagtctgggggaggcttggtccagcctggggggtccctgagactctcctgtgcagcctctggattcaccttcactagttataggatgcattgggtccgccaggctccagggaaggggctggagtgggtctcatcaattactggtggtggtaattatatagagtacgcagactcagtgaagggccggttcaccatctccagagacaacgccaagaactcactggatctgcaaatgaacagcctgagagccgaggacacggctatttattactgtgcgagagatatgtatggtttggggagttattattcgcctaactacttcgacccctggggccagggaaccctggtcaccgtctcctca

SEQ ID NO: 105 - DNA, 333 bp, Artificial Sequence; identical to SEQ ID NO: 101
  clone 29 light chain

SEQ ID NO: 106 - DNA, 357 bp, Artificial Sequence
  clone 30 heavy chain
gaggtgcagctggtggagtctgggggaggcttggtacagcctggggggtccctgagactctcctgtgcagcatctgaattcacctttagcatgaactgggtccgacaggctccagggaaggggctggagtgggtctcatcagttcgaggtggcggtactgaaacatactatgcagactccgtgaagggccggttcaccgtctccagagacaattccaagaacaccctgcatctgcaaatgaacagcctgagagccgaggacacggccctttattactgtgcgggcggccctatagtggaacccaacattgactactttaactcctggggccagggaaccctggtcaccgtctcctca

SEQ ID NO: 107 - DNA, 379 bp, Artificial Sequence
  clone 30 light chain
tgctgtgggtgagtggtacctgtgggcagtctgtgctgacgcagccgccctcagtgtctggggccccagggcagagggtcaccatctcctgcactgggagcagctccaacatcggggcaggttatgatgtacactggtaccagcagcttccaggaagagcccccaaactcctcatctttgctaacaccaatcggccctcaggggtccctgaccgattctctggctccaagtctggcgcctctgcctccctggccatcactggcctccaggctgacgatgaggctgattattactgccagtcttatgacagcaaacatcatgtggtctttggcacagggaccaagctgaccgtcctaggccaacctaaggccaatcccac

SEQ ID NO: 108 - DNA, 333 bp, Artificial Sequence
  clone 30m light chain
cagtctgtgctgacgcagccgccctcagtgtctggggccccagggcagagggtcaccatctcctgcactgggagcagctccaacatcggggcaggttatgatgtacactggtaccagcagcttccaggaagagcccccaaactcctcatctttgctaacaccaatcggccctcaggggtccctgaccgattctctggctccaagtctggcgcctctgcctccctggccatcactggcctccaggctgacgatgaggctgattattactgccagtcttatgacagcaaacatcatgtggtctttggcacagggaccaagctgaccgtcctaggc

SEQ ID NO: 109 - DNA, 384 bp, Artificial Sequence
  clone 31 heavy chain
gaggtgcagctggtggagtctgggggaggcttggtccagcctggggggtccctgagactctcctgtgcagcctctgggttctcctttaggagttattggatgagctggctccgccaggccccagggaaggggctgcagtgggtggccaacataaagccagacggaagtgtggaaagttatgtggactccgtggagggccgattcaccatctccagagacaacgccaagaattcactgtttctgcaaatgaacagcctgtcagccgaggacatggctgtttattactgtgcgaggacggacgacggcagcagctggttcgtgtccaccagtagtttctacggtatggacgtctggggccaagggaccacggtcaccgtctcctca

SEQ ID NO: 110 - DNA, 333 bp, Artificial Sequence
  clone 31 light chain
cagtctgtgttgacgcagccgccctcagtgtctggggccccagggcagagggtcaccatctcctgcactgggagcagctccaacatcggggcaggttatgatgtacactggtaccagcaggttccaggaacagcccccaaactcctcatctttggtagcaccaatcggccctcaggggtccctgaccgattctctggctccaagtctggcgcctcagcctccctggccatcattgggctccagactgaggatgaggctgattattactgccagtcctatgaccgcagcctgagtcatgtcttcggaactggcacccagctgaccgacctcggc

SEQ ID NO: 111 - DNA, 378 bp, Artificial Sequence
  clone 32 heavy chain
gaggtgcagctggtggagtctgggggaggcctggtcaagcctggggggtccctgagactctcctgtgcagcctctggattcaccttcagtagctataccatgcactgggtccgccaggctccagggaaggggctggagtgggtctcgtccataactggtggcagtagttatgtcgactactcagcctcagtgaagggccgattcaccatctcccgagacaattccaagaacacgctgtatctgcaaatgagcagcctgagaactgacgacacggccgtatatttctgtgcgaaagaccaacgtggagacagctatgactcggttatgaactggtacttcgatctctggggccgtggcaccacggtcaccgtctcctca

SEQ ID NO: 112 - DNA, 333 bp, Artificial Sequence
  clone 32 light chain
cagtctgtgttgacgcagccgccctcagtgtctggggccccagggcagagggtcaccatttcctgcactgggaacagctccaacctcggggcaggttatgatgtacactggtaccagcaggttccaggaacagcccccaaactcctcatctttggtagcaccaatcggccctcaggggtccctgaccgattctctggctccaagtctggcgcctcagcctccctgacaatctctgggctccaggctgaggacgaggctgattattactgctgttcgtatgcagctagtagctctagggtcttcggaactggcaccaaggtgaccgtcctcggc

SEQ ID NO: 113 - DNA, 426 bp, Artificial Sequence
  clone 33m heavy chain
tgctgtgggtgagtggtacctgtggggaggtgcagctggtggagtctgggggcggcctggttaagcctggggggtccctgagactctcgtgtgcagcctctgggttcaggctcagtagctatggcatgaactgggtccgccaggctccagggaaggggctggagtgggtctcatccatttctgctagtagtagttttataaactatgcagactcagtgagggaccgattcaccatctccagagacaacgccaagaactcactgtatctgcaaatgaacagcctgagagccgaggacacggctgtgtattactgtgcgagagatgattatggttcggggagttattccaactggttcgacccctggggccagggaaccctggtcaccgtctcctcagcctccaccaagggcccatcggtcttccccc

SEQ ID NO: 114 - DNA, 366 bp, Artificial Sequence
  clone 34 heavy chain
gaggtgcagctggtggagtctgggggaggcctggtcaagcctggggggtccctgagactctcctgtgcagcctctggattcaccttcagtcattatggtattagttgggtgcgccaggcccctggacaaggccttgagtggatggcctggatcagcgcttacaatggtaacacagactccatacagaaggtccagggcagagtcaccatgaccacagacacatccacgaacacagcctacttggaattgaggagcctgagatctgacgacacggccgtgtattactgtgtaagagatgtcccggccacaggaggagctgccacggctgactactggggccaaggaaccctggtcaccgtctcctca

SEQ ID NO: 115 - DNA, 423 bp, Artificial Sequence
  clone 34m heavy chain
tgctgtgggtgagtggtacctgtgggcaggttcagctggtgcagtctggagctgaggtgaagaagcctggggcctcagtgaaggtctcctgcaaggcttctggattcaccttcagtcattatggtattagttgggtgcgccaggcccctggacaaggccttgagtggatggcctggatcagcgcttacaatggtaacacagactccatacagaaggtccagggcagagtcaccatgaccacagacacatccacgaacacagcctacttggaattgaggagcctgagatctgacgacacggccgtgtattactgtgtaagagatgtcccggccacaggaggagctgccacggctgactactggggccaaggaaccctggtcaccgtctcctcagcctccaccaagggcccatcggtcttccccc

SEQ ID NO: 116 - DNA, 336 bp, Artificial Sequence
  clone 34 light chain
cagtctgtgttgacgcagccgccctcagtgtctggggccccagggcagagggtcaccatctcctgcactgggagcagctccaacatcggggcaggttatgatgtacactggtaccagcaggttccaggaacagcccccaaactcctcatctttggtagcaccaatcggccctcaggggtccctgaccgattctctggctccaagtctggcgcctcagcctccctggccatcactgggctccaggctgaggacgaggctgattattactgcagctcatatacaagcagcagcactttcgtggtatttggtggaggaacccagctgatcattttaggc

SEQ ID NO: 117 - DNA, 363 bp, Artificial Sequence
  clone 35 heavy chain
caggtgcagctggtggagtctgggggaggcgtggtccagcctgggaggtccctgagactctcctgtgcagcctctggattcaccttcaatagctatgctatgcactgggtccgccaggctccaggcaacgggctggactgggtggcaggtatatcatatgatggaggcaataaatattacgcagactccgtgaaggaccgattcaccatctcccgagacaattccaagaacacgctgtatctgcaaatgagcagcctgagaactgacgacacggctgtatattactgtgcgaaagatgccggatgggaggcttggtggtacttcgacctctggggccgtggcaccctggtcactgtctcctca

SEQ ID NO: 118 - DNA, 333 bp, Artificial Sequence
  clone 35 light chain
cagtctgccctgactcagcctgcctccgtgtctgggtctcctggacagtcgatcaccatctcctgcactggaaccagcagtgacgttggtggttataactatgtctcctggttccaacagtacccaggcaaagcccccaaactcatgatttatgatgtcagtaagcggccctcaggggtttctaatcgcttctctggctccaagtctggcaacacggcctccctgaccatctctgagctccaggctgaggatgaggctgattattactgcagctcatatacaagcagcagcactggcgtattcggcggagggaccaagctgaccgtcctaggc

SEQ ID NO: 119 - DNA, 369 bp, Artificial Sequence
  clone 36 heavy chain
gaggtgcagctggtggagtctgggggaggcttggtacagcctggggggtccctgagactctcctgtgcagcctctggaacctttaccaaatatgccatgacctgggtccgccaggctctagggaaggggctggagtgggtctcaactattggcagtggtagtgacactcactacgcagactccgtgaagggccgcttcaccatctccagagacaattccaggaacactctgtctctacaaatgaacagcctgagagccgaggacacggccgtatattactgtgccaaatatctgggaataacagtgggtgatacgggttttcggacctttgactactggggccagggaaccacggtcaccgtctcttca

SEQ ID NO: 120 - DNA, 379 bp, Artificial Sequence
  clone 36 light chain
tgctgtgggtgagtggtacctgtgggcagtctgccctgactcagcctcgctcagtgtccgggtctcctggacagtcagtcaccatctcctgcactggaaccagcagtgatgttgggagttataaccttgtctcctggtaccaacagcacccaggcaaagcccccaaactcatgatttatgatgtcagtaagcggccctcaggggtttctaatcgcttctctggctccaagtctggcaacacggcctccctgaccatctccgggctccaggctgaggatgaggctgattattactgcagctcatatgcaggcagcgggaatgtggtatttggtggaggaacccagctgatcattttaggccaacctaaggccaatcccac

SEQ ID NO: 121 - DNA, 354 bp, Artificial Sequence
  clone 37 heavy chain
caggtgcagctggtggagtctgggggaggcgtggtccagcctgggaggtccctgagactctcctgtgcagcctctggattcaccttcagtatctatgctatgcactgggtccgccaggctccaggcaaggggctagagtgggtggcagttgtttcatatgatggaagcgagaaatactacgcagactccgtgcagggccgattcaccatctccagagacaagtccaagaacaccctgtatctgcaaatgaacagcctgacagctgaggacacggctgtctattactgtgcgagagagccctgggtggggacaattggctactggggccagggaaccctggtcaccgtctcctca

SEQ ID NO: 122 - DNA, 336 bp, Artificial Sequence
  clone 37 light chain
cagtctgccctgactcagcctccctccgcgtccgggtctcctggacagtcagtcaccatctcctgcactggaaccagcagtgacgttggtggttataactatgtctcctggtaccgacagcacccaggcaaagcccccaaactcctgatttatgaggtcaataaccggccctcaggggtccctagtcgcttctctggctccaagtctggcaacacggcctccctgaccatctctgggctccaggctgaggatgaggctgattattactgcagctcatatacaagcagcagcactttcgtggtacttggtggaggaacccagctgatcattttaggc

SEQ ID NO: 123 - DNA, 372 bp, Artificial Sequence
  clone 38 heavy chain
caaatgcagctggtgcagtctgggcctgaggtgaagaagcctgggtcctcggtgaaggtctcctgcaaggcttcgggttacacctttaccgactacggtctcagctgggtccggcaggcccccggccacggccttgagtggatgggatggatcaccgcttacaatggcgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcaccgtctcctca

SEQ ID NO: 124 - DNA, 429 bp, Artificial Sequence
  clone 38m heavy chain
tgctgtgggtgagtggtacctgtgggcaggttcagctggtgcagtctggagctgaggtgaagaagcctggggcctcagtgaaggtctcctgcaaggcttcgggttacacctttaccgactacggtctcagctgggtccggcaggcccccggccacggccttgagtggatgggatggatcaccgcttacaatggcgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcaccgtctcctcagcctccaccaagggcccatcggtcttccccc

SEQ ID NO: 125 - DNA, 379 bp, Artificial Sequence
  clone 38 light chain
tgctgtgggtgagtggtacctgtgggcagtctgtgctgactcagccaccctcagcgtctgggacccccgggcagagggtcaccatctcttgttctggaagcagctccaacgtcggaagtaatactgtcaactggtaccagcagctcccaggaacggcccccaaactcctcatctacgataagaatgagcggccctcaggggtcccggaccgattctctgcctccaagtctggcacctcagcctccctggccatcagtggcctccagtctgaagatgaggctgattattactgtgcagcatgggatgacagcctgaatggttgggtgttcggtggagggaccgagctgaccgtcctaggccaacctaaggccaatcccac

SEQ ID NO: 126 - DNA, 333 bp, Artificial Sequence
  clone 38m light chain
cagtctgtgctgactcagccaccctcagcgtctggggcccccgggcagcgggtcatcatctcttgttctggaagcagctccaacgtcggaagtaatactgtcaactggtaccagcagctcccaggaacggcccccaaactcctcatctacgataagaatgagcggccctcaggggtcccggaccgattctctgcctccaagtctggcacctcagcctccctggccatcagtggcctccagtctgaagatgaggctgattattactgtgcagcatgggatgacagcctgaatggttgggtgttcggtggagggaccaagctgaccgtcctaggc

SEQ ID NO: 127 - DNA, 354 bp, Artificial Sequence
  clone 39 heavy chain
gaggtgcagctggtggagtctgggggaggcctggtcaagcctggggggtccctgagactctcctgtgcagcctctgggttcaggctcagtagctatggcatgaactgggtccgccaggctccagggaaggggctggagtgggtctcatccatttctgctagtagtagttttataaactatgcagactcagtgaggggccgattcaccatctccagagacaagtccaagaacaccctgtatctgcaaatgaacagcctgacagctgaggacacggctgtctattactgtgcgagagagccctgggtggggacaattgactactggggccagggaaccctggtcaccgtctcctca

SEQ ID NO: 128 - DNA, 336 bp, Artificial Sequence
  clone 39 light chain
cagtctgccctgactcagcctgcctccgtgtctgggtctcctggacagtcgatcaccatctcctgcactggaaccagcagtgatgttgggagttataaccttgtctcctggtaccaacagcacccaggcaaagcccccaaactcctgatttatgaggacactaagcggccctcagggatcccagaccgattctctggctccagctcaggaaacacagcttccttgaccatcactggggctcaggcggaagatgaggctgagtattactgtagttcccgggacagcagtggtaaccatctggtgttcggcggagggaccaagctgaccgtcctaggc

SEQ ID NO: 129 - DNA, 372 bp, Artificial Sequence
  clone 40 heavy chain
caggtgcagctggtgcagtctggagctgaggtgaagaagcctggggcctcagtgaaggtctcctgcaaggcttcgggttacacctttaccgactacggtctcagctgggtccggcaggcccccggccacggccttgagtggatgggatggatcaccgcttacaatggcgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcaccgtctcctca

SEQ ID NO: 130 - DNA, 333 bp, Artificial Sequence
  clone 40 light chain
tcctatgagctgactcagccaccctcagcgtctggggcccccgggcagcgggtcatcatctcttgttctggaagcagctccaacgtcggaagtaatactgtcaactggtaccagcagcttccaggaagagcccccaaactcctcatctttgctaacaccaatcggccctcaggggtccctgaccgattctctggctccaagtctggcacctcagcctccctggccatcagtggcctccagtctgaagatgaggctgattattactgtgcagcatgggatgacagcctgaatggttgggtgttcggtggagggaccaagctgaccgtcctaggc

SEQ ID NO: 131 - DNA, 333 bp, Artificial Sequence
  clone 40m light chain
cagtctgtgctgactcagccaccctcagcgtctggggcccccgggcagcgggtcatcatctcttgttctggaagcagctccaacgtcggaagtaatactgtcaactggtaccagcagcttccaggaagagcccccaaactcctcatctttgctaacaccaatcggccctcaggggtccctgaccgattctctggctccaagtctggcacctcagcctccctggccatcagtggcctccagtctgaagatgaggctgattattactgtgcagcatgggatgacagcctgaatggttgggtgttcggtggagggaccaagctgaccgtcctaggc

SEQ ID NO: 132 - DNA, 372 bp, Artificial Sequence
  clone 41 heavy chain
caggtgcagctggtgcagtctggggctgaggtgaagaagccgggggcctcagtgaaggtctcctgcaaggcttcgggttacacctttaccgactacggtctcagctgggtccggcaggcccccggccacggccttgagtggatgggatggatcaccgcttacaatggcgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcaccgtctcctca

SEQ ID NO: 133 - DNA, 333 bp, Artificial Sequence
  clone 41 light chain
tcctatgagctgactcagccactctcagcgtctgggacccccgggcagagggtcaccatctcttgttctggaagcagctccaacatcggaagtggtactgtaaactggtaccagcagctctcaggaacggcccccaaactcctcatgcatagtgataatcagcgcccctcaggggtccctgaccgattctctggctccaagtctggcacctcagcctccctggccatcagtggcctccagtctgaagatgaggctgattattactgtgcagcatgggatgacagcctgaatggttgggtgttcggtggagggaccaagctgaccgtcctaggc

SEQ ID NO: 134 - DNA, 333 bp, Artificial Sequence
  clone 41m light chain
cagtctgtgctgactcagccactctcagcgtctgggacccccgggcagagggtcaccatctcttgttctggaagcagctccaacatcggaagtggtactgtaaactggtaccagcagctctcaggaacggcccccaaactcctcatgcatagtgataatcagcgcccctcaggggtccctgaccgattctctggctccaagtctggcacctcagcctccctggccatcagtggcctccagtctgaagatgaggctgattattactgtgcagcatgggatgacagcctgaatggttgggtgttcggtggagggaccaagctgaccgtcctaggc

SEQ ID NO: 135 - DNA, 372 bp, Artificial Sequence
  clone 42 heavy chain
caggtgcagctggtgcagtctggaagtgaggtgaagaagcctggggcctcagtgaaggtctcctgcaaggcttcgggttacacctttaccgactacggtctcagctgggtccggcaggcccccggccacggccttgagtggatgggatggatcaccgcttacaatggtgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcaccgtctcctca

SEQ ID NO: 136 - DNA, 333 bp, Artificial Sequence
  clone 42 light chain
cagcctgtgctgactcagccaccctcagtgtctgggacccccgggcagagggtcaccatctcttgttctggaagcagctccaacatcggaagtgatactgtaaactggtaccagcagctctcaggaacggcccccaaactcctcatgcatagtgataatcagcgcccctcaggggtccctgaccgattctctggctccaagtctggcacctcagcctccctggccatcagtggcctccagtctgaagatgaggctgattattactgtgcagcatgggatgacagcctgaatggttgggtgttcggtggagggaccaagctgaccgtcctaggc

SEQ ID NO: 137 - DNA, 333 bp, Artificial Sequence
  clone 42m light chain
cagtctgtgctgactcagccaccctcagtgtctgggacccccgggcagagggtcaccatctcttgttctggaagcagctccaacatcggaagtgatactgtaaactggtaccagcagctctcaggaacggcccccaaactcctcatgcatagtgataatcagcgcccctcaggggtccctgaccgattctctggctccaagtctggcacctcagcctccctggccatcagtggcctccagtctgaagatgaggctgattattactgtgcagcatgggatgacagcctgaatggttgggtgttcggtggagggaccaagctgaccgtcctaggc

SEQ ID NO: 138 - DNA, 372 bp, Artificial Sequence
  clone 43 heavy chain
caggttcagctggtgcagtctggaagtgaggtgaagaagccgggggcctcagtgaaggtctcctgcaaggcttcgggttacacctttaccgactacggtctcagctgggtccggcaggcccccggccacggccttgagtggatgggatggatcaccgcttacaatggcgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcaccgtctcctca

SEQ ID NO: 139 - DNA, 333 bp, Artificial Sequence
  clone 43 light chain
cagcctgtgctgactcagccaccctcagcgtctgggacccccgggcagagggtcaccatctcttgttctggaagcagctccaacatcggaagtgatactgtaaactggtaccagcagctctcaggaacggcccccaaactcctcatgcatagtgataatcagcgcccctcaggggtccctgaccgattctctggctccaagtctggcacctcagcctccctggccatcagtggcctccagtctgaagatgaggctgattattactgtgcagcatgggatgacagcctgaatggttgggtgttcggtggagggaccaagctgaccgtcctaggc

SEQ ID NO: 140 - DNA, 333 bp, Artificial Sequence; identical to SEQ ID NO: 139
  clone 43 light chain

SEQ ID NO: 141 - DNA, 333 bp, Artificial Sequence
  clone 43m light chain
cagtctgtgctgactcagccaccctcagcgtctgggacccccgggcagagggtcaccatctcttgttctggaagcagctccaacatcggaagtgatactgtaaactggtaccagcagctctcaggaacggcccccaaactcctcatgcatagtgataatcagcgcccctcaggggtccctgaccgattctctggctccaagtctggcacctcagcctccctggccatcagtggcctccagtctgaagatgaggctgattattactgtgcagcatgggatgacagcctgaatggttgggtgttcggtggagggaccaagctgaccgtcctaggc

SEQ ID NO: 142 - DNA, 372 bp, Artificial Sequence
  clone 44 heavy chain
caggtgcagctggtgcagtctggagctgaggtgaagaagccgggggcctcagtgaaagtctcctgcaaggcttcgggttacacctttaccgactacggtctcagctgggtccggcaggcccccggccacggccttgagtggatgggatggatcaccgcttacaatggcgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcaccgtctcctca

SEQ ID NO: 143 - DNA, 333 bp, Artificial Sequence
  clone 44 light chain
cagtctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccacgtctggcacctcagcctccctggccatcagtggactccagtctgaagatgaggctgattattactgtgcagcatgggatgacagcctgaatggttgggtgttcggtggagggaccaagctgaccgtcctaggc

SEQ ID NO: 144 - DNA, 372 bp, Artificial Sequence
  clone 45 heavy chain
caggtgcagctggtgcagtctggaagtgaggtgaagaagcctggggcctcagtgaaggtctcctgcaaggcttcgggttacacctttaccgactacggtctcagctgggtccggcaggcccccggccacggccttgagtggatgggatggatcaccgcttacaatggcgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcaccgtctcctca

SEQ ID NO: 145 - DNA, 333 bp, Artificial Sequence
  clone 45 light chain
tcctatgagctgactcagccactctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggtggagggaccaagctgaccgtcctaggc

SEQ ID NO: 146 - DNA, 333 bp, Artificial Sequence
  clone 45m light chain
cagtctgtgctgactcagccactctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttgggtgttcggtggagggaccaagctgaccgtcctaggc

SEQ ID NO: 147 - DNA, 372 bp, Artificial Sequence
  clone 46 heavy chain
caggtgcagctggtgcagtctgggcctgaggtgaagaagcctgggtcctcggtgaaggtctcctgcaaggcttcgggttacacctttaccgactacggtctcagctgggtccggcaggcccccggccacggccttgagtggatgggatggatcaccgcttacaatggcgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcaccgtctcctca

SEQ ID NO: 148 - DNA, 333 bp, Artificial Sequence
  clone 46 light chain
tcctatgagctgactcagccaccctcagcgtctggggcccccgggcagcgggtcatcatctcttgttctggaagcagctccaacgtcggaagtaatactgtcaactggtaccagcagctcccaggaacggcccccaaactcctcatctacgataagaatgagcggccctcaggggtcccggaccgattctctgcctccaagtctggcacctcagcctccctggccatcagtggcctccagtctgaagatgaggctgattattactgtgcagcatgggatgacagcctgaatggttgggtgttcggtggagggaccaagctgaccgtcctaggc

SEQ ID NO: 149 - DNA, 333 bp, Artificial Sequence; identical to SEQ ID NO: 126
  clone 46m light chain

SEQ ID NO: 150 - DNA, 381 bp, Artificial Sequence
  clone 47 heavy chain
caggttcagctggtgcagtctggagctgaggtgaagaagcctggggcctcagtgaaggtctcctgcaaggcttctggttacaccggttacacctttaccgactacggtctcagctgggtccggcaggcccccggccacggccttgagtggatgggatggatcaccgcttacaatggcgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcaccgtctcctca

SEQ ID NO: 151 - DNA, 333 bp, Artificial Sequence
  clone 47 light chain
tcctatgagctgatacagccaccctcagcgtctggggcccccgggcagcgggtcatcatctcttgttctggaagcagctccaacgtcggaagtaatactgtcaactggtaccagcagctcccaggaacggcccccaaactcctcatctacgataagaatgagcggccctcaggggtcccggaccgattctctgcctccaagtctggcacctcagcctccctggccatcagtggcctccagtctgaagatgaggctgattattactgtgcagcatgggatgacagcctgaatggttgggtgttcggtggagggaccgagctgaccgtcctaggc

SEQ ID NO: 152 - DNA, 333 bp, Artificial Sequence; identical to SEQ ID NO: 126
  clone 47m light chain

SEQ ID NO: 153 - DNA, 372 bp, Artificial Sequence
  clone 48 heavy chain
caggtgcagctggtgcagtctggagctgaggtgaagaagcctgggtcctcggtgaaggtctcctgcaaggcttcgggttacacctttaccgactacggtctcagctgggtccggcaggcccccggccacggccttgagtggatgggatggatcaccgcttacaatggcgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcaccgtctcctca

SEQ ID NO: 154 - DNA, 333 bp, Artificial Sequence
  clone 48 light chain
cagtctgtgctgacgcagccgccctcagtgtctggggccccagggcagagggtcaccatctcttgttctggaagcagctccaacgtcggaagtaatactgtcaactggtaccagcagctcccaggaacggcccccaaactcctcatctacgataagaatgagcggccctcaggggtcccggaccgattctctgcctccaagtctggcacctcatcctccctggccatcggtgggctccggtctgaagatgaggctgattattactgtgggacatgggatgacaacctgaatggttgggtgttcggcggagggaccaagctgaccgtcctaggc

SEQ ID NO: 155 - DNA, 372 bp, Artificial Sequence; identical to SEQ ID NO: 129
  clone 49 heavy chain

SEQ ID NO: 156 - DNA, 333 bp, Artificial Sequence
  clone 49 light chain
tcctatgagctgactcagccaccctcagcgtctggggcccccgggcagcgggtcatcatctcttgttctggaagcagctccaacgtcggaagtaatactgtcaactggtaccagcagctcccaggaacggcccccaaactcctcatctacgataagaatgagcggccctcaggggtcccggaccgattctctgcctccaagtctggcacctcatcctccctggccatcggtgggctccggtctgaagatgaggctgattattactgtgcgacatgggatgacaacctgaatggttgggtgttcggcggagggaccaagctgaccgtcctaggc

SEQ ID NO: 157 - DNA, 333 bp, Artificial Sequence
  clone 49m light chain
cagtctgtgctgactcagccaccctcagcgtctggggcccccgggcagcgggtcatcatctcttgttctggaagcagctccaacgtcggaagtaatactgtcaactggtaccagcagctcccaggaacggcccccaaactcctcatctacgataagaatgagcggccctcaggggtcccggaccgattctctgcctccaagtctggcacctcatcctccctggccatcggtgggctccggtctgaagatgaggctgattattactgtgcgacatgggatgacaacctgaatggttgggtgttcggcggagggaccaagctgaccgtcctaggc

SEQ ID NO: 158 - DNA, 372 bp, Artificial Sequence; identical to SEQ ID NO: 129
  clone 50 heavy chain

SEQ ID NO: 159 - DNA, 333 bp, Artificial Sequence
  clone 50 light chain
tcctatgagctgactcagccaccctcagcgtctggggcccccgggcagcgggtcatcatctcttgttctggaagcagctccaacgtcggaagtaatactgtcaactggtaccagcagctcccaggaacggcccccaaactcctcatctacgataagaatgagcggccctcaggggtcccggaccgattctctgcctccaagtctggcacctcatcctccctggccatcggtgggctccggtctgaagatgaggctgattattactgtgggacatgggatgacaacctgaatggttgggtgttcggcggagggaccaagctgaccgtcctaggc

SEQ ID NO: 160 - DNA, 333 bp, Artificial Sequence
  clone 50m light chain
cagtctgtgctgactcagccaccctcagcgtctggggcccccgggcagcgggtcatcatctcttgttctggaagcagctccaacgtcggaagtaatactgtcaactggtaccagcagctcccaggaacggcccccaaactcctcatctacgataagaatgagcggccctcaggggtcccggaccgattctctgcctccaagtctggcacctcatcctccctggccatcggtgggctccggtctgaagatgaggctgattattactgtgggacatgggatgacaacctgaatggttgggtgttcggcggagggaccaagctgaccgtcctaggc

SEQ ID NO: 161 - DNA, 372 bp, Artificial Sequence
  clone 51 heavy chain
gaggtgcagctggtggagtctgggactgaggtgaagaagcctggggcctcagtgaaggtctcctgcaaggcttcgggttacacctttaccgactacggtctcagctgggtccggcaggcccccggccacggccttgagtggatgggatggatcaccgcttacaatggcgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcactgtctcctca

SEQ ID NO: 162 - DNA, 429 bp, Artificial Sequence
  clone 51m heavy chain
tgctgtgggtgagtggtacctgtggggaggtgcagctggtgcagtctggggctgaggtgaagaagcctggggcctcagtgaaggtctcctgcaaggcttcgggttacacctttaccgactacggtctcagctgggtccggcaggcccccggccacggccttgagtggatgggatggatcaccgcttacaatggcgacacaaactatgcacagaagttccaggacagactgtccgtgaccactgacatatccacgagcacagcctacatggaattacggagcctgaaatctgacgacacggccgtttattattgtgcgagacacgtcttttgcagtggtgatgggtgttactccggccttgggtcctggggccagggaaccctggtcactgtctcctcagcctccaccaagggcccatcggtcttccccc

SEQ ID NO: 163 - DNA, 333 bp, Artificial Sequence; identical to SEQ ID NO: 160
  clone 51 light chain

SEQ ID NO: 164 - DNA, 384 bp, Artificial Sequence
  clone 52 heavy chain
caggtgcagctggtgcagtctgggggaggcttggtacagcctggagggtccctgagaatctcctgtacagcctctggattcaccttcaggaattatgaaatgaattgggtccgccaggctccagggaaggggctggagtgggttgcatacattagtagtagtggtagttccagatactacgcagactctgtgaagggccgattcaccatctccagagacaacgccaagaactcactgtttctgcaaatgaacagcctgagagccgaggacatggctgtttattactgtgcgaggacggacgacggcagcagctggttcgtgtccaccagtagtttctacggtatggacgtctggggccaagggaccacggtcaccgtctcctca

SEQ ID NO: 165 - DNA, 386 bp, Artificial Sequence
  clone 52m heavy chain
gagaggtgcagctggtggagtctgggggaggcttggtacagcctggagggtccctgagaatctcctgtacagcctctggattcaccttcaggaattatgaaatgaattgggtccgccaggctccagggaaggggctggagtgggttgcatacattagtagtagtggtagttccagatactacgcagactctgtgaagggccgattcaccatctccagagacaacgccaagaactcactgtttctgcaaatgaacagcctgagagccgaggacatggctgtttattactgtgcgaggacggacgacggcagcagctggttcgtgtccaccagtagtttctacggtatggacgtctggggccaagggaccacggtcaccgtctcctca

SEQ ID NO: 166 - DNA, 330 bp, Artificial Sequence
  clone 52 light chain
gaaattgtgctgacccagtctccaggcatccagtctttgtctccaggggaaacagccaccctctcctgcagggccagtgagagtattagcagcagttacttcgcctggtaccagcagaaacctggccaggctcccaggctcctcatctatggtgcatccagcagggcctctggcatcccagacaggttcagtggcagtgggtctgggacagacttcactctcaccatcagcagactggagcctgaagattttgcagtgtattactgtcagcagtatggtagctcacctcccatcaccttcggccaagggacacgactggagattaaacga

SEQ ID NO: 167 - DNA, 384 bp, Artificial Sequence
  clone 53 heavy chain
caggtgcagctggtgcagtctgggggaggcttggtacagcctggagggtccctgagaatctcctgtacagcctctggattcaccttcaggaattatgaaatgaattgggtccgccaggctccagggaaggggctggagtgggttgcatacattagtagtagtggtagttccagatactacgcagactctgtgaagggccgattcaccatctccagagacaacgccaagaacacgctgtatctgcaaatgagcagcctgagatctgacgacacggccgtgtattactgtgcgaggacggacgacggcagcagctggttcgtgtccaccagtagtttctacggtatggacgtctggggccaagggaccacggtcaccgtctcctca

SEQ ID NO: 168 - DNA, 386 bp, Artificial Sequence
  clone 53m heavy chain
gagaggtgcagctggtggagtctgggggaggcttggtacagcctggagggtccctgagaatctcctgtacagcctctggattcaccttcaggaattatgaaatgaattgggtccgccaggctccagggaaggggctggagtgggttgcatacattagtagtagtggtagttccagatactacgcagactctgtgaagggccgattcaccatctccagagacaacgccaagaacacgctgtatctgcaaatgagcagcctgagatctgacgacacggccgtgtattactgtgcgaggacggacgacggcagcagctggttcgtgtccaccagtagtttctacggtatggacgtctggggccaagggaccacggtcaccgtctcctca

SEQ ID NO: 169 - DNA, 330 bp, Artificial Sequence
  clone 53 light chain
gatgttgtgatgactcagtctccaggcaccctgtctttgtctccaggggaaagagccaccctctcctgcagggccagtcagagtgttagtagcagctacttagcctggtaccagcagaaacctggccaggctcccaggctcctcatctacggtgtgtccagcagggccactggcatcccagacaggttcagtggcagtgggtctgggacagacttcactctcaccatcagcagactggagcctgaagattttgcagtgtattactgtcagcagtatggtagctcacctcccatcaccttcggccaggggaccaagctggagatcaaacga

SEQ ID NO: 170 - DNA, 330 bp, Artificial Sequence
  clone 53m light chain
gagattgtgctgacccagtctccaggcaccctgtctttgtctccaggggaaagagccaccctctcctgcagggccagtcagagtgttagtagcagctacttagcctggtaccagcagaaacctggccaggctcccaggctcctcatctacggtgtgtccagcagggccactggcatcccagacaggttcagtggcagtgggtctgggacagacttcactctcaccatcagcagactggagcctgaagattttgcagtgtattactgtcagcagtatggtagctcacctcccatcaccttcggccaggggaccaagctggagatcaaacgt

SEQ ID NO: 171 - DNA, 360 bp, Artificial Sequence
  clone 54 heavy chain
gaggtgcagctggtggagtctgggggaggcttggtacagcctggggggtccctgagactctcctgtgcagcctctgaattcacctttagcatgaactgggtccgacaggctccagggaaggggctggagtgggtctcatcagttcgaggtggcggtactgaaacatactatgcagactccgtgaagggccggttcaccgtctccagagacaattccaagaacacactgtatctgcagatgaacagcctgagagtcgaggacacggccgtgtattactgtacggtgtgtgttgttatgtttcggggatggtgcaactggttcgacccctggggccagggaaccctggtcaccgtctcctca

SEQ ID NO: 172 - DNA, 324 bp, Artificial Sequence
  clone 54 light chain
gacatccagttgacccagtctccatcttccgtgtctgcatctgtaggagagagagtcaccatcactcgtcgggcgagtcagaatattgacaggtggttagcttggtatcagcagaaaccagggaaagcccctgacctcctgatctttgctgcatccagtttgcagagtggggtcccatcaaggttcagcggcagtggatctgggacagatttcactctcaccatcagcagcctgcagcctgaagattttgcaacttactattgtcaacagggtaaaactttccctcccacgttcggccaggggaccaagctggagatcaaacga

SEQ ID NO: 173 - DNA, 360 bp, Artificial Sequence
  clone 55 heavy chain
gaggtgcagctgttggagtctgggggaggcttggtacagcctggggggtccctgagactctcctgtgcagcctctgaattcacctttagcatgaactgggtccgacaggctccagggaaggggctggagtgggtctcatcagttcgaggtggcggtactgaaacatactatgcagactccgtgaagggccggttcaccgtctccagagacaattccaagaacacactgtatctgcagatgaacagcctgagagtcgaggacacggccgtgtattactgtacggtgtgtgttgttatgtttcggggatggtgcaactggttcgacccctggggccagggaaccctggtcaccgtctcctca

SEQ ID NO: 174 - DNA, 324 bp, Artificial Sequence
  clone 55m light chain
gatgttgtgatgactcagtctccatcgtccctgtctgcatccgttggggacagagtcaccatcacttgccgggcaagtcagagcctcagtagttatttaaattggtatcagcagaaaccagggaaagcccctaaactgctcatctatgctacaaccaacttgcaaagtggggtcccttcaaggttcagtggcagtggatctgggacacatttcactctcaccatcgggagtctgcaacctgaagattttgcaacttattactgtcaacagagtttccagaccccgctcactttcggcggagggaccaaggtggagatcaaacga

SEQ ID NO: 175 - DNA, 324 bp, Artificial Sequence
  clone 55m light chain
gacatccagatgacccagtctccatcgtccctgtctgcatccgttggggacagagtcaccatcacttgccgggcaagtcagagcctcagtagttatttaaattggtatcagcagaaaccagggaaagcccctaaactgctcatctatgctacaaccaacttgcaaagtggggtcccttcaaggttcagtggcagtggatctgggacacatttcactctcaccatcgggagtctgcaacctgaagattttgcaacttattactgtcaacagagtttccagaccccgctcactttcggcggagggaccaagctggagatcaaacgt

SEQ ID NO: 176 - DNA, 354 bp, Artificial Sequence
  clone 56 heavy chain
caggtgcagctggtggagtctgggggaggcgtggtccagcctgggaggtccctgagactctcctgtgcagcctctggattcaccttcattagctatgtcatgcactgggtccgccaggctccaggcaaggggctggagtgggtggcagttgtttcatatgatggaagcgagaaatactacgcagactccgtgaagggccgattcaccatctccagagacaattccaagaacacgctgtatctgcaaatgaacagcctgacagctgaggacacggctgtctattactgtgcgagagagccctgggtggggacaattgactactggggccagggaaccctggtcaccgtctcctca

SEQ ID NO: 177 - DNA, 324 bp, Artificial Sequence
  clone 56 light chain
gacatccagatgacccagtctccagccaccctgtctgcatctgtaggagacagagtcaccatcacttgccgggcaagtcagagcattaacaacaatttaaattggtatcaacagaaaccagggcaagcccctaagctcctgatctatgctgcatccactttacaaggtggggtcccatcaaggttcagcggcagtggatctgggacagaattcactctcacaatcagcagcctgcagcctgaagattttgcaacttattactgtcaacagcttaatggttaccctctcactttcggcggagggaccaaggtggagatcaaacga

SEQ ID NO: 178 - DNA, 363 bp, Artificial Sequence
  clone 57 heavy chain
gaggtgcagctgttggagtctgggggaggcttggtacagcctggggggtccctgagactctcctgtgcagcctctggattcacctttagcgactatgccatgagttgggtccgccaggctccagggaaggggctggagtgggtctcaagtattactgctaatggacttatgacatactacacagactccgtgaagggccgattcaccatctcccgagacaattccaagaacacgctgtatctgcaaatgagcagcctgagaactgacgacacggctgtatattactgtgcgaaagatgccggatgggaggcttggtggtacttcgacctctggggccgtggcaccctggtcaccgtctcctca

SEQ ID NO: 179 - DNA, 339 bp, Artificial Sequence
  clone 57 light chain
gacatccagatgacccagtctccagactccctggctgtgtctctgggcgagagggccaccatcaactgcaagtccagccagagtgttttatacagctccaacaataagaactacttagcttggtaccagcagaaaccaggacagcctcctaagatgctcatttactgggcatctatccgggaaaccggggtccctgaccgattcagtggcagcgggtctgagacagatttcactctcaccatcagcagcctgcaggctgaagatgtggcagtttattactgtcagcaatattatagtactctcactttcggcggagggaccaaggtggagatcaaacga

SEQ ID NO: 180 - DNA, 384 bp, Artificial Sequence
  clone 58 heavy chain
caggtgcagctggtgcagtctggggctgaggtgaagaagcctggggcctcagtgaaggtttcctgcaaggcatctggatacaccttcaccagctactatatgcgctggctgcgacaggcccctggacaagggcttgagtggatgggaataatcgaccctagtggtggtagcacaacctatgcacagaagttccagggcagagtcagcataaccagggacacgtccacgagcacagtctacatggagctgagcagcctgagatctgaggacacggccgtgtattactgtgcgagagggaggggcgtagtaccagctggcaacccctcaacggggggggtcggcatggacgtctggggccaagggaccacggtcaccgtctcctca

SEQ ID NO: 181 - DNA, 324 bp, Artificial Sequence
  clone 58 light chain
gagattgtgctgacccagtctccagccaccgtgtctctgtctcccggggaaagaaccatgttgtcttgcagggccagtcagaatgttagtaactacttagggtggtatcagcagagacgtggccagcctcccagactcctcatttccgatgcgtccaacagggcctctggcgtcccagccaggttcagtggaagtgggtctgggacagacttcactcttactatcaccagtcttcagcctgaagattttgcagtttatttctgtcagcaccggagcagctggcccgtcactttcggcggagggaccaaggtggagatcaaacga

SEQ ID NO: 182 - DNA, 366 bp, Artificial Sequence
  clone 59 heavy chain
caggtgcagctggtgcagtctggaagtgaggtgaagaagcctggggcctcagtgaaggtctcctgcaaggcctctggatacacctttggtcattatggtattagttgggtgcgccaggcccctggacaaggccttgagtggatggcctggatcagcgcttacaatggtaacacagactccatacagaaggtccagggcagagtcaccatgaccacagacacatccacgaacacagcctacttggaattgaggagcctgagatctgacgacacggccgtgtattactgtgtaagagatgtcccggccacaggaggagctgccacggctgactactggggccagggaaccctggtcaccgtctcctca

SEQ ID NO: 183 - DNA, 339 bp, Artificial Sequence
  clone 59 light chain
gatgttgtgatgactcagtctccactctccctgcccgtcacccttggccagccggcctccatctcctgcaggtctagtcaaagcctcgtcttcagtgatggaaacacctacttgagttggtttcaacagaggccaggccaatctccaaggcgcctaatttataaggtttctaaccgggactctggggtcccagacagattcagcgccagtgggtcaggcactgatttcacactgaaaatcagcagggtggaggctgacgatgttggggtttattactgcatgcaagttacacactggcctcggacttttggccaggggaccaagctggagatcaaacga

SEQ ID NO: 184 - DNA, 366 bp, Artificial Sequence
  clone 60 heavy chain
caggtgcagctggtgcagtctgggcctgaggtgaagaagcctgggtcctcagtgaaggtctcctgcaaggcctctggatacacctttggtcattatggtattagttgggtgcgccaggcccctggacaaggccttgagtggatggcctggatcagcgcttacaatggtaacacagactccatacagaaggtccagggcagagtcaccatgaccacagacacatccacgaacacagcctacttggaattgaggagcctgagatctgacgacacggccgtgtattactgtgtaagagatgtcccggccacaggaggagctgccacggctgactactggggccagggaaccacggtcaccgtctcctca

SEQ ID NO: 185 - DNA, 339 bp, Artificial Sequence
  clone 60 light chain
gatgttgtgatgactcagtctccactctccctgcccgtcacccttggccagccggcctccatctcctgcaggtctagtcaaagcctcgtcttcagtgatggaaacacctacttgagttggtttcaacagaggccaggccaatctccaaggcgcctaatttataaggtttctaaccgggactctggggtcccagacagattcagcgccagtgggtcaggcactgatttcacactgaaaatcagcagggtggaggctgacgatgttggggtttattactgcatgcaagttacacactggcctcggacttttggccaagggacacgactggagattaaacga

SEQ ID NO: 186 - DNA, 366 bp, Artificial Sequence
  clone 61 heavy chain
caggtgcagctggtgcagtctggaagtgaggtgaagaagcctggggcctcagtgaaggtctcctgcaaggcctctggatacacctttggtcattatggtattagttgggtgcgccaggcccctggacaaggccttgagtggatggcctggatcagcgcttacaatggtaacacagactccatacagaaggtccagggcagagtcaccatgaccacagacacatccacgaacacagcctacttggaattgaggagcctgagatctgacgacacggccgtgtattactgtgtaagagatgtcccggccacaggaggagctgccacggctgactactggggccagggaaccacggtcaccgtctcctca

SEQ ID NO: 187 - DNA, 339 bp, Artificial Sequence; identical to SEQ ID NO: 183
  clone 61 light chain

SEQ ID NO: 188 - DNA, 366 bp, Artificial Sequence
  clone 62 heavy chain
caggtacagctgcagcagtctggaagtgaggtgaagaagcctggggcctcagtgaaggtctcctgcaaggcctctggatacacctttggtcattatggtattagttgggtgcgccaggcccctggacaaggccttgagtggatggcctggatcagcgcttacaatggtaacacagactccatacagaaggtccagggcagagtcaccatgaccacagacacatccacgaacacagcctacttggaattgaggagcctgagatctgacgacacggccgtgtattactgtgtaagagatgtcccggccacaggaggagctgccacggctgactactggggccagggaaccctggtcaccgtctcctca

SEQ ID NO: 189 - DNA, 423 bp, Artificial Sequence
  clone 62m heavy chain
tgctgtgggtgagtggtacctgtgggcaggtacagctggtgcagtctggagctgaggtgaagaagcctggggcctcagtgaaggtctcctgcaaggcctctggatacacctttggtcattatggtattagttgggtgcgccaggcccctggacaaggccttgagtggatggcctggatcagcgcttacaatggtaacacagactccatacagaaggtccagggcagagtcaccatgaccacagacacatccacgaacacagcctacttggaattgaggagcctgagatctgacgacacggccgtgtattactgtgtaagagatgtcccggccacaggaggagctgccacggctgactactggggccagggaaccctggtcaccgtctcctcagcctccaccaagggcccatcggtcttccccc

SEQ ID NO: 190 - DNA, 339 bp, Artificial Sequence
  clone 62 light chain
gatgttgtgatgacacagtctccactctccttacctgtcccccttggacagccggcctccatctcctgcaggtctagtcaaagcctcgtacacagtgatggaaacacctacttgagttggtttcagcagaggccaggccaatctccaaggcgcctaatttataaggtttctgaccgggactctggggtcccagacagattcagcggcagtgggtcaggcactgatttcacactgaaaatcagcagggtggaggctgaggatgttggggtttattactgcatgcaagttacacactggcctcggacttttggccaagggacacgactggagattaaacga

SEQ ID NO: 191 - DNA, 366 bp, Artificial Sequence; identical to SEQ ID NO: 182
  clone 63 heavy chain

SEQ ID NO: 192 - DNA, 339 bp, Artificial Sequence
  clone 63 light chain
gatgttgtgatgactcagtctccactctccctgcccgtcacccttggacaaccggcctccatctcctgcaagtctagtcgaagcctcgtacacagtgatggaaacacctacttgagttggtttcaacagaggccaggccaatctccaaggcgcctaatttataaggtttctaaccgggactctggggtcccagacagattcagcgccagtgggtcaggcactgatttcacactgaaaatcagcagggtggaggctgacgatgttggggtttattactgcatgcaagttacacactggcctcggacttttggccaggggaccaagctggagatcaaacga

SEQ ID NO: 193 - protein, 123 aa, Artificial Sequence
  clone 1 heavy chain
EVQLVESGGGLVKPGGSLRLSCAASGFTFSSYTMHWVRQAPGKGLEWVSSITGGSSYVDYSASVKGRFTISRDNAQSSLYLQMNSLRAEDTAVYYCARDDYGSGSYSNWFDPWGQGTLVTVSS

SEQ ID NO: 194 - protein, 111 aa, Artificial Sequence
  clone 1 light chain
QSVLTQPPSVSGAPGQRVTISCTGSSSNIGAGYDVHWYQQVPGTAPKLLIFGSTNRPSGVPDRFSGSKSGTSASLAITGLQADDEADYYCQSYDRSLSHVFGTGTKVTVLG

SEQ ID NO: 195 - protein, 123 aa, Artificial Sequence
  clone 2 heavy chain
EVQLVESGGGVVQSGRSLRLSCAGSGFTFSSYTMHWVRQAPGKGLEWVSSITGGSSYVDYSASVKGRFTISRDNAQSSLYLQMNSLRAEDTAVYYCARDDYGSGSYSNWFDPWGQGTLVTVSS

SEQ ID NO: 196 - protein, 111 aa, Artificial Sequence; identical to SEQ ID NO: 33
  clone 2 light chain

SEQ ID NO: 197 - protein, 123 aa, Artificial Sequence; identical to SEQ ID NO: 29
  clone 3 heavy chain

SEQ ID NO: 198 - protein, 111 aa, Artificial Sequence
  clone 3 light chain
QSVLTQPPSVSGAPGQRVTISCTGSSSNIGAGYDVHWYQQVPGTAPKLLIFGSTNRPSGVPDRFSGSKSGASASLAITGLQADDEADYYCQPYDRSLSHVFGTGTKVTVLR

SEQ ID NO: 199 - protein, 123 aa, Artificial Sequence; identical to SEQ ID NO: 29
  clone 4 heavy chain

SEQ ID NO: 200 - protein, 111 aa, Artificial Sequence
  clone 4 light chain
QSVLTQPPSVSGAPGQRVTISCTGSSSNIGAGYDVHWYQQVPGTAPKLLIFGSTNRPSGVPDRFSGSKSGASASLAITGLQADDEADYYCQSYDRSLSHVFGTGTKVTVLG

SEQ ID NO: 201 - protein, 123 aa, Artificial Sequence; identical to SEQ ID NO: 29
  clone 5 heavy chain

SEQ ID NO: 202 - protein, 111 aa, Artificial Sequence
  clone 5 light chain
QSVLTQPPSVSGAPGQRVTISCTGSSSNIGAGYDVHWYQQLPGAAPRLLMFGNSNRPSGVPDRFSGSKSGTSASLAITGLQAEDEADYYCQSYDRSLSHVFGTGTKVTVLG

SEQ ID NO: 203 - protein, 111 aa, Artificial Sequence
  clone 6 light chain
SYELTQPPSASGTPGQRVTISCTGSSSNIGAGYDVHWYQQLPGAAPRLLMFGNSNRPSGVPDRFSGSKSGTSASLAITGLQADDEADYYCQSYDRSLSHVFGTGTKVTVLG

SEQ ID NO: 204 - protein, 123 aa, Artificial Sequence
  clone 7 heavy chain
QVQLVQSGGGLIQPGGSLRLSCAGSGFTFSSYTMHWVRQAPGKGLEWVSSITGGSSYVDYSASVKGRFTISRDNAQSSLYLQMNSLRAEDTAVYYCARDDYGSGSYSNWFDPWGQGTLVTVSS

SEQ ID NO: 205 - protein, 111 aa, Artificial Sequence
  clone 7 light chain
QSVLTQPPSVSGAPGQRVTISCTGSSSNIGAGYDVHWYQQLPGTAPKLLIFANTNRPSGVPDRFSGSKSGASASLAITGLQADDEADYYCQSYDRSLSHVFGTGTKVTVLG

SEQ ID NO: 206 - protein, 123 aa, Artificial Sequence
  clone 8m heavy chain
EVQLVESGGGVVQSGRSLRLSCAASGFTFSSYTMHWVRQAPGKGLEWVSSITGGSSYVDYSASVKGRFTISRDNAQSSLYLQMNSLRAEDTAVYYCARDDYGSGSYSNWFDPWGQGTLVTVSS

SEQ ID NO: 207 - protein, 123 aa, Artificial Sequence; identical to SEQ ID NO: 29
  clone 10 heavy chain

SEQ ID NO: 208 - protein, 111 aa, Artificial Sequence
  clone 10 light chain
QSVLTQPPSVSGAPGQRVTISCTGNSSNLGAGYDVHWYQQLPGTAPKLLIYANTNRPSGVPDRFSGSKSGASASLAITGLQTEDEADYYCQSYDRSLSHVFGTGTKVTVLG

SEQ ID NO: 209 - protein, 111 aa, Artificial Sequence
  clone 11 light chain
QTVVTQEPSVSGAPGQRVTISCTGSSSNIGAGYDVHWYQQLPGRAPKLLIFANTNRPSGVPDRFSGSKSGASASLAITGLQADDEADYYCQSYDRSLSHVFGTGTKVTVLG

SEQ ID NO: 210 - protein, 111 aa, Artificial Sequence
  clone 12 light chain
QPVLTQPPSVSGAPGQRVTISCTGSSSNIGAGYDVHWYQQVPGTAPKLLIFGSTNRPSGVPDRFSGSKSGASASLAITGLQTEDEADYYCQSYDRSLSHVFGTGTKVTVLG

SEQ ID NO: 211 - protein, 123 aa, Artificial Sequence
  clone 14 heavy chain
QVTLKESGGGLVKPGGSLRLSCAGSGFTFSSYTMHWVRQAPGKGLEWVSSITGGSSYVDYSASVKGRFTISRDNAKNSLFLQMNSLRAEDTAVYYCARDDYGSGSYSNWFDPWGQGTLVTVSS

SEQ ID NO: 212 - protein, 111 aa, Artificial Sequence; identical to SEQ ID NO: 24
  clone 14 light chain

SEQ ID NO: 213 - protein, 125 aa, Artificial Sequence
  clone 15 heavy chain
EVQLVESGGGVVQPGRSLRLACAASGFTFNSYAMHWVRQAPGKGLEWVSSITGGSSYVDYSASVKGRFTISRDNAQSSLYLQMNSLRAEDTAVYYCARDMYGLGSYYSPNYFDPWGQGTLVTVSS

SEQ ID NO: 214 - protein, 111 aa, Artificial Sequence
  clone 15 light chain
SYELTQPPSVSGAPGQRVTISCTGSSSNIGAGYDVHWYQQLPGRAPKLLIFANTNRPSGVPDRFSGSKSGTSASLAITGLQAEDEADYYCQSYDSSLSYVFGTGTKVTVLG

SEQ ID NO: 215 - protein, 111 aa, Artificial Sequence
  clone 15m light chain
QSVLTQPPSVSGAPGQRVTISCTGSSSNIGAGYDVHWYQQLPGRAPKLLIFANTNRPSGVPDRFSGSKSGTSASLAITGLQAEDEADYYCQSYDSSLSYVFGTGTKVTVLG

SEQ ID NO: 216 - protein, 111 aa, Artificial Sequence
  clone 16m light chain
QSVLTQPLSASGTPGQRVTISCTGSSSNIGAGYDVHWYQQLPGRAPKLLIFANTNRPSGVPDRFSGSKSGTSASLAITGLQAEDEADYYCQSYDRSLSHVFGTGTKVTVLG

SEQ ID NO: 217 - protein, 123 aa, Artificial Sequence
  clone 17 heavy chain
EVQLVESGGGLVQPGGSLRLACAASGFTFSSYTMHWVRQAPGKGLEWVSSITGGSSYVDYSASVKGRFTISRDNAQSSLYLQMNSLRAEDTAVYYCARDDYGSGSYSNWFDPWGQGTLVTVSS

SEQ ID NO: 218 - protein, 111 aa, Artificial Sequence
  clone 17 light chain
SYELTQPPSVSGAPGQRVTISCTGSSSNIGAGYDVHWYQQLPGRAPKLLIFANTNRPSGVPDRFSGSKSGASASLAITGLQAEDEADYYCQSYDRSLSHVFGTGTKVTVLG

SEQ ID NO: 219 - protein, 111 aa, Artificial Sequence
  clone 17m light chain
QSVLTQPPSVSGAPGQRVTISCTGSSSNIGAGYDVHWYQQLPGRAPKLLIFANTNRPSGVPDRFSGSKSGASASLAITGLQAEDEADYYCQSYDRSLSHVFGTGTKVTVLG

SEQ ID NO: 220 - protein, 125 aa, Artificial Sequence
  clone 18 heavy chain
QVTLKESGGGLVKPGGSLRLSCAASGFTFTSYRMHWVRQAPGKGLEWVSSITGGGNYIEYADSVKGRFTISRDNAKNSLDLQMNSLRAEDTAIYYCARDMYGLGSYYSPNYFDPWGQGTLVTVSS

SEQ ID NO: 221 - protein, 111 aa, Artificial Sequence
  clone 18 light chain
SYELTQPLSVSGAPGQRVTISCTGSSSNIGAGYDVHWYQQVPGTAPKLLIFGSTNRPSGVPDRFSGSKSGASASLAITGLQADDEADYYCQSYDRSLSHVFGTGTKVTVLG

SEQ ID NO: 222 - protein, 111 aa, Artificial Sequence
  clone 18m light chain
QSVLTQPLSVSGAPGQRVTISCTGSSSNIGAGYDVHWYQQVPGTAPKLLIFGSTNRPSGVPDRFSGSKSGASASLAITGLQADDEADYYCQSYDRSLSHVFGTGTKVTVLG

SEQ ID NO: 223 - protein, 125 aa, Artificial Sequence
  clone 19 heavy chain
EVQLVESGGGLVKPGGSLRLSCAASGFTFTSYRMHWVRQAPGKRLEWVSSITGGGNYIEYADSVKGRFTISRDNAKNSLDLQMNSLRAEDTAIYYCARDMYGLGSYYSPNYFDPWGQGTLVTVSS

SEQ ID NO: 224 - protein, 111 aa, Artificial Sequence; identical to SEQ ID NO: 33
  clone 19 light chain

SEQ ID NO: 225 - protein, 125 aa, Artificial Sequence
  clone 20 heavy chain
EVQLVESGGGLVKPGGSLRLSCAASGFTFTSYRMHWVRQAPGKGLEWVSSITGGGNYIEYADSVKGRFTISRDNAKNSLDLQMNSLRAEDTAIYYCARDMYGLGSYYSPNYFDPWGQGTLVTVSS

SEQ ID NO: 226 - protein, 111 aa, Artificial Sequence; identical to SEQ ID NO: 210
  clone 20 light chain

SEQ ID NO: 227 - protein, 111 aa, Artificial Sequence; identical to SEQ ID NO: 33
  clone 20m light chain

SEQ ID NO: 228 - protein, 125 aa, Artificial Sequence; identical to SEQ ID NO: 220
  clone 21 heavy chain

SEQ ID NO: 229 - protein, 111 aa, Artificial Sequence
  clone 21 light chain
QSVLTQPPSVSGAPGQRVTISCTGSSSNIGAGYDVHWYQQLPGTAPKLLIFGSTNRPSGVPDRFSGSKSGASASLAITGLQTEDEADYYCQSYDRSLSHVFGTGTKVTVLG

SEQ ID NO: 230 - protein, 125 aa, Artificial Sequence; identical to SEQ ID NO: 225
  clone 22 heavy chain

SEQ ID NO: 231 - protein, 111 aa, Artificial Sequence; identical to SEQ ID NO: 24
  clone 22 light chain

SEQ ID NO: 232 - protein, 125 aa, Artificial Sequence
  clone 23 heavy chain
QVQLVQSGGGLVKPGGSLRLSCAASGFTFTSYRMHWVRQAPGKGLEWVSSITGGGNYIEYADSVKGRFTISRDNAKNSLDLQMNSLRAEDTAIYYCARDMYGLGSYYSPNYFDPWGQGTTVTVSS

SEQ ID NO: 233 - protein, 111 aa, Artificial Sequence
  clone 23 light chain
QSVLTQPPSVSGAPGQRVTISCTGSSSNIGAGYDVHWYQQLPGRAPKLLIFANTNRPSGVPDRFSGSKSGASASLAISGLQAEDEAHYYCQSYDRSLNVVFGGGTELTVLG

SEQ ID NO: 234 - protein, 125 aa, Artificial Sequence; identical to SEQ ID NO: 225
  clone 24 heavy chain

SEQ ID NO: 235 - protein, 112 aa, Artificial Sequence
  clone 24 light chain
QSVLTQPPSVSGAPGQRVTISCTGSSSNIGAGYDVHWYQQVPGTAPKLLIFGSTNRPSGVPDRFSGSKSGASASLAITGLQTEDEADYYCQSYDRSLSAWVFGGGTKLTVLG

SEQ ID NO: 236 - protein, 125 aa, Artificial Sequence; identical to SEQ ID NO: 225
  clone 25 heavy chain

SEQ ID NO: 237 - protein, 111 aa, Artificial Sequence
  clone 25 light chain
QSVLTQPPSVSGAPGQRVTISCTGSSSNIGAGYDVHWYQQVPGTAPKLLIFGSTDRPSGVPDRFSGSKSGNTASLTISGLQAEDEADYYCQSYDRSLSHVFGTGTKVTVLG

SEQ ID NO: 238 - protein, 125 aa, Artificial Sequence
  clone 26 heavy chain
EVQLVESGGGLAKPGGSLRLSCAASGFTFTSYRMHWVRQAPGKGLEWVSSITGGGNYIEYADSVKGRFTISRDNAKNSLDLQMNSLRAEDTAIYYCARDMYGLGSYYSPNYFDPWGQGTLVTVSS

SEQ ID NO: 239 - protein, 111 aa, Artificial Sequence; identical to SEQ ID NO: 202
  clone 26 light chain

SEQ ID NO: 240 - protein, 125 aa, Artificial Sequence; identical to SEQ ID NO: 23
  clone 27 heavy chain

SEQ ID NO: 241 - protein, 111 aa, Artificial Sequence
  clone 27 light chain
SYELTQPPSVSGAPGQRVTISCTGSSSNIGAGYDVHWYQQLPGAAPRLLMFGNSNRPSGVPDRFSGSKSGTSASLAITGLQAEDEADYYCQSYDSSLSYVFGTGTKVTVLG

SEQ ID NO: 242 - protein, 111 aa, Artificial Sequence
  clone 27m light chain
QSVLTQPPSVSGAPGQRVTISCTGSSSNIGAGYDVHWYQQLPGAAPRLLMFGNSNRPSGVPDRFSGSKSGTSASLAITGLQAEDEADYYCQSYDSSLSYVFGTGTKVTVLG

SEQ ID NO: 243 - protein, 125 aa, Artificial Sequence; identical to SEQ ID NO: 225
  clone 28 heavy chain

SEQ ID NO: 244 - protein, 111 aa, Artificial Sequence
  clone 28 light chain
QSVLTQPPSVSGAPGQRVTISCTGSSSNIGAGYDVHWYQQLPGAAPRLLMFGNSNRPSGVPDRFSGSKSGTSASLAITGLQAEDEADYYCQSYDSSLSHVFGTGTKVTVLG

SEQ ID NO: 245 - protein, 125 aa, Artificial Sequence
  clone 29 heavy chain
EVQLVESGGGLVQPGGSLRLSCAASGFTFTSYRMHWVRQAPGKGLEWVSSITGGGNYIEYADSVKGRFTISRDNAKNSLDLQMNSLRAEDTAIYYCARDMYGLGSYYSPNYFDPWGQGTLVTVSS

SEQ ID NO: 246 - protein, 111 aa, Artificial Sequence; identical to SEQ ID NO: 242
  clone 29 light chain

SEQ ID NO: 247 - protein, 119 aa, Artificial Sequence
  clone 30 heavy chain
EVQLVESGGGLVQPGGSLRLSCAASEFTFSMNWVRQAPGKGLEWVSSVRGGGTETYYADSVKGRFTVSRDNSKNTLHLQMNSLRAEDTALYYCAGGPIVEPNIDYFNSWGQGTLVTVSS

SEQ ID NO: 248 - protein, 111 aa, Artificial Sequence
  clone 30 light chain
QSVLTQPPSVSGAPGQRVTISCTGSSSNIGAGYDVHWYQQLPGRAPKLLIFANTNRPSGVPDRFSGSKSGASASLAITGLQADDEADYYCQSYDSKHHVVFGTGTKLTVLG

SEQ ID NO: 249 - protein, 111 aa, Artificial Sequence; identical to SEQ ID NO: 248
  clone 30m light chain

SEQ ID NO: 250 - protein, 128 aa, Artificial Sequence
  clone 31 heavy chain
EVQLVESGGGLVQPGGSLRLSCAASGFSFRSYWMSWLRQAPGKGLQWVANIKPDGSVESYVDSVEGRFTISRDNAKNSLFLQMNSLSAEDMAVYYCARTDDGSSWFVSTSSFYGMDVWGQGTTVTVSS

SEQ ID NO: 251 - protein, 111 aa, Artificial Sequence
  clone 31 light chain
QSVLTQPPSVSGAPGQRVTISCTGSSSNIGAGYDVHWYQQVPGTAPKLLIFGSTNRPSGVPDRFSGSKSGASASLAIIGLQTEDEADYYCQSYDRSLSHVFGTGTQLTDLG

SEQ ID NO: 252 - protein, 126 aa, Artificial Sequence
  clone 32 heavy chain
EVQLVESGGGLVKPGGSLRLSCAASGFTFSSYTMHWVRQAPGKGLEWVSSITGGSSYVDYSASVKGRFTISRDNSKNTLYLQMSSLRTDDTAVYFCAKDQRGDSYDSVMNWYFDLWGRGTTVTVSS

SEQ ID NO: 253 - protein, 111 aa, Artificial Sequence
  clone 32 light chain
QSVLTQPPSVSGAPGQRVTISCTGNSSNLGAGYDVHWYQQVPGTAPKLLIFGSTNRPSGVPDRFSGSKSGASASLTISGLQAEDEADYYCCSYAASSSRVFGTGTKVTVLG

SEQ ID NO: 254 - protein, 123 aa, Artificial Sequence
  clone 33m heavy chain
EVQLVESGGGLVKPGGSLRLSCAASGFRLSSYGMNWVRQAPGKGLEWVSSISASSSFINYADSVRDRFTISRDNAKNSLYLQMNSLRAEDTAVYYCARDDYGSGSYSNWFDPWGQGTLVTVSS

SEQ ID NO: 255 - protein, 122 aa, Artificial Sequence
  clone 34 heavy chain
EVQLVESGGGLVKPGGSLRLSCAASGFTFSHYGISWVRQAPGQGLEWMAWISAYNGNTDSIQKVQGRVTMTTDTSTNTAYLELRSLRSDDTAVYYCVRDVPATGGAATADYWGQGTLVTVSS

SEQ ID NO: 256 - protein, 122 aa, Artificial Sequence
  clone 34m heavy chain
QVQLVQSGAEVKKPGASVKVSCKASGFTFSHYGISWVRQAPGQGLEWMAWISAYNGNTDSIQKVQGRVTMTTDTSTNTAYLELRSLRSDDTAVYYCVRDVPATGGAATADYWGQGTLVTVSS

SEQ ID NO: 257 - protein, 112 aa, Artificial Sequence
  clone 34 light chain
QSVLTQPPSVSGAPGQRVTISCTGSSSNIGAGYDVHWYQQVPGTAPKLLIFGSTNRPSGVPDRFSGSKSGASASLAITGLQAEDEADYYCSSYTSSSTFVVFGGGTQLIILG

SEQ ID NO: 258 - protein, 121 aa, Artificial Sequence
  clone 35 heavy chain
QVQLVESGGGVVQPGRSLRLSCAASGFTFNSYAMHWVRQAPGNGLDWVAGISYDGGNKYYADSVKDRFTISRDNSKNTLYLQMSSLRTDDTAVYYCAKDAGWEAWWYFDLWGRGTLVTVSS

SEQ ID NO: 259 - protein, 111 aa, Artificial Sequence
  clone 35 light chain
QSALTQPASVSGSPGQSITISCTGTSSDVGGYNYVSWFQQYPGKAPKLMIYDVSKRPSGVSNRFSGSKSGNTASLTISELQAEDEADYYCSSYTSSSTGVFGGGTKLTVLG

SEQ ID NO: 260 - protein, 123 aa, Artificial Sequence
  clone 36 heavy chain
EVQLVESGGGLVQPGGSLRLSCAASGTFTKYAMTWVRQALGKGLEWVSTIGSGSDTHYADSVKGRFTISRDNSRNTLSLQMNSLRAEDTAVYYCAKYLGITVGDTGFRTFDYWGQGTTVTVSS

SEQ ID NO: 261 - protein, 111 aa, Artificial Sequence
  clone 36 light chain
QSALTQPRSVSGSPGQSVTISCTGTSSDVGSYNLVSWYQQHPGKAPKLMIYDVSKRPSGVSNRFSGSKSGNTASLTISGLQAEDEADYYCSSYAGSGNVVFGGGTQLIILG

SEQ ID NO: 262 - protein, 118 aa, Artificial Sequence
  clone 37 heavy chain
QVQLVESGGGVVQPGRSLRLSCAASGFTFSIYAMHWVRQAPGKGLEWVAVVSYDGSEKYYADSVQGRFTISRDKSKNTLYLQMNSLTAEDTAVYYCAREPWVGTIGYWGQGTLVTVSS

SEQ ID NO: 263 - protein, 112 aa, Artificial Sequence
  clone 37 light chain
QSALTQPPSASGSPGQSVTISCTGTSSDVGGYNYVSWYRQHPGKAPKLLIYEVNNRPSGVPSRFSGSKSGNTASLTISGLQAEDEADYYCSSYTSSSTFVVLGGGTQLIILG

SEQ ID NO: 264 - protein, 124 aa, Artificial Sequence
  clone 38 heavy chain
QMQLVQSGPEVKKPGSSVKVSCKASGYTFTDYGLSWVRQAPGHGLEWMGWITAYNGDTNYAQKFQDRLSVTTDISTSTAYMELRSLKSDDTAVYYCARHVFCSGDGCYSGLGSWGQGTLVTVSS

SEQ ID NO: 265 - protein, 124 aa, Artificial Sequence
  clone 38m heavy chain
QVQLVQSGAEVKKPGASVKVSCKASGYTFTDYGLSWVRQAPGHGLEWMGWITAYNGDTNYAQKFQDRLSVTTDISTSTAYMELRSLKSDDTAVYYCARHVFCSGDGCYSGLGSWGQGTLVTVSS

SEQ ID NO: 266 - protein, 111 aa, Artificial Sequence
  clone 38 light chain
QSVLTQPPSASGTPGQRVTISCSGSSSNVGSNTVNWYQQLPGTAPKLLIYDKNERPSGVPDRFSASKSGTSASLAISGLQSEDEADYYCAAWDDSLNGWVFGGGTELTVLG

SEQ ID NO: 267 - protein, 111 aa, Artificial Sequence
  clone 38m light chain
QSVLTQPPSASGAPGQRVIISCSGSSSNVGSNTVNWYQQLPGTAPKLLIYDKNERPSGVPDRFSASKSGTSASLAISGLQSEDEADYYCAAWDDSLNGWVFGGGTKLTVLG

SEQ ID NO: 268 - protein, 118 aa, Artificial Sequence
  clone 39 heavy chain
EVQLVESGGGLVKPGGSLRLSCAASGFRLSSYGMNWVRQAPGKGLEWVSSISASSSFINYADSVRGRFTISRDKSKNTLYLQMNSLTAEDTAVYYCAREPWVGTIDYWGQGTLVTVSS

SEQ ID NO: 269 - protein, 112 aa, Artificial Sequence
  clone 39 light chain
QSALTQPASVSGSPGQSITISCTGTSSDVGSYNLVSWYQQHPGKAPKLLIYEDTKRPSGIPDRFSGSSSGNTASLTITGAQAEDEAEYYCSSRDSSGNHLVFGGGTKLTVLG

SEQ ID NO: 270 - protein, 124 aa, Artificial Sequence; identical to SEQ ID NO: 265
  clone 40 heavy chain

SEQ ID NO: 271 - protein, 111 aa, Artificial Sequence
  clone 40 light chain
SYELTQPPSASGAPGQRVIISCSGSSSNVGSNTVNWYQQLPGRAPKLLIFANTNRPSGVPDRFSGSKSGTSASLAISGLQSEDEADYYCAAWDDSLNGWVFGGGTKLTVLG

SEQ ID NO: 272 - protein, 111 aa, Artificial Sequence
  clone 40m light chain
QSVLTQPPSASGAPGQRVIISCSGSSSNVGSNTVNWYQQLPGRAPKLLIFANTNRPSGVPDRFSGSKSGTSASLAISGLQSEDEADYYCAAWDDSLNGWVFGGGTKLTVLG

SEQ ID NO: 273 - protein, 124 aa, Artificial Sequence; identical to SEQ ID NO: 265
  clone 41 heavy chain

SEQ ID NO: 274 - protein, 111 aa, Artificial Sequence
  clone 41 light chain
SYELTQPLSASGTPGQRVTISCSGSSSNIGSGTVNWYQQLSGTAPKLLMHSDNQRPSGVPDRFSGSKSGTSASLAISGLQSEDEADYYCAAWDDSLNGWVFGGGTKLTVLG

SEQ ID NO: 275 - protein, 111 aa, Artificial Sequence
  clone 41m light chain
QSVLTQPLSASGTPGQRVTISCSGSSSNIGSGTVNWYQQLSGTAPKLLMHSDNQRPSGVPDRFSGSKSGTSASLAISGLQSEDEADYYCAAWDDSLNGWVFGGGTKLTVLG

SEQ ID NO: 276 - protein, 124 aa, Artificial Sequence
  clone 42 heavy chain
QVQLVQSGSEVKKPGASVKVSCKASGYTFTDYGLSWVRQAPGHGLEWMGWITAYNGDTNYAQKFQDRLSVTTDISTSTAYMELRSLKSDDTAVYYCARHVFCSGDGCYSGLGSWGQGTLVTVSS

SEQ ID NO: 277 - protein, 111 aa, Artificial Sequence
  clone 42 light chain
QPVLTQPPSVSGTPGQRVTISCSGSSSNIGSDTVNWYQQLSGTAPKLLMHSDNQRPSGVPDRFSGSKSGTSASLAISGLQSEDEADYYCAAWDDSLNGWVFGGGTKLTVLG

SEQ ID NO: 278 - protein, 111 aa, Artificial Sequence
  clone 42m light chain
QSVLTQPPSVSGTPGQRVTISCSGSSSNIGSDTVNWYQQLSGTAPKLLMHSDNQRPSGVPDRFSGSKSGTSASLAISGLQSEDEADYYCAAWDDSLNGWVFGGGTKLTVLG

SEQ ID NO: 279 - protein, 124 aa, Artificial Sequence; identical to SEQ ID NO: 276
  clone 43 heavy chain

SEQ ID NO: 280 - protein, 124 aa, Artificial Sequence; identical to SEQ ID NO: 276
  clone 43m heavy chain

SEQ ID NO: 281 - protein, 111 aa, Artificial Sequence
  clone 43 light chain
QPVLTQPPSASGTPGQRVTISCSGSSSNIGSDTVNWYQQLSGTAPKLLMHSDNQRPSGVPDRFSGSKSGTSASLAISGLQSEDEADYYCAAWDDSLNGWVFGGGTKLTVLG

SEQ ID NO: 282 - protein, 111 aa, Artificial Sequence
  clone 43m light chain
QSVLTQPPSASGTPGQRVTISCSGSSSNIGSDTVNWYQQLSGTAPKLLMHSDNQRPSGVPDRFSGSKSGTSASLAISGLQSEDEADYYCAAWDDSLNGWVFGGGTKLTVLG

SEQ ID NO: 283 - protein, 124 aa, Artificial Sequence; identical to SEQ ID NO: 265
  clone 44 heavy chain

SEQ ID NO: 284 - protein, 111 aa, Artificial Sequence
  clone 44 light chain
QSVLTQPPSASGTPGQRVTISCSGSSSNIGSNTVNWYQQLPGTAPKLLIYSNNQRPSGVPDRFSGSTSGTSASLAISGLQSEDEADYYCAAWDDSLNGWVFGGGTKLTVLG

SEQ ID NO: 285 - protein, 124 aa, Artificial Sequence; identical to SEQ ID NO: 276
  clone 45 heavy chain

SEQ ID NO: 286 - protein, 111 aa, Artificial Sequence
  clone 45 light chain
SYELTQPLSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTKLTVLG

SEQ ID NO: 287 - protein, 111 aa, Artificial Sequence
  clone 45m light chain
QSVLTQPLSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTKLTVLG

SEQ ID NO: 288 - protein, 124 aa, Artificial Sequence
  clone 46 heavy chain
QVQLVQSGPEVKKPGSSVKVSCKASGYTFTDYGLSWVRQAPGHGLEWMGWITAYNGDTNYAQKFQDRLSVTTDISTSTAYMELRSLKSDDTAVYYCARHVFCSGDGCYSGLGSWGQGTLVTVSS

SEQ ID NO: 289 - protein, 111 aa, Artificial Sequence
  clone 46 light chain
SYELTQPPSASGAPGQRVIISCSGSSSNVGSNTVNWYQQLPGTAPKLLIYDKNERPSGVPDRFSASKSGTSASLAISGLQSEDEADYYCAAWDDSLNGWVFGGGTKLTVLG

SEQ ID NO: 290 - protein, 111 aa, Artificial Sequence; identical to SEQ ID NO: 267
  clone 46m light chain

SEQ ID NO: 291 - protein, 127 aa, Artificial Sequence
  clone 47 heavy chain
QVQLVQSGAEVKKPGASVKVSCKASGYTGYTFTDYGLSWVRQAPGHGLEWMGWITAYNGDTNYAQKFQDRLSVTTDISTSTAYMELRSLKSDDTAVYYCARHVFCSGDGCYSGLGSWGQGTLVTVSS

SEQ ID NO: 292 - protein, 111 aa, Artificial Sequence
  clone 47 light chain
SYELIQPPSASGAPGQRVIISCSGSSSNVGSNTVNWYQQLPGTAPKLLIYDKNERPSGVPDRFSASKSGTSASLAISGLQSEDEADYYCAAWDDSLNGWVFGGGTELTVLG

SEQ ID NO: 293 - protein, 111 aa, Artificial Sequence; identical to SEQ ID NO: 267
  clone 47m light chain

SEQ ID NO: 294 - protein, 124 aa, Artificial Sequence
  clone 48 heavy chain
QVQLVQSGAEVKKPGSSVKVSCKASGYTFTDYGLSWVRQAPGHGLEWMGWITAYNGDTNYAQKFQDRLSVTTDISTSTAYMELRSLKSDDTAVYYCARHVFCSGDGCYSGLGSWGQGTLVTVSS

SEQ ID NO: 295 - protein, 111 aa, Artificial Sequence
  clone 48 light chain
QSVLTQPPSVSGAPGQRVTISCSGSSSNVGSNTVNWYQQLPGTAPKLLIYDKNERPSGVPDRFSASKSGTSSSLAIGGLRSEDEADYYCGTWDDNLNGWVFGGGTKLTVLG

SEQ ID NO: 296 - protein, 124 aa, Artificial Sequence; identical to SEQ ID NO: 265
  clone 49 heavy chain

SEQ ID NO: 297 - protein, 111 aa, Artificial Sequence
  clone 49 light chain
SYELTQPPSASGAPGQRVIISCSGSSSNVGSNTVNWYQQLPGTAPKLLIYDKNERPSGVPDRFSASKSGTSSSLAIGGLRSEDEADYYCATWDDNLNGWVFGGGTKLTVLG

SEQ ID NO: 298 - protein, 111 aa, Artificial Sequence
  clone 49m light chain
QSVLTQPPSASGAPGQRVIISCSGSSSNVGSNTVNWYQQLPGTAPKLLIYDKNERPSGVPDRFSASKSGTSSSLAIGGLRSEDEADYYCATWDDNLNGWVFGGGTKLTVLG

SEQ ID NO: 299 - protein, 124 aa, Artificial Sequence; identical to SEQ ID NO: 265
  clone 50 heavy chain

SEQ ID NO: 300 - protein, 111 aa, Artificial Sequence; identical to SEQ ID NO: 297
  clone 50 light chain

SEQ ID NO: 301 - protein, 111 aa, Artificial Sequence
  clone 50m light chain
QSVLTQPPSASGAPGQRVIISCSGSSSNVGSNTVNWYQQLPGTAPKLLIYDKNERPSGVPDRFSASKSGTSSSLAIGGLRSEDEADYYCGTWDDNLNGWVFGGGTKLTVLG

SEQ ID NO: 302 - protein, 124 aa, Artificial Sequence
  clone 51 heavy chain
EVQLVESGTEVKKPGASVKVSCKASGYTFTDYGLSWVRQAPGHGLEWMGWITAYNGDTNYAQKFQDRLSVTTDISTSTAYMELRSLKSDDTAVYYCARHVFCSGDGCYSGLGSWGQGTLVTVSS

SEQ ID NO: 303 - protein, 124 aa, Artificial Sequence
  clone 51m heavy chain
EVQLVQSGAEVKKPGASVKVSCKASGYTFTDYGLSWVRQAPGHGLEWMGWITAYNGDTNYAQKFQDRLSVTTDISTSTAYMELRSLKSDDTAVYYCARHVFCSGDGCYSGLGSWGQGTLVTVSS

SEQ ID NO: 304 - protein, 111 aa, Artificial Sequence; identical to SEQ ID NO: 301
  clone 51 light chain

SEQ ID NO: 305 - protein, 128 aa, Artificial Sequence
  clone 52 heavy chain
QVQLVQSGGGLVQPGGSLRISCTASGFTFRNYEMNWVRQAPGKGLEWVAYISSSGSSRYYADSVKGRFTISRDNAKNSLFLQMNSLRAEDMAVYYCARTDDGSSWFVSTSSFYGMDVWGQGTTVTVSS

SEQ ID NO: 306 - protein, 129 aa, Artificial Sequence
  clone 52m heavy chain
EQVQLVESGGGLVQPGGSLRISCTASGFTFRNYEMNWVRQAPGKGLEWVAYISSSGSSRYYADSVKGRFTISRDNAKNSLFLQMNSLRAEDMAVYYCARTDDGSSWFVSTSSFYGMDVWGQGTTVTVSS

SEQ ID NO: 307 - protein, 110 aa, Artificial Sequence
  clone 52 light chain
EIVLTQSPGIQSLSPGETATLSCRASESISSSYFAWYQQKPGQAPRLLIYGASSRASGIPDRFSGSGSGTDFTLTISRLEPEDFAVYYCQQYGSSPPITFGQGTRLEIKR

SEQ ID NO: 308 - protein, 128 aa, Artificial Sequence
  clone 53 heavy chain
QVQLVQSGGGLVQPGGSLRISCTASGFTFRNYEMNWVRQAPGKGLEWVAYISSSGSSRYYADSVKGRFTISRDNAKNTLYLQMSSLRSDDTAVYYCARTDDGSSWFVSTSSFYGMDVWGQGTTVTVSS

SEQ ID NO: 309 - protein, 129 aa, Artificial Sequence
  clone 53m heavy chain
EQVQLVESGGGLVQPGGSLRISCTASGFTFRNYEMNWVRQAPGKGLEWVAYISSSGSSRYYADSVKGRFTISRDNAKNTLYLQMSSLRSDDTAVYYCARTDDGSSWFVSTSSFYGMDVWGQGTTVTVSS

SEQ ID NO: 310 - protein, 110 aa, Artificial Sequence
  clone 53 light chain
DVVMTQSPGTLSLSPGERATLSCRASQSVSSSYLAWYQQKPGQAPRLLIYGVSSRATGIPDRFSGSGSGTDFTLTISRLEPEDFAVYYCQQYGSSPPITFGQGTKLEIKR

SEQ ID NO: 311 - protein, 110 aa, Artificial Sequence
  clone 53m light chain
EIVLTQSPGTLSLSPGERATLSCRASQSVSSSYLAWYQQKPGQAPRLLIYGVSSRATGIPDRFSGSGSGTDFTLTISRLEPEDFAVYYCQQYGSSPPITFGQGTKLEIKR

SEQ ID NO: 312 - protein, 120 aa, Artificial Sequence
  clone 54 heavy chain
EVQLVESGGGLVQPGGSLRLSCAASEFTFSMNWVRQAPGKGLEWVSSVRGGGTETYYADSVKGRFTVSRDNSKNTLYLQMNSLRVEDTAVYYCTVCVVMFRGWCNWFDPWGQGTLVTVSS

SEQ ID NO: 313 - protein, 108 aa, Artificial Sequence
  clone 54 light chain
DIQLTQSPSSVSASVGERVTITRRASQNIDRWLAWYQQKPGKAPDLLIFAASSLQSGVPSRFSGSGSGTDFTLTISSLQPEDFATYYCQQGKTFPPTFGQGTKLEIKR

SEQ ID NO: 314 - protein, 120 aa, Artificial Sequence
  clone 55 heavy chain
EVQLLESGGGLVQPGGSLRLSCAASEFTFSMNWVRQAPGKGLEWVSSVRGGGTETYYADSVKGRFTVSRDNSKNTLYLQMNSLRVEDTAVYYCTVCVVMFRGWCNWFDPWGQGTLVTVSS

SEQ ID NO: 315 - protein, 108 aa, Artificial Sequence
  clone 55 light chain
DVVMTQSPSSLSASVGDRVTITCRASQSLSSYLNWYQQKPGKAPKLLIYATTNLQSGVPSRFSGSGSGTHFTLTIGSLQPEDFATYYCQQSFQTPLTFGGGTKVEIKR

SEQ ID NO: 316 - protein, 108 aa, Artificial Sequence
  clone 55m light chain
DIQMTQSPSSLSASVGDRVTITCRASQSLSSYLNWYQQKPGKAPKLLIYATTNLQSGVPSRFSGSGSGTHFTLTIGSLQPEDFATYYCQQSFQTPLTFGGGTKLEIKR

SEQ ID NO: 317 - protein, 118 aa, Artificial Sequence
  clone 56 heavy chain
QVQLVESGGGVVQPGRSLRLSCAASGFTFISYVMHWVRQAPGKGLEWVAVVSYDGSEKYYADSVKGRFTISRDNSKNTLYLQMNSLTAEDTAVYYCAREPWVGTIDYWGQGTLVTVSS

SEQ ID NO: 318 - protein, 108 aa, Artificial Sequence
  clone 56 light chain
DIQMTQSPATLSASVGDRVTITCRASQSINNNLNWYQQKPGQAPKLLIYAASTLQGGVPSRFSGSGSGTEFTLTISSLQPEDFATYYCQQLNGYPLTFGGGTKVEIKR

SEQ ID NO: 319 - protein, 121 aa, Artificial Sequence
  clone 57 heavy chain
EVQLLESGGGLVQPGGSLRLSCAASGFTFSDYAMSWVRQAPGKGLEWVSSITANGLMTYYTDSVKGRFTISRDNSKNTLYLQMSSLRTDDTAVYYCAKDAGWEAWWYFDLWGRGTLVTVSS

SEQ ID NO: 320 - protein, 113 aa, Artificial Sequence
  clone 57 light chain
DIQMTQSPDSLAVSLGERATINCKSSQSVLYSSNNKNYLAWYQQKPGQPPKMLIYWASIRETGVPDRFSGSGSETDFTLTISSLQAEDVAVYYCQQYYSTLTFGGGTKVEIKR

SEQ ID NO: 321 - protein, 128 aa, Artificial Sequence
  clone 58 heavy chain
QVQLVQSGAEVKKPGASVKVSCKASGYTFTSYYMRWLRQAPGQGLEWMGIIDPSGGSTTYAQKFQGRVSITRDTSTSTVYMELSSLRSEDTAVYYCARGRGVVPAGNPSTGGVGMDVWGQGTTVTVSS

SEQ ID NO: 322 - protein, 108 aa, Artificial Sequence
  clone 58 light chain
EIVLTQSPATVSLSPGERTMLSCRASQNVSNYLGWYQQRRGQPPRLLISDASNRASGVPARFSGSGSGTDFTLTITSLQPEDFAVYFCQHRSSWPVTFGGGTKVEIKR

SEQ ID NO: 323 - protein, 122 aa, Artificial Sequence
  clone 59 heavy chain
QVQLVQSGSEVKKPGASVKVSCKASGYTFGHYGISWVRQAPGQGLEWMAWISAYNGNTDSIQKVQGRVTMTTDTSTNTAYLELRSLRSDDTAVYYCVRDVPATGGAATADYWGQGTLVTVSS

SEQ ID NO: 324 - protein, 113 aa, Artificial Sequence
  clone 59 light chain
DVVMTQSPLSLPVTLGQPASISCRSSQSLVFSDGNTYLSWFQQRPGQSPRRLIYKVSNRDSGVPDRFSASGSGTDFTLKISRVEADDVGVYYCMQVTHWPRTFGQGTKLEIKR

SEQ ID NO: 325 - protein, 122 aa, Artificial Sequence
  clone 60 heavy chain
QVQLVQSGPEVKKPGSSVKVSCKASGYTFGHYGISWVRQAPGQGLEWMAWISAYNGNTDSIQKVQGRVTMTTDTSTNTAYLELRSLRSDDTAVYYCVRDVPATGGAATADYWGQGTTVTVSS

SEQ ID NO: 326 - protein, 113 aa, Artificial Sequence
  clone 60 light chain
DVVMTQSPLSLPVTLGQPASISCRSSQSLVFSDGNTYLSWFQQRPGQSPRRLIYKVSNRDSGVPDRFSASGSGTDFTLKISRVEADDVGVYYCMQVTHWPRTFGQGTRLEIKR

SEQ ID NO: 327 - protein, 122 aa, Artificial Sequence
  clone 61 heavy chain
QVQLVQSGSEVKKPGASVKVSCKASGYTFGHYGISWVRQAPGQGLEWMAWISAYNGNTDSIQKVQGRVTMTTDTSTNTAYLELRSLRSDDTAVYYCVRDVPATGGAATADYWGQGTTVTVSS

SEQ ID NO: 328 - protein, 113 aa, Artificial Sequence; identical to SEQ ID NO: 324
  clone 61 light chain

SEQ ID NO: 329 - protein, 122 aa, Artificial Sequence
  clone 62 heavy chain
QVQLQQSGSEVKKPGASVKVSCKASGYTFGHYGISWVRQAPGQGLEWMAWISAYNGNTDSIQKVQGRVTMTTDTSTNTAYLELRSLRSDDTAVYYCVRDVPATGGAATADYWGQGTLVTVSS

SEQ ID NO: 330 - protein, 122 aa, Artificial Sequence
  clone 62m heavy chain
QVQLVQSGAEVKKPGASVKVSCKASGYTFGHYGISWVRQAPGQGLEWMAWISAYNGNTDSIQKVQGRVTMTTDTSTNTAYLELRSLRSDDTAVYYCVRDVPATGGAATADYWGQGTLVTVSS

SEQ ID NO: 331 - protein, 113 aa, Artificial Sequence
  clone 62 light chain
DVVMTQSPLSLPVPLGQPASISCRSSQSLVHSDGNTYLSWFQQRPGQSPRRLIYKVSDRDSGVPDRFSGSGSGTDFTLKISRVEAEDVGVYYCMQVTHWPRTFGQGTRLEIKR

SEQ ID NO: 332 - protein, 122 aa, Artificial Sequence; identical to SEQ ID NO: 323
  clone 63 heavy chain

SEQ ID NO: 333 - protein, 113 aa, Artificial Sequence
  clone 63 light chain
DVVMTQSPLSLPVTLGQPASISCKSSRSLVHSDGNTYLSWFQQRPGQSPRRLIYKVSNRDSGVPDRFSASGSGTDFTLKISRVEADDVGVYYCMQVTHWPRTFGQGTKLEIKR

SEQ ID NO: 334 - DNA, 18 bp, Artificial Sequence
  RT-IgGKLA primer
gtgtgcacgccgctggtc

SEQ ID NO: 335 - DNA, 22 bp, Artificial Sequence
  RT-IgGKLA primer
gtgggaagtttctggcggtcac

SEQ ID NO: 336 - DNA, 22 bp, Artificial Sequence
  RT-IgGKLA primer
tggagggcgttatccaccttcc

SEQ ID NO: 337 - DNA, 21 bp, Artificial Sequence
  RT-IgGKLA primer
gtgctcccttcatgcgtgacc

SEQ ID NO: 338 - DNA, 19 bp, Artificial Sequence
  primer_1st For
ccatggactggacctggag

SEQ ID NO: 339 - DNA, 24 bp, Artificial Sequence
  primer_1st For
gcttcctcctcctttggatctctg

SEQ ID NO: 340 - DNA, 21 bp, Artificial Sequence
  primer_1st For
atggacatactttgttccacg

SEQ ID NO: 341 - DNA, 17 bp, Artificial Sequence
  primer_1st For
ctsctgctctgggytcc

SEQ ID NO: 342 - DNA, 21 bp, Artificial Sequence
  primer_1st For
atggagtttgggctgagctgg

SEQ ID NO: 343 - DNA, 17 bp, Artificial Sequence
  primer_1st For
gtcctgggcccagtctg

SEQ ID NO: 344 - DNA, 20 bp, Artificial Sequence
  primer_1st For
atggaattggggctgagctg

SEQ ID NO: 345 - DNA, 20 bp, Artificial Sequence
  primer_1st For
cctgggctctgctsctcctc

SEQ ID NO: 346 - DNA, 20 bp, Artificial Sequence
  primer_1st For
atggagttgggactgagctg

SEQ ID NO: 347 - DNA, 23 bp, Artificial Sequence
  primer_1st For
gttctgtggtttcttctgagctg

SEQ ID NO: 348 - DNA, 17 bp, Artificial Sequence
  primer_1st For
atggaactggggctccg

SEQ ID NO: 349 - DNA, 20 bp, Artificial Sequence
  primer_1st For
gtggcctcctatgwgctgac

SEQ ID NO: 350 - DNA, 22 bp, Artificial Sequence
  primer_1st For
atgaaacacctgtggttcttcc

SEQ ID NO: 351 - DNA, 18 bp, Artificial Sequence
  primer_1st For
acagggtctctctcccag

SEQ ID NO: 352 - DNA, 18 bp, Artificial Sequence
  primer_1st For
atggggtcaaccgccatc

SEQ ID NO: 353 - DNA, 19 bp, Artificial Sequence
  primer_1st For
acaggtctctgtgctctgc

SEQ ID NO: 354 - DNA, 17 bp, Artificial Sequence
  primer_1st For
atgcaagtgggggcctc

SEQ ID NO: 355 - DNA, 19 bp, Artificial Sequence
  primer_1st For
attcycagrctgtggtgac

SEQ ID NO: 356 - DNA, 21 bp, Artificial Sequence
  primer_1st For
atgtctgtctccttcctcatc

SEQ ID NO: 357 - DNA, 21 bp, Artificial Sequence
  primer_1st For
gctcactgcacaggttcttgg

SEQ ID NO: 358 - DNA, 17 bp, Artificial Sequence
  primer_1st For
gctcagctcctggggct

SEQ ID NO: 359 - DNA, 18 bp, Artificial Sequence
  primer_1st For
tccctctcscagsctgtg

SEQ ID NO: 360 - DNA, 22 bp, Artificial Sequence
  primer_1st For
cttcctcctgctactctggctc

SEQ ID NO: 361 - DNA, 17 bp, Artificial Sequence
  primer_1st For
cagtggtccaggcaggg

SEQ ID NO: 362 - DNA, 24 bp, Artificial Sequence
  primer_1st For
tttctctgttgctctggatctctg

SEQ ID NO: 363 - DNA, 19 bp, Artificial Sequence
  primer_1st Rev
gcctgagttccacgacacc

SEQ ID NO: 364 - DNA, 25 bp, Artificial Sequence
  primer_1st Rev
ctgtactttggcctctctgggatag

SEQ ID NO: 365 - DNA, 19 bp, Artificial Sequence
  primer_1st Rev
ctgtccgctttcgctccag

SEQ ID NO: 366 - DNA, 20 bp, Artificial Sequence
  primer_1st Rev
ttccactgctcrggcgtcag

SEQ ID NO: 367 - DNA, 23 bp, Artificial Sequence
  primer_Nested For (VH)
caggtscagctggtgcagtctgg

SEQ ID NO: 368 - DNA, 22 bp, Artificial Sequence
  primer_Nested For (VH)
caggtgcagctgcaggagtcgg

SEQ ID NO: 369 - DNA, 26 bp, Artificial Sequence
  primer_Nested For (VH)
caggtcaccttgaaggagtctggtcc

SEQ ID NO: 370 - DNA, 22 bp, Artificial Sequence
  primer_Nested For (VH)
caggtgcagctacagcagtggg

SEQ ID NO: 371 - DNA, 23 bp, Artificial Sequence
  primer_Nested For (VH)
gaggtgcagctggtggagtctgg

SEQ ID NO: 372 - DNA, 23 bp, Artificial Sequence
  primer_Nested For (VH)
gargtgcagctggtgcagtctgg

SEQ ID NO: 373 - DNA, 23 bp, Artificial Sequence
  primer_Nested For (VH)
gaggtgcagctgttggagtctgg

SEQ ID NO: 374 - DNA, 23 bp, Artificial Sequence
  primer_Nested For (VH)
caggtacagctgcagcagtcagg

SEQ ID NO: 375 - DNA, 20 bp, Artificial Sequence
  primer_Nested For (VL)
cagtctgtgytgackcagcc

SEQ ID NO: 376 - DNA, 19 bp, Artificial Sequence
  primer_Nested For (VL)
caggcagggctgactcagc

SEQ ID NO: 377 - DNA, 20 bp, Artificial Sequence
  primer_Nested For (VL)
cagtctgccctgactcagcc

SEQ ID NO: 378 - DNA, 23 bp, Artificial Sequence
  primer_Nested For (VL)
gacatccagwtgacccagtctcc

SEQ ID NO: 379 - DNA, 21 bp, Artificial Sequence
  primer_Nested For (VL)
tcctatgagctgacwcagcca

SEQ ID NO: 380 - DNA, 27 bp, Artificial Sequence
  primer_Nested For (VL)
gatattgtgatgacccagwctccactc

SEQ ID NO: 381 - DNA, 22 bp, Artificial Sequence
  primer_Nested For (VL)
tcttctgagctgactcaggacc

SEQ ID NO: 382 - DNA, 25 bp, Artificial Sequence
  primer_Nested For (VL)
gaaattgtgttgacrcagtctccag

SEQ ID NO: 383 - DNA, 20 bp, Artificial Sequence
  primer_Nested For (VL)
cagcytgtgctgactcaatc

SEQ ID NO: 384 - DNA, 23 bp, Artificial Sequence
  primer_Nested For (VL)
gacatcgtgatgacccagtctcc

SEQ ID NO: 385 - DNA, 20 bp, Artificial Sequence
  primer_Nested For (VL)
cwgsctgtgctgactcagcc

SEQ ID NO: 386 - DNA, 23 bp, Artificial Sequence
  primer_Nested For (VL)
gaaacgacactcacgcagtctcc

SEQ ID NO: 387 - DNA, 24 bp, Artificial Sequence
  primer_Nested For (VL)
aattttatgctgactcagccccac

SEQ ID NO: 388 - DNA, 25 bp, Artificial Sequence
  primer_Nested For (VL)
gaaattgtgctgacwcagtctccag

SEQ ID NO: 389 - DNA, 21 bp, Artificial Sequence
  primer_Nested For (VL)
cagrctgtggtgacycaggag

SEQ ID NO: 390 - DNA, 22 bp, Artificial Sequence
  primer_Nested For (VL)
gacattgtgctgacccagtctc

SEQ ID NO: 391 - DNA, 22 bp, Artificial Sequence
  primer_Nested Rev
gggaagtagtccttgaccaggc

SEQ ID NO: 392 - DNA, 23 bp, Artificial Sequence
  primer_Nested Rev
gcacacaacagaggcagttccag

SEQ ID NO: 393 - DNA, 20 bp, Artificial Sequence
  primer_Nested Rev
tcacactgagtggctcctgg

SEQ ID NO: 394 - DNA, 23 bp, Artificial Sequence
  primer_Nested Rev
tgctggccgcrtacttgttgttg

SEQ ID NO: 395 - DNA, 38 bp, Artificial Sequence
  primer_VH For
ccgtggcccaggcggcccaggtgcagctggtgcagtct

SEQ ID NO: 396 - DNA, 38 bp, Artificial Sequence
  primer_VH For
ccgtggcccaggcggcccaggtgcagctggtggagtct

SEQ ID NO: 397 - DNA, 38 bp, Artificial Sequence
  primer_VH For
ccgtggcccaggcggcccaggtycagctkgtgcagtct

SEQ ID NO: 398 - DNA, 38 bp, Artificial Sequence
  primer_VH For
ccgtggcccaggcggccgaggtacagctcgtggagtcc

SEQ ID NO: 399 - DNA, 38 bp, Artificial Sequence
  primer_VH For
ccgtggcccaggcggcccaggttcagctggtgcagtct

SEQ ID NO: 400 - DNA, 37 bp, Artificial Sequence
  primer_VH For
ccgtggcccaggcggccaggtggagctgatagagtcc

SEQ ID NO: 401 - DNA, 38 bp, Artificial Sequence
  primer_VH For
ccgtggcccaggcggcccaggtccagctggtacagtct

SEQ ID NO: 402 - DNA, 38 bp, Artificial Sequence
  primer_VH For
ccgtggcccaggcggcccaggtrcagctggtggagtct

SEQ ID NO: 403 - DNA, 38 bp, Artificial Sequence
  primer_VH For
ccgtggcccaggcggcccaggtccagctggtgcagtct

SEQ ID NO: 404 - DNA, 38 bp, Artificial Sequence
  primer_VH For
ccgtggcccaggcggccgaggatcagctggtggagtct

SEQ ID NO: 405 - DNA, 38 bp, Artificial Sequence
  primer_VH For
ccgtggcccaggcggcccagatgcagctggtgcagtct

SEQ ID NO: 406 - DNA, 38 bp, Artificial Sequence
  primer_VH For
ccgtggcccaggcggccgaggtgcagctggtggagwct

SEQ ID NO: 407 - DNA, 38 bp, Artificial Sequence
  primer_VH For
ccgtggcccaggcggcccaaatgcagctggtgcagtct

SEQ ID NO: 408 - DNA, 38 bp, Artificial Sequence
  primer_VH For
ccgtggcccaggcggccsaggtgcagctggtggagtct

SEQ ID NO: 409 - DNA, 38 bp, Artificial Sequence
  primer_VH For
ccgtggcccaggcggccgaggtccagctggtacagtct

SEQ ID NO: 410 - DNA, 38 bp, Artificial Sequence
  primer_VH For
ccgtggcccaggcggccgaggtgcagctggtggagtcy

SEQ ID NO: 411 - DNA, 38 bp, Artificial Sequence
  primer_VH For
ccgtggcccaggcggcccagrtcaccttgaaggagtct

SEQ ID NO: 412 - DNA, 38 bp, Artificial Sequence
  primer_VH For
ccgtggcccaggcggcccaggtgcagctgcaggagtyg

SEQ ID NO: 413 - DNA, 38 bp, Artificial Sequence
  primer_VH For
ccgtggcccaggcggcccaggtcaccttgaaggagtct

SEQ ID NO: 414 - DNA, 38 bp, Artificial Sequence
  primer_VH For
ccgtggcccaggcggcccaggtgcagctgcaggagtcg

SEQ ID NO: 415 - DNA, 38 bp, Artificial Sequence
  primer_VH For
ccgtggcccaggcggcccaggtcaccttgarggagtct

SEQ ID NO: 416 - DNA, 38 bp, Artificial Sequence
  primer_VH For
ccgtggcccaggcggcccagctgcagctgcaggagtcc

SEQ ID NO: 417 - DNA, 38 bp, Artificial Sequence
  primer_VH For
ccgtggcccaggcggccgaggtgcagctggtggagtct

SEQ ID NO: 418 - DNA, 38 bp, Artificial Sequence
  primer_VH For
ccgtggcccaggcggcccaggtgcagctgcaggastcg

SEQ ID NO: 419 - DNA, 38 bp, Artificial Sequence
  primer_VH For
ccgtggcccaggcggccgaagtgcagctggtggagtct

SEQ ID NO: 420 - DNA, 38 bp, Artificial Sequence
  primer_VH For
ccgtggcccaggcggcccaggtgcrgctgcaggagtcg

SEQ ID NO: 421 - DNA, 38 bp, Artificial Sequence
  primer_VH For
ccgtggcccaggcggcccaggtgcagctgktggagtct

SEQ ID NO: 422 - DNA, 38 bp, Artificial Sequence
  primer_VH For
ccgtggcccaggcggcccaggtgcagctrcarsagtsg

SEQ ID NO: 423 - DNA, 38 bp, Artificial Sequence
  primer_VH For
ccgtggcccaggcggccgaggtgcakctggtggagtct

SEQ ID NO: 424 - DNA, 38 bp, Artificial Sequence
  primer_VH For
ccgtggcccaggcggcccagstgcagctgcaggagtcg

SEQ ID NO: 425 - DNA, 38 bp, Artificial Sequence
  primer_VH For
ccgtggcccaggcggccgaggtrcarctggtggagtct

SEQ ID NO: 426 - DNA, 38 bp, Artificial Sequence
  primer_VH For
ccgtggcccaggcggccgaagtgcagctggtgcagtcy

SEQ ID NO: 427 - DNA, 38 bp, Artificial Sequence
  primer_VH For
ccgtggcccaggcggccacagtgcagctggtggagtct

SEQ ID NO: 428 - DNA, 38 bp, Artificial Sequence
  primer_VH For
ccgtggcccaggcggccgaggtgcagctggtgcagtct

SEQ ID NO: 429 - DNA, 38 bp, Artificial Sequence
  primer_VH For
ccgtggcccaggcggccgaggtgcarctggtggagtct

SEQ ID NO: 430 - DNA, 38 bp, Artificial Sequence
  primer_VH For
ccgtggcccaggcggcccaggtacagctgcagcagtca

SEQ ID NO: 431 - DNA, 38 bp, Artificial Sequence
  primer_VH For
ccgtggcccaggcggccgaggtgcagctgktggagtct

SEQ ID NO: 432 - DNA, 38 bp, Artificial Sequence
  primer_VH For
ccgtggcccaggcggcccaggtgcagctggtgcaatct

SEQ ID NO: 433 - DNA, 38 bp, Artificial Sequence
  primer_VH For
ccgtggcccaggcggccgagaygcagctggtggagtct

SEQ ID NO: 434 - DNA, 38 bp, Artificial Sequence
  primer_VH For
ccgtggcccaggcggccctgcagctggtgcagtctggg

SEQ ID NO: 435 - DNA, 38 bp, Artificial Sequence
  primer_VH For
ccgtggcccaggcggccgaggtggagctgatagagccc

SEQ ID NO: 436 - DNA, 38 bp, Artificial Sequence; identical to SEQ ID NO: 395
  primer_VH For

SEQ ID NO: 437 - DNA, 55 bp, Artificial Sequence
  primer_JH Rev
gcctgaaccacctccgccagatccgccacctcctgaggagacggtgaccagggtg

SEQ ID NO: 438 - DNA, 54 bp, Artificial Sequence
  primer_JH Rev
gcctgaaccacctccgccagatccgccacctcctgaggagacggtgaccagggt

SEQ ID NO: 439 - DNA, 56 bp, Artificial Sequence
  primer_JH Rev
gcctgaaccacctccgccagatccgccacctcctgaggagacagtgaccagggtgc

SEQ ID NO: 440 - DNA, 55 bp, Artificial Sequence
  primer_JH Rev
gcctgaaccacctccgccagatccgccacctcctgaggagacggtgaccgtggtc

SEQ ID NO: 441 - DNA, 57 bp, Artificial Sequence
  primer_JH Rev
tctggcggaggtggttcaggcggtggaggctcggacatccagatgacccagtctcct

SEQ ID NO: 442 - DNA, 57 bp, Artificial Sequence; identical to SEQ ID NO: 441
  primer_V kappa For

SEQ ID NO: 443 - DNA, 57 bp, Artificial Sequence
  primer_V kappa For
tctggcggaggtggttcaggcggtggaggctcggatgttgtgatgactcagtctcca

SEQ ID NO: 444 - DNA, 57 bp, Artificial Sequence
  primer_V kappa For
tctggcggaggtggttcaggcggtggaggctcggccatccagatgacccagtctcca

SEQ ID NO: 445 - DNA, 57 bp, Artificial Sequence
  primer_V kappa For
tctggcggaggtggttcaggcggtggaggctcggagattgtgatgacccagactcca

SEQ ID NO: 446 - DNA, 57 bp, Artificial Sequence
  primer_V kappa For
tctggcggaggtggttcaggcggtggaggctcggycatcyggatgacccagtctcca

SEQ ID NO: 447 - DNA, 57 bp, Artificial Sequence
  primer_V kappa For
tctggcggaggtggttcaggcggtggaggctcggaaattgtaatgacacagtctcaa

SEQ ID NO: 448 - DNA, 57 bp, Artificial Sequence
  primer_V kappa For
tctggcggaggtggttcaggcggtggaggctcggacatccagttgacccagtctcca

SEQ ID NO: 449 - DNA, 57 bp, Artificial Sequence
  primer_V kappa For
tctggcggaggtggttcaggcggtggaggctcggaaattgtgttgacacagtctcca

SEQ ID NO: 450 - DNA, 57 bp, Artificial Sequence
  primer_V kappa For
tctggcggaggtggttcaggcggtggaggctcggacatccagatgacccagtctcca

SEQ ID NO: 451 - DNA, 57 bp, Artificial Sequence
  primer_V kappa For
tctggcggaggtggttcaggcggtggaggctcggaaatagtgatgasgcagtctcca

SEQ ID NO: 452 - DNA, 57 bp, Artificial Sequence
  primer_V kappa For
tctggcggaggtggttcaggcggtggaggctcggccatccagttgacccagtctcca

SEQ ID NO: 453 - DNA, 57 bp, Artificial Sequence
  primer_V kappa For
tctggcggaggtggttcaggcggtggaggctcggaaattgtgttgacrcagtctcca

SEQ ID NO: 454 - DNA, 57 bp, Artificial Sequence
  primer_V kappa For
tctggcggaggtggttcaggcggtggaggctcgracatccagatgacccagtctcca

SEQ ID NO: 455 - DNA, 57 bp, Artificial Sequence
  primer_V kappa For
tctggcggaggtggttcaggcggtggaggctcggacatcgtgatgacccagtctcca

SEQ ID NO: 456 - DNA, 57 bp, Artificial Sequence
  primer_V kappa For
tctggcggaggtggttcaggcggtggaggctcggacatccagatgayccagtctcca

SEQ ID NO: 457 - DNA, 57 bp, Artificial Sequence
  primer_V kappa For
tctggcggaggtggttcaggcggtggaggctcggaaacgacactcacgcagtctcca

SEQ ID NO: 458 - DNA, 57 bp, Artificial Sequence; identical to SEQ ID NO: 456
  primer_V kappa For

SEQ ID NO: 459 - DNA, 57 bp, Artificial Sequence
  primer_V kappa For
tctggcggaggtggttcaggcggtggaggctcggaaattgtgctgactcagtctcca

SEQ ID NO: 460 - DNA, 57 bp, Artificial Sequence
  primer_V kappa For
tctggcggaggtggttcaggcggtggaggctcggatattgtgatgacccagcatctg

SEQ ID NO: 461 - DNA, 57 bp, Artificial Sequence
  primer_V kappa For
tctggcggaggtggttcaggcggtggaggctcggatgttgtgatgacacagtctcca

SEQ ID NO: 462 - DNA, 57 bp, Artificial Sequence
  primer_V kappa For
tctggcggaggtggttcaggcggtggaggctcggatattgtgatgacccagactcca

SEQ ID NO: 463 - DNA, 57 bp, Artificial Sequence
  primer_V kappa For
tctggcggaggtggttcaggcggtggaggctcggacattgtgctgacccagtctcca

SEQ ID NO: 464 - DNA, 57 bp, Artificial Sequence
  primer_V kappa For
tctggcggaggtggttcaggcggtggaggctcggatattgtgatgactcagtctcca

SEQ ID NO: 465 - DNA, 44 bp, Artificial Sequence
  primer_J kappa Rev
tgctggccggcctggcctcgtttgatctccagcttggtcccctg

SEQ ID NO: 466 - DNA, 44 bp, Artificial Sequence
  primer_J kappa Rev
tgctggccggcctggcctcgtttgatctccaccttggtccctcc

SEQ ID NO: 467 - DNA, 47 bp, Artificial Sequence
  primer_J kappa Rev
tgctggccggcctggcctcgtttgatatccactttggtcccagggcc

SEQ ID NO: 468 - DNA, 47 bp, Artificial Sequence
  primer_V kappa For
tgctggccggcctggcctcgtttaatctccagtcgtgtcccttggcc

SEQ ID NO: 469 - DNA, 56 bp, Artificial Sequence
  primer_V lambda For
tctggcggaggtggttcaggcggtggaggctcgcagtctgtgctgactcagccacc

SEQ ID NO: 470 - DNA, 56 bp, Artificial Sequence
  primer_V lambda For
tctggcggaggtggttcaggcggtggaggctcgcagtctgccctgactcagcctca

SEQ ID NO: 471 - DNA, 54 bp, Artificial Sequence
  primer_V lambda For
tctggcggaggtggttcaggcggtggaggctcgcagtctgtsstgacgcagccg

SEQ ID NO: 472 - DNA, 57 bp, Artificial Sequence
  primer_V lambda For
tctggcggaggtggttcaggcggtggaggctcgtcctatgagctgayrcagccaccc

SEQ ID NO: 473 - DNA, 55 bp, Artificial Sequence
  primer_V lambda For
tctggcggaggtggttcaggcggtggaggctcgcagtctgtgttgacgcagccgc

SEQ ID NO: 474 - DNA, 57 bp, Artificial Sequence
  primer_V lambda For
tctggcggaggtggttcaggcggtggaggctcgtcctccatgctgactcaggagcca

SEQ ID NO: 475 - DNA, 55 bp, Artificial Sequence
  primer_V lambda For
tctggcggaggtggttcaggcggtggaggctcgcagyctgtgctgactcagccac

SEQ ID NO: 476 - DNA, 59 bp, Artificial Sequence
  primer_V lambda For
tctggcggaggtggttcaggcggtggaggctcgtcctatgagctgacacagccaycctc

SEQ ID NO: 477 - DNA, 54 bp, Artificial Sequence
  primer_V lambda For
tctggcggaggtggttcaggcggtggaggctcgcagtctgtgytgacgcagccg

SEQ ID NO: 478 - DNA, 57 bp, Artificial Sequence
  primer_V lambda For
tctggcggaggtggttcaggcggtggaggctcgacatatgagctgtctcagccaccc

SEQ ID NO: 479 - DNA, 57 bp, Artificial Sequence
  primer_V lambda For
tctggcggaggtggttcaggcggtggaggctcgcagcctgtgctgactcagatgacc

SEQ ID NO: 480 - DNA, 57 bp, Artificial Sequence
  primer_V lambda For
tctggcggaggtggttcaggcggtggaggctcgtcctctgagctgagtcaggagcct

SEQ ID NO: 481 - DNA, 55 bp, Artificial Sequence
  primer_V lambda For
tctggcggaggtggttcaggcggtggaggctcgcagtctgccctgaytcagcctc

SEQ ID NO: 482 - DNA, 55 bp, Artificial Sequence
  primer_V lambda For
tctggcggaggtggttcaggcggtggaggctcgtcctctgggccaactcaggtgc

SEQ ID NO: 483 - DNA, 55 bp, Artificial Sequence
  primer_V lambda For
tctggcggaggtggttcaggcggtggaggctcgcartctgccctgactcagccts

SEQ ID NO: 484 - DNA, 54 bp, Artificial Sequence
  primer_V lambda For
tctggcggaggtggttcaggcggtggaggctcgctgcctgtgctgactcagccc

SEQ ID NO: 485 - DNA, 57 bp, Artificial Sequence
  primer_V lambda For
tctggcggaggtggttcaggcggtggaggctcgcagtctgttctgactcagcctcgc

SEQ ID NO: 486 - DNA, 57 bp, Artificial Sequence
  primer_V lambda For
tctggcggaggtggttcaggcggtggaggctcgcagcctgtgctgactcaatcatcc

SEQ ID NO: 487 - DNA, 57 bp, Artificial Sequence
  primer_V lambda For
tctggcggaggtggttcaggcggtggaggctcgtcctatgagctgactcagccaccc

SEQ ID NO: 488 - DNA, 57 bp, Artificial Sequence
  primer_V lambda For
tctggcggaggtggttcaggcggtggaggctcgcagcttgtgctgactcaatcgccc

SEQ ID NO: 489 - DNA, 57 bp, Artificial Sequence
  primer_V lambda For
tctggcggaggtggttcaggcggtggaggctcgtcctctgagctgactcagctgcct

SEQ ID NO: 490 - DNA, 54 bp, Artificial Sequence
  primer_V lambda For
tctggcggaggtggttcaggcggtggaggctcgcagcctgtgctgactcagccr

SEQ ID NO: 491 - DNA, 59 bp, Artificial Sequence
  primer_V lambda For
tctggcggaggtggttcaggcggtggaggctcgtcctatgagcttacacagccaccctc

SEQ ID NO: 492 - DNA, 60 bp, Artificial Sequence
  primer_V lambda For
tctggcggaggtggttcaggcggtggaggctcgaattttatgctgactcagccccactct

SEQ ID NO: 493 - DNA, 57 bp, Artificial Sequence
  primer_V lambda For
tctggcggaggtggttcaggcggtggaggctcgtcctctgagcggactcagttgcct

SEQ ID NO: 494 - DNA, 56 bp, Artificial Sequence
  primer_V lambda For
tctggcggaggtggttcaggcggtggaggctcgcagrctgtggtgactcaggagcc

SEQ ID NO: 495 - DNA, 59 bp, Artificial Sequence
  primer_V lambda For
tctggcggaggtggttcaggcggtggaggctcgtcctatgagctgactcagccactctc

SEQ ID NO: 496 - DNA, 54 bp, Artificial Sequence
  primer_V lambda For
tctggcggaggtggttcaggcggtggaggctcgcagactgtggtgacccaggag

SEQ ID NO: 497 - DNA, 57 bp, Artificial Sequence
  primer_V lambda For
tctggcggaggtggttcaggcggtggaggctcgtcctatgagctgacwcagccacmc

SEQ ID NO: 498 - DNA, 54 bp, Artificial Sequence
  primer_V lambda For
tctggcggaggtggttcaggcggtggaggctcgcagcctgtgctgactcagcca

SEQ ID NO: 499 - DNA, 58 bp, Artificial Sequence
  primer_V lambda For
tctggcggaggtggttcaggcggtggaggctcgtcttctgagctgactcaggaccctg

SEQ ID NO: 500 - DNA, 54 bp, Artificial Sequence
  primer_V lambda For
tctggcggaggtggttcaggcggtggaggctcgcaggcagggctgactcagcca

SEQ ID NO: 501 - DNA, 57 bp, Artificial Sequence
  primer_V lambda For
tctggcggaggtggttcaggcggtggaggctcgtcctatgtgctgacwcagcyaccc

SEQ ID NO: 502 - DNA, 51 bp, Artificial Sequence
  primer_V lambda For
tctggcggaggtggttcaggcggtggaggctcgcggcccgtgctgactcag

SEQ ID NO: 503 - DNA, 44 bp, Artificial Sequence
  primer_J lambda Rev
tgctggccggcctggccgcctaggacggtgaccttggtcccagt

SEQ ID NO: 504 - DNA, 41 bp, Artificial Sequence
  primer_J lambda Rev
tgctggccggcctggccgcctaggacggtcagctcsgtccc

SEQ ID NO: 505 - DNA, 43 bp, Artificial Sequence
  primer_J lambda Rev
tgctggccggcctggccgcctaggacggtcagcttggtccctc

SEQ ID NO: 506 - DNA, 42 bp, Artificial Sequence
  primer_J lambda Rev
tgctggccggcctggccgccgaggacggtcaccttggtgcca

SEQ ID NO: 507 - DNA, 52 bp, Artificial Sequence
  primer_J lambda Rev
tgctggccggcctggccgcctaaaatgatcagctgggttcctccaccaaata

SEQ ID NO: 508 - DNA, 40 bp, Artificial Sequence
  primer_J lambda Rev
tgctggccggcctggccgccgaggycggtcagctgggtgc

SEQ ID NO: 509 - DNA, 48 bp, Artificial Sequence
  IgG_primer_Heavy chain_HC_F1
tgctgtgggtgagtggtacctgtggggaggtgcagctggtggagtctg

SEQ ID NO: 510 - DNA, 50 bp, Artificial Sequence
  IgG_primer_Heavy chain_HC_F2
tgctgtgggtgagtggtacctgtgggcaggtcaccttgaaggagtctggg

SEQ ID NO: 511 - DNA, 48 bp, Artificial Sequence
  IgG_primer_Heavy chain_HC_F3
tgctgtgggtgagtggtacctgtgggcaggtgcagctggtgcagtctg

SEQ ID NO: 512 - DNA, 54 bp, Artificial Sequence
  IgG_primer_Heavy chain_HC_R1
gggggaagaccgatgggcccttggtggaggctgaggagacggtgaccagggttc

SEQ ID NO: 513 - DNA, 55 bp, Artificial Sequence
  IgG_primer_Heavy chain_HC_R2
gggggaagaccgatgggcccttggtggaggctgaggagacagtgaccagggttcc

SEQ ID NO: 514 - DNA, 54 bp, Artificial Sequence
  IgG_primer_Heavy chain_HC_R3
gggggaagaccgatgggcccttggtggaggctgaggagacggtgaccgtggttc

SEQ ID NO: 515 - DNA, 53 bp, Artificial Sequence
  IgG_primer_Heavy chain_HC_R4
gggggaagaccgatgggcccttggtggaggctgaggagacggtgaccgtggtc

SEQ ID NO: 516 - DNA, 47 bp, Artificial Sequence
  IgG_primer_Lambda chain_LC_F1
tgctgtgggtgagtggtacctgtgggcagtctgtgctgacgcagccg

SEQ ID NO: 517 - DNA, 48 bp, Artificial Sequence
  IgG_primer_Lambda chain_LC_F2
tgctgtgggtgagtggtacctgtgggtcctatgagctgactcagccac

SEQ ID NO: 518 - DNA, 48 bp, Artificial Sequence
  IgG_primer_Lambda chain_LC_F3
tgctgtgggtgagtggtacctgtgggcagcctgtgctgactcagccac

SEQ ID NO: 519 - DNA, 49 bp, Artificial Sequence
  IgG_primer_Lambda chain_LC_F4
tgctgtgggtgagtggtacctgtgggcagactgtggtgactcaggagcc

SEQ ID NO: 520 - DNA, 48 bp, Artificial Sequence
  IgG_primer_Lambda chain_LC_F5
tgctgtgggtgagtggtacctgtgggcagtctgccctgactcagcctg

SEQ ID NO: 521 - DNA, 42 bp, Artificial Sequence
  IgG_primer_Lambda chain_LC_R1
gtgggattggccttaggttggcctaggacggtgaccttggtc

SEQ ID NO: 522 - DNA, 42 bp, Artificial Sequence
  IgG_primer_Lambda chain_LC_R2
gtgggattggccttaggttggcctaggacggtcagcttggtc

SEQ ID NO: 523 - DNA, 50 bp, Artificial Sequence
  IgG_primer_Kappa chain_KC_F1
tgctgtgggtgagtggtacctgtggggatgttgtgatgactcagtctcca

SEQ ID NO: 524 - DNA, 50 bp, Artificial Sequence
  IgG_primer_Kappa chain_KC_F2
tgctgtgggtgagtggtacctgtggggatattgtgatgacccagactcca

SEQ ID NO: 525 - DNA, 50 bp, Artificial Sequence
  IgG_primer_Kappa chain_KC_F3
tgctgtgggtgagtggtacctgtggggacatccagatgacccagtctcca

SEQ ID NO: 526 - DNA, 50 bp, Artificial Sequence
  IgG_primer_Kappa chain_KC_F4
tgctgtgggtgagtggtacctgtggggagattgtgctgacccagtctcca

SEQ ID NO: 527 - DNA, 50 bp, Artificial Sequence
  IgG_primer_Kappa chain_KC_F5
tgctgtgggtgagtggtacctgtggggacatccagttgacccagtctcca

SEQ ID NO: 528 - DNA, 44 bp, Artificial Sequence
  IgG_primer_Kappa chain_KC_R1
acgcttggagcggccaccgtacgtttgatctccagcttggtccc

SEQ ID NO: 529 - DNA, 47 bp, Artificial Sequence
  IgG_primer_Kappa chain_KC_R2
acgcttggagcggccaccgtacgtttaatctccagtcgtgtcccttg

SEQ ID NO: 530 - DNA, 44 bp, Artificial Sequence
  IgG_primer_Kappa chain_KC_R3
acgcttggagcggccaccgtacgtttgatctccaccttggtccc